(19) United States
(12) Patent Application Publication
(10) Pub. No.: US 2024/0376170 A1
(43) Pub. Date: Nov. 14, 2024

(54) CONDITIONALLY ACTIVATED PROTEINS AND METHODS OF USE

(71) Applicant: Bright Peak Therapeutics AG, Basel (CH)

(72) Inventors: Vijaya Raghavan PATTABIRAMAN, Volketswil (CH); Bertolt KREFT, Kleinmachnow (DE); Grégory UPERT, Kembs (FR); Matilde ARÉVALO-RUIZ, Basel (CH); Nina RESCHKE, Birsfelden (CH); Davor BAJIC, Basel (CH); Roy MEODED, Basel (CH)

(21) Appl. No.: 18/410,740

(22) Filed: Jan. 11, 2024

Related U.S. Application Data
(60) Provisional application No. 63/438,456, filed on Jan. 11, 2023.
(60) Provisional application No. 63/438,396, filed on Jan. 11, 2023.

(51) Int. Cl. C07K 14/55

(52) U.S. Cl. CPC C07K 14/55; C07K 2319/50

(57) ABSTRACT

The present disclosure relates to activatable proteins which comprise cleavable moieties attached thereto which display an altered activity upon cleavage of the cleavable moieties. The present disclosure also relates to activatable IL-2 polypeptides which comprise cleavable moieties, as well as compositions and methods of use thereof. The present disclosure further relates to cleavable peptides which can be cleaved by multiple proteases, as well as to polypeptides incorporating said cleavable peptides.

### CROSS REFERENCE

This application claims the benefit of U.S. Provisional Application No. 63/438,456, filed Jan. 11, 2023, and U.S. Provisional Application No. 63/438,396 filed Jan. 11, 2023, each of which applications are incorporated herein by reference in its entirety.

### SEQUENCE LISTING

The instant application contains a Sequence Listing which has been submitted electronically in XML format and is hereby incorporated by reference in its entirety. Said XML copy, created on Jan. 31, 2024, is named 94917-0110_730201US_SL.xml and is 866,619 bytes in size.

### BRIEF SUMMARY

Described herein are activatable proteins, including activatable cytokines such as interleukin 2 (IL-2) and derivatives thereof. In some embodiments, the activatable proteins utilize a cleavable moiety, such as a protease cleavable peptide, attached to the protein in a manner such that an activity (e.g., a binding affinity of the protein with its cognate receptor or other ligand) is altered or reduced compared to a corresponding protein without the cleavable moiety. In some embodiments, upon cleavage of the cleavable moiety, the protein is “activated” and at least a portion of the activity of corresponding protein is restored. In some embodiments of the instant disclosure, the cleavable moiety is attached to a side chain of an amino acid residue of the protein, allowing for fine tuning of the activity by placing the cleavable moiety in an optimal position to tune or detune the activity of the protein. In some embodiments, the cleavable moiety is also attached to the protein at an additional point of attachment (e.g., to a second amino acid residue). In some embodiments, having the cleavable moiety attached to the additional point of attachment result in a conformational change in the protein which reduces an activity of the protein. In such cases, cleavage of the cleavable moiety (e.g., by a protease) allows the protein to adopt a more “natural” conformation, thereby restoring the activity (or a portion thereof) of the protein. Activatable proteins of the instant disclosure can be used in a variety of applications, including therapeutic applications. For example, a cleavable moiety can be selected such that it is preferentially or selectively cleaved at a desired tissue site (e.g., the cleavable moiety can take advantage of increased protease activity, lowered pH, or the reducing environment of a tumor microenvironment to selectively activate a therapeutic protein at the tumor site, thereby sparing or minimizing the risk of off target effects of the therapeutic protein).

In some embodiments of the present disclosure, the above-described approach is applied to IL-2 in order to prepare an activatable IL-2 polypeptide. In some embodiments, the activatable IL-2 polypeptide is based off of an IL-2 polypeptide with reduced (e.g., substantially no) ability to bind the IL-2 receptor alpha subunit but which retains the ability to bind to the IL-2 receptor beta and gamma subunits (see, e.g., U.S. Patent Publication No.: US2021/0252157, which is hereby incorporated by reference as if set forth herein in its entirety). Such “alpha-dead” IL-2 variants preferentially activate CD8+ T effector cells and/or natural killer (NK) cells relative to regulatory T cells (Tregs), in contrast to wild type IL-2, which preferentially activates Tregs. This property makes alpha-dead IL-2 variants attractive as potential therapeutics, such as in cancer immunotherapies. However, systemic activity of the IL-2 molecules may have a high risk of side effects on subjects administered said IL-2 polypeptides owing to the high activity of the cytokine. In order to minimize the risk of side effects from systemic and/or off target effects, provided herein in certain embodiments are activatable IL-2 polypeptides which comprise a cleavable moiety attached in such a manner as to lower the activity of the IL-2 polypeptide until cleavage of the cleavable moiety. In some embodiments, the cleavable moiety is a cleavable peptide which is a substrate for a protease which is upregulated or overexpressed in or near a tumor microenvironment, thereby allowing for targeted delivery of an active form of the alpha-dead IL-2 polypeptide to a subject's tumor and sparing the activity of the alpha-dead IL-2 polypeptide in other tissues.

Exemplary illustrations of activatable IL-2 polypeptides as described herein are shown in FIG. 7. In the top example, an IL-2 polypeptide is rendered in an inactive or less active state due to the presence of a mask (e.g., a PEG group) attached to a side chain of a residue of the IL-2 polypeptide through a cleavable peptide (i.e., a protease cleavable peptide in the example depicted). Upon entry of the activatable (or “masked”) IL-2 polypeptide into or near the tumor microenvironment (TME), proteases which are upregulated in the region (e.g., matriptase or MMPs) act to cleave the cleavable peptide, thereby liberating the mask from the IL-2 polypeptide, thereby resulting in an active form of the IL-2 polypeptide. In some instances, the cleavable peptide itself can act as the masking group. In the bottom example depicted in FIG. 7, a cleavable peptide is attached to the activatable IL-2 polypeptide at two points, thereby creating a macrocyclic structure. In preferred embodiments, at least one of the two points of attachment is to a side chain of a residue of the IL-2 polypeptide. Upon entry into or near the TMA, proteases act to cleave peptide, thus disrupting the macrocyclic structure, thereby enhancing the activity of the IL-2 polypeptide and rendering it in an “active” form.

Further provided herein are cleavable peptides which are cleavable by multiple proteases of different classes. In some embodiments, the cleavable peptides comprises multiple protease recognition sites for different proteases which are upregulated in tumors or tumor microenvironments. In some embodiments, such multi-protease cleavable linkers allow for enhanced activation of an activatable protein as there are additional sites which can be cleaved, cleavage of any one of which can be sufficient to activate the protein. Such cleavable peptides can be incorporated into activatable proteins as provided herein or can be used in other ways in artificial proteins, for example use as peptide linkers between domains of a fusion protein which is desired to be cleavable, or as a peptide linker between a blocking moiety and a protein (e.g., between a receptor binding protein and a dummy receptor or bulky protein, or between an antigen-binding domain and a peptide which blocks binding of the antigen-binding domain to its target antigen).

In one aspect described herein is an activatable protein, comprising: a protein comprising a cleavable moiety attached to a side chain of an amino acid residue of the protein, wherein the protein displays an altered activity after cleavage of the cleavable moiety compared to the activity of the activatable protein prior to cleavage of the cleavable moiety. In some embodiments, the cleavable moiety comprises a cleavable peptide. In some embodiments, the cleavable peptide is a protease cleavable peptide. In some embodiments, the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the cleavable peptide is cleavable by multiple proteases. In some embodiments, cleavage of the cleavable peptide leaves 1, 2, 3, 4, 5, 6, 7, 8, 9, or 10 amino acids attached to the side chain of the amino acid residue to which the cleavable peptide is attached. In some embodiments, the cleavable peptide comprises an amino acid sequence having at least about 80%, at least about 90%, or 100% identity to a sequence set forth in any one of Table 1B or Table 1C. In some embodiments, the C-terminus of the cleavable peptide is attached to the side chain of the amino acid residue of the activatable protein, optionally through a linking group.

In some embodiments, the amino acid residue to which the cleavable moiety is attached is lysine, glutamate, glutamine, aspartate, asparagine, tyrosine, serine, threonine, cysteine, or an unnatural amino acid. In some embodiments, the amino acid residue to which the cleavable moiety is attached is a lysine or glutamate. In some embodiments, the amino acid residue to which the cleavable moiety is attached is substituted relative to the amino acid at the corresponding position in the wild type version of the protein.

In some embodiments, the cleavable moiety is attached to the protein at an additional point of attachment. In some embodiments, the additional point of attachment is the N-terminus or the C-terminus of the protein. In some embodiments, the additional point of attachment is to a side chain of another amino acid residue of the protein.

In some embodiments, the cleavable moiety is attached to an additional moiety. In some embodiments, cleavage of the cleavable moiety causes the additional moiety to no longer be attached to the protein. In some embodiments, the cleavable moiety is attached to the side chain of the amino acid residue through a linking group.

In some embodiments, the protein is at least 50, 75, 100, or 125 amino acids in length. In some embodiments, the protein is from about 50 to about 500 amino acids in length, from about 50 to about 300 amino acids in length, from about 50 to about 250 amino acids in length, from about 50 to about 200 amino acids in length, from about 100 to about 500 amino acids in length, from about 100 to about 300 amino acids in length, or from about 100 to about 200 amino acids in length. In some embodiments, the protein is synthetic. In some embodiments, the activatable protein comprises an antibody or antigen binding fragment thereof, a cytokine, a protein hormone, an enzyme, or a fusion protein of any of these. In some embodiments, the altered activity is enhanced binding to a ligand of the protein. In some embodiments, the amino acid residue to which the cleavable moiety is attached interacts with a ligand of the protein.

In an aspect herein is an activatable IL-2 polypeptide, comprising: an IL-2 polypeptide comprising a cleavable moiety attached to a side chain of an amino acid residue of the IL-2 polypeptide, wherein the IL-2 polypeptide displays an enhanced ability to bind to at least one IL-2 receptor subunit after cleavage of the cleavable moiety compared to the activity of the activatable IL-2 polypeptide before cleavage of the cleavable moiety. In another aspect herein is an activatable IL-2 polypeptide, comprising: an IL-2 polypeptide comprising a cleavable moiety attached to a residue in the region of residues 1-35 of the IL-2 polypeptide, wherein residue position numbering is based on SEQ ID NO: 1 as a reference sequence; and wherein the IL-2 polypeptide exhibits a greater affinity for the IL-2 receptor beta subunit after cleavage of the cleavable moiety compared to activatable IL-2 polypeptide before cleavage of the cleavable moiety.

In some embodiments, the cleavable moiety comprises a cleavable peptide. In some embodiments, the cleavable peptide is a protease cleavable peptide. In some embodiments, the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the cleavable peptide is cleavable by multiple proteases. In some embodiments, cleavage of the cleavable peptide leaves 1, 2, 3, 4, 5, 6, 7, 8, 9, or 10 amino acids attached to the side chain of the amino acid residue to which the cleavable peptide is attached. In some embodiments, the cleavable peptide comprises an amino acid sequence having at least about 80%, at least about 90%, or 100% identity to a sequence set forth in any one of Table 1B or Table 1C. In some embodiments, the C-terminus of the cleavable peptide is attached to the side chain of the amino acid residue of the activatable IL-2 polypeptide, optionally through a linking group.

In some embodiments, the amino acid residue to which the cleavable moiety is attached is lysine, glutamate, glutamine, aspartate, asparagine, tyrosine, serine, threonine, cysteine, or an unnatural amino acid. In some embodiments, the amino acid residue to which the cleavable moiety is attached is a lysine or glutamate. In some embodiments, the amino acid residue to which the cleavable moiety is attached is substituted relative to the corresponding residue in SEQ ID NO: 1.

In some embodiments, the cleavable moiety is attached to a residue which contacts the IL-2 receptor beta subunit or the IL-2 receptor gamma subunit during binding to the IL-2 receptor. In some embodiments, the cleavable moiety is attach to a residue selected from residues 9, 11, 13, 15, 16, 19, 20, 22, 23, 26, 29, 32, 84, 88, 91, 123, 126, and 129 of the IL-2 polypeptide, wherein residue position numbering is based on SEQ ID NO: 1 as a reference sequence. In some embodiments, the cleavable moiety is attached to residue 9, 11, 13, 15, 16, 19, 22, 23, 29, or 32 of the IL-2 polypeptide, wherein residue position numbering is based on SEQ ID NO: 1 as a reference sequence.

In some embodiments, the cleavable moiety is attached to the IL-2 polypeptide at an additional point of attachment. In some embodiments, the additional point of attachment is to the N-terminus of the IL-2 polypeptide. In some embodiments, the cleavable moiety is attached to residue 23 of the IL-2 polypeptide and the additional point of attachment is to the N-terminus of the IL-2 polypeptide. In some embodiments, the N-terminus of the IL-2 polypeptide is the amino acid residue at a position corresponding to the first residue of SEQ ID NO: 1. In some embodiments, the cleavable moiety comprises a cleavable peptide. In some embodiments, the C-terminus of the cleavable peptide is attached to the N-terminus of the IL-2 polypeptide and the N-terminus of the cleavable peptide is attached to residue 23 of the IL-2 polypeptide. In some embodiments, the cleavable peptide comprises the sequence set forth in SEQ ID NO: 317 or 333. In some embodiments, residue 23 of the IL-2 polypeptide comprises a carboxylic acid side chain. In some embodiments, residue 23 of the IL-2 polypeptide is glutamate.

In some embodiments, the additional point of attachment is to a side chain of another amino acid residue of the IL-2 polypeptide. In some embodiments, the additional point of attachment is to residue 9 of the IL-2 polypeptide, wherein residue position numbering is based on SEQ ID NO: 1 as a reference sequence. In some embodiments, the cleavable moiety is attached to the side chain of the amino acid residue through a linking group.

In some embodiments, the IL-2 polypeptide exhibits reduced binding to the IL-2 receptor alpha subunit compared to wild type IL-2. In some embodiments, the IL-2 polypeptide comprises at least one modification which reduces the affinity of the IL-2 polypeptide to the IL-2 receptor alpha compared to wild type IL-2. In some embodiments, the IL-2 polypeptide comprises at least one polymer covalently attached to a residue selected from residues 35, 37, 38, 41, 42, 43, 44, 45, 60, 61, 62, 64, 65, 68, 69, 71, 72, 104, 105, and 107, wherein residue position numbering is based on SEQ ID NO: 1 as a reference sequence. In some embodiments, the IL-2 polypeptide comprises at least one polymer covalently attached to a residue selected from residue 42 and 45, wherein residue position numbering is based on SEQ ID NO: 1 as a reference sequence. In some embodiments, the IL-2 polypeptide comprises polymers covalently attached at residues 42 and 45, wherein residue position numbering is based on SEQ ID NO: 1 as a reference sequence. In some embodiments, the IL-2 polypeptide is synthetic. In some embodiments, the IL-2 polypeptide comprises an amino acid sequence having at least about 80%, at least about 85%, at least about 90%, or at least about 95% sequence identity SEQ ID NO: 2 or SEQ ID NO: 3.

In some embodiments, the cleavable moiety is attached to an additional moiety. In some embodiments, cleavage of the cleavable moiety releases the additional moiety from the IL-2 polypeptide. In some embodiments, the activatable IL-2 polypeptide is attached to an additional polypeptide. In some embodiments, the additional polypeptide is an antibody or an antigen binding fragment thereof.

In an aspect herein is a method of manufacturing an activatable protein described herein (e.g., an IL-2 polypeptide), wherein the activatable protein comprises a cleavable moiety attached to a side chain of the protein and an additional point of attachment to the protein. In some embodiments, the method comprises synthesizing at least two fragments of the activatable protein, one of the fragments comprising the cleavable moiety attached to one of the side chain of the protein or the additional point of attachment. In some embodiments, the method comprises performing a cyclization reaction to attach the cleavable moiety to the other point of attachment, wherein the side chain of the protein and the additional point of attachment are present on the same fragment. In some embodiments, the method comprises ligating the at least two fragments to form the activatable protein. In some embodiments, the additional point of attachment is the N-terminus of the protein.

In an aspect herein is a pharmaceutical composition comprising an activatable IL-2 polypeptide as provided herein and a pharmaceutically acceptable carrier.

In another aspect herein is a method of treating cancer in a subject in need thereof, comprising: administering to the subject a pharmaceutically effective amount of an activatable IL-2 polypeptide or a pharmaceutical composition described herein. In some embodiments, the cancer is a solid cancer. In some embodiments, the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer. In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

In an aspect provided herein is an artificial polypeptide comprising a cleavable peptide having an amino acid sequence having at least about 80%, at least about 90%, or 100% identity to a sequence set forth in any one of Table 1B or Table 1C, wherein the cleavable peptide is attached to a side chain of an amino acid residue of the artificial polypeptide. In another aspect herein is an artificial polypeptide comprising a cleavable peptide having an amino acid sequence having at least about 80%, at least about 90%, or 100% identity to a sequence set forth in Table 1C. In some embodiments, the cleavable peptide is attached to a side chain of an amino acid residue of the artificial polypeptide.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “figure” and “FIG.” herein), of which:

FIG. 1A shows a plot comparing the concentration where the indicated IL-2 polypeptide variant shows 50% of maximal activation of IL-2 reporter (EC50) for the indicated IL-2 polypeptides or control IL-2 polypeptide CMP-003.

FIG. 1B shows a plot comparing the concentration where the indicated activatable and cleaved IL-2 polypeptide variants show 50% of maximal activation of the IL-2 reporter (EC50), as well as control IL-2 polypeptide CMP-003. For the IL-2 polypeptide variants shown in FIG. 1B (except CMP-003) a cleavable peptide is attached to a single residue of the IL-2 polypeptide. Activatable IL-2 polypeptides were tested either intact (black) or MMP2 treated (grey).

FIG. 1C shows a plot comparing the concentration where the indicated activatable and cleaved IL-2 polypeptide variants show 50% of maximal activation of the IL-2 reporter (EC50), as well as control IL-2 polypeptide CMP-003. For the IL-2 polypeptide variants shown in FIG. 1C (except CMP-003) a cleavable peptide is attached to the IL-2 polypeptide at 2 residues. Activatable IL-2 polypeptides were tested either intact (black) or MMP2 treated (grey).

FIG. 1D shows a plot comparing the concentration where the indicated activatable and cleave IL-2 polypeptides show 50% of maximal activation of the IL-2 reporter (EC50), as well as corresponding controls.

FIG. 1E shows a plot comparing the concentration where the indicated activatable and cleave IL-2 polypeptides show 50% of maximal activation of the IL-2 reporter (EC50), as well as corresponding controls. Activatable IL-2 polypeptides were tested either intact (black), MMP2 treated (solid grey), matriptase treated (grey and white checkerboard pattern), or uPa treated (dotted pattern).

FIG. 1F shows a plot comparing the concentration where the indicated activatable and cleave IL-2 polypeptides show 50% of maximal activation of the IL-2 reporter (EC50), as well as corresponding controls. Activatable IL-2 polypeptides were tested either intact (black), MMP2 treated (solid grey), matriptase treated (grey and white checkerboard pattern), or uPa treated (dotted pattern).

FIG. 2A shows bilayer interferometry traces for synthetic IL-2 cytokine CMP-003, and overlaid model traces. Binding data is shown for IL-2Rβ (CD122) (left) and IL-2Rβγ (CD122 CD132 dimer) (right).

FIG. 2B shows bilayer interferometry traces for uncleaved activatable IL-2 cytokine CMP-136, and overlaid model traces (smoothed lines). Binding data is shown for IL-2Rβ (left) and IL-2Rβγ (right).

FIG. 2C shows bilayer interferometry traces for uncleaved activatable IL-2 cytokine CMP-145, and overlaid model traces (smoothed lines). Binding data is shown for IL-2Rβ (left) and IL-2Rβγ (right).

FIG. 2D shows bilayer interferometry traces for uncleaved activatable IL-2 cytokine CMP-138, and overlaid model traces (smoothed lines). Binding data is shown for IL-2Rβ (left) and IL-2Rβγ (right).

FIG. 2E shows bilayer interferometry traces for uncleaved activatable IL-2 cytokine CMP-151, and overlaid model traces (smoothed lines). Binding data is shown for IL-2Rβ (left) and IL-2Rβγ (right).

FIG. 3A shows a plot of the average result of pSTAT5 assays for IL-2 polypeptides.

FIG. 3B shows a plot of the average EC50 result of pSTAT5 assays for strategy 2 molecules with a comparison of intact (uncleaved) IL-2 molecules (black) vs MMP2 treated (cleaved) IL-2 molecules (gray).

FIG. 3C shows a plot of the average EC50 result of pSTAT5 assays for the indicatedmolecules with a comparison of intact (uncleaved) IL-2 molecules (black) vs MMP2 treated (cleaved) IL-2 molecules (gray).

FIG. 3D shows a plot of the average EC50 result of pSTAT5 assays for the indicated molecules with a comparison of intact (uncleaved) IL-2 molecules (black) vs MMP2 treated (cleaved) IL-2 molecules (gray).

FIG. 3E shows a plot of the average EC50 result of pSTAT5 assays for the indicated molecules with a comparison of intact (uncleaved) IL-2 molecules (black) vs MMP2 treated (cleaved) IL-2 molecules (gray).

FIG. 4A shows a plot of the average EC50 results of pSTAT5 assays in NK cells for the indicated molecules with a comparison of intact (uncleaved) IL-2 molecules (black) vs MMP2 treated (cleaved) IL-2 molecules (gray).

FIG. 4B shows a plot of the average EC50 results of pSTAT5 assays in NK cells for the indicated molecules with a comparison of intact (black), MMP2 treated (solid grey), matriptase treated (grey and white checkerboard pattern), or uPa treated (dotted pattern).

FIG. 4C shows a plot of the average EC50 results of pSTAT5 assays in NK cells for the indicated molecules with a comparison of intact (black), MMP2 treated (solid grey), matriptase treated (grey and white checkerboard pattern), or uPa treated (dotted pattern).

FIG. 4D shows a plot of the average EC50 results of pSTAT5 assays in NK cells for the indicated molecules.

FIG. 5 shows a plot of % of pSTAT5⁺ CD8 cells in mouse splenocytes observed upon treatment with the indicated concentration of the indicated molecules.

FIG. 6 shows representative SDS-PAGE gels of constructs described herein.

FIG. 7 shows schematics of mechanisms of activation of activatable IL-2 molecules according certain examples of the present disclosure.

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### Activatable Proteins

In one aspect provided herein are activatable proteins. In some embodiments, an activatable protein comprises a protein with a cleavable moiety attached to it. In some embodiments, the cleavable moiety is attached to a side chain of an amino acid residue of the protein. In some embodiments, the presence of the cleavable moiety modulates the activity of the protein. In some embodiments, the presence of the intact cleavable moiety results in an altered activity of the activatable protein compared to the protein after cleavage of the cleavable moiety. In some embodiments, the presence of the intact cleavable moiety results in a reduced activity of the activatable protein compared to the protein after cleavage of the cleavable moiety.

In one aspect, provided herein, is an activatable protein comprising a cleavable moiety attached to a side chain of an amino acid residue of the protein.

In one aspect, provided herein, is an activatable protein comprising a cleavable moiety attached to a side chain of an amino acid residue of the protein, wherein the protein displays an altered activity after cleavage of the cleavable moiety compared to the activity of the activatable protein prior to cleavage of the cleavable moiety.

In some embodiments, the protein is at least 50 amino acids in length. In some embodiments, the protein is at least 50, 75, 100, or 125 amino acids in length. In some embodiments, the protein is from about 50 to about 500 amino acids in length, from about 50 to about 300 amino acids in length, from about 50 to about 250 amino acids in length, from about 50 to about 200 amino acids in length, from about 100 to about 500 amino acids in length, from about 100 to about 300 amino acids in length, or from about 100 to about 200 amino acids in length. In some embodiments, the protein is from about 50 to about 300 amino acids in length. In some embodiments, the protein is from about 50 to about 250 amino acids in length. In some embodiments, the protein is from about 50 to about 200 amino acids in length. In some embodiments, the protein is from about 50 to about 175 amino acids in length. In some embodiments, the protein is from about 50 to about 150 amino acids in length. In some embodiments, the protein is from about 75 to about 300 amino acids in length. In some embodiments, the protein is from about 75 to about 250 amino acids in length. In some embodiments, the protein is from about 75 to about 200 amino acids in length. In some embodiments, the protein is from about 75 to about 175 amino acids in length. In some embodiments, the protein is from about 75 to about 150 amino acids in length. In some embodiments, the protein is from about 100 to about 300 amino acids in length. In some embodiments, the protein is from about 100 to about 250 amino acids in length. In some embodiments, the protein is from about 100 to about 200 amino acids in length. In some embodiments, the protein is from about 100 to about 175 amino acids in length. In some embodiments, the protein is from about 100 to about 150 amino acids in length.

In some embodiments, the protein of the activatable protein is synthetic. In some embodiments, the protein is prepared from one or more chemically synthesized peptides (e.g., by ligation of the one or more chemically synthesized peptides). In some embodiments, the protein is prepared from 2, 3, 4, 5, 6, or more chemically synthesized peptides. In some embodiments, the cleavable moiety is chemically synthesized and incorporated into the protein during the synthesis of the activatable protein.

In some embodiments, the protein of the activatable protein is recombinant. In some embodiments, the protein of the activatable protein is expressed recombinantly and the cleavable moiety is added after expression, including any optional purification steps. In some embodiments, the cleavable moiety is added to the protein by a conjugation reaction.

The activatable protein can include a protein of any type. In some embodiments, the activatable protein comprises an antibody or antigen binding fragment thereof, a cytokine, a protein hormone, an enzyme, or a fusion protein of any of these. In some embodiments, the activatable protein comprises an antibody or antigen binding fragment thereof. In some embodiments, the activatable protein comprises a cytokine. In some embodiments, the activatable protein comprises a protein hormone. In some embodiments, the activatable protein comprises an enzyme. In some embodiments, the protein is a soluble protein. In some embodiments, the protein is a membrane bound protein. In some embodiments, the protein of the activatable protein is not an antibody. In some embodiments, the protein of the activatable protein is not an antibody-drug conjugate.

In some embodiments, the activatable protein comprises a cytokine. In some embodiments, the cytokine is an interferon, an interleukin, a tumor necrosis factor (TNF) family cytokine, a transforming growth factor (TGF) beta family cytokine, or a chemokine. In some embodiments, the cytokine is an interferon. In some embodiments, the interferon is interferon alpha, interferon beta, or interferon gamma. In some embodiments, the cytokine is an interleukin. In some embodiments, the interleukin is an IL-1 family cytokine (e.g., IL-18, IL-1B, IL-33), an IL-2 family cytokine (e.g., IL-2, IL-4, IL-7, IL-15, IL-21), an IL-6 family interleukin (e.g., IL-6, IL-11, IL-31), an IL-10 family cytokine (e.g., IL-10, IL-19, IL-20, IL-22), an IL-12 family cytokine (e.g., IL-12, IL-23, IL-27, IL-35) or an IL-17 family cytokine (e.g., IL-17, IL-17F, IL-25). In some embodiments, the cytokine is a TNF family cytokine (e.g., TNFα, CD70, TNFSF14). In some embodiments, the cytokine is a chemokine (e.g., CCL2, CCL3, CXCL9, CXCL10). In some embodiments, the cytokine is an IL-2.

In some embodiments, the activatable protein comprise a protein hormone. In some embodiments, the protein hormone is a growth hormone, a follicle-stimulating hormone, insulin, a leutinizing hormone, a thyroid stimulating hormone, a chorionic gonadotropin, a parathyroid hormone, a leptin, an asprosin, a placental lactogen, an insulin-like growth factor 1, an erythropoietin, a relaxin, or a thrombopoietin.

In some embodiments, the protein exhibits an altered activity after cleavage of the cleavable moiety compared to the activatable protein with the cleavable moiety intact. In some embodiments, the altered activity is an enhanced activity. In some embodiments, the altered activity is a reduced activity.

In some embodiments, the altered activity is an altered binding of the protein to a ligand of the protein. The ligand to which the protein shows altered binding after cleavage of the cleavable moiety compared to the activatable protein with the cleavable moiety intact will depend on the type of protein. For example, in embodiments where the protein is a signaling protein (e.g., a protein hormone or a cytokine), the ligand can be a cognate receptor of the protein, a subunit of a cognate receptor of the protein, a regulatory factor of the protein (e.g., a kinase required for activation), a cofactor of the protein, an additional subunit of a complex of which the protein is a component, an inhibitor of the protein, or any other ligand. In embodiments wherein the protein is an enzyme, the ligand can be a substrate, a regulatory factor (e.g., a kinase required for activation), a cofactor, an additional subunit of a complex of which the enzyme is a component, an inhibitor (e.g., an allosteric inhibitor), or another ligand.

In some embodiments, cleavage of the cleavable moiety results in protein binding to the ligand which is increased by at least 2-fold, 3-fold, 4-fold, 5-fold, 6-fold, 7-fold, 8-fold, 9-fold, 10-fold, 20-fold, 50-fold, or 100-fold compared to the activatable protein with the cleavable moiety intact. In some embodiments, cleavage of the cleavable moiety results in protein binding to the ligand which is increased by at least 2-fold compared to the activatable protein with the cleavable moiety intact. In some embodiments, cleavage of the cleavable moiety results in protein binding to the ligand which is increased by at least 4-fold compared to the activatable protein with the cleavable moiety intact. In some embodiments, cleavage of the cleavable moiety results in protein binding to the ligand which is increased by at least 5-fold compared to the activatable protein with the cleavable moiety intact. In some embodiments, cleavage of the cleavable moiety results in protein binding to the ligand which is increased by at least 8-fold compared to the activatable protein with the cleavable moiety intact. In some embodiments, cleavage of the cleavable moiety results in protein binding to the ligand which is increased by at least 10-fold compared to the activatable protein with the cleavable moiety intact. In some embodiments, the degree of increased protein binding is determined by comparing the dissociation constant (K_{D}) of the protein with the ligand after cleavage of the cleavable moiety and the activatable protein with the ligand (e.g., for a 2-fold increase in binding to the ligand, the K_{D} of the protein after cleavage of the cleavable moiety is 2-fold lower than the K_{D} of the activatable protein before cleavage).

In some embodiments, the altered activity is an altered functional activity of the protein. In some embodiments, a functional activity of the protein after cleavage of the cleavable moiety is enhanced (e.g., higher activity) compared to the activatable protein with the cleavable moiety intact. The activity which is altered by cleavage of the cleavable moiety will depend on the type of protein. For example, in embodiments where the protein is a signaling protein (e.g., a protein hormone or a cytokine), the activity which is altered can be a signaling activity of the protein. In some embodiments, wherein the protein comprises an antibody or antigen binding fragment thereof, the activity which is altered is not the ability of the antibody to bind to an Fc receptor. In some embodiments, wherein the protein comprises an antibody or antigen binding fragment, the activity which is altered is the ability of the antibody or antigen binding fragment to bind to its antigen. In embodiments wherein the protein is an enzyme, the activity can be the rate at which the protein converts the substrate to its product. The activity of the protein can be measured using an appropriate assay, including without limitation in vitro assays (e.g., in vitro cellular assays, in vitro protein based assays, etc.) and in vivo assays (e.g., determining the effect of an activatable protein and the corresponding cleavage product in a living organism). In some embodiments, assessing if or the degree to which functional activity is enhanced upon cleavage of the cleavable moiety comprises calculating a numerical value representative of the activity of the protein (e.g., half maximal effective concentration (EC50) values, such as for functional assays, or specific activity, such as for enzyme assays). Such values can then be compared between the activatable protein and the protein after cleavage of the cleavable moiety in order to assess the degree to which the activity has changed. In some embodiments, cleavage of the cleavable moiety results in protein activity which is increased by at least 2-fold, 3-fold, 4-fold, 5-fold, 6-fold, 7-fold, 8-fold, 9-fold, 10-fold, 20-fold, 50-fold, or 100-fold compared to the activatable protein with the cleavable moiety intact. In some embodiments, cleavage of the cleavable moiety results in protein activity which is increased by at least 2-fold compared to the activatable protein with the cleavable moiety intact. In some embodiments, cleavage of the cleavable moiety results in protein activity which is increased by at least 3-fold compared to the activatable protein with the cleavable moiety intact. In some embodiments, cleavage of the cleavable moiety results in protein activity which is increased by at least 5-fold compared to the activatable protein with the cleavable moiety intact. In some embodiments, cleavage of the cleavable moiety results in protein activity which is increased by at least 10-fold compared to the activatable protein with the cleavable moiety intact.

### Cleavable Moieties

In some instances, an activatable protein (e.g., an activatable IL-2 polypeptide) as provided herein comprises a cleavable moiety. In some preferred embodiments, the cleavable moiety is attached to a side chain of an amino acid residue of the protein. In some embodiments, the cleavable moiety is attached in a manner such that it alters the activity of the activatable protein relative to the same protein without the cleavable moiety. In some embodiments, cleavage of the cleavable moiety restores at least a portion of the activity of the protein compared to the activatable protein with the cleavable moiety intact (e.g., the relevant activity is enhanced by 2-fold, 3-fold, 5-fold, 10-fold, 20-fold, 50-fold, etc. after cleavage).

In some embodiments, cleavage of the cleavable moiety leaves a residual portion of the cleavable moiety attached to the protein. In such cases, the residual portion of the cleavable moiety preferably has a smaller impact on the activity than the intact cleavable moiety. In some embodiments, both the residual portion of the cleavable moiety and the intact cleavable moiety may have an impact on the activity relative to a version of the protein to which no group is attached (e.g., a wild type version of the protein, or a version of the protein with a substitution at the point of attachment which allows for the attachment of the cleavable moiety). For example, an activatable protein (e.g., the protein with the intact cleavable moiety attached) may have an activity which is reduced by 100-fold compared to the protein with no group attached and the protein with the residual portion attached may have an activity which is reduced by only 5-fold compared to the protein with no group attached.

In some embodiments, the cleavable moiety comprises a specific cleavage site. In some embodiments, the specific cleavage site is a site which is amenable to cleavage (e.g., breaking a bond) under certain specified, known, and/or desired conditions. Non-limiting examples of specific cleavage sites include protease cleavage sites, sites amenable to cleavage at certain pH ranges (e.g., acid labile bonds), sites amenable to cleavage via oxidation or reduction (e.g., disulfide bonds), photocleavable bonds, and others.

In some embodiments, the cleavable moiety is selected such that it is preferentially cleaved (e.g., cleaved at a faster rate or cleaved in more abundance) at a designated target tissue of a subject. In some embodiments, the cleavable moiety is preferentially at or near a target tissue of the subject such that the specific cleavage site is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the cleavable moiety at a different tissue. In some embodiments, the target tissue is diseased tissue of the subject. In some embodiments, the target tissue is cancer tissue of the subject. In some embodiments, the target tissue is a tumor microenvironment. In some embodiments, the target tissue is a tumor. In some embodiments, the target tissue is at or near a tumor.

### Linking Groups

In some embodiments, the cleavable moiety is attached directly to the protein (e.g., by a bond between the cleavable moiety itself (e.g., a portion of the cleavable moiety necessary for its desired or optimal cleavage (e.g., an amino acid residue of a protease recognition sequence) and the protein). In some embodiments, the cleavable moiety is attached to the protein via a linking group. In some embodiments, the cleavable moiety is attached to a side chain of an amino acid residue of the protein through a linking group. The linking group can be any suitable structure that provides a connection via a chain of atoms between the point of attachment to the protein (e.g., the side chain of an amino acid residue) and the cleavable moiety. In some embodiments, the linking group is attached to the protein via a reaction with a suitable reactive group capable of reacting with a side chain of the protein to form a bond (e.g., an amide, an ester, a carbamate, a carbonate, a carbamide, a thioether, or a disulfide bond is formed). In some embodiments, the linking group is attached to the protein via an amide, an ester, a carbamate, a carbonate, a carbamide, a thioether, or a disulfide bond. The linking group can be selected to impart desired properties to the final activatable protein (e.g., with the intact cleavable moiety attached) or to the protein with a residual portion of the cleavable moiety attached (e.g., after cleavage). In some embodiments, the linking group remains attached to the protein after cleavage of the cleavable moiety.

In some embodiments, the linking group is a chemical linking group. In some embodiments, the linking group comprises at least one portion which is not comprised of amino acid residues. In some embodiments, the linking group comprises a polymer. In some embodiments, the linking group comprises a non-polymer. In some embodiments, the linking group comprises a polymer and a non-polymer (e.g., a polymeric portion and a non-polymeric portion)

In some embodiments, the linking group comprises a polymer. In some embodiments, the linking group comprises a water soluble polymer. In some embodiments, the linking group comprises poly(alkylene oxide), polysaccharide, poly(vinyl pyrrolidone), poly(vinyl alcohol), polyoxazoline, poly(acryloylmorpholine), or a combination thereof. In some embodiments, the linking group comprises poly(alkylene oxide). In some embodiments, the poly(alkylene oxide) is polyethylene glycol or polypropylene glycol, or a combination thereof. In some embodiments, the poly(alkylene oxide) is polyethylene glycol. In some embodiments, the polymer is linear. In some embodiments, the polymer is branched.

In some embodiments, the linking group comprises polyethylene glycol. In some embodiments, the linking group comprises from 2-100 ethylene glycol units in a polyethylene glycol chain. In some embodiments, the linking group comprises from 2 to 50, 2 to 40, 2 to 30, 2 to 20, 2 to 10, 5 to 50, 5 to 40, 5 to 30, 5 to 20, 5 to 10, 10 to 50, 10 to 40, 10 to 30, or 10 to 20 ethylene glycol units in a polyethylene glycol chain. In some embodiments, the linking group comprises 2, 3, 4, 5, 6, 7, 8, 9, 10, 11, 12, 13, 14, 15, 16, 17, 18, 19, or 20 ethylene glycol units in a polyethylene glycol chain. In some embodiments, the linking group comprises 2 to 6 ethylene glycol units. In some embodiments, the linking group comprises 4 ethylene glycol units. In some embodiments, the linking group comprises 6 to 12 ethylene glycol units. In some embodiments, the linking group comprises 9 ethylene glycol units. In some embodiments, the linking group comprises 12 to 20 ethylene glycol units. In some embodiments, the linking group comprises 16 ethylene glycol units. In some embodiments, the linking group comprises 20 to 36 ethylene glycol units. In some embodiments, the linking group comprises 24 ethylene glycol units.

In some embodiments, the linking group comprises a non-polymer. In some embodiments, the non polymer comprises a dicarboxylic acid group (e.g., a malonyl, a succinyl, a glutaryl, an adipiyl, a pimelyl, or a suberyl, group), an amino acid group (e.g., a glycyl, a 3-amino-propanyl, a 4-amino-butanyl, a 5-amino pentanyl, or a 6-amino hexyanyl group), or a diamino group (e.g., a ethylene diaminyl, a propylene diaminyl, a butylene diaminyl, a pentylene diaminyl, or hexylene diaminyl group). In some embodiments, the non-polymer comprises succinimidyl 4-(N-maleimidomethyl)cyclohexane-1-carboxylate; Malcimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminocthyl)-4-{2-[4-(3-azidopropoxy)phenyl]diazenyl} benzamide.

In some embodiments, the linking group comprises a structure

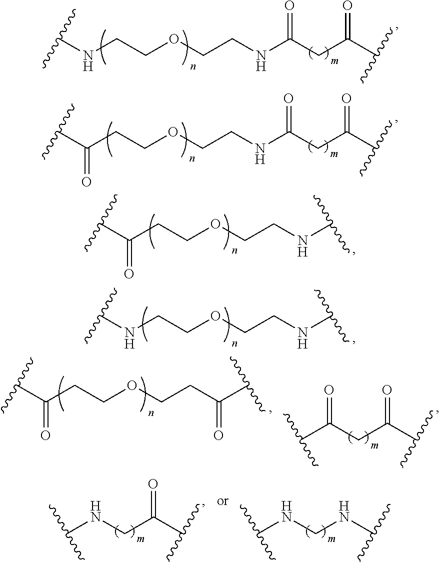

wherein m (if present) is an integer from 1 to 12 and n (if present) is an integer from 2 to 50. In some embodiments, m is 2, 3, or 4. In some embodiments, m is 3. In some embodiments, n is an integer from 2 to 30. In some embodiments, n is 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is an integer from 2 to 6, from 6 to 12, from 12 to 20, or from 20 to 30. In some embodiments, n is 4. In some embodiments, n is 9. In some embodiments, n is 16. In some embodiments, n is 27. In some embodiments, each

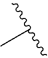

is a point of attachment to either the protein or the cleavable moiety (e.g., the cleavable peptide).

In some embodiments, the linking group is a linking peptide group. In some embodiments, the linking peptide group is attached the N-terminus or the C-terminus of the protein. In some embodiments, the linking peptide group is attached to the N-terminus or the C-terminus of a cleavable peptide.

In some embodiments, the linking group comprises a linear chain of from 1 to 100 atoms between the protein and the cleavable moiety. In some embodiments, the linking group comprises a linear chain of from 1 to 100, 1 to 50, 1 to 40, 1 to 30, 1 to 20, 5 to 100, 10 to 50, 10 to 20, 20 to 100, or 20 to 50 atoms between the protein and the cleavable moiety.

### Cleavable Peptides

In some embodiments, the cleavable moiety comprises a cleavable peptide. In some embodiments, the cleavable peptide can be cleaved by a protease. In some embodiments, the cleavable peptide contains a site of cleavage that can be cleaved specifically by one or more proteases. In some embodiments, the cleavable peptide contains a site of cleavage that can be cleaved at a site preferred by one or more proteases. In some embodiments, the specific cleavage site is a protease cleavage site. In some embodiments, the cleavable peptide comprises multiple cleavage sites (e.g., multiple sites that can be cleaved either by the same protease or by different proteases).

In some embodiments, the protease which cleaves the cleavable peptide is found at higher concentrations and/or demonstrates higher proteolytic activity at or near a target tissue of a subject. In some embodiments, the target tissue is disease tissue. In some embodiments, the target tissue is a cancer. In some embodiments, the target tissue is a tumor microenvironment.

In some embodiments, the cleavable peptide is cleaved by a protease which is found at higher concentrations and/or demonstrates higher proteolytic activity at or near the tumor microenvironment relative to non-tumor tissue. In some embodiments, the cleavable peptide is cleaved by a protease which is found at higher concentrations at or near the tumor microenvironment relative to non-tumor tissue. In some embodiments, the cleavable peptide is cleaved by a protease which demonstrates higher proteolytic activity at or near the tumor microenvironment relative to non-tumor tissue.

In some embodiments, the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the cleavable peptide is cleavable by an MMP. In some embodiments, the cleavable peptide is cleavable by a matriptase. In some embodiments, the cleavable peptide is cleavable by a plasminogen activator. In some embodiments, the cleavable peptide is cleavable by a legumain. In some embodiments, the cleavable peptide is cleavable by a protease set forth in Table 1A, or a combination thereof.

TABLE 1A
Tissue/Tumor specific proteases

| | | |
|---|---|---|
| Protease | Specificity | Other Aspects |

Secreted by CD8⁺ Cytotoxic T cells

| | | |
|---|---|---|
| Granzyme B (grB) | Cleaves after Asp | Type of serine protease; Strongly |
| | | implicated in inducing perforin-dependent |
| | | target cell apoptosis |
| Granzyme A (grA) | trypsin-like, cleaves after | Type of serine protease. |
| | basic residues | |
| Granzyme H (grH) | Unknown substrate specificity | Type of serine protease; |
| | | Other granzymes are also secreted by killer |
| | | T cells, but not all are present in humans |
| Caspase-8 | Cleaves after Asp residue | Type of cysteine protease; plays and |
| | | essential role in TCR-induced cellular |
| | | expansion. |
| Mucosa-associated | Cleaves after Arg residues | Type of cysteine protease; likely acts both |
| lymphoid tissue | | as a scaffold and proteolytically active |
| (MALT1) | | enzyme in the CBM-dependent signaling |
| | | pathway. |
| Tryptase | Target: angiotensin I, | Type of mast cell-specific serine protease; |
| | fibrinogen, prourokinase, | trypsin-like; resistant to inhibition by |
| | TGFβ; preferentially cleaves | macromolecular protease inhibitors |
| | proteins after lysine or | expressed in mammals due to their |
| | arginine residues. | tetrameric structure, with all sites facing |
| | | narrow central pore; also associated with |
| | | inflammation. |

Associated with Inflammation:

| | | |
|---|---|---|
| Thrombin | Targets: FGF-2 | Type of serine protease; modulates activity |
| | HB-EGF, Osteo-pontin, | of vascular growth factors, chemokines |
| | PDGF, VEGF | and extracellular proteins; strengthens |
| | | VEGF-induced proliferation; induces cell |
| | | migration; angiogenic factor; regulates |
| | | hemostasis |
| Chymase | Exhibit chymotrypsin-like | Type of mast cell-specific serine protease. |
| | specificity, cleaving proteins | |
| | after aromatic amino acid | |
| | residues. | |
| Carboxypeptidase A | Cleaves amino acid residues | Type of zinc-dependent metalloproteinase |
| (MC-CPA) | from C-terminal end of | |
| | peptides and proteins | |
| Kallikreins | Targets: high molecular | Type of serine protease; modulate |
| | weight kininogen, | relaxation response; contribute to |
| | prourokinase | inflammatory response; activates |
| | | proapoptotic signaling |
| Elastase | Targets: E-cadherin, GM-CSF, | Type of neutrophil serine protease; |
| | IL-1, IL-2, IL-6, IL-8, | degrades ECM components; regulates |
| | p38^{MAPK}, TNFα, VE-cadherin. | inflammatory response; activates |
| | | proapoptotic signaling. |
| Cathepsin G | Targets: ENA-78, IL-8, MCP- | Type of serine protease; degrades ECM |
| | 1, MMP-2, MT1-MMP, PAI- | components; chemo-attractant of |
| | 1, RANTES, TGFβ, TNFα | leukocytes; regulates inflammatory |
| | | response; promotes apoptosis. |
| PR-3 | Targets: ENA-78, IL-8, IL-18, | Type of serine protease; promotes |
| | JNK, p38^{MAPK}, TNFα | inflammatory response; activates |
| | | proapoptotic signaling. |
| Granzyme M (GrM) | Cleaves after Met and other | Type of serine protease; only expressed |
| | long, unbranched hydrophobic | in NK cells. |
| | residue. | |
| Calpains | Cleave between Arg and Gly | Family of cysteine proteases; calcium- |
| | | dependent; activation is involved in the |
| | | process of numerous inflammation- |
| | | associated diseases. |

In some embodiments, the cleavable peptide is cleavable by multiple proteases. In some embodiments, the cleavable peptide is cleavable by multiple classes of proteases. In some embodiments, the cleavable peptide is cleavable by 2, 3, or 4 different proteases. In some embodiments, the cleavable peptide comprises multiple cleavage sites. In some embodiments, the cleavable peptide comprises 2, 3, 4, or more cleavage sites. In some embodiments, the cleavable peptide comprises 2 cleavage sites. In some embodiments, the cleavable peptide comprises 3 cleavage sites. In some embodiments, the cleavable peptide comprises 4 cleavage sites. In some embodiments, each of the cleavage sites is cleavable by a different protease.

In some embodiments, the cleavable peptide is cleavable by a matrix metalloprotease and a legumain. In some embodiments, the cleavable peptide is cleavable by a matrix metalloprotease and a matriptase. In some embodiments, the cleavable peptide is cleavable by a matrix metalloprotease and a plasminogen activator. In some embodiments, the cleavable peptide is cleavable by a legumain and a matriptase. In some embodiments, the cleavable peptide is cleavable by a legumain and a plasminogen activator. In some embodiments, the cleavable peptide is cleavable by a matriptase and a plasminogen activator. In some embodiments, the cleavable peptide is cleavable by a matrix metalloprotease, a legumain, and a matriptase. In some embodiments, the cleavable peptide is cleavable by a matrix metalloprotease, a matriptase, and a plasminogen activator. In some embodiments, the cleavable peptide is cleavable by a matrix metalloprotease, a legumain, and a plasminogen activator.

In some embodiments, the cleavable peptide has a length of from 2 to 30 amino acids. In some embodiments, the cleavable peptide has a length of at most 30, 25, 20, 19, 18, 17, 16, 15, 14, 13, 12, 11, 10, 9, 8, 7, 6, or 5 amino acids. In some embodiments, the cleavable peptide has a length of at least 4, 5, 6, 7, 8, 9, or 10 amino acids. In some embodiments, the cleavable peptide has a length of from 4 to 30, 4 to 25, 4 to 20, 4 to 18, 4 to 16, 4 to 15, 4 to 12, 4 to 10, 5 to 30, 5 to 25, 5 to 20, 5 to 18, 5 to 16, 5 to 15, 5 to 12, or 5 to 10 amino acids.

In some embodiments, cleavage of the cleavable peptide leaves a residual portion of the cleavable peptide attached to the protein. In some embodiments, cleavage of the cleavable peptide leaves a residual portion of the cleavable peptide attached to the side chain of the amino acid residue to which the cleavable peptide is attached. In some embodiments, cleavage of the cleavable peptide leaves at least one amino acid of the cleavable peptide still attached to the protein. In some embodiments, cleavage of the cleavable peptide leaves at least one amino acid of the cleavable peptide still attached to the side chain of the amino acid residue to which the cleavable peptide is attached. In some embodiments, cleavage of the cleavable peptide leaves 1, 2, 3, 4, 5, 6, 7, 8, 9, or 10 amino acids attached to the side chain of the amino acid residue to which the cleavable moiety is attached. In some embodiments, cleavage of the cleavable peptide leaves at most 10 amino acids of the cleavable peptide attached to the protein. In some embodiments, cleavage of the cleavable peptide leaves at most 5 amino acids of the cleavable peptide attached to the protein. In some embodiments, cleavage of the cleavable peptide leaves 1 amino acid of the cleavable peptide attached to the protein. In some embodiments, cleavage of the cleavable peptide leaves 2 amino acids of the cleavable peptide attached to the protein. In some embodiments, cleavage of the cleavable peptide leaves 3 amino acids of the cleavable peptide attached to the protein. In some embodiments, cleavage of the cleavable peptide leaves 4 amino acids of the cleavable peptide attached to the protein. In some embodiments, cleavage of the cleavable peptide leaves 5 amino acids of the cleavable peptide attached to the protein. In some embodiments, the reference to amino acids attached to the protein described in this paragraph refers only to those at a single point of attachment (e.g., in a case where the cleavable peptide is attached to the protein at two points of attachment, the number of amino acids attached in this paragraph refers to the number of amino acids attached to either or both of the first and second points of attachment).

Exemplary cleavable peptide sequences which can be incorporated into an activatable protein as provided herein can be found in any one of U.S. Patent Publication Nos: US2010/0189651, US2016/0289324, US2018/0125988, US2019/0153115, US2020/0385469, US2021/0260163, US2022/0048949, US2022/0267400, US2021/0115102, US2022/0002370, US2021/0163562, US20200392235, US2021/0139553, US2021/0317177, US2020/0283489, US2021/0002343, US2021/0292421, US2021/0284728, US2021/0269530, US2022/0054544, US2021/0355219, US2022/0073613, US2021/0047406, and/or Patent Cooperation Treaty Publication Nos: WO2021/202675, WO2021/062406, WO2021/142471, WO2021/216468, WO2021/119516, WO2021/253360, WO2021/146455, WO2021/202678, WO2021/202673, WO2021/189139, WO2020/232303, WO2022/115865, WO2021/202678, and/or Chen et. al., J Bio Chem, 277, V6 P4485-4491. (2002), each of which is hereby incorporated by reference as if set forth herein in its entirety.

In some embodiments, the cleavable peptide comprises an amino acid sequence having at least about 80%, at least about 90%, or 100% identity to a sequence set forth in Table 1B or Table 1C below. In some embodiments, the cleavable peptide comprises an amino acid sequence set forth in Table 1B below. In some embodiments, the cleavable peptide comprises an amino acid sequence set forth in Table 1C below.

TABLE 1B
Exemplary Cleavage Sequences

| SEQ | Cleavable | |
|---|---|---|
| ID NO: | Peptide Sequence | Protease |
| 101 | SGGPGPAGMKGLPGS | MMP9 |
| 102 | EAGRSANHEPLGLVAT | MMP2-7-9-14 + matriptase + |
| | | uPA + Legumain |
| 103 | PQASTGRSGG | MMP9 + matriptase + uPA |
| 104 | PQGSTGRAAG | MMP9 + matriptase + uPA |
| 105 | PPASSGRAGG | MMP9 + matriptase + uPA |
| 106 | RSGVPLSLYSGSGGGK | MMP7-9-10 |
| 107 | RSGMPYDLYHPSGK | MMP7-9-10 |
| 108 | RGPDSGGFMLTSGK | MMP7-9-10 |
| 109 | RGSGHEQLTVSGGSK | MMP7-9-10 |
| 110 | RSGRAAAVKSPSGK | MMP7-9-10 |
| 111 | RGSGISSGLLSGRSDNHSGK | MMP7-9-10 |
| 112 | RGDLLAVVAASGGK | MMP7-9-10 |
| 113 | RGGISSGLLSGRSGK | MMP7-9-10 |
| 114 | SGGGKKLADEPEGGS | Meprin A/B |
| 115 | GGGKFLADEPEGG | Meprin A/B (High Efficiency) |
| 116 | ARLQSAAP | Cathepsin S, K, L |
| 117 | ARLQSAAPAGLKGA | Cathepsin S + MMP9 + Meprin A |
| 118 | GSGGPGPAGMHGLPGGS | MMP9 (High Efficiency) |
| 119 | GGGSHTGRSAYFGGGS | uPA |
| 120 | SGGPGPAGLKGAPGS | MMP9-2 |
| 121 | VPLSLYSG | MMP2-7-9 |
| 122 | SGLLSGRSDNH | uPA + Matriptase + legumain |
| 123 | SGRSDNIGGGS | uPA |
| 124 | LQESLRSKESGRSDI | MMP-14 + uPA |
| 125 | ISSGLLSGRSDNH | uPA |
| 126 | LSGRSDDH | uPA |
| 127 | ISSGLLSGRSDQH | uPA |
| 128 | ISSGLLSGRSDNI | uPA |
| 129 | PLGLAG | MMP2-7-9 |
| 130 | GPAGMKGL | MMP |
| 131 | LSGRSDQH | MMP |
| 132 | LSGRSDNI | MMP |
| 133 | ISSGLLSGRSDNH | MMP |
| 134 | GPLGVRG | MMP |
| 135 | GPLGLAR | MMP |
| 136 | GPAALVGA | MMP |
| 137 | GPAALIGG | MMP |
| 138 | GPLNLVGR | MMP |
| 139 | GPAGLVAD | MMP |
| 140 | GPANLVAP | MMP |
| 141 | VPLSLYSG | MMP |
| 142 | SGESPAYYTA | MMP |
| 143 | GGPRGLG | MMP |
| 144 | HSSKLQ | MMP |
| 145 | HSSKLQL | MMP |
| 146 | KRALGLPG | MMP7 |
| 147 | LEATA | MMP9 |
| 148 | GGAANLVRGG | MMP11 |
| 149 | SGRIGFLRTA | MMP14 |
| 150 | PLGLA | MMP |
| 151 | ESPAYYTA | MMP |
| 152 | RLQLKL | MMP |
| 153 | RLQLKAC | MMP |
| 154 | SGRSA | uPA |
| | (D-Ala)FK | uPA |
| 156 | GGGRR | uPA |
| 157 | GFLG | Lysosomal Enzyme |
| 158 | HSSKLQEDA | Prostate Specific Antigen |
| 159 | LVLASSSFGY | HSV Protease |
| 160 | GVSQNYPIVG | HIV Protease |
| 16 | GVVQASCRLA | CMV Protease |
| 162 | DPRSFL | Thrombin |
| 163 | PPRSFL | Thrombin |
| 164 | DEVD | Caspase-3 |
| 165 | DEVDP | Caspase-3 |
| 166 | KGSGDVEG | Caspase-3 |
| 167 | GWEHDG | Interleukin 1β converting enzyme |
| 168 | EDDDDKA | Enterokinase |
| 169 | KQEQNPGST | FAP |
| 170 | GKAFRR | Kallikrein 2 |
| 171 | DAFK | Plasmin |
| 172 | DVLK | Plasmin |
| 173 | DAFK | Plasmin |
| 174 | ALLLALL | TOP |
| 175 | SGAKPRALTA | MMP A3 |
| 176 | SGLRLAAITA | MMP B49 |
| 177 | SGESLAYYTA | MMP B74 |
| 178 | SGESPAYYTA | MMP B74P |
| 179 | SGESLRYYTA | MMP B74R |
| 180 | SGRSLSRLTA | MMP C9 |
| 181 | SGRSLRRLTA | MMP C9R |
| 182 | SGAVSWLLTA | MMP A13 |
| 183 | SGAPSWLLTA | MMP A13P |
| 184 | SGAVRWLLTA | MMP A13R |
| 185 | SGANISDLTA | MMP B37 |
| 186 | SGNRYSSLTA | MMP A34 |
| 187 | SGHMHKALTA | MMP A21 |
| 188 | SGHMHKALTA | MMP A21A |
| | EVCit | Cathepsin B |
| | (p-aminobenzyloxycarbonyl) | |
| 190 | GEEGEEPLGLAG | |
| 191 | GPLGLAG | |
| 192 | EAGRSANHTPAGLTGP | |
| 193 | GEAGRSANHTPAGLTGP | |

TABLE 1C
Additional Exemplary Cleavage Sequences

| SEQ ID NO: | Cleavable Peptide Sequence |
|---|---|
| 201 | AGDKSPLGLAG |
| 202 | AGDRSPLGLAG |
| 203 | AGDRSAPLGLAG |
| 204 | AWGRSPLGLAG |
| 205 | AWGRSAPLGLAG |
| 206 | AWGKSPLGLAG |
| 207 | GAFKSPLGLAG |
| 208 | SGRSAPLALAG |
| 209 | SGRSPLGLAG |
| 328 | RQRRSAAPLGLAG |
| 309 | RQRRSAPLGLAG |
| 310 | RQRRSPLGLAG |
| 325 | RQRRS-Nle-PLGLAG |
| 214 | RSGRSAPLGLAG |
| 215 | RSGKSAPLGLAG |
| 216 | FTARSAPLGLAG |
| 218 | FTAKSPLGLAG |
| 219 | RYGRSAPLGLAG |
| 220 | RYGRSPLGLAG |
| 327 | RYGKSAPLGLAG |
| 222 | KYGRSAPLGLAG |
| 223 | KWGRSAPLGLAG |
| 326 | KWGKSAPLGLAG |
| 225 | KWGRSPLGLAG |
| 311 | SGRVLTLRKAGPAGLVG |
| 312 | SGRVLTLRKAGPANLVG |
| 313 | SGRVLRKAGPAGLVG |
| 314 | SGRVLRKAGPANLVG |
| 305 | SGRVLGPAGLVG |
| 306 | SGRVLGPANLVG |
| 307 | SGRVLPAGLVG |
| 315 | SGRVLPANLVG |
| 316 | SGRVAGLVG |
| 317 | SGRVANLVG |
| 236 | SSRGRRGPLGLAG |
| 237 | SSRGPLGLAG |
| 238 | SSRGPRGLAG |
| 301 | SSRGPASNRRLPLGLAG |
| 318 | PASNRRLPLGLAG |
| 302 | SSRAVFRKNLGPLGLAG |
| 319 | SSRVFRKPANLAG |
| 304 | SGRVLTLRKAPWGLLE |
| 320 | SGRVLTLRKAALPLAM |
| 303 | SGRVLRKAGPQPLVD |
| 246 | SGRVLPLNLSG |
| 308 | SGRVLGPLNLSG |
| 321 | SSRGRRGPYMLQG |
| 322 | SSRGPYMLQG |
| 250 | SGRVLPLGLAG |
| 251 | SGRVLPMSLRM |
| 323 | SGRVLPLGMRA |
| 253 | SGRVLPLGLRA |
| 324 | SGRVLPYAMTA |
| 255 | SGRVLPLGFMG |

TABLE 1D
Further Exemplary Cleavage Sequences
and portions thereof

| SEQ ID NO: | Sequence |
|---|---|
| 301 | SSRGPASNRRLPLGLAG |
| 302 | SSRAVFRKNLGPLGLAG |
| 303 | SGRVLRKAGPQPLVD |
| 304 | SGRVLTLRKAPWGLLE |
| 305 | SGRVLGPAGLVG |
| 306 | SGRVLGPANLVG |
| 307 | SGRVLPAGLVG |
| 308 | SGRVLGPLNLSG |
| 309 | RQRRSAPLGLAG |
| 310 | RQRRSPLGLAG |
| 311 | SGRVLTLRKAGPAGLVG |
| 312 | SGRVLTLRKAGPANLVG |
| 313 | SGRVLRKAGPAGLVG |
| 314 | SGRVLRKAGPANLVG |
| 315 | SGRVLPANLVG |
| 316 | SGRVAGLVG |
| 317 | SGRVANLVG |
| 318 | PASNRRLPLGLAG |
| 319 | SSRVFRKPANLAG |
| 320 | SGRVLTLRKAALPLAM |
| 321 | SSRGRRGPYMLQG |
| 322 | SSRGPYMLQG |
| 323 | SGRVLPLGMRA |
| 324 | SGRVLPYAMTA |
| 325 | RQRRS-Nle-PLGLAG |
| 326 | KWGKSAPLGLAG |
| 327 | RYGKSAPLGLAG |
| 328 | RQRRSAAPLGLAG |
| 329 | RQRRSVVGG |
| 330 | SPLGLAGS |
| 331 | RGRKVANLVG |
| 332 | RQRKVANLVG |
| 333 | RGRRVANLVG |
| 334 | RGRKSPANLVG |
| 335 | RGRKPYMLQG |
| 336 | RGRKPY-Nle-LQG |
| 337 | RGRKSPYMLQG |
| 338 | RGRKSPY-Nle-LQG |
| 339 | RGRKPQPLVD |
| 340 | RGRKSPQPLVD |
| 341 | RGRKSQPLVD |
| 342 | SGRVAPYMLQG |
| 343 | SGRVAPY-Nle-LQG |
| 344 | SGRVYMLQG |
| 345 | SGRVY-Nle-LQG |
| 346 | SGRVAPQPLVD |
| 347 | SGRVQPLVD |
| 348 | RGRRGP |
| 349 | RGRRPLGLAG |
| 350 | RGRRVANPLGLAGSG |
| 351 | RGRRPLGLAGGSG |
| 352 | RGRRHSSKLQ |
| 353 | SGRVANPLGGSG |
| 354 | SGRVANYFGKL |
| 355 | RGRRVANYFGKL |
| 356 | SGRPLGYFGKL |
| 357 | RGRRPLGYFGKL |
| 358 | RGRRVANPLGYFGKL |
| 359 | RGRRSGRAANLVRPLGYFGKL |
| 360 | RGRRAANLVRPLGYFGKL |
| 361 | HSSKLQYFGKL |
| 362 | RGRRHSSKLQPLGYFGKL |
| 363 | SGRHSSKLQPLGYFGKL |
| 364 | GSGSGSGS |
| 365 | SSLYSSPG |
| 366 | SSLQSSPG |
| 367 | SQYQSSPG |
| 368 | SQLYSSPG |
| 369 | SSQYSSPG |
| 370 | ISQYSSAT |
| 371 | KLYSSKQ |
| 372 | KLFSSKQ |
| 373 | RRLHYSL |
| 374 | RRLNYSL |
| 375 | RSSYRSL |
| 376 | RSSYYSL |
| 377 | KSKQHSL |
| 378 | HSSKLQL |
| 379 | GSSYYSGA |
| 380 | GSSVYSGR |
| 381 | SS-Nle-YSSAG |

The cleavable peptide can be attached to the protein (e.g., a side chain of an amino acid residue of the protein) in a variety of ways. In some embodiments, the cleavable peptide is covalently attached to the protein (e.g., a side chain of an amino acid residue of the protein). In some embodiments, the cleavable peptide is attached to the protein through its C-terminal carboxyl, its N-terminal amine, or through a side chain of the cleavable peptide. In some embodiments, the cleavable peptide is directly attached to the protein. In some embodiments, the cleavable peptide is directly attached to a side chain of an amino acid residue of the protein.

In some embodiments, the cleavable peptide is attached to the protein through the C-terminus of the cleavable peptide. In some embodiments, the C-terminal carboxyl group of the cleavable peptide is directly attached to a side chain of an amino acid residue of the protein. In some embodiments, the C-terminal carboxyl group of the cleavable peptide is directly attached to a side chain amine of an amino acid residue of the protein (e.g., as an amide bond). In some embodiments, the side chain amine is of a lysine residue of the protein. In some embodiments, the side chain amine is of an unnatural amino acid residue of the protein (e.g., ornithine, homolysine, 2,4-diamobutyric acid (Dab) or another amine containing unnatural amino acid). In some embodiments, the C-terminal carboxyl group of the cleavable peptide is attached to a side chain of an amino acid residue of the protein through a linking group (e.g., any of the linking groups provided herein). In some embodiments, the C-terminal carboxyl group of the cleavable peptide attached to the linking group by an amide bond formed between the C-terminal carboxyl and an amine group of the linking group.

In some embodiments, the cleavable peptide is attached to the protein through the N-terminus of the cleavable peptide. In some embodiments, the N-terminal amine group of the cleavable peptide is directly attached to a side chain of an amino acid residue of the protein. In some embodiments, the N-terminal amine group of the cleavable peptide is directly attached to a side chain carboxyl of an amino acid residue of the protein (e.g., as an amide bond). In some embodiments, the side chain carboxyl is of a glutamate or aspartate residue of the protein. In some embodiments, the side chain carboxyl is of an unnatural amino acid residue of the protein (e.g., 2-amino adipic acid, 2-amino pimelic acid, or another carboxyl containing unnatural amino acid). In some embodiments, the N-terminal amine group of the cleavable peptide is attached to a side chain of an amino acid residue of the protein through a linking group (e.g., any of the linking groups provided herein). In some embodiments, the N-terminal carboxyl group of the cleavable peptide attached to the linking group by an amide bond formed between the N-terminal amine and a carboxyl group of the linking group.

In some embodiments, the cleavable peptide is attached to the protein through a side chain of an amino acid residue of the cleavable peptide. In some embodiments, a side chain of an amino acid residue of the cleavable peptide is directly attached the protein (e.g., to a side chain of an amino acid residue of the protein). In some embodiments, a side chain of an amino acid residue of the cleavable peptide is attached to the protein through a linking group (e.g., any of the linking groups provided herein).

### Redox Cleavable Moieties

In some instances, different tissues of a subject can exhibit different redox potentials depending on the activity of the tissue. For example, tumors and tumor microenvironments are associated with having substantially greater reduction potentials than other healthy tissues. Thus, in some embodiments, cleavable moieties provided herein utilize this property to allow preferential cleavage and activation of a protein at the tissue site.

In some embodiments, the cleavable moiety can be cleaved by a reduction or oxidation reaction. In some embodiments, the cleavable moiety contains a site of cleavage that can be cleaved specifically by a reduction or oxidation reaction. In some embodiments, the cleavable moiety contains a site of cleavage that can be cleaved at a site preferred by a reduction or oxidation reaction. In some embodiments, the specific cleavage site is a redox sensitive cleavage site.

In some embodiments, the redox sensitive cleavage site is preferentially cleaved at or near a target tissue of the subject such that the specific cleavage site is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the specific cleavage site at a different tissue. In some embodiments, the redox cleavage site is preferentially cleaved in a reducing environment. In some embodiments, the redox cleavage site is preferentially cleaved in a reducing environment relative to blood. In some embodiments, the redox cleavage site is preferentially cleaved in a reducing environment relative to interstitial fluids. In some embodiments, the redox cleavage site is preferentially cleaved in a reducing environment relative to lymphatic fluid. In some embodiments, the redox cleavage site is preferentially cleaved in a reducing environment of a tumor microenvironment.

In some embodiments, the redox sensitive cleavage site is a disulfide. In some embodiments, the cleavable moiety is not a disulfide bond between two cysteines in the native protein. In some embodiments, the cleavable moiety is not a disulfide bond between the side chains of two amino acids of the protein.

### pH Cleavable Moieties

The pH of circulating blood is generally buffered in a narrow range of between 7.31 to 7.45. Variances outside this range typically result in acidosis or alkalosis, which are serious medical conditions. The tumor microenvironment is characteristically more acidic than circulating blood pH due to a metabolic dysregulation in tumor cells known as the Warburg effect: Growing tumor cells demonstrate a high rate of glycolysis followed by fermentation of pyruvate to lactic acid in the cytoplasm rather than oxidation of pyruvate in the mitochondrial TCA cycle. To maintain the pH of their cytoplasm, tumor cells transport hydrogen ions to the extracellular environment, resulting in an acidic tumor microenvironment.

In some embodiments, the cleavable moiety comprises a pH sensitive cleavage site. In some embodiments, the pH sensitive cleavage site is selected to preferentially cleave at a target tissue. In some embodiments, the target tissue is associated with a certain pH or a difference in pH compared to other local tissues.

In some embodiments, the pH sensitive cleavage site is cleaved at a pH below physiological blood pH (e.g., below about 7.3). In some embodiments, the pH sensitive cleavage site is preferentially cleaved at a pH below 7.3, below 7.2, below 7.1, or below 7.0. In some embodiments, the pH sensitive cleavage site is preferentially cleaved at acidic pHs. In some embodiments, the pH sensitive cleavage site is preferentially cleaved at a pH of below 7, 6.5, 6, 5.5, or 5.

In some embodiments, the pH sensitive cleavage site is preferentially cleaved at or near a target tissue of the subject such that the specific cleavage site is cleaved at a rate of least 2-fold, at least 4-fold, at least 8-fold, at least 10-fold, at least 15-fold, at least 20-fold, at least 50-fold, at least 100-fold, at least 200-fold, at least 500-fold, or at least 1000-fold greater than cleavage of the specific cleavage site at a different tissue. In some embodiments, the tissue is a tumor microenvironment.

### Points of Attachment of Cleavable Moieties to Proteins

The activatable proteins and other polypeptides (e.g., activatable IL-2 polypeptides) comprise cleavable moieties attached to the base protein. In some embodiments, the cleavable moiety is attached to a side chain of an amino acid residue of the protein. In some instances, attachment of the cleavable moiety to a side chain of an amino acid residue of the protein (rather than attachment to the N- or C-terminus, as in a traditional fusion protein) allows for attachment of the cleavable moiety to the protein at a location that is best suited to modulate the activity in a desired manner. For example, a cleavable moiety can be attached to the protein at or near an amino acid residue which interacts with a ligand of the activatable protein, thereby reducing binding of the activatable protein until the cleavable moiety is cleaved, at which point the protein becomes “activated” and binding with the ligand is increased.

The cleavable moiety can be attached to a side chain of a wide variety of different amino acid residues. In some embodiments, the amino acid residue to which the cleavable moiety is attached is lysine, glutamate, glutamine, aspartate, asparagine, tyrosine, serine, threonine, cysteine, or an unnatural amino acid. In some embodiments, the amino acid residue to which the cleavable moiety is attached is lysine, glutamate, glutamine, aspartate, asparagine, tyrosine, serine, threonine, cysteine, or an unnatural amino acid. In some embodiments, the amino acid residue to which the cleavable moiety is attached is lysine, glutamate, aspartate, tyrosine, serine, threonine, cysteine, or an unnatural amino acid. In some embodiments, the amino acid residue to which the cleavable moiety is attached is lysine, glutamate, aspartate, tyrosine, cysteine, or an unnatural amino acid. In some embodiments, the amino acid residue to which the cleavable moiety is attached is lysine, glutamate, aspartate, cysteine, or an unnatural amino acid. In some embodiments, the amino acid residue to which the cleavable moiety is attached is lysine, glutamate, aspartate, or cysteine. In some embodiments, the amino acid residue to which the cleavable moiety is attached is a lysine. In some embodiments, the amino acid residue to which the cleavable moiety is attached is a lysine or glutamate. In some embodiments, the amino acid residue to which the cleavable moiety is attached is a glutamate. In some embodiments, the amino acid residue to which the cleavable moiety is attached is an aspartate. In some embodiments, the amino acid residue to which the cleavable moiety is attached is a cysteine.

In some embodiments, the amino acid residue to which the cleavable moiety is attached is a lysine. In some embodiments, the cleavable moiety is attached to the lysine via an amide bond between the side chain amine of the lysine and a carboxyl group of the cleavable moiety or a linking group.

In some embodiments, the amino acid residue to which the cleavable moiety is attached is a glutamate. In some embodiments, the cleavable moiety is attached to the glutamate via an amide bond between the side chain carboxyl of the glutamate and an amine group of the cleavable moiety or a linking group. In some embodiments, the cleavable moiety is attached to the glutamate via an ester bond between the side chain carboxyl of the glutamate and a hydroxyl group of the cleavable moiety or a linking group.

In some embodiments, the amino acid residue to which the cleavable moiety is attached is an aspartate. In some embodiments, the cleavable moiety is attached to the aspartate via an amide bond between the side chain carboxyl of the aspartate and an amine group of the cleavable moiety or a linking group. In some embodiments, the cleavable moiety is attached to the aspartate via an ester bond between the side chain carboxyl of the glutamate and a hydroxyl group of the cleavable moiety or a linking group.

In some embodiments, the amino acid residue to which the cleavable moiety is attached is a cysteine. In some embodiments, the cleavable moiety is attached to the cysteine via thioether bond between the side chain thiol of the cysteine and an alkyl or aryl group of the cleavable moiety or a linking group. In some embodiments, the bond between the cleavable moiety or linking group and the cysteine sulfhydryl is formed from a reaction of the sulfhydryl with a suitable reactive group (e.g., a maleimide, an α,β-unsaturated carbonyl, an α-halo carbonyl).

In some embodiments, the amino acid residue to which the cleavable moiety is attached is a tyrosine. In some embodiments, the cleavable moiety is attached to the tyrosine via an ether bond between the side chain phenol of the tyrosine and an alkyl or aryl group of the cleavable moiety or a linking group.

In some embodiments, the amino acid residue to which the cleavable moiety is attached is the amino acid present at that position in a wild type version of the protein on which the activatable protein is based. In some embodiments, the amino acid residue to which the cleavable moiety is attached is substituted relative to the amino acid at the corresponding position in the wild type version of the protein. In some embodiments, the amino acid residue to which the cleavable moiety is attached is substituted to a lysine, glutamate, glutamine, aspartate, asparagine, tyrosine, serine, threonine, cysteine, or an unnatural amino acid.

In some embodiments, the amino acid residue to which the cleavable moiety is attached is an unnatural amino acid. In some embodiments, the unnatural amino acid comprises a side chain having a functional group that allows for attachment of the cleavable moiety or linking group to the unnatural amino acid. In some embodiments, the unnatural amino acid comprises a side chain functional group which is the same as that of a natural amino acid (e.g., carboxyl, amine, phenol, thiol). In some embodiments, unnatural amino acid comprises a side chain amine. In some embodiments, the unnatural amino acid is a lysine analog (e.g., ornithine, homolysine, 2,4-diaminobutyric acid, etc.). In some embodiments, the unnatural amino acid comprises a side chain carboxyl. In some embodiments, the unnatural amino acid is a glutamate analog (e.g., 2-amino adipic acid, 2-amino pimelic acid). In some embodiments, the unnatural amino acid comprises a side chain thiol. In some embodiments, the unnatural amino acid is a cysteine analog (e.g., homocysteine, another amino acid containing an alkyl group side chain with a thiol, etc.).

In some embodiments, the unnatural amino acid residue to which the cleavable moiety is attached comprises a conjugation handle for the attachment of the cleavable moiety or a linking group. In some embodiments, the conjugation handle facilitates the attachment of the cleavable moiety to the protein through a reaction with a complementary conjugation handle on the cleavable moiety or linking group. Thus, in such cases, the activatable protein will comprise a reaction product of the conjugation handle and the complementary conjugation handle. The conjugation handle can be any suitable conjugation handle, including any of the conjugation handles provided herein. Non-limiting examples of unnatural amino acid residues comprising conjugation handles can be found, for example, in Patent Cooperation Treaty Pub. Nos. WO2015/054658, WO2014/036492, WO2021/133839, WO2006/069246, and WO2007/079130, each of which is incorporated by reference as if set forth in its entirety.

In some embodiments, the amino acid to which the cleavable moiety is attached (e.g., at the side chain) is an internal amino acid of the protein. In some embodiments, the amino acid to which the cleavable moiety is attached is a non-terminal residue. In some embodiments, the cleavable moiety is attached to the side chain of the N-terminal or C-terminal residue of the protein.

In some embodiments, the cleavable moiety is site specifically attached to a specified or pre-determined amino acid residue. In some embodiments, the cleavable moiety is attached such that a population of the activatable protein contains substantially all of the cleavable moieties attached to the same residue position of individual proteins. In some embodiments, a population of activatable proteins comprises at least 90%, at least 95%, at least 96%, at least 97%, at least 98%, or at least 99% of the individual proteins having the cleavable moiety attached to the same amino acid position.

In some embodiments, the amino acid residue to which the cleavable moiety is attached is at or near an amino acid residue which interacts with a ligand of the protein. In some embodiments, the cleavable moiety is attached to an amino acid residue which interacts with a ligand of the protein. In some embodiments, determination of which amino acid residues interact with a ligand is determined by X-ray crystallography (e.g., by examining an X-ray co-crystal structure of the protein with the ligand (or a suitable portion thereof)), by nuclear magnetic resonance (NMR), by a mutagenesis-based approach, or any combination thereof. Determination of which amino acid residues interact with a ligand can also be performed by computational modelling methods. In some embodiments, the cleavable moiety is attached to a residue which is within 1, 2, 3, 4, 5, 6, 7, 8, 9, or 10 amino acids of the amino acid residue which interacts with (e.g., binds to) to a ligand. In some embodiments, the cleavable moiety is attached to a residue which is within 10, 20, 30, 40, 50, 60, 70, 80, 90, or 100 amino acids of the amino acid residue which interacts with (e.g., binds to) to a ligand.

In some embodiments, the cleavable moiety is attached to a residue selected for being in proximity to an amino acid residue which interacts with a ligand of the protein when in a folded state but may not be in close proximity in terms of primary structure of the protein (e.g., the amino acid residue to which the cleavable moiety is attached is not close in terms of sequence but is close in terms of 3D structure of the folded protein). In some embodiments, proximity to the amino acid residue which interacts with the ligand is determined by X-ray crystallography, NMR, a computational modelling method, or any combination thereof. In some embodiments, the cleavable moiety is attached to an amino acid residue which is within 5 angstroms, 10 angstroms, 15 angstroms, 20 angstroms, 25 angstroms, 50 angstroms, 75 angstroms, or 100 angstroms of the amino acid residue which interacts with (e.g., binds to) to a ligand.

### Additional Points of Attachment of Cleavable Moieties to Proteins

In some embodiments, the cleavable moiety is attached to the protein at an additional point of attachment (e.g., at a second point of attachment which is different than the point of attachment to the side chain of the amino acid residue of the protein). In such cases, the cleavable moiety forms a macrocyclic structure by linking the two points of attachment. In some embodiments, attachment of the cleavable moiety to two points of the protein (e.g., the side chain of an amino acid residue of the protein and the additional point of attachment) can act to lock the activatable protein in a less active conformation than a wild type version of the protein. In some embodiments, cleavage of the cleavable moiety breaks the macrocyclic structure. In some embodiments, breaking of the macrocyclic structure frees the “activated” protein to adopt a new conformation. The additional point of attachment can be to any amino acid residue, in particular those described above for points of attachment of cleavable moieties (e.g., to any of the types of residues described above), and can optionally be attached through an additional linking group as described above. The additional linking group can be attached to the cleavable moiety at an additional point in much the same manner as described above, and the additional linking group can have any of the structures or compositions described herein (e.g., any of the structures or compositions for the first linking group). Where the cleavable moiety is attached to the protein at two sites by two linking groups, the two linking groups can be the same or different.

In some embodiments, the additional point of attachment is to a side chain of another amino acid residue of the protein (e.g., a different amino acid residue from the first point at which the cleavable moiety is attached). In some embodiments, the cleavable moiety is attached directly to a side chain of a first amino acid residue and is further attached directly to a side chain of a second amino acid residue. In some embodiments, the cleavable moiety is attached directly to a side chain of a first amino acid residue and is further attached to a side chain of a second amino acid residue through a linking group. In some embodiments, the cleavable moiety is attached to a side chain of a first amino acid residue through a linking group and is further directly attached to a side chain of a second amino acid residue. In some embodiments, the cleavable moiety is attached to a side chain of a first amino acid residue through a first linking group and is further attached to a side chain of a second amino acid residue through a second linking group.

In some embodiments, the additional point of attachment is to the N-terminus or the C-terminus protein. In some embodiments, the additional point of attachment is to the N-terminus of the protein. In some embodiments, the additional point of attachment is to the C-terminus of the protein. In some embodiments, the cleavable moiety is attached directly to a side chain of an amino acid residue and is further attached directly to the N-terminus or the C-terminus of the protein. In some embodiments, the cleavable moiety is attached directly to a side chain of an amino acid residue and is further attached to the N-terminus or the C-terminus of the protein through a linking group. In some embodiments, the cleavable moiety is attached to a side chain of an amino acid residue through a linking group and is further directly attached to N-terminus or the C-terminus of the protein. In some embodiments, the cleavable moiety is attached to a side chain of an amino acid residue through a first linking group and is further attached to the N-terminus or the C-terminus of the protein through a second linking group.

In some embodiments, the cleavable moiety comprises a cleavable peptide which is attached directly to the N-terminus or the C-terminus of the protein (e.g., the cleavable moiety comprises a cleavable peptide in which the C-terminal carboxyl of the cleavable peptide forms an amide with the N-terminal amine of the protein, or the cleavable moiety comprises a cleavable peptide in which the N-terminal amine of the cleavable peptide forms an amide with the C-terminal carboxyl of the protein). In such cases, the cleavable peptide is also attached to the protein at a side chain of an amino acid elsewhere on the protein, optionally via a linking group. In such cases, determination of the additional point of attachment for the purposes of the instant disclosure can be determined by identifying the amino acid residue of the activatable protein which corresponds with the N-terminus or the C-terminus of the base protein on which the activatable protein is based (e.g., the N-terminus or C-terminus of the wild type version of the protein). Alternatively, the additional point of attachment can be determined by identifying the amino acid residue closest to the relevant terminus of the contiguous polypeptide which contains the cleavable peptide and the protein sequence which corresponds to the protein on which the activatable protein is based (e.g., the last amino acid in the sequence of the protein on which the activatable protein is based which matches the corresponding amino acid in the activatable protein). For example, where the protein portion of an activatable protein comprises a truncation of amino acids compared to the wild type version of the corresponding protein, the point of attachment of the cleavable peptide is the most terminal residue of the activatable protein which is also present on the wild type version.

In embodiments where a cleavable peptide is attached to the N-terminus or the C-terminus of the protein, the cleavable peptide can be linked to the protein via a peptide linking group. Non-limiting examples of peptide linking groups include, but are not limited to (GS), (SEQ ID NO: 84), (GGS), (SEQ ID NO: 85), (GGGS)ₙ (SEQ ID NO: 86), (GGSG)ₙ (SEQ ID NO: 87), or (GGSGG)ₙ (SEQ ID NO: 88), (GGGGS)ₙ (SEQ ID NO: 89), wherein n is 1, 2, 3, 4, 5, 6, 7, 8, 9, or 10. For example, a peptide linking group can be (GGGGS)₃ (SEQ ID NO: 90) or (GGGGS)₄ (SEQ ID NO: 91).

The point of attachment of the cleavable moiety to the side chain of an amino acid of the protein and the additional point of attachment are separated by a plurality of amino acids of the protein. In some embodiments, the point of attachment of the cleavable moiety to the side chain of the amino acid of the protein and the additional point of attachment are separated by at least about 5, 10, 15, or 20 amino acids. In some embodiments, the point of attachment of the cleavable moiety to the side chain of the amino acid of the protein and the additional point of attachment are separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids. In some embodiments, the point of attachment of the cleavable moiety to the side chain of the amino acid of the protein and the additional point of attachment are separated by at most 100, 90, 80, 70, 60, 50, 40, 30, 25, or 20 amino acids. In some embodiments, the point of attachment of the cleavable moiety to the side chain of the amino acid of the protein and the additional point of attachment are separated by 5 to 100, 5 to 75, 5 to 50, 5 to 40, 5 to 30, 5 to 25, 5 to 20, 10 to 100, 10 to 75, 10 to 50, 10 to 40, 10 to 30, 10 to 20, 15 to 100, 15 to 75, 15 to 50, 15 to 40, 15 to 30, 15 to 25, 15 to 20, 20 to 100, 20 to 75, 20 to 50, 20 to 40, or 20 to 30 amino acids.

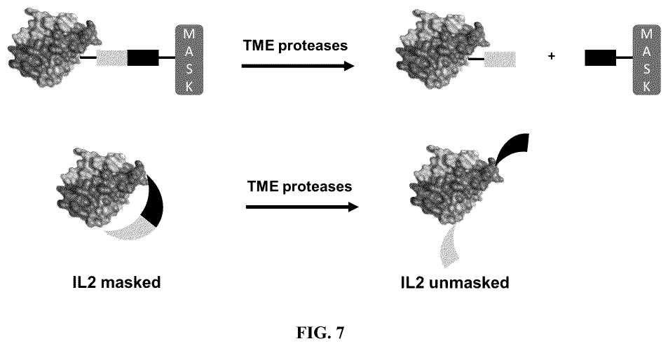

In embodiments wherein the cleavable moiety comprises multiple (e.g., 2) points of attachment to the protein, cleavage of the cleavable linker can leave a portion of the cleavable linker attached to each point of attachment (or to one point of attachment). The portion attached to each point of attachment can be any of the portions remaining attached provided herein. For example, where a cleavable peptide is the cleavable moiety and comprises two points of attachment to the protein, cleavage of the cleavable peptide can leave a number of amino acids attached to the first point of attachment (e.g., 1, 2, 3, 4, 5, 6, 7, 8, 9, 10, or more amino acids) and can leave a number of amino acids attached to the second point of attachment (e.g., 1, 2, 3, 4, 5, 6, 7, 8, 9, 10, or more amino acids). Schematics of mechanisms of actions of activatable IL-2 molecule activation according certain examples of the present disclosure are shown in FIG. 7.

### Additional Moieties Attached to Cleavable Moieties

In some embodiments, the cleavable moiety is further attached to an additional moiety. Non-limiting examples of additional moieties to which the cleavable moiety can also be attached include additional polypeptides (e.g., antibodies or antigen binding fragments thereof, targeting peptides, Fc domains, etc.), polymers (e.g., water soluble polymers, such as poly(ethylene glycol)), lipids, nanoparticles, nucleic acids (e.g., aptamers), small molecules, or other functionalities. In some embodiments, the attachment additional moiety to the cleavable moiety is positioned such that cleavage of the cleavable moiety breaks the connection between the additional moiety and the protein. In some embodiments, cleavage of the cleavable moiety causes the additional moiety to no longer be attached to the protein. The additional moiety can be attached to the cleavable moiety directly (e.g., attached directly to one of the amino acids of a cleavable peptide, such as the N-terminus or C-terminus of the cleavable peptide) or through a suitable linking group.

In some embodiments, the cleavable moiety is further attached to an additional polypeptide. In some embodiments, the additional polypeptide is an antibody or antigen binding fragment thereof.

In some embodiments, the cleavable moiety is further attached to a polymer. In some embodiments, the polymer comprises a water soluble polymer. In some embodiments, the polymer comprises poly(alkylene oxide), polysaccharide, poly(vinyl pyrrolidone), poly(vinyl alcohol), polyoxazoline, poly(acryloylmorpholine), or a combination thereof. In some embodiments, the polymer comprises poly(alkylene oxide). In some embodiments, the polymer comprises polyethylene glycol or polypropylene glycol, or a combination thereof. In some embodiments, the polymer comprises polyethylene glycol. In some embodiments, the polymer is linear. In some embodiments, the polymer is branched. In some embodiments, the polymer has a molecular weight of at least about 0.1 kDa, 0.5 kDa, 1 kDa, 2 kDa, 3 kDa, 5 kDa, 10 kDa, 15 kDa, 20 kDa, 30 kDa. In some embodiments, the polymer has a molecular weight of at most about 50 kDa, 40 kDa, 30 kDa, 20 kDa, 15 kDa, 10 kDa, 5 kDa, 3 kDa, 2 kDa, or 1 kDa. In some embodiments, the polymer has a molecular weight of from about 0.1 kDa to about 50 kDa, about 0.1 kDa to about 40 kDa, about 0.1 kDa to about 30 kDa, about 0.1 kDa to about 20 kDa, about 0.1 kDa to about 15 kDa, about 0.1 kDa to about 10 kDa, about 0.1 kDa to about 5 kDa, about 0.1 kDa to about 3 kDa, about 0.1 kDa to about 2 kDa, about 0.1 kDa to about 1 kDa, 0.5 kDa to about 50 kDa, about 0.5 kDa to about 40 kDa, about 0.5 kDa to about 30 kDa, about 0.5 kDa to about 20 kDa, about 0.5 kDa to about 15 kDa, about 0.5 kDa to about 10 kDa, about 0.5 kDa to about 5 kDa, about 0.5 kDa to about 3 kDa, about 0.5 kDa to about 2 kDa, about 0.5 kDa to about 1 kDa, about 1 kDa to about 50 kDa, about 1 kDa to about 30 kDa, about 1 kDa to about 15 kDa, about 1 kDa to about 10 kDa, about 1 kDa to about 5 kDa about 1 kDa to about 3 kDa, about 1 kDa to about 2 kDa, about 3 kDa to about 50 kDa, about 3 kDa to about 30 kDa, about 3 kDa to about 15 kDa, about 3 kDa to about 10 kDa, about 5 kDa to about 50 kDa, about 5 kDa to about 30 kDa, about 5 kDa to about 20 kDa, about 10 kDa to about 50 kDa, or about 10 kDa to about 30 kDa.

In some embodiments, the polymer is an end-capped polymer. In some embodiments, the polymer is an end-capped polyethylene glycol. In some embodiments, the polymer is end-capped with a functional group selected from amine, alkoxy (e.g., methoxy, ethoxy, propoxy, etc.), hydroxyl, amide (e.g., —NH(C═O)(C₁-C₄ alkyl)), carboxylate, and ester (e.g., methyl ester, ethyl ester, etc.). In some embodiments, the polymer as an amine end-capped PEG. In some embodiments, the polymer is an acetyl end-capped PEG.

In some embodiments, the presence of the additional moiety enhances the plasma half-life of the activatable protein. In some embodiments, the presence of the additional moiety enhances the plasma half-life of the activatable protein by at least about 10%, 20%, 30%, 40%, 50%, 75%, 100%, 200%, 300%, 500%, 750%, or 1000% compared to the activatable protein with no additional moiety attached. In some embodiments, the plasma half-life of the activatable protein by at least about 2-fold, 5-fold, 10-fold, 50-fold, 100-fold, 500-fold, 1000-fold, 5000-fold, or 10000-fold compared to the activatable protein with no additional moiety attached.

In some embodiments, the presence of the additional moiety alters the activity of the activatable protein relative to the protein (e.g., after cleavage of the cleavable moiety) to a greater degree than is observed without the additional moiety. For example, the presence of an additional moiety such as a polymer attached to the cleavable moiety could lead to the activatable protein displaying a 100-fold lower activity compared to the protein after cleavage of the cleavable peptide, whereas the activatable protein without the additional moiety may only display a 20-fold lower activity compared to the protein after cleavage of the cleavable peptide. In some embodiments, the presence of the additional moiety alters the activity of the activatable protein by at least 2-fold, 3-fold, 4-fold, or 5-fold more than is observed for the activatable protein without the additional moiety attached.

### Methods of Manufacturing Activatable Proteins

Also provided herein are methods of manufacturing activatable proteins. The activatable proteins provided herein may be prepared using any suitable method.

In one aspect, provided herein, is a method of manufacturing an activatable protein, comprising: synthesizing two or more precursor peptides, wherein one of the precursor peptides comprises a cleavable moiety as provided herein attached to a side chain of the precursor peptide, and ligating the two or more precursor peptides to provide the activatable protein.

In another aspect provided herein is a method of manufacturing an activatable protein provided herein, comprising: providing a protein as provided herein and attaching a cleavable moiety to a side chain of an amino acid of the protein. In another aspect provided herein is a method of manufacturing an activatable protein by providing a protein with a cleavable moiety (e.g., a cleavable peptide) attached to the N- or C-terminus of the protein. In some embodiments, the cleavable moiety is then attached to a side chain of an amino acid of the protein (e.g., to form a cyclic structure).

In some embodiments, providing the protein comprises providing a recombinant or synthetic protein. In some embodiments, providing the protein comprises providing a recombinant protein. In some embodiments, providing the protein comprises providing a synthetic protein.

In some embodiments, attaching the cleavable moiety to a side chain of an amino acid of the protein comprises conjugating the cleavable moiety to the side chain of the amino acid of the protein. In some embodiments, conjugating the cleavable moiety to the side chain of the amino acid of the protein comprises performing a conjugation reaction (e.g., reacting two complementary conjugation handles, such as two complementary conjugation handles provided herein) between the cleavable moiety and the protein. In some embodiments, the protein comprises a substitution to incorporate a conjugatable amino acid at a desired point of attachment. In some embodiments, the conjugatable amino acid is an unnatural amino acid comprising a conjugation handle. Unnatural amino acids with conjugation handles can be incorporated into synthetic proteins (e.g., by incorporation during synthesis of the protein or a precursor peptide) or into recombinant proteins using methods known in the art. For example, recombinant proteins with unnatural amino acids can be made using methods as described in Patent Cooperation Treaty Publication Nos. WO2016/115168, WO2002/085923, WO2005/019415, and WO2005/003294, each of which is incorporated by reference as if set forth herein in its entirety. Alternatively or in combination, unnatural or modified natural amino acids can be incorporated into chemically synthesized proteins during synthesis. In some embodiments, the conjugatable amino acid is a cysteine.

In some embodiments, the cleavable moiety is configured to be conjugatable to a desired amino acid. In some embodiments, the cleavable moiety comprises a conjugation handle for attachment to the side chain of the amino acid residue. In some embodiments, the cleavable moiety comprises a conjugation handle reactive with a sulfhydryl group (e.g., maleimide, α,β-unsaturated carbonyl, α-halo carbonyl, etc.). Cleavable moieties with conjugation handles reactive with sulfhydryls can be readily employed with proteins which contain cysteine residues, either at natural positions or positions which have been substituted with the cysteine. In some embodiments, use of sulfhydryl reactive conjugation handles is preferred for use with recombinant proteins because it eliminates the need to design a system for incorporation of an unnatural amino acid.

### Activatable IL-2 Polypeptides

Also provided herein are activatable IL-2 polypeptides. The activatable IL-2 polypeptides provided herein utilize the strategies and components (e.g., cleavable moieties (including cleavable peptides), linking groups, methods of manufactures, etc.) provided herein in order to provide an IL-2 polypeptide which is deactivated with the cleavable moiety attached and which shows greater activity upon cleavage of the cleavable moiety. It is expressly contemplated that any of the features provided above for activatable proteins are applicable to activatable IL-2 polypeptides, and inclusion of any features below should not be taken to imply that any of the above referenced features are inapplicable to activatable IL-2 polypeptides.

In some embodiments, the IL-2 polypeptide comprises modification(s) which diminish the ability of the IL-2 polypeptide to bind to or signal through the IL-2 receptor alpha unit (or the IL-2 receptor αβγ complex) but spare the ability of the IL-2 polypeptide to bind to and signal through the IL-2 receptor beta subunit (or the IL-2 receptor βγ complex). Signaling through the IL-2 receptor alpha subunit (or the IL-2 receptor αβγ complex) is associated with activation of regulatory T cells (Tregs), whereas signaling through the IL-2 receptor beta subunit (or the IL-2 receptor βγ complex) is associated with activation of effector T cells (Teffs) (e.g., CD8+ effector T cells) and/or natural killer (NK) cells. Thus, in some embodiments, an IL-2 polypeptide of an activatable IL-2 polypeptide provided herein exhibits an ability to enhance Teff and/or NK cell proliferation while sparing Tregs after cleavage of the cleavable moiety. In some embodiments, the activatable IL-2 polypeptide with the cleavable moiety attached exhibits a reduced ability enhance Teff and/or NK cell proliferation compared to the IL-2 polypeptide after cleavage of the cleavable moiety. In some embodiments, the cleavable moiety is configured to be selectively or preferentially cleaved in or near a microenvironment of a tumor

In some embodiments, the activatable IL-2 polypeptide comprises a cleavable moiety attached to the IL-2 polypeptide (e.g., at a side chain of an amino acid residue) that reduces the activity of the IL-2 polypeptide. In some embodiments, the intact cleavable moiety reduces the ability of the IL-2 polypeptide to signal through the IL-2 receptor beta subunit (or the IL-2 receptor βγ complex). In some embodiments, cleavage of the cleavable moiety enhances the ability of the IL-2 polypeptide to signal through the IL-2 receptor beta subunit (or the IL-2 receptor βγ complex).

In one aspect, provided herein, is an IL-2 polypeptide which displays substantially no binding to the IL-2 receptor alpha (or the IL-2 receptor αβγ complex) and a cleavable moiety which reduces the ability of the IL-2 polypeptide to bind to the IL-2 receptor beta (or the IL-2 receptor βγ complex), and wherein cleavage of the cleavable moiety enhances the ability of the IL-2 polypeptide to bind to the IL-2 receptor beta (or the IL-2 receptor βγ complex).

In one aspect provided herein is an activatable IL-2 polypeptide, comprising: an IL-2 polypeptide comprising a cleavable moiety attached to a side chain of an amino acid residue of the IL-2 polypeptide.

In one aspect provided herein is an activatable IL-2 polypeptide, comprising: an IL-2 polypeptide comprising a cleavable moiety attached to a side chain of an amino acid residue of the IL-2 polypeptide, wherein the IL-2 polypeptide displays an altered activity after cleavage of the cleavable moiety compared to the activity of the activatable IL-2 polypeptide before cleavage of the cleavable moiety.

In one aspect provided herein is an activatable IL-2 polypeptide, comprising: an IL-2 polypeptide comprising a cleavable moiety attached to a side chain of an amino acid residue of the IL-2 polypeptide, wherein the IL-2 polypeptide displays an enhanced ability to bind to at least one IL-2 receptor subunit after cleavage of the cleavable moiety compared to the activity of the activatable IL-2 polypeptide before cleavage of the cleavable moiety.

In one aspect provided herein is an activatable IL-2 polypeptide, comprising an IL-2 polypeptide comprising a cleavable moiety attached to a residue in the region of residues 1-35 of the IL-2 polypeptide, wherein residue position numbering is based on SEQ ID NO: 1 as a reference sequences, and wherein the IL-2 polypeptide exhibits a greater affinity for the IL-2 receptor beta subunit after cleavage of the cleavable moiety compared to the activatable IL-2 polypeptide before cleavage of the cleavable moiety.

In one aspect provided herein is an activatable IL-2 polypeptide comprising an IL-2 polypeptide comprising a cleavable moiety, wherein the IL-2 polypeptide exhibits a greater affinity for the IL-2 receptor beta subunit after cleavage of the cleavable moiety compared to the activatable IL-2 polypeptide before cleavage of the cleavable moiety.

### Cleavable Moieties Attached to IL-2 Polypeptides

In some embodiments, the IL-2 polypeptide of the activatable IL-2 polypeptide comprises a cleavable moiety covalently attached (e.g., to a side chain of an amino acid residue). The cleavable moiety can be any suitable cleavable moiety, including those provided herein above (e.g., a comprising a cleavable peptide set forth in Table 1B or Table 1C), and can be linked to the IL-2 polypeptide by any suitable linking group, including any of the linking groups described herein above.

In some embodiments, the cleavable moiety attached to the IL-2 polypeptide is a cleavable peptide (e.g., any of the cleavable peptides discussed supra). In some embodiments, the cleavable peptide is cleavable by one or more proteases. In some embodiments, the cleavable peptide is cleavable by one or more proteases associated with a tumor or tumor microenvironment. In some embodiments, the cleavable peptide is preferentially or selectively cleaved in or near a tumor microenvironment. In some embodiments, the cleavable peptide is preferentially or selectively cleaved by one or more proteases associated with a tumor or tumor microenvironment.

In some embodiments, the cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof. In some embodiments, the cleavable peptide is cleavable by an MMP. In some embodiments, the cleavable peptide is cleavable by a matriptase. In some embodiments, the cleavable peptide is cleavable by a plasminogen activator. In some embodiments, the cleavable peptide is cleavable by a legumain. In some embodiments, the cleavable peptide is cleavable by a protease set forth in Table 1A.

In some embodiments, the cleavable peptide is cleavable by multiple proteases. In some embodiments, the cleavable peptide is cleavable by multiple classes of proteases. In some embodiments, the cleavable peptide is cleavable by 2, 3, or 4 different proteases. In some embodiments, the cleavable peptide comprises multiple cleavage sites. In some embodiments, the cleavable peptide comprises 2, 3, 4, or more cleavage sites. In some embodiments, the cleavable peptide comprises 2 cleavage sites. In some embodiments, the cleavable peptide comprises 3 cleavage sites. In some embodiments, the cleavable peptide comprises 4 cleavage sites. In some embodiments, each of the cleavage sites is cleavable by a different protease.

In some embodiments, the cleavable peptide is cleavable by a matrix metalloprotease and a legumain. In some embodiments, the cleavable peptide is cleavable by a matrix metalloprotease and a matriptase. In some embodiments, the cleavable peptide is cleavable by a matrix metalloprotease and a plasminogen activator. In some embodiments, the cleavable peptide is cleavable by a legumain and a matriptase. In some embodiments, the cleavable peptide is cleavable by a legumain and a plasminogen activator. In some embodiments, the cleavable peptide is cleavable by a matriptase and a plasminogen activator. In some embodiments, the cleavable peptide is cleavable by a matrix metalloprotease, a legumain, and a matriptase. In some embodiments, the cleavable peptide is cleavable by a matrix metalloprotease, a matriptase, and a plasminogen activator. In some embodiments, the cleavable peptide is cleavable by a matrix metalloprotease, a legumain, and a plasminogen activator.

In some embodiments, the cleavable peptide has a length of from 4 to 30 amino acids. In some embodiments, the cleavable peptide has a length of at most 30, 25, 20, 19, 18, 17, 16, 15, 14, 13, 12, 11, 10, 9, 8, 7, 6, or 5 amino acids. In some embodiments, the cleavable peptide has a length of at least 4, 5, 6, 7, 8, 9, or 10 amino acids. In some embodiments, the cleavable peptide has a length of from 4 to 30, 4 to 25, 4 to 20, 4 to 18, 4 to 16, 4 to 15, 4 to 12, 4 to 10, 5 to 30, 5 to 25, 5 to 20, 5 to 18, 5 to 16, 5 to 15, 5 to 12, or 5 to 10 amino acids.

In some embodiments, cleavage of the cleavable peptide leaves a residual portion of the cleavable peptide attached to the IL-2 polypeptide. In some embodiments, cleavage of the cleavable peptide leaves a residual portion of the cleavable peptide attached to the side chain of the amino acid residue to which the cleavable peptide is attached. In some embodiments, cleavage of the cleavable peptide leaves at least one amino acid of the cleavable peptide still attached to the IL-2 polypeptide. In some embodiments, cleavage of the cleavable peptide leaves at least one amino acid of the cleavable peptide still attached to the side chain of the amino acid residue to which the cleavable peptide is attached. In some embodiments, cleavage of the cleavable peptide leaves 1, 2, 3, 4, 5, 6, 7, 8, 9, or 10 amino acids attached to the side chain of the amino acid residue to which the cleavable moiety is attached. In some embodiments, cleavage of the cleavable peptide leaves at most 10 amino acids of the cleavable peptide attached to the IL-2 polypeptide. In some embodiments, cleavage of the cleavable peptide leaves at most 5 amino acids of the cleavable peptide attached to the IL-2 polypeptide. In some embodiments, cleavage of the cleavable peptide leaves 1 amino acid of the cleavable peptide attached to the IL-2 polypeptide. In some embodiments, cleavage of the cleavable peptide leaves 2 amino acids of the cleavable peptide attached to the IL-2 polypeptide. In some embodiments, cleavage of the cleavable peptide leaves 3 amino acids of the cleavable peptide attached to the IL-2 polypeptide. In some embodiments, cleavage of the cleavable peptide leaves 4 amino acids of the cleavable peptide attached to the IL-2 polypeptide. In some embodiments, cleavage of the cleavable peptide leaves 5 amino acids of the cleavable peptide attached to the IL-2 polypeptide. In some embodiments, the reference to amino acids attached to the IL-2 polypeptide described in this paragraph refers only to those at a single point of attachment (e.g., in a case where the cleavable peptide is attached to the IL-2 polypeptide at two points of attachment, the number of amino acids attached in this paragraph refers to the number of amino acids attached to either or both of the first and second points of attachment).

In some embodiments, the cleavable peptide comprises an amino acid sequence having at least about 80%, at least about 90%, or 100% identity to a sequence set forth in Table 1B or Table 1C. In some embodiments, the cleavable peptide comprises an amino acid sequence set forth in Table 1B. In some embodiments, the cleavable peptide comprises an amino acid sequence set forth in Table 1C.

The cleavable peptide can be attached to the IL-2 polypeptide (e.g., a side chain of an amino acid residue of the IL-2 polypeptide) in a variety of ways. In some embodiments, the cleavable peptide is covalently attached to the IL-2 polypeptide (e.g., a side chain of an amino acid residue of the IL-2 polypeptide). In some embodiments, the cleavable peptide is attached to the IL-2 polypeptide through its C-terminal carboxyl, its N-terminal amine, or through a side chain of the cleavable peptide. In some embodiments, the cleavable peptide is directly attached to the IL-2 polypeptide. In some embodiments, the cleavable peptide is directly attached to a side chain of an amino acid residue of the IL-2 polypeptide.

In some embodiments, the cleavable peptide is attached to the IL-2 polypeptide through the C-terminus of the cleavable peptide. In some embodiments, the C-terminal carboxyl group of the cleavable peptide is directly attached to a side chain of an amino acid residue of the IL-2 polypeptide. In some embodiments, the C-terminal carboxyl group of the cleavable peptide is directly attached to a side chain amine of an amino acid residue of the IL-2 polypeptide (e.g., as an amide bond). In some embodiments, the side chain amine is of a lysine residue of the IL-2 polypeptide. In some embodiments, the side chain amine is of an unnatural amino acid residue of the IL-2 polypeptide (e.g., ornithine, homolysine, or another amine containing unnatural amino acid). In some embodiments, the C-terminal carboxyl group of the cleavable peptide is attached to a side chain of an amino acid residue of the IL-2 polypeptide through a linking group (e.g., any of the linking groups provided herein). In some embodiments, the C-terminal carboxyl group of the cleavable peptide attached to the linking group by an amide bond formed between the C-terminal carboxyl and an amine group of the linking group.

In some embodiments, the cleavable peptide is attached to the IL-2 polypeptide through the N-terminus of the cleavable peptide. In some embodiments, the N-terminal amine group of the cleavable peptide is directly attached to a side chain of an amino acid residue of the IL-2 polypeptide. In some embodiments, the N-terminal amine group of the cleavable peptide is directly attached a side chain carboxyl of an amino acid residue of the IL-2 polypeptide (e.g., as an amide bond). In some embodiments, the side chain carboxyl is of a glutamate or aspartate residue of the IL-2 polypeptide. In some embodiments, the side chain carboxyl is of an unnatural amino acid residue of the IL-2 polypeptide (e.g., 2-amino adipic acid, 2-amino pimelic acid, or another carboxyl containing unnatural amino acid). In some embodiments, the N-terminal amine group of the cleavable peptide is attached to a side chain of an amino acid residue of the IL-2 polypeptide through a linking group (e.g., any of the linking groups provided herein). In some embodiments, the N-terminal carboxyl group of the cleavable peptide attached to the linking group by an amide bond formed between the N-terminal amine and a carboxyl group of the linking group.

In some embodiments, the cleavable peptide is attached to the IL-2 polypeptide through a side chain of an amino acid residue of the cleavable peptide. In some embodiments, a side chain of an amino acid residue of the cleavable peptide is directly attached to the IL-2 polypeptide (e.g., to a side chain of an amino acid residue of the protein). In some embodiments, a side chain of an amino acid residue of the cleavable peptide is attached to the IL-2 polypeptide through a linking group (e.g., any of the linking groups provided herein).

### Points of Attachment of Cleavable Moieties to IL-2 Polypeptides

In some embodiments, the cleavable moiety is attached to a side chain of an amino acid residue of the IL-2 polypeptide (either directly or through a suitable linking group). The cleavable moiety can be attached to the side chain of an amino acid at any desired position, and can be attached to any suitable amino acid type (e.g., any of the points of attachment discussed supra, such as attachment to any of the amino acid types provided above).

In some embodiments, the cleavable moiety is attached to the IL-2 polypeptide at a residue of the IL-2 polypeptide such that binding of the IL-2 polypeptide to at least one IL-2 receptor subunit is reduced (e.g., compared to a corresponding IL-2 polypeptide without the cleavable moiety attached). In some embodiments, the cleavable moiety is attached to the IL-2 polypeptide at a residue of the IL-2 polypeptide such that binding of the IL-2 polypeptide to an IL-2 receptor beta and/or gamma subunit is reduced. In some embodiments, the cleavable moiety is attached to the IL-2 polypeptide at a residue of the IL-2 polypeptide such that binding of the IL-2 polypeptide to an IL-2 receptor beta subunit is reduced. In some embodiments, the cleavable moiety is attached to the IL-2 polypeptide at a residue of the IL-2 polypeptide such that binding of the IL-2 polypeptide to an IL-2 receptor gamma subunit is reduced.

In some embodiments, the cleavable moiety is attached to the IL-2 polypeptide at a residue of the IL-2 polypeptide such that binding to an IL-2 receptor complex is reduced (e.g., compared to a corresponding IL-2 polypeptide without the cleavable moiety attached). In some embodiments, the cleavable moiety is attached to the IL-2 polypeptide at a residue of the IL-2 polypeptide such that binding to an IL-2 receptor βγ complex is reduced.

In some embodiments, the cleavable moiety is attached to the IL-2 polypeptide at or near a residue of the IL-2 polypeptide which interacts with an IL-2 receptor beta or gamma subunit, or an IL-2 receptor βγ complex. Interactions of IL-2 with the IL-2 receptor and its associated subunits have been explored and analyzed by X-ray co-crystal structures, for example at least Stauber et al. in “Crystal structure of the IL-2 signaling complex: Paradigm for a heterotrimeric cytokine receptor” (Proc Natl Acad Sci USA. 2006 Feb. 21; 103 (8): 2788-279), which is hereby incorporated by reference as if set forth herein in its entirety. In some embodiments, the cleavable moiety is attached to an amino acid residue which is within at most 20, 15, 10, 9, 8, 7, 6, 5, 4, 3, 2, or 1 amino acids of a residue which interacts with the IL-2 receptor beta or gamma subunit, or the IL-2 receptor βγ complex. In some embodiments, the cleavable moiety is attached to an amino acid residue which interacts with the IL-2 receptor beta or gamma subunit, or the IL-2 receptor βγ complex.

In some embodiments, the cleavable moiety is attached to a residue selected from residues 9, 11, 13, 15, 16, 19, 20, 22, 23, 26, 29, 32, 84, 88, 91, 123, 126, and 129 of the IL-2 polypeptide, wherein residue position numbering is based on SEQ ID NO: 1 as a reference sequence. In some embodiments, the cleavable moiety is attached to residue 9, 11, 13, 15, 16, 19, 22, 23, 29, or 32 of the IL-2 polypeptide. In some embodiments, the cleavable moiety is attached to residue 9 of the IL-2 polypeptide. In some embodiments, the cleavable moiety is attached to residue 11 of the IL-2 polypeptide. In some embodiments, the cleavable moiety is attached to residue 13 of the IL-2 polypeptide. In some embodiments, the cleavable moiety is attached to residue 15 of the IL-2 polypeptide. In some embodiments, the cleavable moiety is attached to residue 16 of the IL-2 polypeptide. In some embodiments, the cleavable moiety is attached to residue 20 of the IL-2 polypeptide. In some embodiments, the cleavable moiety is attached to residue 22 of the IL-2 polypeptide. In some embodiments, the cleavable moiety is attached to residue 23 of the IL-2 polypeptide. In some embodiments, the cleavable moiety is attached to residue 26 of the IL-2 polypeptide. In some embodiments, the cleavable moiety is attached to residue 29 of the IL-2 polypeptide. In some embodiments, the cleavable moiety is attached to residue 32 of the IL-2 polypeptide. In some embodiments, the cleavable moiety is attached to residue 84 of the IL-2 polypeptide. In some embodiments, the cleavable moiety is attached to residue 88 of the IL-2 polypeptide. In some embodiments, the cleavable moiety is attached to residue 91 of the IL-2 polypeptide. In some embodiments, the cleavable moiety is attached to residue 123 of the IL-2 polypeptide. In some embodiments, the cleavable moiety is attached to residue 126 of the IL-2 polypeptide. In some embodiments, the cleavable moiety is attached to residue 129 of the IL-2 polypeptide.

In some embodiments, the cleavable moiety is attached to a residue in the region of residues 1-35 of the IL-2 polypeptide, wherein residue position numbering is based on SEQ ID NO: 1 as a reference sequence. In some embodiments, the cleavable moiety is attached to any one of residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35. In some embodiments, the cleavable moiety is attached to a residue in the region of residues 5-35, 5-32, 5-30, 5-25, 5-23, 9-35, 9-32, 9-30, 9-25, 9-23, 15-35, 15-32, 15-30, 15-25, or 15-23 of the IL-2 polypeptide.

In some embodiments, the amino acid residue to which the cleavable moiety is attached is substituted relative to the corresponding amino acid residue of SEQ ID NO: 1. The substitution can be any suitable amino acid, including any of those provided supra (e.g., lysine, glutamate, glutamine, aspartate, asparagine, tyrosine, serine, threonine, cysteine, an unnatural amino acid, etc.).

### Additional Points of Attachment of Cleavable Moieties to IL-2 Polypeptides

In some embodiments, the cleavable moiety is further attached to the IL-2 polypeptide at an additional point of attachment to the IL-2 polypeptide (e.g., a second point of attachment distinct from the side chain of the amino acid residue to which the cleavable moiety is attached discussed above). In some embodiments, cleavage of the cleavable moiety breaks the connection between the cleavable moiety and the two points of attachment.

In some embodiments, attachment of the cleavable moiety to the additional point of attachment results in a macrocyclic structure being formed. In some embodiments, the macrocyclic structure is formed between the cleavable moiety, any intervening linking groups (if present) between the cleavable moiety and either or both points of attachment to the IL-2 polypeptide, and any intervening amino acids of the IL-2 polypeptide. In some embodiments, cleavage of the cleavable moiety breaks the macrocyclic structure.

In some embodiments, the additional point of attachment is to a terminal reside of the IL-2 polypeptide. In some embodiments, the additional point of attachment is to the N-terminus or the C-terminus of the IL-2 polypeptide. In some embodiments, the additional point of attachment is to the N-terminus of the IL-2 polypeptide. In some embodiments, the additional point of attachment is to the C-terminus of the IL-2 polypeptide.

In some embodiments, the additional point of attachment of the cleavable moiety is to a side chain of another amino acid residue of the IL-2 polypeptide. In some embodiments, the additional point of attachment is to a side chain of another amino acid residue which is less than 30, 25, 20, or 15 amino acids away from the first amino acid connected to the cleavable moiety.

In some embodiments, the additional point of attachment is to a residue in the region of residues 1-40 of the IL-2 polypeptide. In some embodiments, the additional point of attachment is to a residue in the region of residues 5-40, 5-35, 5-30, 5-25, 5-20, 5-15, or 5-10. In some embodiments, the additional point of attachment is to any one of residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 of the IL-2 polypeptide. In some embodiments, the additional point of attachment is to residue 9 of the IL-2 polypeptide.

In some embodiments, the cleavable moiety is attached to residue 23 of the IL-2 polypeptide and comprises an additional point of attachment to residue 9 of the IL-2 polypeptide. In some embodiments, the cleavable moiety is attached to residue 23 of the IL-2 polypeptide and comprises an additional point of attachment to the N-terminus of the IL-2 polypeptide.

In some embodiments, the cleavable moiety is attached to residue 11 of the IL-2 polypeptide and comprises an additional point of attachment to residue 9 of the IL-2 polypeptide. In some embodiments, the cleavable moiety is attached to residue 11 of the IL-2 polypeptide and comprises an additional point of attachment to the N-terminus of the IL-2 polypeptide.

In some embodiments, the cleavable moiety is attached to residue 13 of the IL-2 polypeptide and comprises an additional point of attachment to residue 9 of the IL-2 polypeptide. In some embodiments, the cleavable moiety is attached to residue 13 of the IL-2 polypeptide and comprises an additional point of attachment to the N-terminus of the IL-2 polypeptide.

In some embodiments, the cleavable moiety is attached to residue 15 of the IL-2 polypeptide and comprises an additional point of attachment to residue 9 of the IL-2 polypeptide. In some embodiments, the cleavable moiety is attached to residue 15 of the IL-2 polypeptide and comprises an additional point of attachment to the N-terminus of the IL-2 polypeptide.

In some embodiments, the cleavable moiety is attached to residue 19 of the IL-2 polypeptide and comprises an additional point of attachment to residue 9 of the IL-2 polypeptide. In some embodiments, the cleavable moiety is attached to residue 19 of the IL-2 polypeptide and comprises an additional point of attachment to the N-terminus of the IL-2 polypeptide.

In some embodiments, the cleavable moiety is attached to residue 22 of the IL-2 polypeptide and comprises an additional point of attachment to residue 9 of the IL-2 polypeptide. In some embodiments, the cleavable moiety is attached to residue 22 of the IL-2 polypeptide and comprises an additional point of attachment to the N-terminus of the IL-2 polypeptide.

In some embodiments, the cleavable moiety is attached to residue 26 of the IL-2 polypeptide and comprises an additional point of attachment to residue 9 of the IL-2 polypeptide. In some embodiments, the cleavable moiety is attached to residue 26 of the IL-2 polypeptide and comprises an additional point of attachment to the N-terminus of the IL-2 polypeptide.

In some embodiments, the cleavable moiety is attached to residue 29 of the IL-2 polypeptide and comprises an additional point of attachment to residue 9 of the IL-2 polypeptide. In some embodiments, the cleavable moiety is attached to residue 29 of the IL-2 polypeptide and comprises an additional point of attachment to the N-terminus of the IL-2 polypeptide.

In some embodiments, the cleavable moiety is attached to residue 32 of the IL-2 polypeptide and comprises an additional point of attachment to residue 9 of the IL-2 polypeptide. In some embodiments, the cleavable moiety is attached to residue 32 of the IL-2 polypeptide and comprises an additional point of attachment to the N-terminus of the IL-2 polypeptide.

Exemplary points of attachment, additional points of attachment, and orientations of cleavable peptides to IL-2 polypeptides as provided herein are provided in the table below.

| | First | Position of | | Second | Position of | Linking Group |
|---|---|---|---|---|---|---|
| | Residue | Cleavable | Linking Group | Residue | Cleavable | Between |
| | Position | Peptide | Between First | Position | Peptide | Second |
| | of IL-2 | Attached | Residue | of IL-2 | Attached to | Residue |
| | Attached | to First | Position | Attached | Second | Position |
| Exemplary | to | Residue | of IL-2 and | to | Residue | of IL-2 and |
| Orientation | Cleavable | Position of | Cleavable | Cleavable | Position of | Cleavable |
| Number | Peptide | IL-2 | Peptide | Peptide | IL-2 | Peptide |
| 1 | 32 | N-terminus | PEG9-Glutaryl | 1 (N-terminus | C-terminus | PEG9 |
| | | | | of IL-2) | | |
| 2 | 32 | N-terminus | PEG4-Glutaryl | 1 (N-terminus | C-terminus | PEG4 |
| | | | | of IL-2) | | |
| 3 | 32 | N-terminus | PEG16-Glutaryl | 1 (N-terminus | C-terminus | PEG16 |
| | | | | of IL-2) | | |
| 4 | 32 | N-terminus | PEG9-Glutaryl | 1 (N-terminus | C-terminus | PEG24 |
| | | | | of IL-2) | | |
| 5 | 32 | N-terminus | PEG9-Glutaryl | 1 (N-terminus | C-terminus | None |
| | | | | of IL-2) | | |
| 6 | 32 | N-terminus | Glutaryl | 1 (N-terminus | C-terminus | PEG9 |
| | | | | of IL-2) | | |
| 7 | 29 | N-terminus | PEG9 | 1 (N-terminus | C-terminus | PEG9 |
| | | | | of IL-2) | | |
| 8 | 29 | N-terminus | PEG24 | 1 (N-terminus | C-terminus | PEG24 |
| | | | | of IL-2) | | |
| 9 | 29 | N-terminus | PEG4 | 1 (N-terminus | C-terminus | PEG4 |
| | | | | of IL-2) | | |
| 10 | 29 | N-terminus | PEG16 | 1 (N-terminus | C-terminus | PEG16 |
| | | | | of IL-2) | | |
| 11 | 29 | N-terminus | PEG9 | 1 (N-terminus | C-terminus | None |
| | | | | of IL-2) | | |
| 12 | 29 | N-terminus | None | 1 (N-terminus | C-terminus | PEG9 |
| | | | | of IL-2) | | |
| 13 | 19 | N-terminus | None | 1 (N-terminus | C-terminus | PEG4 |
| | | | | of IL-2) | | |
| 14 | 19 | N-terminus | PEG4 | 1 (N-terminus | C-terminus | None |
| | | | | of IL-2) | | |
| 15 | 22 | N-terminus | None | 1 (N-terminus | C-terminus | PEG4 |
| | | | | of IL-2) | | |
| 16 | 22 | N-terminus | PEG4 | 1 (N-terminus | C-terminus | None |
| | | | | of IL-2) | | |
| 17 | 26 | N-terminus | None | 1 (N-terminus | C-terminus | PEG9 |
| | | | | of IL-2) | | |
| 18 | 26 | N-terminus | PEG9 | 1 (N-terminus | C-terminus | None |
| | | | | of IL-2) | | |
| 19 | 23 | C-terminus | None | 1 (N-terminus | N-terminus | PEG9-Glutaryl |
| | | | | of IL-2) | | |
| 20 | 23 | C-terminus | None | 1 (N-terminus | N-terminus | PEG4-Glutaryl |
| | | | | of IL-2) | | |
| 21 | 23 | C-terminus | None | 1 (N-terminus | N-terminus | Glutaryl |
| | | | | of IL-2) | | |
| 22 | 23 | C-terminus | PEG4 | 9 | N-terminus | None |
| 23 | 23 | C-terminus | None | 9 | N-terminus | PEG4 |
| 24 | 23 | C-terminus | None | 9 | N-terminus | None |
| 25 | 23 | N-terminus | None | 1 (N-terminus | C-terminus | PEG4 |
| | | | | of IL-2) | | |
| 26 | 23 | N-terminus | PEG4 | 1 (N-terminus | C-terminus | None |
| | | | | of IL-2) | | |
| 27 | 23 | N-terminus | None | 1 (N-terminus | C-terminus | PEG9 |
| | | | | of IL-2) | | |
| 28 | 23 | N-terminus | PEG9 | 1 (N-terminus | C-terminus | None |
| | | | | of IL-2) | | |
| 29 | 23 | N-terminus | None | 1 (N-terminus | C-terminus | None |
| | | | | of IL-2) | | |

In some embodiments, the additional point of attachment of the cleavable moiety is to a residue which is substituted relative to the corresponding amino acid residue of SEQ ID NO: 1. The substitution can be any suitable amino acid, including any of those provided supra (e.g., lysine, glutamate, glutamine, aspartate, asparagine, tyrosine, serine, threonine, cysteine, an unnatural amino acid, etc.). In some embodiments, the additional point of attachment of the cleavable moiety is to a residue which is the natural residue at the corresponding position in SEQ ID NO: 1.

In some embodiments, an activatable IL-2 polypeptide herein comprises a cleavable moiety (e.g., a cleavable peptide) attached to residue 23 and the N-terminus (e.g., at a position corresponding to residue 1 of SEQ ID NO: 1, or another residue of SEQ ID NO: 1 wherein the sequence of SEQ ID NO: 1 has been truncated). In some embodiments, the N-terminus of the IL-2 polypeptide is the amino acid residue at a position corresponding to the first residue of SEQ ID NO: 1. In some embodiments, the cleavable moiety comprises a cleavable peptide. In some embodiments, the cleavable peptide comprises any one of the sequences described herein (e.g., in Table 1C or Table 1D, such as SEQ ID NO: 317 or 333). In some embodiments, the cleavable peptide is one cleavable by matriptase and/or an MMP. In some embodiments, the cleavable peptide is directly attached to the N-terminus of the IL-2 polypeptide (e.g., the C-terminus of the cleavable peptide forms an amide bond with the N-terminus of the IL-2 polypeptide). In some embodiments, the cleavable peptide is directly attached to residue 23 of the IL-2 polypeptide (e.g., the N-terminus of the cleavable peptide forms an amide bond with a carboxylic acid group of the side chain of residue 23). In some embodiments, residue 23 comprises a side chain with a carboxylic acid. In some embodiments, residue 23 of the IL-2 polypeptide is glutamate.

### Additional Moieties Attached to the Cleavable Moieties

In some embodiments, the cleavable moiety attached to the IL-2 polypeptide is further attached to an additional moiety. The additional moiety to which the cleavable moiety is attached can be any of the additional moieties discussed supra (e.g., a polymer, an additional polypeptide, etc.). In some embodiments, the additional moiety is positioned on the cleavable moiety such that cleavage of the cleavable moiety causes the additional moiety to no longer be attached to the IL-2 polypeptide.

In some embodiments, the cleavable moiety is further attached to a polymer. In some embodiments, the polymer is a chemical polymer. In some embodiments, the polymer comprises a water soluble polymer. In some embodiments, the polymer comprises poly(alkylene oxide), polysaccharide, poly(vinyl pyrrolidone), poly(vinyl alcohol), polyoxazoline, poly(acryloylmorpholine), or a combination thereof. In some embodiments, the polymer comprises poly(alkylene oxide). In some embodiments, the polymer is polyethylene glycol or polypropylene glycol, or a combination thereof. In some embodiments, the polymer is polyethylene glycol.

In some embodiments, the polymer attached to the cleavable moiety is linear. In some embodiments, the polymer is branched. In some embodiments, a branched polymer comprises a plurality of polymeric chains attached to a support structure with multiple points of attachment. In some embodiments, the branched polymer comprises a lysine with two polymers covalently attached. In some embodiments, the support structure comprises one or more lysine residues. In some embodiments, the support structure comprises a plurality of lysines coupled together. In some embodiments, the support structure comprises 1, 2, 3, 4, or more lysines coupled together, either through the alpha amine or the side chain amine to the carboxyl of another lysine. In some embodiments, the branched polymer comprises 2, 3, 4, or more polymeric chains. In some embodiments, the branched polymer comprises a structure of

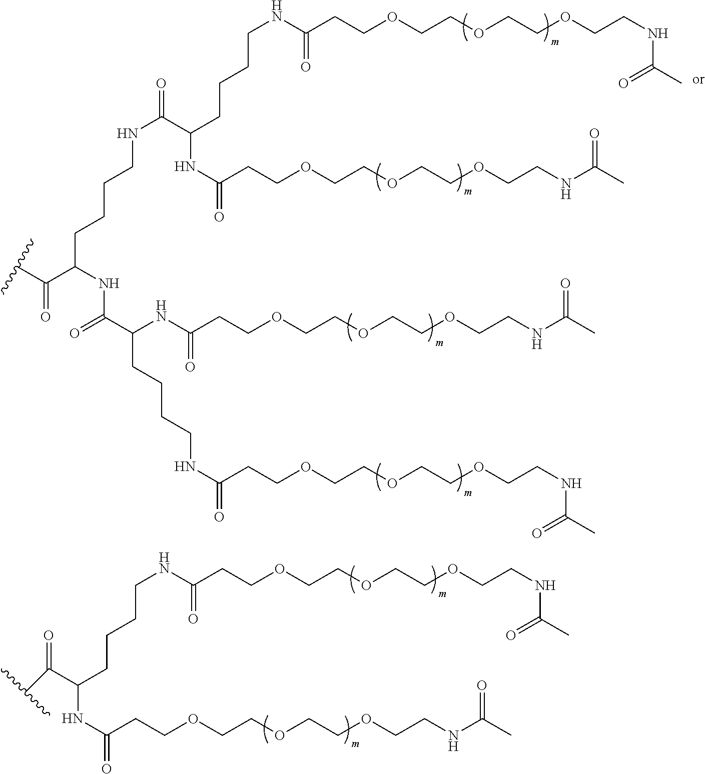

wherein each m is independently an integer from 2-30.

In some embodiments, the polymer attached to the cleavable moiety has a molecular weight of from about 0.1 kDa to about 50 kDa. In some embodiments, the polymer has a molecular weight of at least 0.1 kDa, at least 0.5 kDa, at least 1 kDa, at least 2 kDa, at least 3 kDa, at least 5 kDa, at least 10 kDa, at least 20 kDa, or at least 30 kDa. In some embodiments, the polymer has a molecular weight of at most about 50 kDa, 40 kDa, 30 kDa, 20 kDa, 15 kDa, 10 kDa, 5 kDa, 3 kDa, 2 kDa, or 1 kDa. In some embodiments, the polymer has a molecular weight of from about 0.1 kDa to about 50 kDa, about 0.1 kDa to about 40 kDa, about 0.1 kDa to about 30 kDa, about 0.1 kDa to about 20 kDa, about 0.1 kDa to about 15 kDa, about 0.1 kDa to about 10 kDa, about 0.1 kDa to about 5 kDa, about 0.1 kDa to about 3 kDa, about 0.1 kDa to about 2 kDa, about 0.1 kDa to about 1 kDa, 0.5 kDa to about 50 kDa, about 0.5 kDa to about 40 kDa, about 0.5 kDa to about 30 kDa, about 0.5 kDa to about 20 kDa, about 0.5 kDa to about 15 kDa, about 0.5 kDa to about 10 kDa, about 0.5 kDa to about 5 kDa, about 0.5 kDa to about 3 kDa, about 0.5 kDa to about 2 kDa, about 0.5 kDa to about 1 kDa, about 1 kDa to about 50 kDa, about 1 kDa to about 30 kDa, about 1 kDa to about 15 kDa, about 1 kDa to about 10 kDa, about 1 kDa to about 5 kDa about 1 kDa to about 3 kDa, about 1 kDa to about 2 kDa, about 3 kDa to about 50 kDa, about 3 kDa to about 30 kDa, about 3 kDa to about 15 kDa, about 3 kDa to about 10 kDa, about 5 kDa to about 50 kDa, about 5 kDa to about 30 kDa, about 5 kDa to about 20 kDa, about 10 kDa to about 50 kDa, or about 10 kDa to about 30 kDa.

### Additional Modifications to IL-2 Polypeptides

In some embodiments, the IL-2 polypeptide comprises additional modifications in addition to the attachment of the cleavable moiety (e.g., the one or two points of attachment of the cleavable moiety and corresponding amino acid substitutions to effectuate the attachment of the cleavable moiety). Additional modifications to the IL-2 polypeptide can included without limitation amino acid substitutions, deletions, additions, attachment of polymer moieties, conjugation to other moieties (e.g., additional polypeptides), and/or fusions to other polypeptides. Unless otherwise specified, modifications to indicated amino acid residue numbers of an IL-2 polypeptide described herein utilize SEQ ID NO: 1 as a reference sequence (wild type IL-2).

In some embodiments, the IL-2 polypeptide comprises modifications which bias IL-2 polypeptide in favor of signaling through the IL-2 receptor beta subunit (or the IL-2 receptor βγ complex) over the IL-2 receptor alpha subunit (or the IL-2 receptor αβγ complex). Wild type IL-2 (SEQ ID NO: 1) displays a greater ability to bind and signal through the IL-2 receptor alpha subunit (or the IL-2 receptor αβγ complex) than the IL-2 receptor beta subunit (or the IL-2 receptor βγ complex), thereby generally biasing IL-2 in favor of stimulating Treg cells. Conversely, in some embodiments, the IL-2 polypeptides of the activatable IL-2 polypeptides provided herein are biased in favor binding to the IL-2 receptor beta subunit (or the IL-2 receptor βγ complex). In some embodiments, this is accomplished through modification(s) which enhance the binding of the IL-2 polypeptide to the IL-2 receptor beta subunit (or the IL-2 receptor βγ complex), through modification(s) which reduce the binding of the IL-2 polypeptide to the IL-2 receptor alpha subunit (or the IL-2 receptor αβγ complex), or a combination of these modifications. Non-limiting examples of modifications which bias IL-2 polypeptides towards IL-2 receptor beta signaling are described in, for example, Patent Cooperation Treaty Publication Nos. WO2021/140416, WO2012/065086, WO2019/028419, WO2012/107417, WO2018/119114, WO2012/062228, WO2019/104092, WO2012/088446, and WO2015/164815, each of which is hereby incorporated by reference as if set forth herein in its entirety. It is contemplated any suitable modifications provided therein are compatible with embodiments of the instant disclosure.

In addition to modifications of IL-2 which may affect binding to one or more IL-2 receptor subunits (such as the alpha subunit), the IL-2 polypeptide provided herein may also comprises one or more modifications which improve the stability or pharmacokinetic properties of the IL-2 polypeptide. For example, the IL-2 polypeptide provided herein can comprise the modifications relative to SEQ NO: 1 which are contained in aldesleukin (Proleukin®), namely a deletion of the N-terminal A residue and a C125S substitution relative to SEQ ID NO: 1 (Wild type IL-2).

In some embodiments, the IL-2 polypeptide of the activatable IL-2 polypeptide described herein contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more modified amino acid residues compared to SEQ ID NO: 1. In some embodiments, the IL-2 polypeptide of the activatable IL-2 polypeptide composition comprises an amino acid sequence having at least 80%, 85%, 90%, 91%, 92%, 93%, 94%, 95%, 96%, 97%, 98%, or 99% sequence identity to the sequence set forth in SEQ ID NO: 1. In some embodiments, the IL-2 polypeptide of the activatable IL-2 polypeptide comprises an amino acid sequence having at least 80%, 85%, 90%, 91%, 92%, 93%, 94%, 95%, 96%, 97%, 98%, or 99% sequence identity to the sequence set forth in SEQ ID NO: 2. In some embodiments, the IL-2 polypeptide of the activatable IL-2 polypeptide comprises an amino acid sequence having at least 80%, 85%, 90%, 91%, 92%, 93%, 94%, 95%, 96%, 97%, 98%, or 99% sequence identity to the sequence set forth in SEQ ID NO: 3.

In some embodiments, the IL-2 polypeptide of the activatable IL-2 polypeptide described herein comprises at least 1, at least 2, at least 3, at least 4, at least 5, at least 6, at least 7, or at least 9 amino acid substitutions, wherein the amino acid substitutions are relative to SEQ ID NO: 1. In some embodiments, the IL-2 polypeptide comprises 1 to 9 amino acid substitutions. In some embodiments, the IL-2 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-2 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-2 polypeptide comprises at most 4 amino acid substitutions, 5 amino acid substitutions, 6 amino acid substitutions, 7 amino acid substitutions, or 9 amino acid substitutions.

In some embodiments, the IL-2 polypeptide of the activatable IL-2 polypeptide described herein comprises at least 1, at least 2, at least 3, at least 4, at least 5, at least 6, at least 7, or at least 9 amino acid substitutions, wherein the amino acid substitutions are relative to SEQ ID NO: 2. In some embodiments, the IL-2 polypeptide comprises 1 to 9 amino acid substitutions. In some embodiments, the IL-2 polypeptide comprises 1 or 2 amino acid substitutions, 1 to 3 amino acid substitutions, 1 to 4 amino acid substitutions, 1 to 5 amino acid substitutions, 1 to 6 amino acid substitutions, 1 to 7 amino acid substitutions, 1 to 8 amino acid substitutions, 2 to 3 amino acid substitutions, 2 to 4 amino acid substitutions, 2 to 5 amino acid substitutions, 2 to 6 amino acid substitutions, 2 to 7 amino acid substitutions, 2 to 8 amino acid substitutions, 2 to 9 amino acid substitutions 3 or 4 amino acid substitutions, 3 to 5 amino acid substitutions, 3 to 6 amino acid substitutions, 3 to 7 amino acid substitutions, 3 to 9 amino acid substitutions, 4 or 5 amino acid substitutions, 4 to 6 amino acid substitutions, 4 to 7 amino acid substitutions, 4 to 9 amino acid substitutions, 5 or 6 amino acid substitutions, 5 to 7 amino acid substitutions, 5 to 9 amino acid substitutions, 6 or 7 amino acid substitutions, 6 to 9 amino acid substitutions, or 7 to 9 amino acid substitutions. In some embodiments, the IL-2 polypeptide comprises 3 amino acid substitutions, 4 amino acid substitutions, 5 amino acid substitutions, 6 amino acid substitutions, 7 amino acid substitutions, or 9 amino acid substitutions. In some embodiments, the IL-2 polypeptide comprises at most 4 amino acid substitutions, 5 amino acid substitutions, 6 amino acid substitutions, 7 amino acid substitutions, or 9 amino acid substitutions.

Non-limiting examples of modifications to IL-2 polypeptides include amino acid substitutions shown in Table 2A below. In some embodiments, the IL-2 polypeptide comprises 1, 2, 3, 4, 5, or more of the amino acid substitutions set forth in Table 2A.

TABLE 2A

| WT IL-2 | | |
|---|---|---|
| Residue | WT IL-2 | |
| Number* | Residue | Mutations |
| 35 | K | D, I, L, M, N, P, Q, T, Y |
| 36 | L | A, D, E, F, G, H, I, K, M, N, P, R, S, W, Y |
| 38 | R | A, D, G, K, N, P, S, Y |
| 40 | L | D, G, N, S, Y |
| 41 | T | E, G, Y |
| 42 | F | A, D, E, G, I, K, L, N, Q, R, S, T, V, Y |
| 43 | K | H, Y |
| 44 | F | K, Y |
| 45 | Y | A, D, E, G, K, L, N, Q, R, S, T, V |
| 46 | M | I, Y |
| 61 | E | K, M, R, Y |
| 62 | E | D, L, T, Y |
| 64 | K | D, E, G, L, Q, R, Y |
| 65 | P | D, E, F, G, H, I, K, L, N, Q, R, S, T, V, W, Y |
| 66 | L | A, F, Y |
| 67 | E | A, Y |
| 68 | E | V, Y |
| 72 | L | A, D, E, G, K, N, Q, R, S, T, Y |
| 125 | C | S |

*Residue position numbering based on SEQ ID NO: 1 as a reference sequence

In some embodiments, the IL-2 polypeptide comprises 1, 2, 3, 4, 5, or more of the amino acid substitutions set forth in Table 2B.

TABLE 2B

| WT IL-2 | | |
|---|---|---|
| Residue | WT IL-2 | |
| Number* | Residue | Mutations |
| 20 | D | T, Y |
| 35 | K | D, I, L, M, N, P, Q, T Y |
| 38 | R | A, D, G, K, N, P, S, Y |
| 42 | F | A, D, E, G, I, K, L, N, Q, R, S, T, V, Y |
| 43 | K | H, Y |
| 45 | Y | A, D, E, G, K, L, N, Q, R, S, T, V, Y |
| 62 | E | D, L, T, Y |
| 65 | P | D, E, F, G, H, I, K, L, N, Q, R, S, T, V, W, Y |
| 68 | E | V, Y |
| 72 | L | A, D, E, G, K, N, Q, R, S, T, Y |
| 125 | C | S |

*Residue position numbering based on SEQ ID NO: 1 as a reference sequence

In some embodiments, the IL-2 polypeptide comprises at least one modification is in the range of amino acid residues 30-75 of SEQ ID NO: 1. In some embodiments, the IL-2 polypeptide comprises at least one polymer attachment to the residue at position 42 and/or 45 and/or an amino acid substitution at residue position 42 and/or 45. In some embodiments, one modification is at amino acid residue 42. In some embodiments, one modification is a F42Y substitution. In some embodiments, one modification is a polymer attached to residue F42Y. In some embodiments, one modification is at residue 45. In some embodiments, the modification at residue 45 is a polymer attached to residue 45. In some embodiments, the modification at residue 45 is a polymer attached to residue Y45. In some embodiments, the IL-2 polypeptide comprises a first polymer attached at residue F42Y and a second polymer attached at residue Y45. In some embodiments, at least one of the first polymer or the second polymer comprises a conjugation handle covalently attached thereto. In some embodiments, the conjugation handle is attached to the polymer attached at residue F42Y. In some embodiments, the conjugation handle comprises an azide. In some embodiments, the IL-2 polypeptide comprises a deletion of residue 1 from SEQ ID NO: 1. In some embodiments, the IL-2 polypeptide comprises a C125S substitution. In some embodiments, the IL-2 polypeptide further comprises one or more substitutions of a synthetic IL-2 polypeptide as provided herein (e.g., Hse or Nle substitutions).

In one aspect, provided herein, is an IL-2 polypeptide of an activatable IL-2 polypeptide as provided herein (e.g., with a cleavable moiety attached to at least one residue as provided herein), comprising a first polymer covalently attached at residue 42 and a second polymer covalently attached at residue 45, wherein residue position numbering of the IL-2 polypeptide is based on SEQ ID NO: 1 as a reference sequence. In some embodiments, the first polymer and the second polymer are the same. In some embodiments, the first polymer and the second polymer are different. In some embodiments, at least one of the first polymer or the second polymers comprises a conjugation handle (e.g., an azide). In some embodiments, each polymer is attached through a tyrosine residue. In some embodiments, the IL-2 polypeptide comprises an amino acid sequence at least 80%, at least 85%, at least 90%, at least 95%, at least 98%, or at least 99% identical to the amino acid sequence of SEQ ID NO: 1. In some embodiments, the IL-2 polypeptide comprises an amino acid sequence at least 80%, at least 85%, at least 90%, at least 95%, at least 98%, or at least 99% identical to the amino acid sequence of SEQ ID NO: 2. In some embodiments, the IL-2 polypeptide comprises a C125S or C125A substitution relative to SEQ ID NO: 1. In some embodiments, the IL-2 polypeptide comprises a deletion of A1 from the sequence of SEQ ID NO: 1. In some embodiments, the IL-2 polypeptide comprises amino acid substitutions at 1, 2, 3, or 4 methionine residues from SEQ ID NO: 1. In some embodiments, the IL-2 polypeptide further comprises unnatural amino acid substitutions at residues M23, M39, and/or M46. In some embodiments, the unnatural amino acid residues substituted for the methionines are each independently norleucine or O-methyl-homoserine. In some embodiments, the IL-2 polypeptide further comprises homoserine Hse 41, Hse 71, and Hse 104. In some embodiments, the IL-2 polypeptide.

In some embodiments, the IL-2 polypeptide of the activatable IL-2 polypeptide comprises a polymer attached to a residue of the IL-2 polypeptide (e.g., a polymer in addition one which may attach the cleavable moiety). In some embodiments, the polymer is attached to a different residue than the residue to which the cleavable moiety is attached.

In some embodiments, the polymer (e.g., a polymer which is not attached to the cleavable moiety) is attached to an amino acid residue of the IL-2 polypeptide. In some embodiments, the polymer is attached to any amino acid residue of the IL-2 polypeptide (e.g., at a position corresponding to any one of positions 1-133 of SEQ ID NO: 1). In some embodiments, the polymer is attached at a non-terminal residue (e.g., a residue other than the C-terminal residue or N-terminal residue) of the IL-2 polypeptide (e.g., a residue at position corresponding to any one of positions 2-132 of SEQ ID NO: 1). In some embodiments, the polymer is attached at a terminal residue of the IL-2 polypeptide, wherein the IL-2 polypeptide has been extended or truncated by one or more amino acids relative to SEQ ID NO: 1 (e.g., the linker is attached to a residue corresponding to residue 2 of SEQ ID NO: 1 and residue 1 of SEQ ID NO: 1 has been deleted). In some embodiments, the polymer is attached to the N-terminal residue of the IL-2 polypeptide. In some embodiments, the polymer is attached to the N-terminal amine of the IL-2 polypeptide. In some embodiments, the polymer is attached to the C-terminal residue of the IL-2 polypeptide. In some embodiments, the polymer is attached to the C-terminal carboxyl group of the IL-2 polypeptide.

In some embodiments, the polymer (e.g., a polymer which is not attached to the cleavable moiety) is attached to the IL-2 polypeptide at a residue in a region comprising residues 2-132, wherein residue position numbering is based on SEQ ID NO: 1 as a reference sequence. In some embodiments, the polymer is attached to the IL-2 polypeptide at a residue in a region comprising residues 30-75. In some embodiments, the polymer is attached to the IL-2 polypeptide at a residue in a region comprising residues 35-55, residues 35-50, residues 35-45, residues 30-50, residues 40-45, residues 60-75, residues 60-70, residues 65-70, or residues 2-5. In some embodiments, the polymer is attached to the IL-2 polypeptide at a residue selected from residue 65, 66, 67, 68, 69, and 70. In some embodiments, the polymer is attached to the IL-2 polypeptide at a residue selected from residue 40, 41, 42, 43, 44, and 45. In some embodiments, the polymer is attached to the IL-2 polypeptide at residue 42 or 45. In some embodiments, the polymer is attached to the IL-2 polypeptide at residue 42. In some embodiments, the polymer is attached to the IL-2 polypeptide at residue 45.

In some embodiments, the polymer (e.g., a polymer which is not attached to the cleavable moiety) is attached to the IL-2 polypeptide at a residue which disrupts binding of the IL-2 polypeptide with the IL-2 receptor alpha subunit (IL-2Rx). Examples of these residues include residues 3, 5, 34, 35, 36, 37, 38, 40, 41, 42, 43, 44, 45, 60, 61, 62, 63, 64, 65, 67, 68, 69, 71, 72, 103, 104, 105, and 107, as described in, for example, PCT Pub. Nos. WO2019/028419, WO2020/056066, WO2021/140416, and WO2021/216478 each of which is hereby incorporated by reference as if set forth in its entirety. In some embodiments, the polymer is covalently attached at a residue selected from residues corresponding to residues 3, 34, 35, 36, 37, 38, 40, 41, 42, 43, 44, 45, 60, 61, 62, 63, 64, 65, 67, 68, 69, 71, 72, 103, 104, 105, and 107 of SEQ ID NO: 1. In some embodiments, the polymer is covalently attached at residue 1, 35, 37, 38, 41, 42, 43, 44, 45, 60, 61, 62, 64, 65, 68, 69, 71, 72, 104, 105, or 107 of the IL-2 polypeptide. In some embodiments, the polymer is covalently attached at residue 5. In some embodiments, the polymer is covalently attached at residue 38. In some embodiments, the polymer is covalently attached at residue 42. In some embodiments, the polymer is covalently attached at residue 45. In some embodiments, the polymer is covalently attached at residue 61. In some embodiments, the polymer is covalently attached at residue 65. In some embodiments, the polymer is covalently attached at residue 68.

In some embodiments, the residue to which the polymer (e.g., a polymer which is not attached to the cleavable moiety) is attached is a natural amino acid residue. In some embodiments, the residue to which the polymer is covalently attached is selected from cysteine, aspartate, asparagine, glutamate, glutamine, serine, threonine, lysine, and tyrosine. In some embodiments, the residue to which the polymer is covalently attached is selected from asparagine, aspartic acid, cysteine, glutamic acid, glutamine, lysine, and tyrosine. In some embodiments, the polymer is covalently attached to a cysteine. In some embodiments, the polymer is covalently attached to a lysine. In some embodiments, the polymer is covalently attached to a glutamine. In some embodiments, the polymer is covalently attached to an asparagine. In some embodiments, the residue to which the polymer is attached is a tyrosine. In some embodiments, the residue to which the polymer is attached is the natural amino acid in that position in SEQ ID NO: 1 (e.g., Y45 or A1).

In some embodiments, the polymer (e.g., a polymer which is not attached to the cleavable moiety) is attached to a different natural amino acid which is substituted at the relevant position. The substitution can be for a naturally occurring amino acid which is more amenable to attachment of additional functional groups (e.g., aspartic acid, cysteine, glutamic acid, lysine, serine, threonine, or tyrosine), a derivative of modified version of any naturally occurring amino acid, or any unnatural amino acid (e.g., an amino acid containing a desired reactive group, such as a CLICK chemistry reagent such as an azide, alkyne, etc.). In some embodiments, the polymer is covalently attached site-specifically to a natural amino acid.

In some embodiments, the polymer (e.g., a polymer which is not attached to the cleavable moiety) is attached to a tyrosine residue. In some embodiments, the polymer attached to the tyrosine residue has a structure

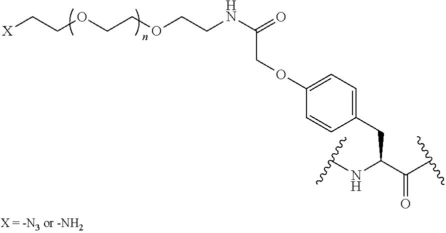

wherein n is an integer from 1-30. In some embodiments, n is an integer from 1-20, 1-10, 2-30, 2-20, 2-10, 5-30, 5-20, or 5-10. In some embodiments, n is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, n is 10. In some embodiments, n is 9. In some embodiments, n is 8. In some embodiments, n is 6. In some embodiments, n is 12. In some embodiments, the polymer attached to the tyrosine residue is at residue F42Y. In some embodiments, the polymer attached to the tyrosine residue is at Y45. In some embodiments, the IL-2 polypeptide comprises two polymers attached to tyrosine residues at F42Y and Y45. In some embodiments, the two polymers are the same size. In some embodiments, one of the two polymers comprises the azide and the other polymer comprises the amine. In some embodiments, the polymer at F42Y comprises the azide and the polymer at Y45 comprises the amine.

In some embodiments, the polymer (e.g., a polymer which is not attached to the cleavable moiety) is attached at an unnatural amino acid residue. In some embodiments, the unnatural amino acid residue comprises a conjugation handle. In some embodiments, the conjugation handle facilitates the addition of the polymer to the modified IL-2 polypeptide. The conjugation handle can be any of the conjugation handles provided herein and is preferably a different conjugation handle which is non-reactive with a conjugation handle used to attach or form part of the linker (where a conjugation handle is used to form the linker). In some embodiments, the polymer is covalently attached site-specifically to the unnatural amino acid. Non-limiting examples of amino acid residues comprising conjugation handles can be found, for example, in PCT Pub. Nos. WO2015/054658, WO2014/036492, and WO2021/133839, WO2006/069246, and WO2007/079130, each of which is incorporated by reference as if set forth in its entirety. In some embodiments, the polymer is attached to an unnatural amino acid residue without use of a conjugation handle.

In some embodiments, the polymer (e.g., a polymer which is not attached to the cleavable moiety) is covalently attached at residue 42. In some embodiments, the polymer is covalently attached at residue F42E, F42D, F42Q, F42K, F42N, or F42Y. In some embodiments, the polymer is covalently attached at residue F42Y. In some embodiments, the polymer is covalently attached to an unnatural amino acid at residue 42.

In some embodiments, the polymer (e.g., a polymer which is not attached to the cleavable moiety) is covalently attached at residue 45. In some embodiments, the polymer is covalently attached at residue Y45, Y45E, Y45C, Y45D, Y45Q, Y45K, or Y45N. In some embodiments, the polymer is covalently attached at residue Y45. In some embodiments, the polymer is covalently attached to an unnatural amino acid at residue 45.

In some embodiments, the polymer (e.g., a polymer which is not attached to the cleavable moiety) is covalently attached at residue 65. In some embodiments, the polymer is covalently attached at residue P65C, P65D, P65Q, P65E, P65N, P65K, or P65Y. In some embodiments, the polymer is covalently attached to an unnatural amino acid at residue 65.

In some embodiments, the polymer (e.g., a polymer which is not attached to the cleavable moiety) is covalently attached at residue 5. In some embodiments, the polymer is covalently attached at residue S5C, S5D, S5Q, S5K, S5N, S5K, or S5Y. In some embodiments, the polymer is covalently attached to an unnatural amino acid at residue 5.

In some embodiments, the polymer (e.g., a polymer which is not attached to the cleavable moiety) is covalently attached at residue 1. In some embodiments, the polymer is covalently attached at residue A1. In some embodiments, the polymer is covalently attached to the N-terminal amine of the IL-2 polypeptide.

In some embodiments, the polymer (e.g., a polymer which is not attached to the cleavable moiety) comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is polysaccharide. In some embodiments, the water-soluble polymer is poly(ethylene oxide) (PEG).

In some embodiments, the polymer (e.g., a polymer which is not attached to the cleavable moiety) has a molecular weight of from about 0.1 kDa to about 50 kDa. In some embodiments, the polymer has a molecular weight of from about 0.1 kDa to about 0.5 kDa from about 0.1 kDa to about 1 kDa, from about 0.1 kDa to about 2 kDa, from about 0.1 kDa to about 5 kDa from about 0.2 kDa to about 1 kDa, from about 0.2 kDa to about 2 kDa, from about 0.2 kDa to about 5 kDa, from about 0.2 kDa to about 10 kDa, from about 0.2 kDa to about 30 kDa, from about 0.5 kDa to about 2 kDa, from about 0.5 kDa to about 5 kDa, from about 0.5 kDa to about 10 kDa, from about 0.5 kDa to about 30 kDa, from about 1 kDa to about 5 kDa, from about 1 kDa to about 10 kDa, from about 1 kDa to about 30 kDa, from about 1 kDa to about 50 kDa, from about 2 kDa to about 10 kDa, from about 2 kDa to about 30 kDa, from about 2 kDa to about 50 kDa, from about 5 kDa to about 30 kDa, or from about 5 kDa to about 50 kDa. In some embodiments, the polymer has a molecular weight of at least about 0.2 kDa, at least about 0.5 kDa, at least about 1 kDa, at least about 2 kDa, at least about 5 kDa, at least about 10 kDa, or at least about 30 kDa. In some embodiments, the polymer has a molecular weight of at most about 30 kDa, at most about 10 kDa, at most about 5 kDa, at most about 2 kDa, at most about 1 kDa, at most about 0.5 kDa or at most about 0.2 kDa. In some embodiments, the polymer has a molecular weight of about 0.5 kDa, about 1 kDa, about 2 kDa, about 3 kDa, about 4 kDa, about 5 kDa, about 7.5 kDa, about 10 kDa, about 12.5 kDa, about 15 kDa, about 20 kDa, about 25 kDa, about 30 kDa, about 35 KDa, about 40 kDa, about 45 kDa, or about 50 kDa. In some embodiments, the polymer is a PEG polymer.

In some embodiments, the polymer (e.g., a polymer which is not attached to the cleavable moiety) is an end-capped polymer. In some embodiments, the polymer is an end-capped polyethylene glycol. In some embodiments, the polymer is end-capped with a functional group selected from amine, alkoxy (e.g., methoxy, ethoxy, propoxy, etc.), hydroxyl, amide (e.g., —NH(C═O) (C₁-C₄ alkyl), carboxylate, and ester (e.g., methyl ester, ethyl ester, etc.). In some embodiments, the polymer as an amine end-capped PEG.

In some embodiments, the IL-2 polypeptide comprises two polymers (e.g., polymers not attached to the cleavable moiety) covalently attached to two separate residues of the IL-2 polypeptide. In some embodiments, the two polymers are a first polymer and a second polymer. Each of the first polymer and the second polymer can be attached to the IL-2 polypeptide at any of the residues as provided herein and can be any of the polymers provided herein (e.g., having any combination of sizes as provided herein). In some embodiments, both of the first polymer and the second polymer are the same size or about the same size. In some embodiments, both polymers are at most about 1 kDa. In some embodiments, one polymer is substantially larger than the other. In some embodiments, one polymer is at most about 1 kDa and the other polymer is at least about 5 kDa. In some embodiments, one polymer comprises a conjugation handle and the second polymer does not.

In some embodiments, the IL-2 polypeptide of the activatable IL-2 polypeptide provided herein is a synthetic IL-2 polypeptide. In some embodiments, the synthetic IL-2 polypeptide is prepared from one or more chemically synthesized fragments. Synthetic IL-2 polypeptides have been previously described, at least in PCT Publication No. WO2021/140416, US Patent Application Publication No. US2021/0155665, and Asahina et al., Angew. Chem. Int. Ed. 2015, 54, 8226-8230, each of which is incorporated by reference as if set forth herein in its entirety.

Any IL-2 polypeptide (or activatable IL-2 polypeptide) provided herein may be prepared as a synthetic IL-2 polypeptide (e.g., having any of the amino acid substitutions (e.g., F42Y, C125S, etc.) or other modifications (e.g., polymer attachment) provided herein in conjunction with the substitutions provided herein for synthetic IL-2 polypeptides, such as homoserine or norleucine residues). In some embodiments, the IL-2 polypeptide comprises any of the amino acid substitutions present in a synthetic IL-2 polypeptide as provided herein (e.g., one or more homoserine or norleucine residues as provided herein). In some embodiments, a synthetic IL-2 polypeptide exhibits a similar or substantially identical activity to a corresponding recombinant IL-2 (e.g., a synthetic IL-2 polypeptide having the same functional modifications to the structure or sequence of the IL-2 polypeptide).

In some embodiments, the synthetic IL-2 polypeptide comprises a homoserine (Hse) residue located in any one of residues 35-45. In some embodiments, the synthetic IL-2 polypeptide comprises a Hse residue located in any one of residues 61-81. In some embodiments, the synthetic IL-2 polypeptide comprises a Hse residue located in any one of residues 94-114. In some embodiments, the synthetic IL-2 polypeptide comprises 1, 2, 3, or more Hse residues. In some embodiments, the synthetic IL-2 polypeptide comprises Hse41, Hse71, Hse104, or a combination thereof. In some embodiments, the synthetic IL-2 polypeptide comprises Hse41, Hse71, and Hse104. In some embodiments, the synthetic IL-2 polypeptide comprises at least two amino acid substitutions, wherein the at least two amino acid substitutions are selected from (a) a homoserine (Hse) residue located in any one of residues 35-45; (b) a homoserine residue located in any one of residues 61-81; and (c) a homoserine residue located in any one of residues 94-114. In some embodiments, the synthetic IL-2 polypeptide comprises Hse41 and Hse71. In some embodiments, the synthetic IL-2 polypeptide comprises Hse41 and Hse104. In some embodiments, the synthetic IL-2 polypeptide comprises Hse71 and Hse104. In some embodiments, the synthetic IL-2 polypeptide comprises Hse41. In some embodiments, the synthetic IL-2 polypeptide comprises Hse71. In some embodiments, the synthetic IL-2 polypeptide comprises Hse104. In some embodiments, the synthetic IL-2 polypeptide comprises 1, 2, 3, or more norleucine (Nle) residues. In some embodiments, the synthetic IL-2 polypeptide comprises a Nle residue located in any one of residues 18-28. In some embodiments, the synthetic IL-2 polypeptide comprises one or more Nle residues located in any one of residues 34-50. In some embodiments, the synthetic IL-2 polypeptide comprises a Nle residue located in any one of residues 20-60. In some embodiments, the synthetic IL-2 polypeptide comprises three Nle substitutions. In some embodiments, the synthetic IL-2 polypeptide comprises Nle23, Nle39, and Nle46.

In some embodiments, the IL-2 polypeptide of an activatable IL-2 polypeptide has an amino acid sequence having at least about 80%, at least about 85%, at least about 90%, at least about 95%, at least about 96%, at least about 97%, at least about 98%, or at least about 99% sequence identity to the sequence set forth in SEQ ID NO: 1 (wild type IL-2). In some embodiments, the IL-2 polypeptide of an activatable IL-2 polypeptide has an amino acid sequence having at least about 80% sequence identity to the sequence set forth in SEQ ID NO: 1. In some embodiments, the IL-2 polypeptide of an activatable IL-2 polypeptide has an amino acid sequence having at least about 85% sequence identity to the sequence set forth in SEQ ID NO: 1. In some embodiments, the IL-2 polypeptide of an activatable IL-2 polypeptide has an amino acid sequence having at least about 90% sequence identity to the sequence set forth in SEQ ID NO: 1. In some embodiments, the IL-2 polypeptide of an activatable IL-2 polypeptide has an amino acid sequence having at least about 95% sequence identity to the sequence set forth in SEQ ID NO: 1.

In some embodiments, the IL-2 polypeptide of an activatable IL-2 polypeptide has an amino acid sequence having at least about 80%, at least about 85%, at least about 90%, at least about 95%, at least about 96%, at least about 97%, at least about 98%, at least about 99%, or 100% sequence identity to the sequence set forth in SEQ ID NO: 2. In some embodiments, the IL-2 polypeptide of an activatable IL-2 polypeptide has an amino acid sequence having at least about 80% sequence identity to the sequence set forth in SEQ ID NO: 2. In some embodiments, the IL-2 polypeptide of an activatable IL-2 polypeptide has an amino acid sequence having at least about 85% sequence identity to the sequence set forth in SEQ ID NO: 2. In some embodiments, the IL-2 polypeptide of an activatable IL-2 polypeptide has an amino acid sequence having at least about 90% sequence identity to the sequence set forth in SEQ ID NO: 2. In some embodiments, the IL-2 polypeptide of an activatable IL-2 polypeptide has an amino acid sequence having at least about 95% sequence identity to the sequence set forth in SEQ ID NO: 2. In some embodiments, the IL-2 polypeptide of an activatable IL-2 polypeptide has an amino acid sequence having at least about 96% sequence identity to the sequence set forth in SEQ ID NO: 2. In some embodiments, the IL-2 polypeptide of an activatable IL-2 polypeptide has an amino acid sequence having at least about 97% sequence identity to the sequence set forth in SEQ ID NO: 2. In some embodiments, the IL-2 polypeptide of an activatable IL-2 polypeptide has an amino acid sequence having at least about 98% sequence identity to the sequence set forth in SEQ ID NO: 2. In some embodiments, the IL-2 polypeptide of an activatable IL-2 polypeptide has an amino acid sequence having at least about 99% sequence identity to the sequence set forth in SEQ ID NO: 2. In some embodiments, the IL-2 polypeptide of an activatable IL-2 polypeptide comprises the sequence of SEQ ID NO: 2. In some embodiments, the IL-2 polypeptide having the above indicated sequence identity to SEQ ID NO: 2 comprises one or more polymers attached to the IL-2 polypeptide as provided herein (e.g., at residues F42Y and Y45).

In some embodiments, the IL-2 polypeptide of an activatable IL-2 polypeptide has an amino acid sequence having at least about 80%, at least about 85%, at least about 90%, at least about 95%, at least about 96%, at least about 97%, at least about 98%, at least about 99%, or 100% sequence identity to the sequence set forth in SEQ ID NO: 3. In some embodiments, the IL-2 polypeptide of an activatable IL-2 polypeptide has an amino acid sequence having at least about 80% sequence identity to the sequence set forth in SEQ ID NO: 3. In some embodiments, the IL-2 polypeptide of an activatable IL-2 polypeptide has an amino acid sequence having at least about 85% sequence identity to the sequence set forth in SEQ ID NO: 3. In some embodiments, the IL-2 polypeptide of an activatable IL-2 polypeptide has an amino acid sequence having at least about 90% sequence identity to the sequence set forth in SEQ ID NO: 3. In some embodiments, the IL-2 polypeptide of an activatable IL-2 polypeptide has an amino acid sequence having at least about 95% sequence identity to the sequence set forth in SEQ ID NO: 3. In some embodiments, the IL-2 polypeptide of an activatable IL-2 polypeptide has an amino acid sequence having at least about 96% sequence identity to the sequence set forth in SEQ ID NO: 3. In some embodiments, the IL-2 polypeptide of an activatable IL-2 polypeptide has an amino acid sequence having at least about 97% sequence identity to the sequence set forth in SEQ ID NO: 3. In some embodiments, the IL-2 polypeptide of an activatable IL-2 polypeptide has an amino acid sequence having at least about 98% sequence identity to the sequence set forth in SEQ ID NO: 3. In some embodiments, the IL-2 polypeptide of an activatable IL-2 polypeptide has an amino acid sequence having at least about 99% sequence identity to the sequence set forth in SEQ ID NO: 3. In some embodiments, the IL-2 polypeptide of an activatable IL-2 polypeptide comprises the sequence of SEQ ID NO: 3.

### Further Attachment of IL-2 Polypeptides to Additional Moieties

In some embodiments, the IL-2 polypeptide of the activatable IL-2 polypeptide can be attached to an additional moiety in a manner which is not through and does not involve the cleavable linker (e.g., the additional moiety is attached at a point of attachment distinct from and unrelated to the cleavable moiety). Attachment of an additional moiety to the IL-2 polypeptide can be either as an alternative to attachment of an additional moiety through the cleavable moiety or can be in addition to attachment of an additional moiety through the cleavable moiety (e.g., the IL-2 polypeptide is attached to two independent additional moieties, one through the cleavable moiety and a second by another point of attachment). In some embodiments, an additional moiety is conjugated to the IL-2 polypeptide (e.g., through a conjugation reaction).

In some embodiments, the IL-2 polypeptide of the activatable IL-2 polypeptide is attached to an additional polypeptide. In some embodiments, the additional polypeptide is an antibody or an antigen binding fragment thereof. In some embodiments, the additional polypeptide is attached to the IL-2 polypeptide through a conjugation reaction. In some embodiments, the additional polypeptide is attached to the IL-2 polypeptide through a conjugation reaction with a conjugation handle attached to the IL-2 polypeptide. In some embodiments, the conjugation handle is one attached to a residue at which a polymer is attached (e.g., a polymer which is not attached to the cleavable moiety, including any of those residues discussed above). In some embodiments, the additional polypeptide is attached at residue 42 or 45 of the IL-2 polypeptide. In some embodiments, the additional polypeptide is attached at residue 42 of the IL-2 polypeptide.

### Activity of Activatable IL-2 Polypeptides and “Activated” IL-2 Polypeptides

In some instances, at least one activity of the activatable IL-2 polypeptides provided herein is altered upon cleavage of the cleavable moiety. In some embodiments, an ability of the IL-2 polypeptide to bind to at least one IL-2 receptor subunit (or a complex thereof) (e.g., the beta or gamma subunits) is enhanced upon cleavage of the cleavable moiety. In some embodiments, an ability of the IL-2 polypeptide to bind to a different IL-2 receptor subunit (e.g., the alpha subunit) is substantially unaffected (e.g., when both the activatable IL-2 polypeptide and the IL-2 polypeptide after cleavage of the cleavable moiety show insubstantial binding to the IL-2 receptor alpha subunit).

In some embodiments, the activatable IL-2 polypeptide exhibits reduced binding to the IL-2 receptor alpha subunit compared to wild type IL-2. In some embodiments, the IL-2 polypeptide after cleavage of the cleavable moiety exhibits reduced binding to the IL-2 receptor alpha subunit compared to wild type IL-2. In some embodiments, the activatable IL-2 polypeptide and the IL-2 polypeptide after cleavage of the cleavable moiety exhibit reduced binding to the IL-2 receptor alpha subunit compared to wild type IL-2. In some embodiments, one or both of the activatable IL-2 polypeptide and/or the IL-2 polypeptide after cleavage of the cleavable moiety exhibit binding to the IL-2 receptor alpha subunit (or the IL-2 receptor αβγ complex) which is reduced by at least 5-fold, 10-fold, 20-fold, 50-fold, 100-fold, 200-fold, 500-fold, or 1000-fold compared to wild type IL-2. In some embodiments, one or both of the activatable IL-2 polypeptide and/or the IL-2 polypeptide after cleavage of the cleavable moiety exhibit binding to the IL-2 receptor alpha subunit (or the IL-2 receptor αβγ complex) with a K_{D} of at least 100 nM, at least 500 nM, at least 1 micromolar, at least 10 micromolar, or at least 100 micromolar. In some embodiments, the IL-2 polypeptide after cleavage of the cleavable moiety exhibit binding to the IL-2 receptor alpha subunit (or the IL-2 receptor αβγ complex) with a K_{D} of at least 1 micromolar. In some embodiments, the activatable IL-2 polypeptide exhibits binding to the IL-2 receptor alpha subunit (or the IL-2 receptor αβγ complex) with a K_{D} of at least 1 micromolar. In some embodiments, one or both of the activatable IL-2 polypeptide and/or the IL-2 polypeptide after cleavage of the cleavable moiety exhibit substantially no binding to the IL-2 receptor alpha subunit (or the IL-2 receptor αβγ complex). In some embodiments, one or both of the activatable IL-2 polypeptide and the IL-2 polypeptide after cleavage of the cleavable moiety exhibit reduced ability to signal (e.g., activate the JAK-STAT pathway) through the IL-2 receptor alpha subunit or the IL-2 receptor αβγ complex (e.g., as measured by a reporter assay). In some embodiments, the ability of one or both of the IL-2 polypeptide and the IL-2 polypeptide after cleavage of the cleavable moiety to signal (e.g., activate the JAK-STAT pathway) through the IL-2 receptor alpha subunit or the IL-2 receptor αβγ complex (e.g., as measured by a reporter assay) is reduced by at least 5-fold, 10-fold, 50-fold, 100-fold, 500-fold, or 1000-fold (e.g., as determined by comparing EC50 values).

In some embodiments, the activatable IL-2 polypeptide exhibits reduced binding to the IL-2 receptor beta subunit compared to the IL-2 polypeptide after cleavage of the cleavable moiety. In some embodiments, cleavage of the cleavable moiety enhances binding of the IL-2 polypeptide to the IL-2 receptor beta subunit by a factor of at least 2-fold, at least 3-fold, at least 4-fold, at least 5-fold, at least 6-fold, at least 7-fold, at least 8-fold, at least 9-fold, or at least 10-fold compared to the activatable IL-2 polypeptide. In some embodiments, cleavage of the cleavable moiety enhances binding of the IL-2 polypeptide by a factor of at least 2-fold compared to the activatable IL-2 polypeptide. In some embodiments, cleavage of the cleavable moiety enhances binding of the IL-2 polypeptide by a factor of at least 5-fold compared to the activatable IL-2 polypeptide. In some embodiments, cleavage of the cleavable moiety enhances binding of the IL-2 polypeptide by a factor of at least 10-fold compared to the activatable IL-2 polypeptide. In some embodiments, binding ability of the IL-2 polypeptide and the activatable IL-2 polypeptide with the IL-2 receptor beta subunit is determined by measuring and/or comparing the K_{D} values. In some embodiments, the IL-2 polypeptide after cleavage of the cleavable moiety exhibits a K_{D} value for binding to the IL-2 receptor beta subunit which is at most 10 nM, at most 20 nM, at most 50 nM, at most 100 nM, at most 200 nM, or at most 500 nM. In some embodiments, the IL-2 polypeptide after cleavage of the cleavable moiety exhibits a K_{D} value for binding to the IL-2 receptor beta subunit which is at most 50 nM. In some embodiments, the IL-2 polypeptide after cleavage of the cleavable moiety exhibits a K_{D} value for binding to the IL-2 receptor beta subunit which is at most 200 nM.

In some embodiments, the activatable IL-2 polypeptide exhibits reduced binding to the IL-2 receptor βγ complex compared to the IL-2 polypeptide after cleavage of the cleavable moiety. In some embodiments, cleavage of the cleavable moiety enhances binding of the IL-2 polypeptide to the IL-2 receptor βγ complex by a factor of at least 2-fold, at least 3-fold, at least 4-fold, at least 5-fold, at least 6-fold, at least 7-fold, at least 8-fold, at least 9-fold, or at least 10-fold compared to the activatable IL-2 polypeptide. In some embodiments, cleavage of the cleavable moiety enhances binding of the IL-2 polypeptide by a factor of at least 2-fold compared to the activatable IL-2 polypeptide. In some embodiments, cleavage of the cleavable moiety enhances binding of the IL-2 polypeptide by a factor of at least 5-fold compared to the activatable IL-2 polypeptide. In some embodiments, cleavage of the cleavable moiety enhances binding of the IL-2 polypeptide by a factor of at least 10-fold compared to the activatable IL-2 polypeptide. In some embodiments, binding ability of the IL-2 polypeptide and the activatable IL-2 polypeptide with the IL-2 receptor βγ complex is determined by measuring and/or comparing the K_{D} values. In some embodiments, the IL-2 polypeptide after cleavage of the cleavable moiety exhibits a K_{D} value for binding to the IL-2 receptor βγ complex which is at most 10 nM, at most 20 nM, at most 50 nM, at most 100 nM, at most 200 nM, or at most 500 nM. In some embodiments, the IL-2 polypeptide after cleavage of the cleavable moiety exhibits a K_{D} value for binding to the IL-2 receptor βγ complex which is at most 20 nM. In some embodiments, the IL-2 polypeptide after cleavage of the cleavable moiety exhibits a K_{D} value for binding to the IL-2 receptor βγ complex which is at most 50 nM. In some embodiments, the IL-2 polypeptide after cleavage of the cleavable moiety exhibits a K_{D} value for binding to the IL-2 receptor βγ complex which is at most 200 nM.

In some embodiments, the activatable IL-2 polypeptide exhibits reduced ability to signal through the IL-2 receptor βγ complex (e.g., activate the JAK-STAT pathway) compared to the IL-2 polypeptide after cleavage of the cleavable moiety. In some embodiments, cleavage of the cleavable moiety enhances the ability of the IL-2 polypeptide to signal through the IL-2 receptor βγ complex by a factor of at least 2-fold, at least 3-fold, at least 4-fold, at least 5-fold, at least 6-fold, at least 7-fold, at least 8-fold, at least 9-fold, or at least 10-fold compared to the activatable IL-2 polypeptide. In some embodiments, cleavage of the cleavable moiety enhances the ability of the IL-2 polypeptide to signal through the IL-2 receptor βγ complex by a factor of at least 2-fold compared to the activatable IL-2 polypeptide. In some embodiments, cleavage of the cleavable moiety enhances the ability of the IL-2 polypeptide to signal through the IL-2 receptor βγ complex by a factor of at least 5-fold compared to the activatable IL-2 polypeptide. In some embodiments, cleavage of the cleavable moiety enhances the ability of the IL-2 polypeptide to signal through the IL-2 receptor βγ complex by a factor of at least 10-fold compared to the activatable IL-2 polypeptide. In some embodiments, the ability of the IL-2 polypeptide and the activatable IL-2 polypeptide to signal through the IL-2 receptor βγ complex is determined by measuring and/or comparing half-maximal effective concentrations (EC50s).

In some embodiments, EC₅₀s are determined using a reporter assay (e.g., HEK-Blue™ IL-2 Cell assay from Invitrogen). In some embodiments, the IL-2 polypeptide after cleavage of the cleavable moiety exhibits an EC₅₀ value for signaling through the IL-2 receptor βγ complex which is at most 0.01 nM, at most 0.02 nM, at most 0.05 nM, at most 0.1 nM, at most 0.2 nM, at most 0.5 nM, at most 1 nM, at most 2 nM, or at most 5 nM. In some embodiments, the IL-2 polypeptide after cleavage of the cleavable moiety exhibits an EC₅₀ value for signaling through the IL-2 receptor βγ complex which is at most 0.01 nM. In some embodiments, the IL-2 polypeptide after cleavage of the cleavable moiety exhibits an EC₅₀ value for signaling through the IL-2 receptor βγ complex which is at most 0.05 nM. In some embodiments, the IL-2 polypeptide after cleavage of the cleavable moiety exhibits an EC₅₀ value for signaling through the IL-2 receptor βγ complex which is at most 0.1 nM.

In some embodiments, EC₅₀s are determined using a STAT5 activation assay in T cells. In some embodiments, the T cells are Teff cells. In some embodiments, the IL-2 polypeptide after cleavage of the cleavable moiety exhibits an EC₅₀ value for STAT5 activation which is at most 1 nM, at most 2 nM, at most 5 nM, at most 10 nM, at most 20 nM, or at most 50 nM. In some embodiments, the IL-2 polypeptide after cleavage of the cleavable moiety exhibits an EC₅₀ value for STAT5 activation which is at most 1 nM. In some embodiments, the IL-2 polypeptide after cleavage of the cleavable moiety exhibits an EC₅₀ value for STAT5 activation which is at most 5 nM. In some embodiments, the IL-2 polypeptide after cleavage of the cleavable moiety exhibits an EC₅₀ value for STAT5 activation which is at most 10 nM.

### Exemplary Activatable IL-2 Polypeptides

The instant disclosure contains numerous examples of IL-2 polypeptides, including those shown in the table below. In some embodiments, the activatable IL-2 polypeptide is any of those activatable IL-2 polypeptides shown in Table 3 below (e.g., any one of SEQ ID NOs: 4-52, 54, or 55), or any analogous activatable IL-2 polypeptide (e.g., an IL-2 polypeptide having a cleavable peptide attached at the indicated point of attachments, by the indicated linking groups, etc.).

TABLE 3
IL-2 Polypeptides, Including Activatable IL-2 Polypeptides

| | Ex- | | |
|---|---|---|---|
| SEQ | am- | Substi- | |
| ID | ple | tute | |
| NO: | # | Ref | Sequence |
| 1 | | | APTSSSTKKTQLQLEHLLLDLQMILNGINNYKNPKLTRMLTFKFYMPK |
| (WT- | | | KATELKHLQCLEEELKPLEEVLNLAQSKNFHLRPRDLISNINVIVLELKG |
| IL-2) | | | SETTFMCEYADETATIVEFLNRWITFCQSIISTLT |
| 2 | | | APTSSSTKKTQLQLEHLLLDLQ(Nle)ILNGINNYKNPKLTR(Nle)L(Hse)Y |
| | | | KFY(Nle)PKKATELKHLQCLEEELKPLEEVL(Hse)LAQSKNFHLRPRDLI |
| | | | SNINVIVLELKGSETTF(Hse)CEYADETATIVEFLNRWITFSQSIISTLT |
| 3 | Ref | CMP- | APTSSSTKKTQLQLEHLLLDLQ-Nle-ILNGINNYKNPKLTR-Nle-L-Hse-Yn3-K |
| | | 003 | F-Ygp-NlePKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 4 | 3 | CMP- 118 | 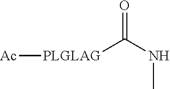 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 5 | 4 | CMP- 119 | 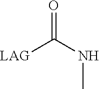 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 6 | 5 | CMP- 120 | 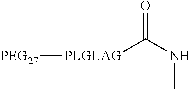 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 7 | 6 | CMP- 121 | 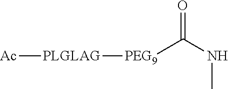 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 8 | 7 | CMP- 122 | 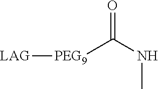 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 9 | 8 | CMP- 123 | 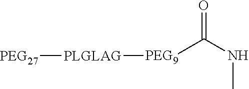 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 10 | 9 | CMP- 124 | 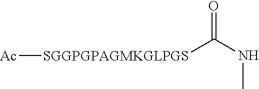 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 11 | 10 | CMP- 125 | 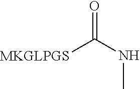 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 12 | 11 | CMP- 126 | 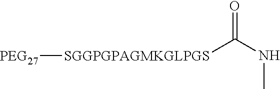 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 13 | 12 | CMP- 127 | 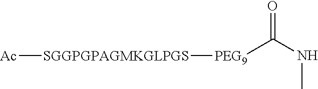 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 14 | 13 | CMP- 128 | 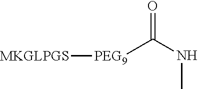 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 15 | 14 | CMP- 129 | 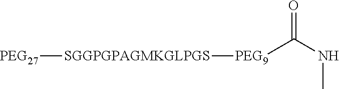 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 16 | 15 | CMP- 141 | 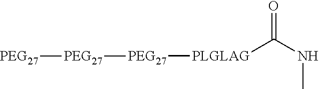 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 17 | 16 | CMP- 142 | 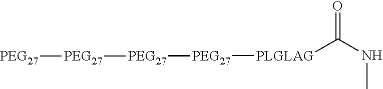 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 18 | 17 | CMP- 143 | 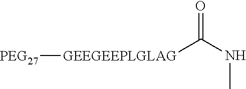 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 19 | 18 | CMP- 144 | 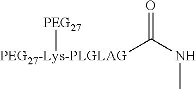 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 20 | 19 | CMP- 145 | 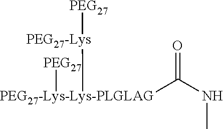 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 21 | 20 | CMP- 146 | 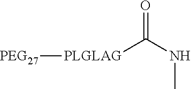 |
| | | | APTSSSTKKT-Lys-LQLEHLLLDLQ-Nle-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNINV |
| | | | IVLELKGSETTF-Hse-CEYADETATIVEFLNRWITFSQSIISTLT |
| 22 | 21 | CMP- 147 | 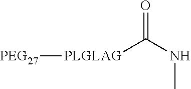 |
| | | | APTSSSTKKTQL-Lys-LEHLLLDLQ-Nle-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNINV |
| | | | IVLELKGSETT F-Hse-CEYADETATIVEFLNRWITFSQSIISTLT |
| 23 | 22 | CMP- 148 | 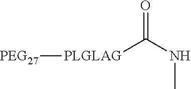 |
| | | | APTSSSTKKTQLQL-Lys-HLLLDLQ-Nle-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNINV |
| | | | IVLELKGSETTF-Hse-CEYADETATIVEFLNRWITFSQSIISTLT |
| 24 | 23 | CMP- 149 | 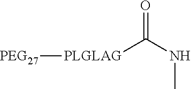 |
| | | | APTSSSTKKTQLQLEHLL-Lys-DLQ-Nle-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNINV |
| | | | IVLELKGSETTF-Hse-CEYADETATIVEFLNRWITFSQSIISTLT |
| 25 | 24 | CMP- 150 | 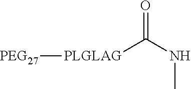 |
| | | | APTSSSTKKTQLQLEHLLLDL-Lys-Nle-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNINV |
| | | | IVLELKGSETTF-Hse-CEYADETATIVEFLNRWITFSQSIISTLT |
| 26 | 25 | CMP- 162 | 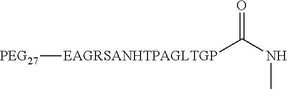 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 27 | 26 | CMP- 133 | 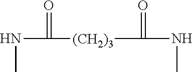 |
| | | | PEG₉-PLGLAG-PEG₉-APTSSSTKKTQLQLEHLLLDLQ-Nle-ILNGINNY-Lys-NPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 28 | 27 | CMP- 134 | 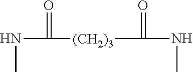 |
| | | | PEG₄-SGGPGPAGMKGLPGS-PEG₄-APTSSSTKKTQLQLEHLLLDLQ-Nle-ILNGINNY-Lys-NP |
| | | | KLTR-Nle-L-Hse-Yn3-KF-Ygp-Nle-PKKATELKHLQCLEEELKPLEEVL-Hse-LAQSKNFHLRP |
| | | | RDLISNINVIVLELKGSETTF-Hse-CEYADETATIVEFLNRWITFSQSIISTLT |
| 29 | 28 | CMP- 135 | 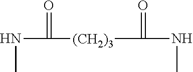 |
| | | | PEG₁₆-SGGPGPAGMKGLPGS-PEG₁₆-APTSSSTKKTQLQLEHLLLDLQ-Nle-ILNGINNY-Lys-NP |
| | | | KLTR-Nle-L-Hse-Yn3-KF-Ygp-Nle-PKKATELKHLQCLEEELKPLEEVL-Hse-LAQSKNFHLRPR |
| | | | DLISNINVIVLELKGSETTF-Hse-CEYADETATIVEFLNRWITFSQSIISTLT |
| 30 | 29 | CMP- 140 | 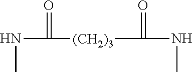 |
| | | | PEG₉-PLGLAG-PEG₂₄-APTSSSTKKTQLQLEHLLLDLQ-Nle-ILNGINNY-Lys-NPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 31 | 30 | CMP- 152 | 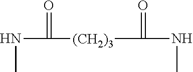 |
| | | | PEG₉-PLGLAG-APTSSSTKKTQLQLEHLLLDLQ-Nle-ILNGINNY-Lys-NPKLTR-Nle-L-Hse-Yn3-K |
| | | | F-Ygp-Nle-PKKATELKHLQCLEEELKPLEEVL-Hse-LAQSKNFHLRPRDLISNINVIVLELKGSETT |
| | | | F-Hse-CEYADETATIVEFLNRWITFSQSIISTLT |
| 32 | 31 | CMP- 153 | 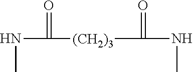 |
| | | | GPLGLAG-PEG₉-APTSSSTKKTQLQLEHLLLDLQ-Nle-ILNGINNY-Lys-NPKLTR-Nle-L-Hse- |
| | | | Yn3-KF-Ygp-Nle-PKKATELKHLQCLEEELKPLEEVL-Hse- |
| | | | FLAQSKNFHLRPRDLISNINVIVLELKGSETT-Hse-CEYADETATIVEFLNRWITFSQSIISTLT |
| 33 | 32 | CMP- 136 | 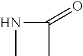 |
| | | | PEG₉-PLGLAG-PEG₉-APTSSSTKKTQLQLEHLLLDLQ-Nle-ILNGI-Glu-NYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 34 | 33 | CMP- 137 | 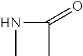 |
| | | | PEG₂₄-PLGLAG-PEG₂₄-APTSSSTKKTQLQLEHLLLDLQ-Nle-ILNGI-Glu-NYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 35 | 34 | CMP- 138 | 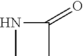 |
| | | | PEG₄-SGGPGPAGMKGLPGS-PEG₄-APTSSSTKKTQLQLEHLLLDLQ-Nle-ILNGI-Glu-N |
| | | | YKNPKLTR-Nle-L-Hse-Yn3-KF-Ygp-Nle-PKKATELKHLQCLEEELKPLEEVL-Hse-LAQ |
| | | | SKNFHLRPRDLISNINVIVLELKGSETTF-Hse-CEYADETATIVEFLNRWITFSQSIISTLT |
| 36 | 35 | CMP- 139 | 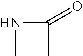 |
| | | | PEG₁₆-SGGPGPAGMKGLPGS-PEG₁₆-APTSSSTKKTQLQLEHLLLDLQ-Nle-ILNGI-Glu-N |
| | | | YKNPKLTR-Nle-L-Hse-Yn3-KF-Ygp-Nle-PKKATELKHLQCLEEELKPLEEVL-Hse-LAQS |
| | | | KNFHLRPRDLISNINVIVLELKGSETTF-Hse-CEYADETATIVEFLNRWITFSQSIISTLT |
| 37 | 36 | CMP- | 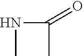 |
| | | | PEG₉-PLGLAG-APTSSSTKKTQLQLEHLLLDLQ-Nle-ILNGI-Glu-NYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 38 | 37 | CMP- 155 | 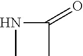 |
| | | | GPLGLAG-PEG₉-APTSSSTKKTQLQLEHLLLDLQ-Nle-ILNGI-Glu-NYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 39 | 38 | CMP- 156 | 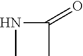 |
| | | | GPLGLAG-PEG₄-APTSSSTKKTQLQLEHLL-Glu-DLQ-Nle-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 40 | 39 | CMP- 157 | 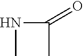 |
| | | | PEG₄-PLGLAG-APTSSSTKKTQLQLEHLL-Glu-DLQ-Nle-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 41 | 40 | CMP- 158 | 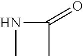 |
| | | | GPLGLAG-PEG₄-APTSSSTKKTQLQLEHLLLDL-Glu-Nle-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 42 | 41 | CMP- 159 | 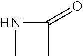 |
| | | | PEG₄-PLGLAG-APTSSSTKKTQLQLEHLLLDL-Glu-Nle-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 43 | 42 | CMP- 160 | 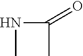 |
| | | | GPLGLAG-PEG₉-APTSSSTKKTQLQLEHLLLDLQ-Nle-IL-Glu-GINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 44 | 43 | CMP- 161 | 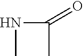 |
| | | | PEG₉-PLGLAG-APTSSSTKKTQLQLEHLLLDLQ-Nle-IL-Glu-GINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 45 | 44 | CMP- 162 | 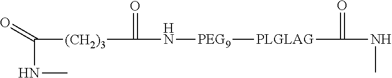 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 46 | 45 | CMP- 163 | 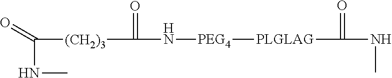 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 47 | 46 | CMP- 164 | 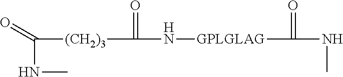 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 48 | 47 | CMP- 165 | 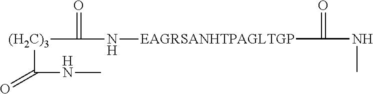 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 49 | 48 | CMP- 166 | 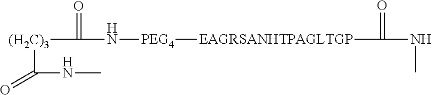 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 50 | 49 | CMP- 167 | 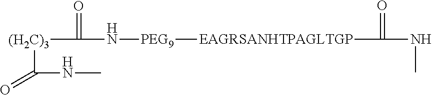 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 51 | 50 | CMP- 168 | 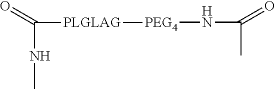 |
| | | | APTSSSTK-Lys-TQLQLEHLLLDLQ-Glu-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 52 | 51 | CMP- 169 | 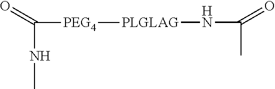 |
| | | | APTSSSTK-Lys-TQLQLEHLLLDLQ-Glu-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 53 | 52 | CMP- | APTSSSTKKTQLQLE-Dab-LLLDLQ-Nle-ILNGINNYKNPKLTR-Nle-L-Hse-Yn3-KF-Ygp-Nle- |
| | | 130 | PKKATELKHLQCLEEELKPLEEVL-Hse-LAQSKNFHLRPRDLISNINVIVLELKGSETT |
| | | | F-Hse-CEYADETATIVEFLNRWITFSQSIISTLT |
| 54 | 53 | CMP- 131 | 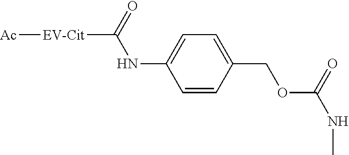 |
| | | | APTSSSTKKTQLQLE-Dab-LLLDLQ-Nle-ILNGINNYKNPKLTR-Nle-L-Hse-Yn3-KF-Ygp-Nle- |
| | | | PKKATELKHLQCLEEELKPLEEVL-Hse-LAQSKNFHLRPRDLISNINVIVLELKGSETT |
| | | | F-Hse-CEYADETATIVEFLNRWITFSQSIISTLT |
| 55 | 54A | CMP- 132 | 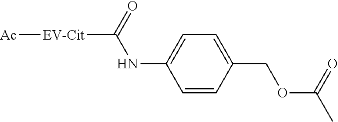 |
| | | | APTSSSTKKTQLQL-Glu-HLLLDLQ-Nle-ILNGINNYKNPKLTR-Nle-L-Hse-Yn3-KF-Ygp-Nle- |
| | | | PKKATELKHLQCLEEELKPLEEVL-Hse-LAQSKNFHLRPRDLISNINVIVLELKGSETT |
| | | | F-Hse-CEYADETATIVEFLNRWITFSQSIISTLT |
| 56 | 59A | CMP- 300 | 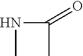 |
| | | | GPLGLAG-PEG₄-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 57 | 59B | CMP- 301 | 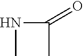 |
| | | | GPLGLAG-PEG₉-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 58 | 59C | CMP- 302 | 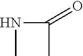 |
| | | | GPLGLAGAPTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 59 | 59D | CMP- 303 | 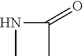 |
| | | | PEG₄-PLGLAGAPTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 60 | 59E | CMP- 304 | 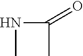 |
| | | | PEG₉-PLGLAGAPTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 61 | 59F | CMP- 305 | 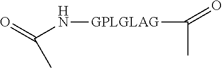 |
| | | | APTSSSTKKTQLQL-Glu-HLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 62 | 54B | CMP- 310 | 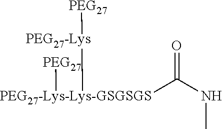 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 63 | 54C | CMP- 311 | 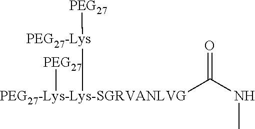 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 64 | 54D | CMP- 312 | 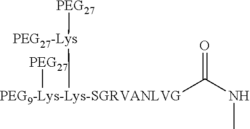 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 65 | 54E | CMP- 313 | 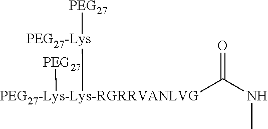 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 66 | 54F | CMP- 314 | 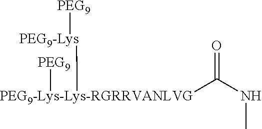 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 67 | 54G | CMP- 315 | 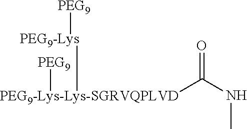 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 68 | 54H | CMP- 316 | 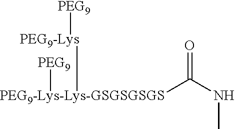 |
| | | | APTSSSTKKTQLQLEHLLLDLQ-Lys-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 69 | 541 | CMP- 317 | 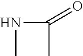 |
| | | | GSGSGSG-PEG₄-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 70 | 54J | CMP- | 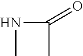 |
| | | | SGRVANLVG-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 71 | 54K | CMP- 319 | 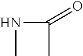 |
| | | | RGRRVANLVG-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 72 | 54L | CMP- 320 | 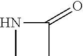 |
| | | | SGRVAPQPLVD-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 73 | 54M | CMP- 321 | 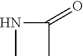 |
| | | | SGRVQPLVD-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 74 | 54N | CMP- 322 | 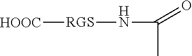 |
| | | | LVG-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 75 | 54O | CMP- 323 | 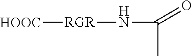 |
| | | | LVG-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 76 | 54P | CMP- 324 | 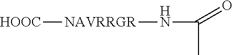 |
| | | | LVG-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 77 | 54Q | CMP- 325 | 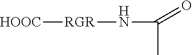 |
| | | | RVANLVG-APTSSSTKKTQLQLEHLLLDLQ-Glu-ILNGINNYKNPKLT |
| | | | R-Nle-L-Hse-Yn3-KF-Ygp-Nle-PKKATELKHLQCLEEELKPLEEVL-Hse-LAQSKNFH |
| | | | LRPRDLISNINVIVLELKGSETTF-Hse-CEYADETATIVEFLNRWITFSQSIISTLT |
| 78 | 54R | CMP- 326 | 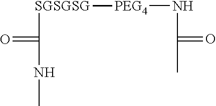 |
| | | | APTSSSTK-Lys-TQLQLEHLLLDLQ-Glu-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 79 | 54S | CMP- 327 | 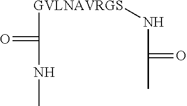 |
| | | | APTSSSTK-Lys-TQLQLEHLLLDLQ-Glu-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 80 | 54T | CMP- 328 | 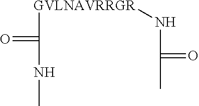 |
| | | | APTSSSTK-Lys-TQLQLEHLLLDLQ-Glu-ILNGINNYKNPKLTR-Nle-L-Hse-Yn3-K |
| | | | F-Ygp-Nle-PKKATELKHLQCLEEELKPLEEVL-Hse-LAQSKNFHLRPRDLISNI |
| | | | NVIVLELKGSETTF-Hse-CEYADETATIVEFLNRWITFSQSIISTLT |
| 81 | 54U | CMP- | |
| | | 329 | |
| 82 | 54V | CMP- | |
| | | 330 | |
| 83 | 54W | CMP- | |
| | | 331 | |

In Table 3 above, Nle is a norleucine residue, Hse is a homoserine residue, Dab is 2,4-diamino butyric acid, Cit is a citrulline residue, Yn3 is a tyrosine residue modified with an azide-capped PEG9 group (see below), and Ygp is a tyrosine residue modified with an amino-capped PEG8 group (see below).

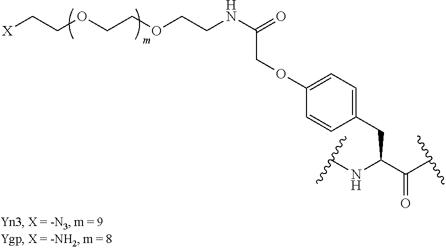

### Methods of Treatment

In one aspect, described herein, is a method of treating cancer in a subject in need thereof, comprising: administering to the subject an effective amount of an activatable IL-2 polypeptide or a pharmaceutical composition as described herein.

In another aspect, described herein, is an activatable IL-2 polypeptide provided herein for use in treatment of cancer in a subject in need thereof. In another aspect, described herein, is an activatable IL-2 polypeptide provided herein for in the manufacture of a medicament for treatment of cancer in a subject in need thereof.

In some embodiments, the cancer is a solid cancer. In some embodiments, wherein the solid cancer is adrenal cancer, anal cancer, bile duct cancer, bladder cancer, bone cancer, brain cancer, breast cancer, carcinoid cancer, cervical cancer, colorectal cancer, esophageal cancer, eye cancer, gallbladder cancer, gastrointestinal stromal tumor, germ cell cancer, head and neck cancer, kidney cancer, liver cancer, lung cancer, nasal cavity and paranasal sinus cancer, nasopharyngeal cancer, neuroblastoma, neuroendocrine cancer, oral cancer, oropharyngeal cancer, ovarian cancer, pancreatic cancer, pediatric cancer, penile cancer, pituitary cancer, prostate cancer, skin cancer, soft tissue cancer, spinal cord cancer, stomach cancer, testicular cancer, thymus cancer, thyroid cancer, ureteral cancer, uterine cancer, vaginal cancer, or vulvar cancer.

In some embodiments, the cancer is a blood cancer. In some embodiments, the cancer is a blood cancer. In some embodiments, the blood cancer is leukemia, non-Hodgkin lymphoma, Hodgkin lymphoma, an AIDS-related lymphoma, multiple myeloma, plasmacytoma, post-transplantation lymphoproliferative disorder, or Waldenstrom macroglobulinemia.

### Pharmaceutical Compositions

In one aspect, described herein is a pharmaceutical composition comprising: an activatable IL-2 polypeptide described herein; and a pharmaceutically acceptable carrier or excipient. In some embodiments, the pharmaceutical composition comprises a plurality of the activatable IL-2 polypeptides. In some embodiments, the pharmaceutical compositions further comprises one or more excipient selected from a carbohydrate, an inorganic salt, an antioxidant, a surfactant, or a buffer.

In some embodiments, the pharmaceutical composition further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

In some embodiments, the pharmaceutical composition comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

In certain embodiments, the pharmaceutical composition comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

In certain embodiments, the pharmaceutical composition comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

In certain embodiments, the pharmaceutical composition comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, CHAPS, or combinations thereof.

In some embodiments, the pharmaceutical composition is formulated for parenteral or enteral administration. In some embodiments, the pharmaceutical composition is formulated for intravenous or subcutaneous administration. In some embodiments, the pharmaceutical composition is in a lyophilized form.

In one aspect, described herein is a liquid or lyophilized composition that comprises a described activatable IL-2 polypeptide. In some embodiments, the activatable IL-2 polypeptide is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising phosphate buffered saline solution (Ph 7.4) with 50 mg/Ml mannitol. In some embodiments, the pharmaceutical composition is a lyophilized composition which is reconstituted shortly before administration to a subject.

The activatable IL-2 polypeptides described herein can be in a variety of dosage forms. In some embodiments, the activatable IL-2 polypeptide is dosed as a reconstituted lyophilized powder. In some embodiments, the activatable IL-2 polypeptide is dosed as a suspension. In some embodiments, the activatable IL-2 polypeptide is dosed as a solution. In some embodiments, the activatable IL-2 polypeptide is dosed as an injectable solution. In some embodiments, the activatable IL-2 polypeptide is dosed as an IV solution.

### Artificial Polypeptides

Also provided herein new cleavable peptides which are cleavable by multiple proteases. In some embodiments, the cleavable peptide comprises 2, 3, 4, or more individual protease cleavage sites, wherein each site is cleavable by a different protease (e.g., any of the proteases provided herein).

In one aspect provided herein is an artificial polypeptide comprising a cleavable peptide having at least about 80%, at least about 90%, or 100% identity to a sequence set forth in Table 1B or Table 1C.

Also provided herein in one aspect is an artificial polypeptide comprising a cleavable peptide having an amino sequence having at least about 80%, at least about 90%, or 100% identity to a sequence set forth in Table 1C. In some embodiments, the artificial polypeptide comprises a cleavable peptide having amino acid sequence set forth in Table 1C.

The artificial polypeptide can be of any type in which it is desirable or advantageous to include a cleavable sequence, such as in an activatable polypeptide, a deactivatable polypeptide, or a polypeptide for which cleavage is desired for any other reason. The polypeptide can take the form of a full length, folded protein, or can be a shorter synthetic peptide (E.g., <50 amino acids in length). In some embodiments, the artificial polypeptide is chemically synthesized. In some embodiments, the artificial polypeptide is a synthetic protein or a recombinant protein. In some embodiments, the artificial polypeptide is a recombinant protein.

In some embodiments, the cleavable peptide is attached to a side chain of an amino acid residue of the artificial polypeptide as provided herein. In some embodiments, the cleavable peptide is comprised internally within the artificial polypeptide. In some embodiments, the cleavable peptide separates two domains of the artificial polypeptide. In some embodiments, the artificial polypeptide is a fusion protein. In some embodiments, the cleavable peptide is comprised between the two fusion partners of the fusion protein. In some embodiments, the cleavable peptide separates an active polypeptide (e.g., a cytokine) and a blocking group (e.g., a cytokine receptor, a steric hindering polypeptide, or other suitable group which blocks interaction of the active polypeptide with its ligand). In some embodiments, the cleavable peptide is at the C-terminus or the N-terminus of the artificial polypeptide.

In some embodiments, the artificial polypeptide comprises a cleavable peptide of any one of SEQ ID Nos: 201-255.

### Certain Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are groups which are “attached” or “covalently attached” to residues of proteins or polypeptides (e.g., IL-2 polypeptides). As used herein, “attached” or “covalently attached” means that the group is tethered to the indicated residue, and such tethering can include a linking group. Thus, for a group “attached” or “covalently attached” to a residue, it is expressly contemplated that such linking groups are also encompassed, unless the group is said to be “directly attached.” In each instance where a group is described as “attached” or “covalently attached”, unless otherwise specified, it is contemplated that the group can be directly attached as well.

As used herein, the term “activatable protein,” or the phrase “activatable” placed before the name of a polypeptide (e.g., “activatable IL-2 polypeptide”) is intended to refer to the protein or polypeptide with the cleavable moiety attached in its intact form (e.g., the cleavable moiety is uncleaved). After cleavage of the cleavable moiety, the remaining entity is referred to as the “protein” or by the name of the relevant polypeptide (e.g., an IL-2 polypeptide). It is expressly intended that reference to the protein or relevant polypeptide after cleavage can include portions of the cleavable moiety which are still attached to the protein or polypeptide. For example, for an activatable protein having a cleavable peptide of sequence PLGLAG (SEQ ID NO: 129) attached to a side chain of a protein (e.g., by an amide bond of the C-terminal G with an appropriate amine-containing side chain, or linked in the same manner by a suitable linking group), the version of the protein with the intact PLGLAG sequence (SEQ ID NO: 129) attached is the “activatable protein,” and the protein or polypeptide by itself (e.g., “the IL-2 polypeptide”) refers to the protein without the intact cleavable peptide, such as with a portion of the cleavable peptide remaining (e.g., the LAG portion of the sequence is still attached to the side chain, either directly or through a linking group).

In some instances, an activity of a protein herein “after cleavage of a cleavable moiety” (or similar terminology) is referred to herein. In determining this activity, it is expressly contemplated that it can be measured by either cleaving the cleavable moiety attached to the protein and then measuring the activity, or the activity can be ascertained by creating the protein with the putative cleavage product attached in lieu of the full cleavable moiety and measuring the activity. Thus, any reference to an activity “after cleavage of the cleavable moiety” or similar such terminology can be replaced with “with the cleavage product of the cleavable moiety attached to the protein.”

Binding affinity refers to the strength of a binding interaction between a single molecule and its ligand/binding partner. A higher binding affinity refers to a higher strength bond than a lower binding affinity. In some instances, binding affinity is measured by the dissociation constant (K_{D})) between the two relevant molecules. When comparing K_{D} values, a binding interaction with a lower value will have a higher binding affinity than a binding interaction with a higher value. For a protein-ligand interaction, K_{D} is calculated according to the following formula:

$${K}_{D} = \frac{[ L ] [ P ]}{[ LP ]}$$

where [L] is the concentration of the ligand, [P] is the concentration of the protein, and [LP] is the concentration of the ligand/protein complex. In some embodiments, K_{D} values as provided herein are measured using bio-layer interferometry.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence: 11, Extension: 1, and Compositional Adjustments Conditional Compositional Score Matrix Adjustment. This alignment algorithm is also used to assess if a residue is at a “corresponding” position through an analysis of the alignment of the two sequences being compared.

The term “pharmaceutically acceptable” refers to approved or approvable by a regulatory agency of the Federal or a state government or listed in the U.S. Pharmacopeia or other generally recognized pharmacopcia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n—COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from cither end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

As used herein, “unnatural” amino acids can refer to amino acid residues in D- or L-form that are not among the 20 canonical amino acids generally incorporated into naturally occurring proteins.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

Table of Conjugation Handles

| Conjugation | | Reaction |
|---|---|---|
| Handle | Complementary Conjugation Handle | Product |
| Sulfhydryl | alpha-halo-carbonyl (e.g., | thioether |
| | bromoacetamide), alpha-beta | |
| | unsaturated carbonyl (e.g., | |
| | maleimide, acrylamide) | |
| Azide | alkyne (e.g., terminal alkyne, | triazole |
| | substituted cyclooctyne (e.g., | |
| | dibenzocycloocytne (DBCO), | |
| | difluorocyclooctyne, | |
| | bicyclo[6.1.0]nonyne, etc.)) | |
| Phosphine | Azide/ester pair | amide |
| Tetrazine | trans-cyoclooctene | dihydropyridazine |
| Amine | Activated ester (e.g., N- | amide |
| | hydroxysuccinimide ester, | |
| | pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine | amide |
| trifluoroborate | (e.g., O-carbamoylhydroxylamine) | |

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### Example 1: Synthesis of Modified IL-2 Polypeptides—General Procedures

### Preparation IL-2 Linear Protein (Representative Protocols)

General strategy: An activatable IL-2 polypeptide or an IL-2 polypeptide as described herein, such as an IL-2 polypeptide having an amino acid sequence of, for example, SEQ ID NOs: 2-61, or an activatable IL-2 polypeptide otherwise described herein, can be synthesized by ligating individual peptide segments prepared by solid phase peptide synthesis (SPPS). Individual peptides were synthesized on an automated peptide synthesizer using the methods described below. The below described methods were used to prepare the exemplified activatable IL-2 polypeptides described herien. Analogous procedures can be used to prepare other activatable IL-2 polypeptides according to the instant disclosure, including those with other cleavable moieties described herein (e.g., any one of the cleavable peptides described in Table 1C, or other suitable cleavable moiety) attached as provided herein.

Materials and solvents: Fmoc-amino acids with suitable side chain protecting groups for Fmoc-SPPS, resins polyethylene glycol derivatives used for peptide functionalization and reagents were commercially available and were used without further purification. HPLC grade CH₃CN was used for analytical and preparative RP-HPLC purification. The following Fmoc-amino acids with side-chain protecting groups were used: Fmoc-Ala-OH, Fmoc-Arg (Pbf)-OH, Fmoc-Asn (Trt)-OH, Fmoc-Asp (OrBu)-OH, Fmoc-Cit-OH, Fmoc-Cys (Acm)-OH, Fmoc-Dab (Alloc)-OH, Fmoc-Dab (Boc)-OH, Fmoc-Gln (Trt)-OH, Fmoc-Glu-OAll, Fmoc-Glu (OrBu)-OH, Fmoc-Glu (OAll)-OH, Fmoc-Gly-OH, Fmoc-His (Trt)-OH, Fmoc-Ile-OH, Fmoc-Leu-OH, Fmoc-Lys (Boc)-OH, Fmoc-Lys (Alloc)-OH, Fmoc-Lys (ivDde)-OH, Fmoc-Nle-OH, Fmoc-Phe-OH, Fmoc-Pro-OH, Fmoc-Ser (tBu)-OH, Fmoc-Thr (tBu)-OH, Fmoc-Trp (Boc)-OH, Fmoc-Tyr (+Bu)-OH, Fmoc-Val-OH, Fmoc or Boc-Opr-OH(Opr=5-(S)-oxaproline), Fmoc-Ygp-OH, Fmoc-Yn3-OH. Fmoc-pseudoproline dipeptides were incorporated in the synthesis if necessary.

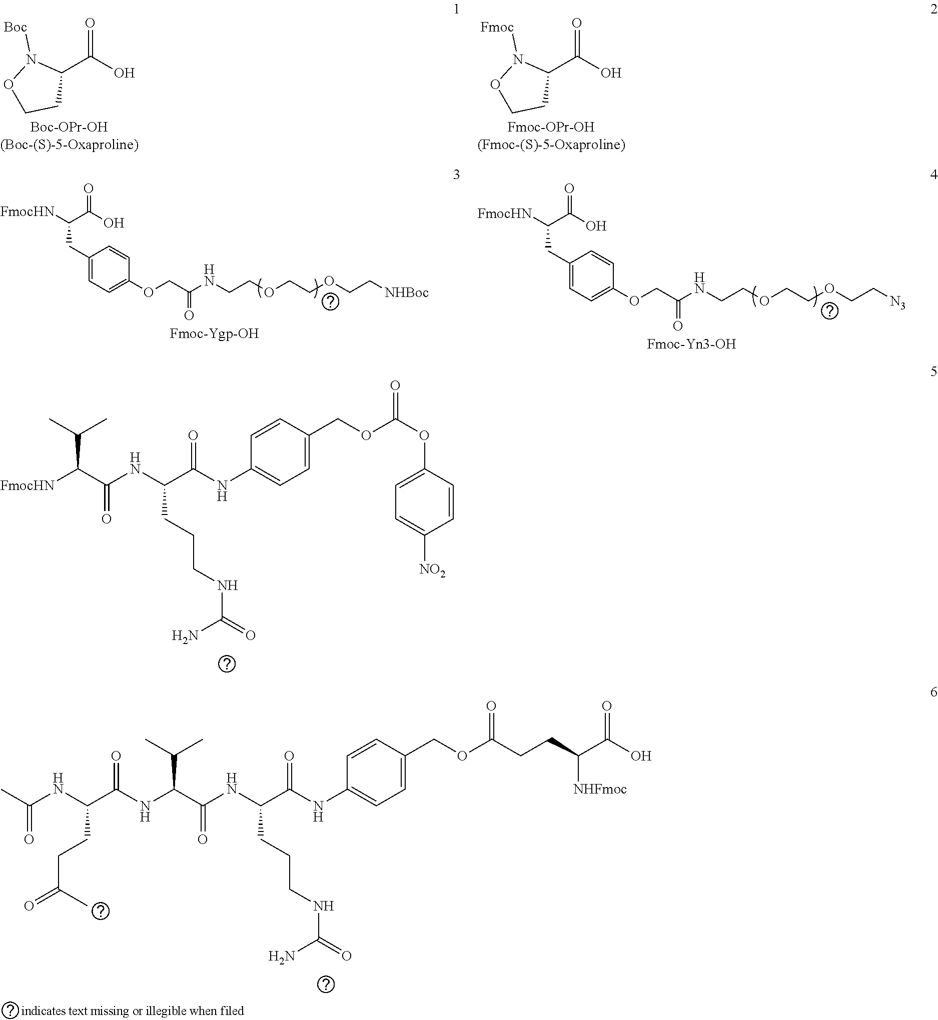

### Special Building Blocks Structures

Protocol 1-Loading of protected ketoacid derivatives (segment 1-3) on amine-based resin: 5 g of Rink-amide MBHA or Sieber Resin (1.8 mmol scale) was swollen in DMF for 30 min. Fmoc-deprotection was performed when needed by treating the resin twice with 20% piperidine in DMF (v/v) at r.t. for 10 min followed by several washes with DMF. Fmoc-AA-protected-α-ketoacid (1.8 mmol, 1.00 equiv.) was dissolved in 20 mL DMF and pre-activated with HATU (650 mg, 1.71 mmol, 0.95 equiv.) and DIPEA (396 μL, 3.6 mmol, 2.00 equiv.). The reaction mixture was added to the swollen resin. It was let to react for 6 h at r.t. under gentle agitation. The resin was rinsed thoroughly with DMF. Capping of unreacted amines on the resin was performed by addition of a solution of acetic anhydride (1.17 mL) and DIPEA (2.34 mL) in DMF (20 mL). It was let to react at r.t. for 15 min under gentle agitation. The resin was rinsed thoroughly with DCM followed by diethyl ether and dried. The loading of the resin was determined by UV quantification of dibenzofulvene to be 0.25 mmol/g.

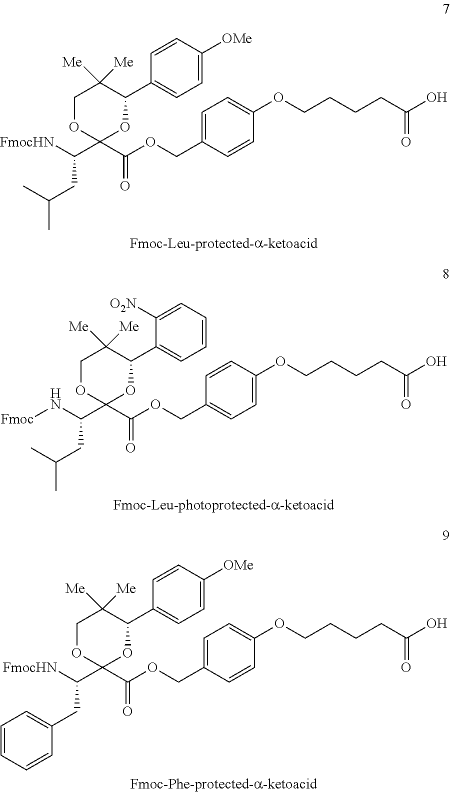

### Protected Ketoacid Used

Protocol 2-Loading of Fmoc-Thr (tBu)-OH on Wang resin (segment4): Preloading of Fmoc-Thr-OH was performed on a Wang resin. 4 g of resin (loading: 0.56 mmol/g, 2.24 mmol scale) was swollen in DMF for 15 min. The resin was treated with 20% (v/v) piperidine in DMF at r.t. for 20 min. The resin was washed several times with DMF. Fmoc-Thr (tBu)-OH (638 mg, 1.68 mmol, 0.75 equiv) and HATU (638 mg, 1.68 mmol, 0.75 equiv) were dissolved in DMF (12 mL). Pre-activation was performed at r.t. for 3 min by addition of DIPEA (585 μL, 3.36 mmol, 1.5 equiv). The reaction mixture was added to the swollen resin. It was let to react overnight at r.t. under gentle agitation. The resin was rinsed thoroughly with DMF. Capping of unreacted amines on the resin was initiated by addition of a solution of acetic anhydride (1.27 mL) and DIPEA (2.34 mL) in DMF (12 mL). It was let to react at r.t. for 15 min under gentle agitation. The resin was rinsed thoroughly with DCM and dried. The loading of the resin was measured (0.34 mmol/g).

Protocol 3-Elongation of the segment 1: The peptide segments were synthesized on an automated peptide synthesizer using Fmoc-SPPS chemistry. Couplings were performed with Fmoc-amino acid (2-16 equiv. to resin substitution), HCTU or HATU (2-16 equiv.) as coupling reagents and DIPEA or NMM (4-32 equiv.) in DMF or NMP (20-60 mL/mmol resin substitution) at r.t. or at 50° C. After pre-activation for 3 min, the solution containing the reagents was added to the resin and allowed to react for 30 min or 2 h depending on the amino acid. In some cases, double couplings were required. In some cases, the resin was treated with 20% acetic anhydride (10 equiv.) in DMF in presence of NMM (10 equiv.) for capping any unreacted free amine. Fmoc deprotections were performed with 20% piperidine or 4-Methylpiperidine in DMF (2×2 min, 40 mL/mmol resin substitution).

Protocol 4-Elongation of segment 2, 3 and 4: Fmoc-Amino acid (2-3 equiv.), DIC (3-7 equiv.), and Oxyma (2-3 equiv.) in DMF (20-60 mL/mmol resin substitution) were stirred for 5-10 min and added to the resin in one portion. The reaction mass was gently agitated under nitrogen bubbling for 2 h at 25-30° C. The solvent was drained, and the resin was washed with DMF (40 mL/mmol resin substitution, 3×5 min), DCM (40 mL/mmol resin substitution; 3×5 min). In some cases, double couplings were required. In some cases, the resin was treated with 20% acetic anhydride (10 equiv.) in DMF in presence of NMM (10 equiv.) for capping any unreacted free amine (20-60 mL/mmol resin substitution). Fmoc deprotections were performed twice 5 or 15 min with 20% piperidine in DMF (40 mL/mmol resin substitution).

Protocol 5-Alloc protection on-resin: A solution of allyl chloroformate (3 equiv.) and Cl-HOBt (6 equiv.) in DCM/DMF (1/1, v/v, 25 mL/mmol) was mixed and DIPEA (6 equiv.) was added to the mixture. After 45 min of agitation, the reaction mixture was added to the resin preswollen in DMF. The reaction was gently agitated for 1 h and then washed twice with DCM (40 mL/mmol resin substitution), twice with DMF (40 mL/mmol resin substitution) and twice with DCM (50 mL/mmol resin substitution).

Protocol 6-Fmoc deprotection of multiple PEG27 derivatives: Fmoc deprotections were performed with 20% piperidine or 4-Methylpiperidine in NMP (4×10 min, 50 mL/mmol resin substitution). The resin was washed with twice with NMP (50 mL/mmol resin substitution), twice with twice with IPA (50 mL/mmol resin substitution) and twice with NMP (50 mL/mmol resin substitution).

Protocol 7-Alloc/All deprotection on resin: DMBA (20 equiv.) dissolved in DMSO (25 mL/mmol) were added to the resin preswollen in DCM followed by Pd[PPh₃]₄ (0.2-0.5 equiv.) dissolved in dry DCM (10 mL/mmol resin substitution). The resin was shaken for 15-30 min and then washed twice with DCM (50 mL/mmol resin substitution), twice with DMF (50 mL/mmol resin substitution), twice with DCM (50 mL/mmol resin substitution), twice with IPA (50 mL/mmol resin substitution), and twice with Et₂O (50 mL/mmol resin substitution). The reaction and the wash were repeated once.

Protocol 8-ivDde deprotection on resin: The resin was swollen in DMF for 10 min. 10% (v/v) hydrazine monohydrate in DMF containing 0.05 M allyl alcohol (40 mL/mmol resin substitution) were added to the resin and it was let to react at room temperature for 30 min. This step was repeated three more times. The resin was then washed with DMF and DCM.

Protocol 9-Glutaric acid incorporation: dihydro-2H-pyran-2,6 (3H)-dione (5 equiv.) was dissolved in DMF (37.5 mL/mmol resin substitution) and DIPEA (7 equiv.) was added. The mixture was transferred to the resin and let it shake for 2 hours at room temperature. The resin was washed twice with DMF (40 mL/mmol resin substitution), twice with DCM (10 mL/mmol resin substitution) and twice with IPA (40 mL/mmol resin substitution).

Protocol 10-2-Cl-Trityl glutamic acid protection: A solution of DIPEA (6 equiv.) in dry DCM (20 mL/mmol resin substitution) was added to the resin followed by a solution of 2-Cl-Trityl chloride (3 equiv.) in dry DCM (20 mL/mmol resin substitution) and shaken for 2 h. The resin was filtered, washed with DCM (40 mL/mmol resin substitution) and the protection step was repeated twice in the same conditions. The resin was washed with DCM (3×40 mL/mmol resin substitution), IPA (2×40 mL/mmol resin substitution) and NMP (3×40 mL/mmol resin substitution).

Protocol 11-2-Cl-Trityl protected glutamic acid deprotection: A solution of 40% HFIP in DCM (100 mL/mmol resin substitution) was added to the resin. The reaction mixture was shaken for 30 min. The deprotection reaction was repeated twice. The resin was washed with DCM (3×40 mL/mmol resin substitution), Et₂O (2×40 mL/mmol resin substitution), DMF (3×40 mL/mmol resin substitution).

Protocol 12-Resin cleavage (Sieber resin) of protected peptide: The resin was preswollen in DCM. A solution of 3% TFA in DCM (v/v, 40 mL/mmol resin substitution) was added on the resin and the mixture was gently agitated for 3 min. The cleavage reaction was repeated 5 times. After each reaction, the filtrates were combined and directly quenched in a 40% DIPEA in DCM solution (40 mL/mmol resin substitution). The resin was washed 3 times with DCM and the combined filtrates were added to the previous filtrates. The mother solution was concentrated under vacuum to dryness and solubilized in ACN. The solution was poured in water and the protected linear peptide as precipitate was filtered and dried.

Protocol 13-Cyclization of protected peptide in solution: The linear protected peptide was dissolved in DMF (300 mL/mmol resin substitution) with 4 equiv. of DIPEA. The peptide solution was added dropwise to a solution of HATU (2 equiv., 350 mL/mmol) in DMF over 30 min. After addition, the reaction mixture was evaporated to dryness.

Protocol 14-Resin cleavage (Rink amide resin) and/or side chain deprotection of the peptides: Once the peptide synthesis was completed, the peptides were cleaved from the resin using a cleavage cocktail (see table below) at room temperature for 2 h. The resin was filtered off, and the filtrate was concentrated and treated with cold diethyl ether, triturated and centrifuged. The ether layer was carefully decanted, the residue was suspended again in diethyl ether, triturated and centrifuged. Ether washings were repeated twice. The resulting crude peptide was dried under vacuum and stored at −20° C. An aliquot of the solid obtained was solubilized in 1:1 CH₃CN/H₂O with 0.1% TFA (v/v) and analyzed by analytical RP-HPLC using C18 column (4.6×150 mm) at 50° C. The molecular weight of the product was identified using MALDI-TOF or LC-MS.

TABLE 4
Rink amide resin cleavage cocktails

| Segment | Cleavage cocktail |
|---|---|
| Segment 1 | TFA:DODT:H₂O (95:2.5:2.5) (v/v/v) |
| Segment 2 | TFA:DODT:H₂O (95:2.5:2.5) (v/v/v) |
| Segment 3 | TFA:DODT:H₂O (95:2.5:2.5) (v/v/v) |
| Segment 4 | TFA:Phenol:TIPS:H2O (82:8:5:5) (v/v/v/v) |

Protocol 15-Ligation of IL-2 segments 1 and 2 and photodeprotection: IL-2 Seg1 (1.2 equiv) and IL-2 Seg2 (1 equiv) were dissolved in DMSO: H₂O (9:1, v/v) containing 0.1 M oxalic acid (20 mM peptide concentration) and allowed to react at 60° C. for 22 h. The ligation vial was protected from light by wrapping it in aluminum foil. The progress of the KAHA ligation was monitored by HPLC using a C18 column (4.6×150 mm) at 60° C. with CH₃CN/H₂O containing 0.1% TFA as mobile phase, with a gradient of 5 to 95% CH₃CN in 7 min. After completion of the ligation the mixture was diluted with CH₃CN/H₂O (1:1) containing 0.1% TFA and irradiated at a wavelength of 365 nm for 1 h. The completion of photolysis reaction was confirmed by injecting a sample on HPLC using previously described method. The sample was then purified by preparative HPLC.

Protocol 16-Ligation of IL-2 segments 3 and 4 and Fmoc deprotection: IL-2-Seg3 (1.2 equiv.) and IL-2-Seg4 (1 equiv.) were dissolved in DMSO/H₂O (9.8:0.2) containing 0.1 M oxalic acid (15 mM) and allowed to react for 20 h at 60° C. The progress of the KAHA ligation was monitored by HPLC using a C18 column (4.6×150 mm) at 60° C. using ACN/H₂O containing 0.1% TFA as mobile phase, with a gradient of 30 to 70% ACN in 7 min. After completion of ligation, the reaction mixture was diluted with DMSO (6 mL) and 5% of diethylamine (300 μL) was added to the reaction mixture and shaken for 7 min at room temperature. To prepare the sample for purification, it was diluted with DMSO containing TFA. The sample was purified by preparative HPLC.

Protocol 17-Final ligation: IL-2-Seg12 (1.2 equiv.) and IL-2-Seg34 (1 equiv.) were dissolved in DMSO/H₂O (9:1) or (9.8:0.2) containing 0.1 M oxalic acid (15 mM peptide concentration) and the ligation was allowed to proceed for 24 h at 60° C. The progress of the KAHA ligation was monitored by analytical HPLC using a C18 column (4.6×250 mm) at 60° C. and ACNI/H₂O containing 0.1% TFA as mobile phase, with a gradient of 30 to 95% CH₃CN in 14 min. After completion of ligation, the reaction mixture was diluted with DMSO followed by further dilution with a mixture of (1:1, v/v) ACN: H₂O containing 0.1% TFA. The sample was purified by preparative HPLC.

Protocol 18-Acm deprotection: IL-2 linear protein with 2× Acm was dissolved in AcOH/H₂O (1:1) (0.25 mM protein concentration) and AgOAc (1% m/v) was added to the solution. The mixture was shaken for 2.5 h at 50° C. protected from light. After completion of reaction as ascertained by HPLC, the sample was diluted with ACN: H₂O (15/85, v/v) containing 0.1% TFA, and purified by preparative HPLC.

Protocol 19-Rearrangement and Folding: IL-2 linear protein (10 mg, 0.611 μmol, 15 μM) was dissolved in 6M Gu·HCl containing 0.1 M Tris pH 8.0 and 30 mM reduced glutathione. The mixture was shaken for 2 h at 45° C. to allow for rearrangement of the ester bond to yield an amide, resulting in the α-homoserine scar from the KAHA ligation. The progress of the rearrangement reaction was monitored by analytical reverse phase HPLC. After completion of rearrangement reaction as ascertained by HPLC, the sample was allowed to cool to room temperature and diluted 3-fold slowly (around 0.25 mL/min) with 0.1 M Tris and 1.5 mM oxidized glutathione, pH 8.0, while the solution was stirred. The folding was allowed to proceed for 20 h at room temperature. This resulted in dilution of the protein to a final concentration of 5 μM and oxidizing conditions that allow disulfide bonds to form. After completion of the folding reaction as ascertained by reverse phase, the sample was acidified with 10% TFA in MQ-H₂O (˜4.5% of total folding volume) until the solution reached pH 3-4. The sample was purified by preparative HPLC using a Waters Xbridge Protein BEH C4 column (20×250 mm) at room temperature (25° C.). A two-step gradient of 5 to 40 to 95% ACN with 0.1% TFA in 60 min, flow rate: 10.0 mL/min, with ACN and MQ-H₂O containing 0.1% TFA as the eluents. The fractions containing the product were pooled and lyophilized to give pure folded protein. The purity and identity of the folded protein powder was initially confirmed by high-resolution mass spectrometry. The lyophilized powder was the reconstituted in formulation buffer (10 mM sodium acetate pH 5.2, 8.4% sucrose and 0.02% PS80).

Protocol 20-Purification of the peptides and proteins: Peptide segments, ligated peptides and linear proteins were purified by RP-HPLC. Different gradients were applied for the different peptides. The mobile phase was MilliQ-H₂O with 0.1% TFA (v/v) (Buffer A) and HPLC grade ACN with 0.1% TFA (v/v) (Buffer B). Preparative HPLC was performed on a C4 (50×250 mm) or on a C18 column (50×250 mm) at a flow rate of 40 or 55 mL/min at 40° C. or 50° C. A representative gradient used for the purification is given in the tables below. For the methods the buffers were Buffer: A: H₂O 0.1% TFA (v/v), B: ACN 0.1% TFA (v/v)

Column: C18 5 μm; 50×250 mm; Temperature: 50° C.

TABLE 5
RP-HPLC Method 1 Gradient

| Time | Flow | | |
|---|---|---|---|
| (min) | (ml/min) | A % | B % |
| 0 | 55 | 95 | 5 |
| 2 | 55 | 90 | 10 |
| 10 | 55 | 90 | 10 |
| 15.1 | 55 | 68 | 32 |
| 51 | 55 | 58 | 42 |
| 52 | 55 | 5 | 95 |
| 60 | 55 | 5 | 95 |
| 61 | 55 | 95 | 5 |
| 65 | 55 | 95 | 5 |

Characterization of the peptides: Peptide segments, ligated peptides and linear proteins were analyzed by RP-HPLC (see methods below). Peptides and proteins were characterized by high resolution Fourier-transform mass spectrometry (FTMS) using a SolariX (9.4T magnet) spectrometer (Bruker, Billerica, USA) equipped with a dual ESI/MALDI-FTICR source, using 4-hydroxy-α-cyanocinnamic acid (HCCA) as matrix. Full proteins are also characterized by SDS-PAGE (see FIG. 6 for representative gels).

### HPLC Methods

Buffers used: A=H₂O with 0.1% TFA (v/v), B=ACN with 0.1% TFA (v/v)

Method 1: Column: Waters XBridge C18 3.5 μm; 3×150 mm; Temperature: 50° C.

TABLE 6
RP-HPLC Method 1 Gradient

| Time | Flow | | |
|---|---|---|---|
| (min) | (mL/min) | % A | % B |
| 0 | 0.8 | 70 | 30 |
| 2 | 0.8 | 70 | 30 |
| 17 | 0.8 | 40 | 60 |
| 17.1 | 0.8 | 5 | 95 |
| 19 | 0.8 | 5 | 95 |
| 19.1 | 0.8 | 70 | 30 |
| 21 | 0.8 | 70 | 30 |

Method 2: Column: Waters XBridge C18 3.5 μm; 3×150 mm; Temperature: 50° C.; gradient:

TABLE 7
RP-HPLC Method 2 Gradient

| Time | Flow | | |
|---|---|---|---|
| (min) | (mL/min) | % A | % B |
| 0 | 0.8 | 80 | 20 |
| 2 | 0.8 | 80 | 20 |
| 17 | 0.8 | 30 | 70 |
| 17.1 | 0.8 | 5 | 95 |
| 19 | 0.8 | 5 | 95 |
| 19.1 | 0.8 | 80 | 20 |
| 21 | 0.8 | 80 | 20 |

Method 3: Column: Phenomenex Aeris 3.6 μm um Widepore XB-C18; 4.6×150 mm; Temperature: 50° C.; gradient:

TABLE 8
RP-HPLC Method 3 Gradient

| Time | Flow | | |
|---|---|---|---|
| (min) | (mL/min) | % A | % B |
| 0.02 | 1 | 70 | 30 |
| 1.5 | 1 | 70 | 30 |
| 8 | 1 | 40 | 60 |
| 8.5 | 1 | 5 | 95 |
| 10 | 1 | 5 | 95 |
| 10.5 | 1 | 95 | 5 |
| 11.95 | 1 | 95 | 5 |
| 12 | 1 | 95 | 5 |

Method 4: Column: Waters XBridge C18 3.5 μm; 3×150 mm; Temperature: 50° C.

TABLE 9
RP-HPLC Method 4 Gradient

| Time | Flow | | |
|---|---|---|---|
| (min) | (mL/min) | % A | % B |
| 0 | 0.8 | 95 | 5 |
| 2 | 0.8 | 95 | 5 |
| 17 | 0.8 | 5 | 95 |
| 17.1 | 0.8 | 5 | 95 |
| 19 | 0.8 | 5 | 95 |
| 19.1 | 0.8 | 95 | 5 |
| 21 | 0.8 | 95 | 5 |

Method 5: Column: Phenomenex Aeris 3.6 μm Widepore C4 200 Å: 4.6×150 mm; Temperature: 50° C.

TABLE 10
RP-HPLC Method 5 Gradient

| Time | Flow | | |
|---|---|---|---|
| (min) | (mL/min) | % A | % B |
| 0 | 2 | 80 | 20 |
| 1.5 | 2 | 80 | 20 |
| 5.4 | 2 | 30 | 70 |
| 5.5 | 2 | 5 | 95 |
| 6.9 | 2 | 5 | 95 |
| 7 | 2 | 80 | 20 |
| 9 | 2 | 80 | 20 |
| 9.1 | 2 | 80 | 20 |

Method 6: Column: Waters Xbridge Protein BEH: C4 300 Å: 2.5 μm: 3×150 mm; Temperature: room temperature

TABLE 11
RP-HPLC Method 6 Gradient

| Time | Flow | | |
|---|---|---|---|
| (min) | (mL/min) | % A | % B |
| 0 | 0.8 | 90 | 10 |
| 1.9 | 0.8 | 90 | 10 |
| 2 | 0.8 | 80 | 20 |
| 17 | 0.8 | 30 | 70 |
| 17.1 | 0.8 | 5 | 95 |
| 19 | 0.8 | 5 | 95 |
| 19.1 | 0.8 | 90 | 10 |
| 21 | 0.8 | 90 | 10 |

Method 7: Column: Waters XBridge C18 3.5 μm; 3×150 mm; Temperature: 50° C.

TABLE 12
RP-HPLC Method 7 Gradient

| Time | Flow | | |
|---|---|---|---|
| (min) | (mL/min) | % A | % B |
| 0 | 0.5 | 90 | 10 |
| 8 | 0.5 | 90 | 10 |
| 20 | 0.5 | 50 | 50 |
| 25 | 0.5 | 20 | 80 |
| 30 | 0.5 | 5 | 95 |
| 30.1 | 0.5 | 5 | 95 |
| 35 | 0.5 | 90 | 10 |

Method 8: Column: Waters XBridge C18 3.5 μm; 3×150 mm; Temperature: 50° C.

TABLE 13
RP-HPLC Method 8 Gradient

| Time | Flow | | |
|---|---|---|---|
| (min) | (mL/min) | % A | % B |
| 0 | 0.8 | 90 | 10 |
| 2 | 0.8 | 90 | 10 |
| 17 | 0.8 | 45 | 55 |
| 17.1 | 0.8 | 5 | 95 |
| 19 | 0.8 | 5 | 95 |
| 19.1 | 0.8 | 90 | 10 |
| 21 | 0.8 | 90 | 10 |

Method 9: Column: Phenomenex Aeris 3.6 μm um Widepore XB-C18; 4.6×150 mm; Temperature: 50° C.

TABLE 14
RP-HPLC Method 9 Gradient

| Time | Flow | | |
|---|---|---|---|
| (min) | (mL/min) | % A | % B |
| 1.40 | 1 | 70 | 30 |
| 1.50 | 1 | 70 | 30 |
| 7.50 | 1 | 30 | 70 |
| 7.60 | 1 | 5 | 95 |
| 10 | 1 | 5 | 95 |
| 10.50 | 1 | 95 | 5 |
| 11.80 | 1 | 95 | 5 |

Method 10: Column: Phenomenex Aeris 3.6 μm um Widepore XB-C18; 4.6×150 mm; Temperature: 50° C.

TABLE 15
RP-HPLC Method 10 Gradient

| Time | Flow | | |
|---|---|---|---|
| (min) | (mL/min) | % A | % B |
| 0 | 1 | 80 | 20 |
| 1.50 | 1 | 80 | 20 |
| 7.50 | 1 | 5 | 95 |
| 10.00 | 1 | 5 | 95 |
| 10.50 | 1 | 80 | 20 |
| 11.80 | 1 | 80 | 20 |

Method 11: Column: Waters XBridge C18 3.5 μm; 3×150 mm; Temperature: 50° C.

TABLE 16
RP-HPLC Method 11 Gradient

| Time | Flow | | |
|---|---|---|---|
| (min) | (mL/min) | % A | % B |
| 0 | 0.5 | 90 | 10 |
| 4 | 0.5 | 70 | 30 |
| 22 | 0.5 | 30 | 70 |
| 25 | 0.5 | 5 | 95 |
| 30 | 0.5 | 5 | 95 |
| 30.1 | 0.5 | 90 | 10 |
| 35 | 0.5 | 90 | 10 |

Loading of the 2-Chloro-Chirotrityl resin (2-CTC) with Fmoc-Cit-OH

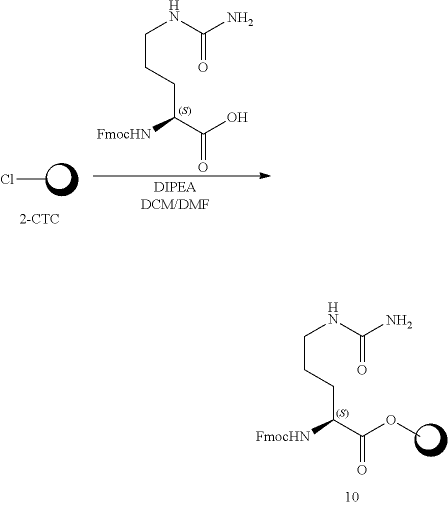

In a 500 mL-Merrifield vessel, the 2-Chlorotrityl Chloride Resin (30.32 g, 1 equiv., 48.51 mmol) was washed with DCM (300 mL) and swollen in DMF (300 mL) for 1 hour. The pre-activated solution (3 min) of Fmoc-Cit-OH (21.21 g, 1.1 Eq, 53.36 mmol), DIPEA (31.34 g, 42.2 mL, 5 equiv., 242.6 mmol) in DMF (300 mL) was added to the resin and reacted by mixing under N₂ bubbling. After filtration, the resin was washed with DMF (300 mL) and DCM (300 mL), and the unreacted resin was capped by adding of a solution of DCM/MeOH/DIPEA 17/2/1 (300 mL) and N₂ bubbling for 15 min. The resin was washed twice with DMF (300 mL), twice with DCM (300 mL), twice with DMF (300 mL), twice with IPA (300 mL) and once with DMF (300 mL).

Elongation of the peptide and cleavage of 12 from the resin:

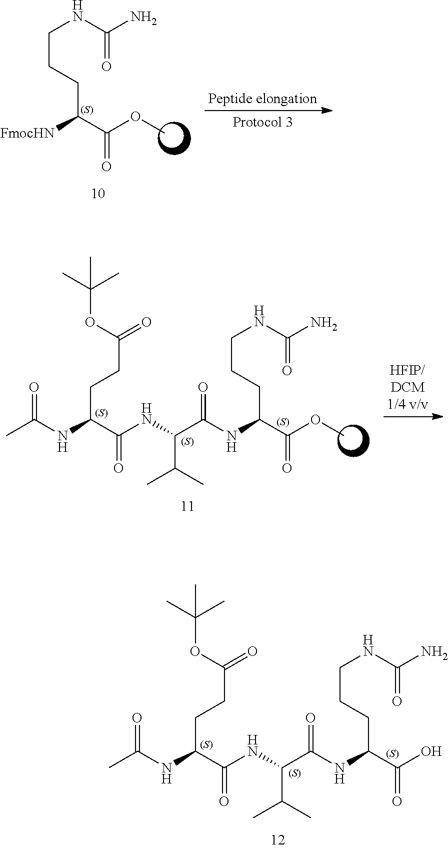

After Fmoc deprotection, elongation of the peptide was performed as described in protocol 3. Compound 12 was cleaved from the resin using the following procedure. After swelling the resin in DCM (300 mL) for 10 min, a solution of HFIP (60 mL) in DCM (240 mL) was added to the resin and shake by N₂ bubbling for 30 min. The resin was filtered and the cleavage was repeated once. The resin was then washed with DCM (300 mL and the combined filtrates were evaporated until dryness, and co-evaporated twice with DCM (100 mL). 12.8 g of pure compound 12 were obtained.

Coupling of 4-aminobenzyl alcohol on compound 12:

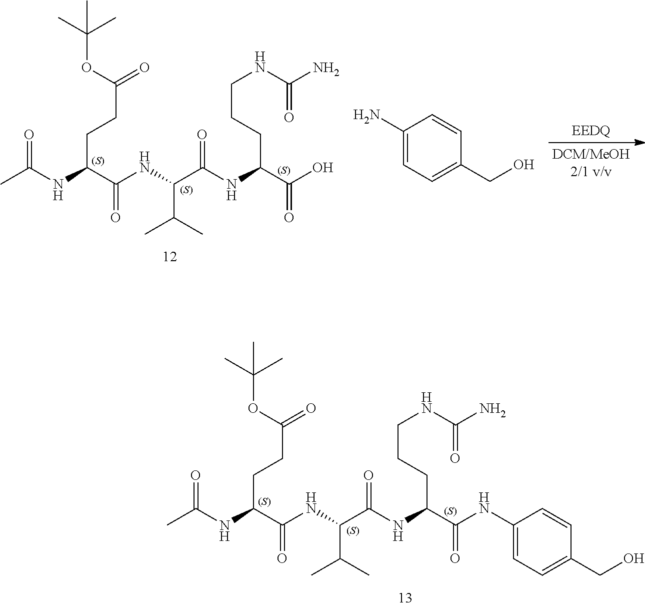

Compound 12 (12.8 g, 1 equiv., 25.5 mmol) was dissolved in a mixture of DCM (120 mL) and MeOH (60 mL). 4-aminobenzyl alcohol (3.46 g, 1.1 equiv., 28.1 mmol) was added followed by EEDQ (12.6 g, 2 equiv., 51.0 mmol). The reaction mixture was stirred, protected from light for 3 h. The reaction mixture was concentrated and suspended in Et₂O (300 mL). After filtration, the white solid was washed twice with Et₂O (100 mL). The white solid was dried under high vacuum overnight yielding 11.8 g of compound 13 (18.5 mmol, 73% yield, 95.1% purity) as a white solid.

Coupling of Fmoc-Glu-OAll on compound 13:

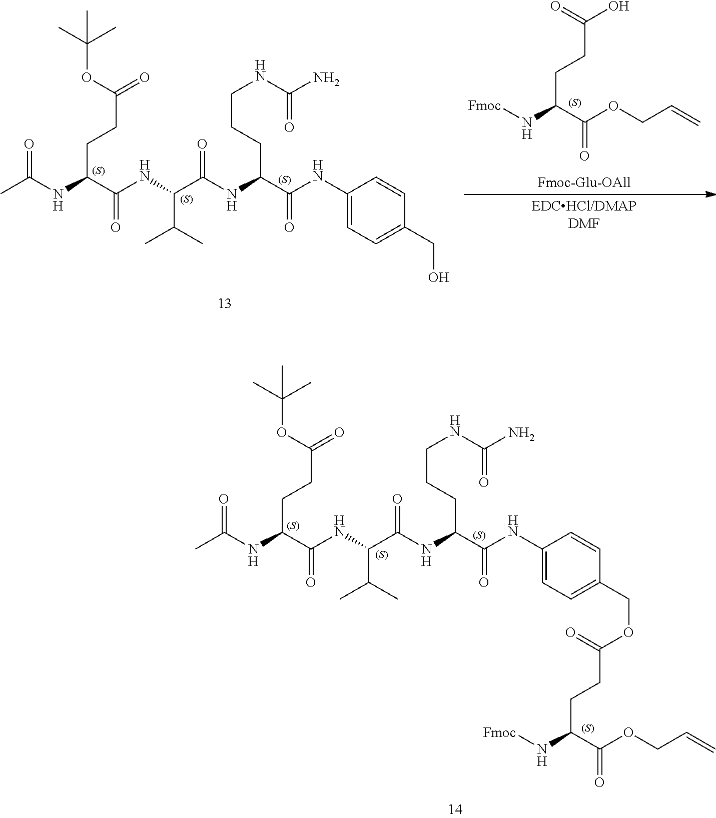

Compound 13 (11.8 g, 1 equiv., 19.4 mmol) and Fmoc-Glu-OAll (9.56 g, 1.2 equiv., 23.3 mmol) were dissolved in DMF (150 mL). EDC·HCl (7.46 g, 2 equiv., 38.9 mmol) and DMAP (0.475 g, 0.2 equiv., 3.89 mmol) were then added. The reaction was stirred overnight. The reaction mixture was then poured dropwise onto cold water (1.2 L). After 15 min stirring, the solution was filtered and the solid was washed with 3 times with water (200 mL). After 15 min drying on the frit, the solid was washed thrice with Et₂₀ (100 mL). After overnight drying under vacuum, compound 14 was obtained (17.6 g, 82% yield, 16 mmol, 90% purity).

Synthesis of compound 6:

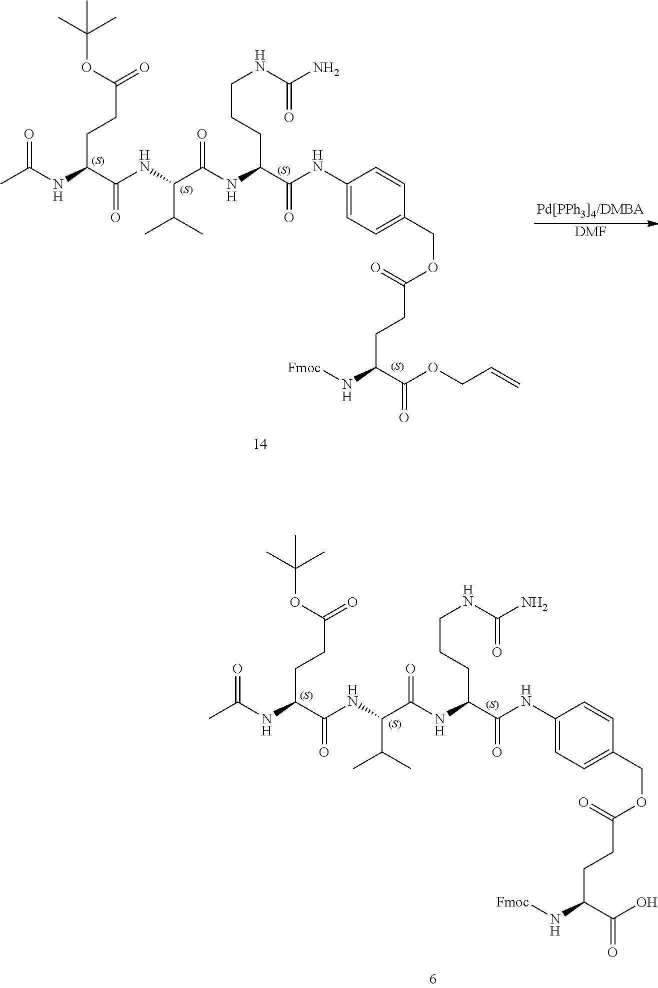

17.6 g of compound 14 (1 equiv., 17.6 mmol) and dimethylbarbituric acid (27.5 g, 10 equiv., 176 mmol) were dissolved in DMF (200 mL). Pd [PPh3]4 (408 mg, 0.02 equiv., 0.353 mmol) were added. After 45 min, L-cysteine hydrochloride (556 mg, 0.2 equiv., 3.53 mmol) were added to complex the palladium in order to remove it at the next step. After 15 min, the mixture was poured dropwise onto cold (4° C.) solution of HCl 0.001 M (pH 3) (1.8 L). After 15 min of stirring, the solution was filtered and the solid was washed 3 times with water (300 mL). After drying under high vacuum, 13.1 g of compound 6 were obtained (12 mmol, 68% yield, 90% purity).

### Example 3: Synthesis of CMP-118

Segment 1: Loading of the first KAHA monomer 7 was performed following protocol 1. monomer 7 Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain was deprotected following protocol 14. The crude peptide was purified by preparative HPLC (protocol 20). The purified segment 1 was analyzed using HPLC method 1 (tR=6.58 min) and by HRMS (Formula: C₂₃₀H₃₈₉N₆₃O₆₈, found: 5123.9349 Da, theoretical: 5123.8975 Da).

Segment 2: Loading of the first KAHA monomer 8 was performed following protocol 1. Elongation of the peptide chain was performed following protocol 4 until position 41 (Boc-Opr). Segment 2 was then released from the resin and the side chain deprotected following protocol 14. The crude peptide was purified by preparative HPLC (protocol 20). The purified segment 2 was analyzed using HPLC method 9 (tR=5.39 min) and by HRMS (Formula: C₂₃₀H₃₇₆N₄₆O₇₄S, found: 5000.6732 Da, theoretical: 5000.6852 Da).

Segment 3: Loading of the first KAHA monomer 9 was performed following protocol 1. Elongation of the peptide chain was performed following protocol 4 until position 71 (Fmoc-Opr). Segment 3 was then released from the resin and the side chain deprotected following protocol 14. The crude peptide was purified by preparative HPLC (protocol 20). The purified segment 3 was analyzed using HPLC method 10 (tR=5.39 min) and by ESI-MS (Formula: C₁₈₄H₂₈₈N₄₇O₅₃, found: 1334.7088 Da, theoretical (M+3H) 3+/3:1334.7090 Da).

Segment 4: Loading of Fmoc-Thr (tBu)-OH was performed following protocol 2. Elongation of the peptide chain was performed following protocol 4 until position 104 (Boc-Opr). Segment 4 was then released from the resin and the side chain deprotected following protocol 14. The crude peptide was purified by preparative HPLC (protocol 20). The purified segment 3 was analyzed using HPLC method 10 (tR=7.03 min) and by ESI-MS (Formula: C₁₅₈H₂₄₄N₃₇O₅₂S, found (M+3H) 3+/3:1174.5748 Da, theoretical (M+3H) 3+/3:1174.5763 Da)

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.942 min) and by HRMS (Formula: C₄₄₉H₇₅₃N₁₀₇O₁₃₇S, found: 9875.5047 Da, theoretical: 9874.5059 Da).

Segment 34: Ligation of segments 3 and 4 and Fmoc-deprotection were performed as described in protocol 16. The ligation/deprotection sample was purified by preparative HPLC (protocol 20). The purified segment 34 was analyzed using HPLC method 10 (tR=6.460 min) and by HRMS (Formula: C₃₂₆H₅₁₆N₈₄O₁₀₁S, found: 7259.7696 Da, theoretical: 7259.7655 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.375 min) and by HRMS (Formula: C₇₇₄H₁₂₇₀N₁₉₂O₂₃₅S2, found: 17091.2714 Da, theoretical: 17091.2842 Da).

Linear protein: Acm protection on cysteine residues were removed using protocol 18. The ligation sample was purified by preparative HPLC (protocol 20). The purified linear protein was analyzed using HPLC method 2 (tR=13.425 min) and by HRMS (Formula: C₇₆₇H₁₂₅₈N₁₉₀O₂₃₄S₂, found: 16949.1952 Da, theoretical: 16949.2099 Da).

Folded protein: The linear protein was rearranged, folded and purified following protocol 19. The folded protein was analyzed using HPLC method 6 (tR=13.758 min) and by HRMS (Formula: C₇₆₇H₁₂₅₆N₁₉₀O₂₃₄S₂, found: 16.947 Da, theoretical: 16.947 Da).

### Example 4: Synthesis of CMP-119

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 1 (tR=6.16 min) and by HRMS (Formula: C₂₁₅H₃₆₆N₆₀O₆₄, found: 4814.7742 Da, theoretical: 4814.7286 Da).

Segments 2, 3, 4 and ligated segment 34 were synthesized as described in Synthesis of CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.425 min) and by HRMS (Formula: C₄₃₃H₇₂₉N₁₀₅O₁₃₃S, found: 9566.3500 Da, theoretical: 9565.3370 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.225 min) and by HRMS (Formula: C₇₅₈H₁₂₄₅N₁₈₉O₂₃₂S₂, found: 16782.0630 Da, theoretical: 16782.1152 Da).

Linear protein: Acm protection on cysteine residues were removed using protocol 18. The ligation sample was purified by preparative HPLC (protocol 20). The purified linear protein was analyzed using HPLC method 2 (tR=12.875 min) and by HRMS (Formula: C₇₅₂H₁₂₃₅N₁₈₇O₂₃₀S, found: 16639.0306 Da, theoretical: 16640.3510 Da).

Folded protein: The linear protein was rearranged, folded and purified following protocol 19. The folded protein was analyzed using HPLC method 6 (tR=13.675 min) and by HRMS (Formula: C₇₅₂H₁₂₃₃N₁₈₇O₂₃₀S₂, found: 16638.0456 Da, theoretical: 16637.0227 Da).

### Example 5: Synthesis of CMP-120

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 1 (tR=6.59 min) and by HRMS (Formula: C₂₈₇H₅₀₄N₆₄O₉₆, found: 6386.6925 Da, theoretical: 6386.6611 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 ((R=10.808 min) and by HRMS (Formula: C₅₀₅H₈₆₇N₁₀₉O₁₆₅S, found: 11137.3008 Da, theoretical: 11136.2743 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.175 min) and by HRMS (Formula: C₈₃₀H₁₃₈₃N₁₉₃O₂₆₄S₂, found: 18354.0163 Da, theoretical: 18353.0526 Da).

Linear protein: Acm protection on cysteine residues were removed using protocol 18. The ligation sample was purified by preparative HPLC (protocol 20). The purified linear protein was analyzed using HPLC method 2 (tR=12.875 min) and by HRMS (Formula: C₈₂₄H₁₃₇₃N₁₉₁O₂₆₂S₂, found: 18211.9393 Da, theoretical: 18209.9756 Da).

Folded protein: The linear protein was rearranged, folded and purified following protocol 19. The folded protein was analyzed using HPLC method 6 (tR=13.642 min) and by HRMS (Formula: C₈₂₄H₁₃₇₁N₁₉₁O₂₆₂S₂, found: 18209.9772 Da, theoretical: 18208.9552 Da).

### Example 6: Synthesis of CMP-121

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.208 min) and by HRMS (Formula: C₂₅₂H₄₃₁N₆₃O₇₈, found: 5592.2092 Da, theoretical: 5591.1782 Da).

Segments 2, 3, 4 and ligated segment 34 were synthesized as described in Synthesis of CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.8 min) and by HRMS (Formula: C₄₇₀H₇₉₄N₁₀₈O₁₄₇S, found: 10341.7929 Da, theoretical: 10341.7866 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.34 min) and by HRMS (Formula: C₇₉₄H₁₃₀₉N₁₉₃O₂₄₆S, found: 17558.5330 Da, theoretical: 17557.5621 Da).

Linear protein: Acm protection on cysteine residues were removed using protocol 18. The ligation sample was purified by preparative HPLC (protocol 20). The purified linear protein was analyzed using HPLC method 2 (R=13.425 min) and by HRMS (Formula: C₇₈₈H₁₂₉₉N₁₉₁O₂₄₄S₂, found: 17416.4625 Da, theoretical: 17415.4879 Da).

folded protein was analyzed using HPLC method 6 (tR=13.725 min) and by HRMS (Formula: C₇₈₈H₁₂₉₇N₁₉₁O₂₄₄S₂, found: 17414.4875 Da, theoretical: 17414.4675 Da).

### Example 7: Synthesis of CMP-122

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.54 min) and by HRMS (Formula: C₂₃₇H₄₀₈N₆₀O₇₄, found: 5283.0474 Da, theoretical: 5282.0093 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 ((R=10.408 min) and by HRMS (Formula: C₄₅₅H₇₇₁N₁₀₅O₁₄₃S, found: 10033.6230 Da, theoretical: 10031.6150 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.075 min) and by HRMS (Formula: C₇₇₉H₁₂₈₆N₁₉₀O₂₄₂S₂, found: 17249.3891 Da, theoretical: 17248.3932 Da).

Linear protein: Acm protection on cysteine residues were removed using protocol 18. The ligation sample was purified by preparative HPLC (protocol 20). The purified linear protein was analyzed using HPLC method 2 (tR=13.525 min) and by HRMS (Formula: C₇₇₃H₁₂₇₆N₁₈₈O₂₄₀S₂, found: 17107.2793 Da, theoretical: 17106.3190 Da).

Folded protein: The linear protein was rearranged, folded and purified following protocol 19. The folded protein was analyzed using HPLC method 6 (tR=13.642 min) and by HRMS (Formula: C₇₇₃H₁₂₇₄N₁₈₈O₂₄₀S₂, found: 17105.3338 Da, theoretical: 17104.3033 Da).

### Example 8: Synthesis of CMP-123

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 ((R=11.108 min) and by HRMS (Formula: C₃₀₉H₅₄₆N₆₄O₁₀₆, found: 6853.9628 Da, theoretical: 6852.9390 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.775 min) and by HRMS (Formula: C₅₂₆H₉₀₈N₁₁₀O₁₇₅S, found: 11605.5988 Da, theoretical: 11603.5475 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.258 min) and by HRMS (Formula: C₈₀₁H₁₄₂₃N₁₉₄O₂₇₄S₂, found: 18821.3076 Da, theoretical: 18821.9570 Da).

Linear protein: Acm protection on cysteine residues were removed using protocol 18. The ligation sample was purified by preparative HPLC (protocol 20). The purified linear protein was analyzed using HPLC method 2 (tR=13.325 min) and by HRMS (Formula: C₈₄₅H₁₄₁₄N₁₉₂O₂₇₂S₂, found: 18679.2064 Da, theoretical: 18678.2515 Da).

Folded protein: The linear protein was rearranged, folded and purified following protocol 19. The folded protein was analyzed using HPLC method 6 (RT=13.608 min) and by HRMS (Formula: C₈₄₅H₁₄₁₂N₁₉₂O₂₇₂S₂, found: 18634.2064 Da, theoretical: 18633.2048 Da).

### Example 9: Synthesis of CMP-124

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 1 (tR=6.11 min) and by HRMS (Formula: C₂₅₉H₄₃₅N₇₃O₇₉S, found: 5867.2515 Da, theoretical: 5867.2068 Da).

Segment 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.408 min) and by HRMS (Formula: C₄₇₇H₇₉₈N₁₁₈O₁₄₈S₂, found: 10616.838 Da, theoretical: 10616.819 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.22 min) and by HRMS (Formula: C₈₀₂H₁₃₁₄N₂₀₂O₂₄₇S₃, found: 17833.5698 Da, theoretical: 17832.5954 Da).

Linear protein: Acm protection on cysteine residues were removed using protocol 18. The ligation sample was purified by preparative HPLC (protocol 20). The purified linear protein was analyzed using HPLC method 2 (tR=13.25 min) and by HRMS (Formula: C₇₉₆H₁₃₀₄N₂₀₀O₂₄₅S₃, found: 17691.4516 Da, theoretical: 17690.5212 Da).

Folded protein: The linear protein was rearranged, folded and purified following protocol 19. The folded protein was analyzed using HPLC method 6 (tR=13.558 min) and by HRMS (Formula: C₇₉₆H₁₃₀₂N₂₀₀O₂₄₅S₃, found: 17690.5703 Da, theoretical: 17689.5007 Da).

### Example 10: Synthesis of CMP-125

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 1 (tR=6.05 min) and by HRMS (Formula: C₂₃₃H₃₉₇N₆₅O₆₉S, found: 5244.9606 Da, theoretical: 5245.1850 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.258 min) and by HRMS (Formula: C₄₅₂H₇₆₁N₁₀₉O₁₃₈S₂, found: 9995.5548 Da, theoretical: 9994.5488 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.658 min) and by HRMS (Formula: C₇₇₆H₁₂₇₆N₁₉₄O₂₃₇S₃, found: 17211.2889 Da, theoretical: 17210.3243 Da).

Linear protein: Acm protection on cysteine residues were removed using protocol 18. The ligation sample was purified by preparative HPLC (protocol 20). The purified linear protein was analyzed using HPLC method 2 (tR=13.25 min) and by HRMS (Formula: C₇₇₀H₁₂₆₆N₁₉₂₀₂₃₅S₃, found: 17069.1853 Da, theoretical: 17068.2500 Da).

Folded protein: The linear protein was rearranged, folded and purified following protocol 19. The folded protein was analyzed using HPLC method 6 (tR=13.558 min) and by HRMS (Formula: C₇₇₀H₁₂₆₄N₁₉₂O₂₃₅S₃, found: 17067.2894 Da, theoretical: 17066.2344 Da).

### Example 11: Synthesis of CMP-126

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (R=10.492 min) and by HRMS (Formula: C₃₁₆H₅₅₀N₇₄O₁₀₇S, found: 7129.9836 Da, theoretical: 7129.9704 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.392 min) and by ESI Formula: C₅₃₄H₉₁₃N₁₁₉₀₁₇₆S, m/z found: 1081.20 Da (M+11H)¹¹⁺/11, m/z theoretical: 1081.08 (M+11H)¹¹⁺/11 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.025 min) and by HRMS (Formula: C₈₅₉H₁₄₂₉N₂₀₃O₂₇₅S₃, found: 19096.4256 Da, theoretical: 19096.3542 Da).

Linear protein: Acm protection on cysteine residues were removed using protocol 18. The ligation sample was purified by preparative HPLC (protocol 20). The purified linear protein was analyzed using HPLC method 2 (tR=13.09 min) and by HRMS (Formula: C₈₅₃H₁₄₁₉N₂₀₁O₂₇₃S₃, found: 18954.2934 Da, theoretical: 18953.2848 Da).

Folded protein: The linear protein was rearranged, folded and purified following protocol 19. The folded protein was analyzed using HPLC method 6 (tR=13.308 min) and by HRMS (Formula: C₈₅₃H₁₄₁₇N₂₀₁O₂₇₃S₃, found: 18952.2909 Da, theoretical: 18952.2643 Da).

### Example 12: Synthesis of CMP-127

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 3 (tR=6.14 min) and by HRMS (Formula: C₂₈₁H₄₇₇N₇₃O₈₉S, found: 6334.5246 Da, theoretical: 6333.4848 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.358 min) and by HRMS (Formula: C₄₉₈H₈₄₁N₁₁₉O₁₅₈S₂, found: 11086.0995 Da, theoretical: 11084.0931 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.975 min) and by HRMS (Formula: C₈₂₃H₁₃₅₅N₂₀₃O₂₅₇S₃, found: 18301.7978 Da, theoretical: 18300.8712 Da).

Linear protein: Acm protection on cysteine residues were removed using protocol 18. The ligation sample was purified by preparative HPLC (protocol 20). The purified linear protein was analyzed using HPLC method 2 (tR=13.47 min) and by HRMS (Formula: C₈₁₇H₁₃₄₅N₂₀₁O₂₅₅S₃, found: 18157.7944 Da, theoretical: 18159.7617 Da).

Folded protein: The linear protein was rearranged, folded and purified following protocol 19. The folded protein was analyzed using HPLC method 6 (tR=13.592 min) and by HRMS (Formula: C₈₁₇H₁₃₄₃N₂₀₁O₂₅₅S₃, found: 18155.7831 Da, theoretical: 18156.7739 Da).

### Example 13: Synthesis of CMP-128

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 3 (tR=6.238 min) and by HRMS (Formula: C₂₅₅H₄₃₉N₆₅O₇₉S, found: 5711.2545 Da, theoretical: 5211.2137 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.308 min) and by HRMS (Formula: C₄₇₂H₈₀₁N₁₁₁O₁₄₈S₂, found: 10462.8460 Da, theoretical: 10461.8219 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.125 min) and by HRMS (Formula: C₇₉₇H₁₃₁₇N₁₉₅O₂₄₇S₃, found: 17678.5650 Da, theoretical: 17677.5975 Da).

Linear protein: Acm protection on cysteine residues were removed using protocol 18. The ligation sample was purified by preparative HPLC (protocol 20). The purified linear protein was analyzed using HPLC method 2 (tR=13.208 min) and by HRMS (Formula: C₇₉₁H₁₃₀₇N₁₉₃O₂₄₅S₃, found: 17536.4918 Da, theoretical: 17535.5232 Da).

Folded protein: The linear protein was rearranged, folded and purified following protocol 19. The folded protein was analyzed using HPLC method 6 (tR=13.592 min) and by HRMS (Formula: C₇₉₁H₁₃₀₅N₁₉₃O₂₄₅S₃, found: 17534.5026 Da, theoretical: 17533.5076 Da).

### Example 14: Synthesis of CMP-129

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.575 min) and by HRMS (Formula: C₃₃₇H₅₉₁N₇₅O₁₁₇S, found: 7597.2576 Da, theoretical: 7596.2484 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.342 min) and by HRMS (Formula: C₅₅₅H₉₅₄N₁₂₀O₁₈₆S₂, found: 12347.8766 Da, theoretical: 12347.8519 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.042 min) and by HRMS (Formula: C₈₈₀H₁₄₇₀N₂₀₄O₂₈₅S₃, found: 19563.6957 Da, theoretical: 19563.6275 Da).

Linear protein: Acm protection on cysteine residues were removed using protocol 18. The ligation sample was purified by preparative HPLC (protocol 20). The purified linear protein was analyzed using HPLC method 2 (tR=13.075 min) and by HRMS (Formula: C₈₇₄H₁₄₆₀N₂₀₂O₂₈₃S₃, found: 19421.6056 Da, theoretical: 19421.5532 Da).

Folded protein: The linear protein was rearranged, folded and purified following protocol 19. The folded protein was analyzed using HPLC method 6 (tR=13.258 min) and by HRMS (Formula: C₈₇₄H₁₄₅₈N₂₀₂O₂₈₃S₃, found: 19419.5512 Da, theoretical: 19419.5375 Da).

### Example 15: Synthesis of CMP-141

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.342 min) and by HRMS (Formula: C₄₀₅H₇₃₈N₆₆O₁₅₄, found: 8996.2404 Da, theoretical: 8996.2096 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.842 min) and by HRMS (Formula: C₆₂₃H₁₁₀₁N₁₁₁O₂₂₃, found: 13746.8636 Da, theoretical: 13746.8180 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.125 min) and by HRMS (Formula: C₉₄₈H₁₆₁₇N₁₉₅O₃₂₂S₂, found: 20962.5670 Da, theoretical: 20962.5935 Da).

Linear protein: Acm protection on cysteine residues were removed using protocol 18. The ligation sample was purified by preparative HPLC (protocol 20). The purified linear protein was analyzed using HPLC method 2 (tR=13.125 min) and by HRMS (Formula: C₉₄₂H₁₆₀₇N₁₉₃O₃₂₀S₂, found: 20820.4559 Da, theoretical: 20820.5193 Da).

Folded protein: The linear protein was rearranged, folded and purified following protocol 19. The folded protein was analyzed using HPLC method 6 (tR=13.325 min) and by HRMS (Formula: C₉₄₂H₁₆₀₅N₁₉₃O₃₂₀S₂, found: 20818.4898 Da, theoretical: 20818.5036 Da).

### Example 16: Synthesis of CMP-142

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.425 min) and by HRMS (Formula: C₄₆₄H₈₅₅N₆₇O₁₈₃, found: 10300.0333 Da, theoretical: 10299.9825 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.342 min) and by HRMS (Formula: C₆₈₂H₁₂₁₈N₁₁₂O₂₅₂S, found: 15050.6483 Da, theoretical: 15050.5894 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.125 min) and by HRMS (Formula: C₁₀₀₇H₁₇₃₄N₁₉₆O₃₅₁S₂, found: 22267.2959 Da, theoretical: 22267.3677 Da).

Linear protein: Acm protection on cysteine residues were removed using protocol 18. The ligation sample was purified by preparative HPLC (protocol 20). The purified linear protein was analyzed using HPLC method 2 (tR=13.142 min) and by HRMS (Formula: C₁₀₀₁H₁₇₂₄N₁₉₄O₃₄₉S₂, found: 22125.2096 Da, theoretical: 22125.2935 Da).

Folded protein: The linear protein was rearranged, folded and purified following protocol 19. The folded protein was analyzed using HPLC method 6 (tR=13.275 min) and by HRMS (Formula: C₁₀₀₁H₁₇₂₂N₁₉₄O₃₄₉S₂, found: 22123.2044 Da, theoretical: 22123.2778 Da).

### Example 17: Synthesis of CMP-143

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.708 min) and by HRMS (Formula: C₃₁₁H₅₃₈N₇₀O₁₁₀, found: 7017.9051 Da, theoretical: 7017.8773 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.525 min) and by HRMS (Formula: C₅₂₉H₉₀₁N₁₁₅O₁₇₉S, found: 11768.4916 Da, theoretical: 11767.4829 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.908 min) and by HRMS (Formula: C₈₅₄H₁₄₁₇N₁₉₉O₂₇₈S₂, found: 18984.3084 Da, theoretical: 18984.2611 Da).

Linear protein: Acm protection on cysteine residues were removed using protocol 18. The ligation sample was purified by preparative HPLC (protocol 20). The purified linear protein was analyzed using HPLC method 2 (tR=13.075 min) and by HRMS (Formula: C₈₄₈H₁₄₀₇N₁₉₇O₂₇₆S₂, found: 18842.1387 Da, theoretical: 18842.1869 Da).

Folded protein: The linear protein was rearranged, folded and purified following protocol 19. The folded protein was analyzed using HPLC method 6 (tR=13.375 min) and by HRMS (Formula: C₈₄₈H₁₄₀₅N₁₉₇O₂₇₆S₂, found: 18840.1711 Da, theoretical: 18840.1712 Da).

### Example 18: Synthesis of CMP-144

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Final Fmoc deprotection was performed following protocol 6. Segment 1 was then released from the resin and the side chain deprotected following protocol 14. The crude peptide was purified by preparative HPLC (protocol 20). The purified segment 1 was analyzed using HPLC method 2 (tR=10.758 min) and by HRMS (Formula: C₃₅₂H₆₃₃N₆₇O₁₂₆, found: 7819.5596 Da, theoretical: 7819.5303 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.575 min) and by HRMS (Formula: C₅₇₀H₉₉₆N₁₁₂O₁₉₅S, found: 12570.1473 Da, theoretical: 12570.89 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.875 min) and by HRMS (Formula: C₈₉₅H₁₅₁₂N₁₉₆O₂₉₄S, found: 19785.9195 Da, theoretical: 19785.9142 Da).

Linear protein: Acm protection on cysteine residues were removed using protocol 18. The ligation sample was purified by preparative HPLC (protocol 20). The purified linear protein was analyzed using HPLC method 2 (tR=13.025 min) and by HRMS (Formula: C₈₈₉H₁₅₀₂N₁₉₄O₂₉₂S₂, found: 19644.7655 Da, theoretical: 19643.8400 Da).

Folded protein: The linear protein was rearranged, folded and purified following protocol 19. The folded protein was analyzed using HPLC method 6 (tR=13.325 min) and by HRMS (Formula: C₈₈₉H₁₅₀₀N₁₉₄O₂₉₂S₂, found: 19641.7505 Da, theoretical: 19641.8244 Da).

### Example 19: Synthesis of CMP-145

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Final Fmoc deprotection was performed following protocol 6. Segment 1 was then released from the resin and the side chain deprotected following protocol 14. The crude peptide was purified by preparative HPLC (protocol 20). The purified segment 1 was analyzed using HPLC method 2 (tR=10.575 min) and by HRMS (Formula: C₄₈₂H₈₉₁N₇₃₀₁₈₆, found: 10684.2903 Da, theoretical: 10685.2687 Da).

Segment 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.375 min) and by HRMS (Formula: C₇₀₀H₁₂₅₄N₁₁₈O₂₅₅S, found: 15435.907 Da, theoretical: 15435.877 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.033 min) and by HRMS (Formula: C₁₀₂₅H₁₇₇₀N₂₀₂O₃₅₄S₂, found: 22652.5701 Da, theoretical: 22651.6526 Da).

Linear protein: Acm protection on cysteine residues were removed using protocol 18. The ligation sample was purified by preparative HPLC (protocol 20). The purified linear protein was analyzed using HPLC method 2 (tR=12.925 min) and by HRMS (Formula: C₁₀₁₉H₁₇₆₀N₂₀₀₀₃₅₂S₂, found: 22509.4591 Da, theoretical: 22509.5784 Da).

Folded protein: The linear protein was rearranged, folded and purified following protocol 19. The folded protein was analyzed using HPLC method 6 (tR=13.175 min) and by HRMS (Formula: C₁₀₁₉H₁₇₅₈N₂₀₀O₃₅₂S₂, found: 22507.4974 Da, theoretical: 22507.5627 Da).

### Example 20: Synthesis of CMP-146

Segment 1: Loading of the first KAHA monomer 7 was performed following protocol 1. Elongation of the peptide chain was performed following protocol 3 until position 1 (Boc-Ala). Side chain Alloc deprotection of Lys (position 11) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.62 min) and by HRMS (Formula: C₂₈₈H₅₀₇N₆₃O₉₅, found: 6371.7382 Da, theoretical: 6371.6867 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.975 min) and by HRMS (Formula: C₅₀₆H₈₇₀N₁₀₈O₁₆₄S, found: 11123.2968 Da, theoretical: 11122.2951 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.025 min) and by HRMS (Formula: C₈₃₁H₁₃₈₆N₁₉₂O₂₆₃S₂, found: 18339.0991 Da, theoretical: 18339.0733 Da).

Linear protein: Acm protection on cysteine residues were removed using protocol 18. The ligation sample was purified by preparative HPLC (protocol 20). The purified linear protein was analyzed using HPLC method 2 (tR=13.25 min) and by HRMS (Formula: C₈₂₅H₁₃₇₆N₁₉₀O₂₆₁S₂, found: 18196.9540 Da, theoretical: 18195.9964 Da).

Folded protein: The linear protein was rearranged, folded and purified following protocol 19. The folded protein was analyzed using HPLC method 6 (tR=13.458 min) and by HRMS (Formula: C₈₂₅H₁₃₇₄N₁₉₀O₂₆₁S₂, found: 18193.9964 Da, theoretical: 18193.9807 Da).

### Example 21: Synthesis of CMP-147

Segment 1: Loading of the first KAHA monomer 7 was performed following protocol 1. Elongation of the peptide chain was performed following protocol 3 until position 1 (Boc-Ala). Side chain Alloc deprotection of Lys (position 13) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.808 min) and by HRMS (Formula: C₂₈₈H₅₀₇N₆₃O₉₅, found: 6371.7347 Da, theoretical: 6371.6867 Da).

Segments 2, 3, 4 and ligated segment 34 were synthesized as described in Synthesis of—CMP-118.

For Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.592 min) and by HRMS (Formula: C₅₀₆H₈₇₀N₁₀₈O₁₆₄S, found: 11122.3235 Da, theoretical: 11122.2951 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.092 min) and by HRMS (Formula: C₈₃₁H₁₃₈₆N₁₉₂O₂₆₃S₂, found: 18339.0889 Da, theoretical: 18339.0733 Da).

Linear protein: Acm protection on cysteine residues were removed using protocol 18. The ligation sample was purified by preparative HPLC (protocol 20). The purified linear protein was analyzed using HPLC method 2 (tR=13.308 min) and by HRMS (Formula: C₈₂₅H₁₃₇₆N₁₉₀O₂₆₁S₂, found: 18196.9363 Da, theoretical: 18195.9964 Da).

Folded protein: The linear protein was rearranged, folded and purified following protocol 19. The folded protein was analyzed using HPLC method 6 (tR=13.475 min) and by HRMS (Formula: C₈₂₅H₁₃₇₄N₁₉₀O₂₆₁S₂, found: 18194.9525 Da, theoretical: 18193.9807 Da).

### Example 22: Synthesis of CMP-148

Segment 1: Loading of the first KAHA monomer 7 was performed following protocol 1. Elongation of the peptide chain was performed following protocol 3 until position 1 (Boc-Ala). Side chain Alloc deprotection of Lys (position 15) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.292 min) and by HRMS (Formula: C₂₈₈H₅₀₈N₆₄O₉₄, found: 6370.7417 Da, theoretical: 6370.7026 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.625 min) and by HRMS (Formula: C₅₀₆H₈₇₁N₁₀₉O₁₆₃S, found: 11122.3330 Da, theoretical: 11121.3110 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.142 min) and by HRMS (Formula: C₈₃₁H₁₃₈₇N₁₉₃O₂₆₂S₂, found: 18338.0174 Da, theoretical: 18338.0893 Da).

Linear protein: Acm protection on cysteine residues were removed using protocol 18. The ligation sample was purified by preparative HPLC (protocol 20). The purified linear protein was analyzed using HPLC method 2 (tR=13.225 min) and by HRMS (Formula: C₈₂₅H₁₃₇₇N₁₉₁O₂₆₀S₂, found: 18196.0136 Da, theoretical: 18195.0123 Da).

Folded protein: The linear protein was rearranged, folded and purified following protocol 19. The folded protein was analyzed using HPLC method 6 (tR=13.442 min) and by HRMS (Formula: C₈₂₅H₁₃₇₅N₁₉₁O₂₆₀S₂, found: 18193.9222 Da, theoretical: 18192.9967 Da).

### Example 23: Synthesis of CMP-149

Segment 1: Loading of the first KAHA monomer 7 was performed following protocol 1. Elongation of the peptide chain was performed following protocol 3 until position 1 (Boc-Ala). Side chain Alloc deprotection of Lys (position 19) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 ((R=10.975 min) and by HRMS (Formula: C₂₈₇H₅₀₄N₆₄O₉₆, found: 6386.6980 Da, theoretical: 6386.6611 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.458 min) and by HRMS (Formula: C₅₀₅H₈₆₇N₁₀₉O₁₆₅S, found: 11137.2718 Da, theoretical: 11137.2696 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.108 min) and by HRMS (Formula: C₈₃₀H₁₃₈₃N₁₉₃O₂₆₄S₂, found: 18354.0052 Da, theoretical: 18354.0478 Da).

Linear protein: Acm protection on cysteine residues were removed using protocol 18. The ligation sample was purified by preparative HPLC (protocol 20). The purified linear protein was analyzed using HPLC method 2 (tR=13.175 min) and by HRMS (Formula: C₈₂₄H₁₃₇₃N₁₉₁O₂₆₂S₂, found: 18211.9716 Da, theoretical: 18210.9709 Da).

Folded protein: The linear protein was rearranged, folded and purified following protocol 19. The folded protein was analyzed using HPLC method 6 (tR=13.408 min) and by HRMS (Formula: C₈₂₄H₁₃₇₁N₁₉₁O₂₆₂S₂, found: 18209.891 Da, theoretical: 18208.9552 Da).

### Example 24: Synthesis of CMP-150

Segment 1: Loading of the first KAHA monomer 7 was performed following protocol 1. Elongation of the peptide chain was performed following protocol 3 until position 1 (Boc-Ala). Side chain Alloc deprotection of Lys (position 22) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=11.508 min) and by HRMS (Formula: C₂₈₈H₅₀₇N₆₃O₉₅, found: 6371.7267 Da, theoretical: 6371.6867 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.825 min) and by HRMS (Formula: C₅₀₆H₈₇₀N₁₀₈O₁₆₄S, found: 11122.3188 Da, theoretical: 11122.2951 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.068 min) and by HRMS (Formula: C₈₃₁H₁₃₈₆N₁₉₂O₂₆₃S₂, found: 18339.0376 Da, theoretical: 18339.0733 Da).

Linear protein: Acm protection on cysteine residues were removed using protocol 18. The ligation sample was purified by preparative HPLC (protocol 20). The purified linear protein was analyzed using HPLC method 2 (tR=13.242 min) and by HRMS (Formula: C₈₂₅H₁₃₇₆N₁₉₀O₂₆₁S₂, found: 18196.9646 Da, theoretical: 18195.9964 Da).

Folded protein: The linear protein was rearranged, folded and purified following protocol 19. The folded protein was analyzed using HPLC method 6 (tR=13.425 min) and by HRMS (Formula: C₈₂₅H₁₃₇₄N₁₉₀O₂₆₁S₂, found: 18194.9580 Da, theoretical: 18193.9807 Da).

### Example 25: Synthesis of CMP-162

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using analytical method 2 (tR=10.092 min) and by HRMS (Formula: C₃₂₆H₅₆₄N₈₀O₁₁₂, found: 7396.1270 Da, theoretical: 7396.1012 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using analytical method 2 (tR=9.925 min) and by HRMS (Formula: C₅₄₄H₉₂₇N₁₂₅O₁₈₁S, found: 12146.7060 Da, theoretical: 12146.7095 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using analytical method 2 (tR=12.592 min) and by HRMS (Formula: C₈₆₉H₁₄₄₃N₂₀₉O₂₈₀S₂, found: 19362.4650 Da, theoretical: 19362.4850 Da).

Linear protein: Acm protection on cysteine residues were removed using protocol 18. The ligation sample was purified by preparative HPLC (protocol 20). The purified linear protein was analyzed using analytical method 11 (tR=16.008 min) and by HRMS (Formula: C₈₆₃H₁₄₃₃N₂₀₇O₂₇₈S₂, found: 19220.4268 Da, theoretical: 19220.4108 Da).

Folded protein: The linear protein was rearranged, folded and purified following protocol 19. The folded protein was analyzed using HPLC method 6 (tR=13.308 min) and by HRMS (Formula: C₈₆₃H₁₄₃₁N₂₀₇O₂₇₈S₂, found: 19218.3851 Da, theoretical: 19218.3952 Da).

### Example 26: Synthesis of CMP-133

Segment 1: Loading of the first KAHA monomer 7 was performed following protocol 1. Elongation of the peptide chain was performed following protocol 3 until position PEG9 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 4 (tR=11.092 min) and by HRMS (Formula: C₂₇₅H₄₇₂N₆₄O₈₉, found: 6098.4761 Da, theoretical: 6098.4463 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.492 min) and by HRMS (Formula: C₄₉₃H₈₃₅N₁₀₉O₁₅₈S, found: 10850.0695 Da, theoretical: 10849.0547 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.625 min) and by HRMS (Formula: C₈₁₈H₁₃₅₁N₁₉₃O₂₅₇S₂, found: 18067.8077 Da, theoretical: 18064.8302 Da).

Linear protein: Acm protection on cysteine residues were removed using protocol 18. The ligation sample was purified by preparative HPLC (protocol 20). The purified linear protein was analyzed using HPLC method 7 (tR=12.158 min) and by HRMS (Formula: C₈₁₂H₁₃₄₁N₁₉₁O₂₅₅S₂, found: 17922.7039 Da, theoretical: 17922.7560 Da).

Folded protein: The linear protein was rearranged, folded and purified following protocol 19. The folded protein was analyzed using HPLC method 6 (tR=13.675 min) and by HRMS (Formula: C₈₁₂H₁₃₃₉N₁₉₁O₂₅₅S₂, found: 17920.7535 Da, theoretical: 17920.7403 Da).

### Example 27: Synthesis of CMP-134

Segment 1: Loading of the first KAHA monomer 7 was performed following protocol 1. Elongation of the peptide chain was performed following protocol 3 until position PEG4 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 4 (tR=10.625 min) and by HRMS (Formula: C₂₈₄H₄₇₈N₇₄O₉₀S, found: 6400.5330 Da, theoretical: 6400.4906 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.842 min) and by HRMS (Formula: C₅₀₂H₈₄₁N₁₁₉O₁₅₉S₂, found: 11151.1083 Da, theoretical: 11151.0989 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 7 (tR=11.792 min) and by HRMS (Formula: C₈₂₇H₁₃₅₇N₂₀₃O₂₅₈S₃, found: 18367.8602 Da, theoretical: 18367.877 Da).

Linear protein: Acm protection on cysteine residues were removed using protocol 18. The ligation sample was purified by preparative HPLC (protocol 20). The purified linear protein was analyzed using HPLC method 2 (tR=13.358 min) and by HRMS (Formula: C₈₂₁H₁₃₄₇N₂₀₁₀₂₅₆S₃, found: 18224.7887 Da, theoretical: 18225.8028 Da).

Folded protein: The linear protein was rearranged, folded and purified following protocol 19. The folded protein was analyzed using HPLC method 6 (tR=13.508 min) and by HRMS (Formula: C₈₂₁H₁₃₄₅N₂₀₁O₂₅₆S₃, found: 18223.7852 Da, theoretical: 18223.7871 Da).

### Example 28: Synthesis of CMP-135

Segment 1: Loading of the first KAHA monomer 7 was performed following protocol 1. Elongation of the peptide chain was performed following protocol 3 until position PEG16 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 4 (tR=10.775 min) and by HRMS (Formula: C₃₃₂H₅₇₄N₇₄O₁₁₄S, found: 7458.1518 Da, theoretical: 7458.1227 Da).

Segments 2, 3, 4 and ligated segment 34 were synthesized as described in Synthesis of—CMP-118.

For Segment 12: Ligation of segments 1 and 2 and photodeprotection were performed as described in protocol 15. The ligation/deprotection sample was purified by preparative HPLC (protocol 20). The purified segment 12 was analyzed using HPLC method 4 (tR=10.425 min) and by HRMS (Formula: C₅₅₀H₉₃₇N₁₁₉O₁₈₃S₂, found: 12209.7625 Da, theoretical: 12208.7310 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 7 (tR=11.792 min) and by HRMS (Formula: C₈₅₇H₁₄₅₃N₂₀₃O₂₈₂S₃, found: 19424.4226 Da, theoretical: 19424.5065 Da).

Linear protein: Acm protection on cysteine residues were removed using protocol 18. The ligation sample was purified by preparative HPLC (protocol 20). The purified linear protein was analyzed using HPLC method 2 (tR=13.208 min) and by HRMS (Formula: C₈₆₉H₁₄₄₃N₂₀₁O₂₈₀S₃, found: 19282.3817 Da, theoretical: 19282.4323 Da).

Folded protein: The linear protein was rearranged, folded and purified following protocol 19. The folded protein was analyzed using HPLC method 6 (tR=13.508 min) and by HRMS (Formula: C₈₆₉H₁₄₄₁N₂₀₁O₂₈₀S₃, found: 19280.4035 Da, theoretical: 19280.4167 Da).

### Example 29: Synthesis of CMP-140

Segment 1: Loading of the first KAHA monomer 7 was performed following protocol 1. Elongation of the peptide chain was performed following protocol 3 until position PEG9 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 4 (tR=11.208 min) and by HRMS (Formula: C₃₀₅H₅₃₂N₆₄O₁₀₄, found: 6758.8663 Da, theoretical: 6758.8396 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.608 min) and by HRMS (Formula: C₅₂₃H₈₉₅N₁₀₉O₁₇₃S, found: 11510.4607 Da, theoretical: 11509.4481 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.558 min) and by HRMS (Formula: C₈₄₈H₁₄₁₁N₁₉₃O₂₇₂S₂, found: 18726.1753 Da, theoretical: 18726.2263 Da).

Linear protein: Acm protection on cysteine residues were removed using protocol 18. The ligation sample was purified by preparative HPLC (protocol 20). The purified linear protein was analyzed using HPLC method 2 (tR=13.642 min) and by HRMS (Formula: C₈₄₂H₁₄₀₁N₁₉₁O₂₇₀S₂, found: 18584.1364 Da, theoretical: 18584.1521 Da).

Folded protein: The linear protein was rearranged, folded and purified following protocol 19. The folded protein was analyzed using HPLC method 6 (tR=13.708 min) and by HRMS (Formula: C₈₄₂H₁₃₉₉N₁₉₁O₂₇₀S₂, found: 18582.1193 Da, theoretical: 18582.1364 Da).

### Example 30: Synthesis of CMP-152

Segment 1: Loading of the first KAHA monomer 7 was performed following protocol 1. Elongation of the peptide chain was performed following protocol 3 until position PEG9 from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.992 min) and by MALDI-TOF (Formula: C₂₅₄H₄₃₁N₆₃O₇₉, found: 5631.101 Da, theoretical: 5631.600 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.225 min) and by HRMS (Formula: C₄₇₂H₇₉₄N₁₀₈O₁₄₈S, found: 10380.7899 Da, theoretical: 10381.7815 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.135 min) and by MALDI-TOF (Formula: C₇₉₇H₁₃₁₀N₁₉₂O₂₄₇S₂, found: 17601.908 Da, theoretical: 17598.464 Da).

Linear protein: Acm protection on cysteine residues were removed using protocol 18. The ligation sample was purified by preparative HPLC (protocol 20). The purified linear protein was analyzed using HPLC method 2 (tR=13.508 min) and by HRMS (Formula: C₇₉₁H₁₃₀₀N₁₉₀O₂₄₅S₂, found: 17455.456 Da, theoretical: 17455.4828 Da).

Folded protein: The linear protein was rearranged, folded and purified following protocol 19. The folded protein was analyzed using HPLC method 6 (tR=13.825 min) and by HRMS (Formula: C₇₉₁H₁₂₉₈N₁₉₀O₂₄₅S₂, found: 17453.473 Da, theoretical: 17453.97 Da).

### Example 31: Synthesis of CMP-153

Segment 1: Loading of the first KAHA monomer 7 was performed following protocol 1. Elongation of the peptide chain was performed following protocol 3 until position Gly from the linker part. Glutaric acid was then incorporated for protocol 9. Side chain Alloc deprotection of Lys (position 32) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.908 min) and by HRMS (Formula: C₂₅₆H₄₃₄N₆₄O₈₀, found: 5688.2219 Da, theoretical: 5688.1946 Da).

Segments 2, 3, 4 and ligated segment 34 were synthesized as described in Synthesis of CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.192 min) and by HRMS (Formula: C₄₇₄H₇₉₇N₁₀₉O₁₄₆S, found: 10437.8241 Da, theoretical: 10438.803 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.136 min) and by MALDI-TOF (Formula: C₇₉₉H₁₃₁₃N₁₉₃O₂₄₈S₂, found: 17652.662 Da, theoretical: 17655.516 Da).

Linear protein: Acm protection on cysteine residues were removed using protocol 18. The ligation sample was purified by preparative HPLC (protocol 20). The purified linear protein was analyzed using HPLC method 2 (tR=13.542 min) and by HRMS (Formula: C₇₉₃H₁₃₀₃N₁₉₁O₂₄₆S₂, found: 17512.4840 Da, theoretical: 17512.5043 Da).

Folded protein: The linear protein was rearranged, folded and purified following protocol 19. The folded protein was analyzed using HPLC method 6 (tR=13.808 min) and by HRMS (Formula: C₇₉₃H₁₃₀₁N₁₉₁O₂₄₆S₂, found: 17510.5274 Da, theoretical: 17510.4886 Da).

### Example 32: Synthesis of CMP-136

Segment 1: Loading of the first KAHA monomer 7 was performed following protocol 1. Elongation of the peptide chain was performed following protocol 3 until position PEG9 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.992 min) and by HRMS (Formula: C₂₇₁H₄₆₇N₆₃O₈₇, found: 5999.4505 Da, theoretical: 5999.4143 Da).

Segments 2, 3, 4 and ligated segment 34 were synthesized as described in Synthesis of CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.408 min) and by HRMS (Formula: C₄₈₉H₈₃₀N₁₀₈O₁₅₆S, found: 10751.0533 Da, theoretical: 10750.0226 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.137 min) and by MALDI-TOF (Formula: C₈₁₄H₁₃₄₆N₁₉₂O₂₅₅S₂, found: 17966.026 Da, theoretical: 17966.931 Da).

Linear protein: Acm protection on cysteine residues were removed using protocol 18. The ligation sample was purified by preparative HPLC (protocol 20). The purified linear protein was analyzed using HPLC method 2 (tR=13.608 min) and by HRMS (Formula: C₈₀₈H₁₃₃₆N₁₉₀O₂₅₃S₂, found: 17823.6979 Da, theoretical: 17823.7239 Da).

Folded protein: The linear protein was rearranged, folded and purified following protocol 19. The folded protein was analyzed using HPLC method 6 (tR=13.625 min) and by HRMS (Formula: C₈₀₈H₁₃₃₄N₁₉₀O₂₅₃S₂, found: 17822.7601 Da, theoretical: 17821.7083 Da).

### Example 33: Synthesis of CMP-137

Segment 1: Loading of the first KAHA monomer 7 was performed following protocol 1. Elongation of the peptide chain was performed following protocol 3 until position PEG24 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=12.242 min) and by HRMS (Formula: C₃₃₁H₅₈₇N₆₃O₁₁₇, found: 7321.2314 Da, theoretical: 7321.2038 Da).

Segments 2, 3, 4 and ligated segment 34 were synthesized as described in Synthesis of—CMP-118.

For segments 12: Ligation of segments 1 and 2 and photodeprotection were performed as described in protocol 15. The ligation/deprotection sample was purified by preparative HPLC (protocol 20). The purified segments 12 was analyzed using HPLC method 2 (tR=10.925 min) and by HRMS (Formula: C₅₄₆H₉₃₂N₁₁₈O₁₈₁S₂, found: 12109.7186 Da, theoretical: 12109.699 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.104 min) and by MALDI-TOF (Formula: C₈₇₄N₁₄₆₆N₁₉₂O₂₈₅S₂, found: 19288.371 Da, theoretical: 19288.521 Da).

Linear protein: Acm protection on cysteine residues were removed using protocol 18. The ligation sample was purified by preparative HPLC (protocol 20). The purified linear protein was analyzed using HPLC method 2 (tR=13.592 min) and by HRMS (Formula: C₈₆₈H₁₄₅₆N₁₉₀O₂₈₃S₂, found: 19145.488 Da, theoretical: 19145.5135 Da).

Folded protein: The linear protein was rearranged, folded and purified following protocol 19. The folded protein was analyzed using HPLC method 6 (tR=13.642 min) and by HRMS (Formula: C₈₆₈H₁₄₅₄N₁₉₀O₂₈₃S₂, found: 19143.5463 Da, theoretical: 19143.4978 Da).

### Example 34: Synthesis of CMP-138

Segment 1: Loading of the first KAHA monomer 7 was performed following protocol 1. Elongation of the peptide chain was performed following protocol 3 until position PEG4 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=10.942 min) and by HRMS (Formula: C₂₈₀H₄₇₃N₇₃O₈₈S, found: 6301.4654 Da, theoretical: 6301.4586 Da).

Segment 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.642 min) and by HRMS (Formula: C₄₉₈H₈₃₆N₁₁₈O₁₅₇S₂, found: 11052.0573 Da, theoretical: 11052.0669 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.153 min) and by MALDI-TOF (Formula: C₈₂₃H₁₃₅₂N₂₀₂O₂₅₆S₃, found: 18262.385 Da, theoretical: 18269.207 Da).

Linear protein: Acm protection on cysteine residues were removed using protocol 18. The ligation sample was purified by preparative HPLC (protocol 20). The purified linear protein was analyzed using HPLC method 2 (R=13.242 min) and by HRMS (Formula: C₈₁₇H₁₃₄₂N₂₀₀O₂₅₄S₃, found: 18126.7783 Da, theoretical: 18125.7681 Da).

Folded protein: The linear protein was rearranged, folded and purified following protocol 19. The folded protein was analyzed using HPLC method 6 (tR=13.492 min) and by HRMS (Formula: C₈₁₇H₁₃₄₀N₂₀₀O₂₅₄S₃, found: 18124.7931 Da, theoretical: 18123.7525 Da).

### Example 35: Synthesis of CMP-139

Segment 1: Loading of the first KAHA monomer 7 was performed following protocol 1. Elongation of the peptide chain was performed following protocol 3 until position PEG16 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.358 min) and by HRMS (Formula: C₃₂₈H₅₆₉N₇₃O₁₁₂S, found: 7359.1165 Da, theoretical: 7359.0907 Da).

Segments 2, 3, 4 and ligated segment 34 were synthesized as described in Synthesis of CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.642 min) and by HRMS (Formula: C₅₄₉H₉₅₀N₁₀₈O₁₈₆S, found: 12071.8242 Da, theoretical: 12071.8122 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.111 min) and by MALDI-TOF (Formula: C₈₇₁H₁₄₄₈N₂₀₂O₂₈₀S₃, found: 19326.067 Da, theoretical: 19326.479 Da).

Linear protein: Acm protection on cysteine residues were removed using protocol 18. The ligation sample was purified by preparative HPLC (protocol 20). The purified linear protein was analyzed using HPLC method 2 (tR=13.308 min) and by HRMS (Formula: C₈₆₅H₁₄₃₈N₂₀₀O₂₇₈S₃, found: 19183.3659 Da, theoretical: 19183.4003 Da).

Folded protein: The linear protein was rearranged, folded and purified following protocol 19. The folded protein was analyzed using HPLC method 6 (tR=13.475 min) and by HRMS (Formula: C₈₆₅H₁₄₃₆N₂₀₀O₂₇₈S₃, found: 19181.4007 Da, theoretical: 19171.3846 Da).

### Example 36: Synthesis of CMP-154

Segment 1: Loading of the first KAHA monomer 7 was performed following protocol 1. Elongation of the peptide chain was performed following protocol 3 until position PEG9 from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=10.992 min) and by HRMS (Formula: C₂₅₀H₄₂₆N₆₂O₇₇, found: 5532.1633 Da, theoretical: 5532.1411 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 5 (tR=4.477 min) and by HRMS (Formula: C₄₆₈H₇₈₉N₁₀₇O₁₄₆S, found: 10282.7779 Da, theoretical: 10282.7495 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.094 min) and by HRMS (Formula: C₇₉₃H₁₃₀₅N₁₉₁O₂₄₅S₂, found: 17498.5038 Da, theoretical: 17498.525 Da).

Linear protein: Acm protection on cysteine residues were removed using protocol 18. The ligation sample was purified by preparative HPLC (protocol 20). The purified linear protein was analyzed using HPLC method 5 (tR=5.151 min) and by HRMS (Formula: C₇₈₇H₁₂₉₅N₁₈₉O₂₄₃S₂, found: 17356.4531 Da, theoretical: 17356.4508 Da).

Folded protein: The linear protein was rearranged, folded and purified following protocol 19. The folded protein was analyzed using HPLC method 6 (tR=13.742 min) and by HRMS (Formula: C₇₈₇H₁₂₉₃N₁₈₉O₂₄₃S₂, found: 17354.4205 Da, theoretical: 17354.4351 Da).

### Example 37: Synthesis of CMP-155

Segment 1: Loading of the first KAHA monomer 7 was performed following protocol 1. Elongation of the peptide chain was performed following protocol 3 until position Gly from the linker part. Side chain All deprotection of Glu (position 29) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 5 (tR=4.63 min) and by HRMS (Formula: C₂₅₂H₄₂₉N₆₃O₇₈, found: 5589.2068 Da, theoretical: 5589.1626 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.525 min) and by HRMS (Formula: C₄₇₀H₇₉₂N₁₀₈O₁₄₇S, found: 10339.7723 Da, theoretical: 10339.7709 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=11.708 min) and by HRMS (Formula: C₇₉₅H₁₃₀₈N₁₉₂O₂₄₆S₂, found: 17555.512 Da, theoretical: 17555.5465 Da).

Linear protein: Acm protection on cysteine residues were removed using protocol 18. The ligation sample was purified by preparative HPLC (protocol 20). The purified linear protein was analyzed using HPLC method 2 (tR=11.725 min) and by HRMS (Formula: C₇₈₉H₁₂₉₈N₁₉₀O₂₄₄S₂, found: 17413.451 Da, theoretical: 17413.4722 Da).

Folded protein: The linear protein was rearranged, folded and purified following protocol 19. The folded protein was analyzed using HPLC method 6 (tR=13.742 min) and by HRMS (Formula: C₇₈₉H₁₂₉₆N₁₉₀O₂₄₄S₂, found: 17411.4358 Da, theoretical: 17411.4566 Da).

### Example 38: Synthesis of CMP-156

Segment 1: Loading of the first KAHA monomer 7 was performed following protocol 1. Elongation of the peptide chain was performed following protocol 3 until position Gly from the linker part. Side chain All deprotection of Glu (position 19) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 5 (R=4.558 min) and by HRMS (Formula: C₂₄₀H₄₀₄N₆₄O₇₄, found: 5370.029 Da, theoretical: 5369.99 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 5 (tR=4.428 min) and by HRMS (Formula: C₄₅₈H₇₆₇N₁₀₉O₁₄₃S, found: 10120,6362 Da, theoretical: 10120,5986 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.077 min) and by HRMS (Formula: C₇₈₃H₁₂₈₃N₁₉₃O₂₄₂S₂ found: 17336,3652 Da, theoretical: 17336,3741 Da).

Linear protein: Acm protection on cysteine residues were removed using protocol 18. The ligation sample was purified by preparative HPLC (protocol 20). The purified linear protein was analyzed using HPLC method 2 (tR=12.642 min) and by HRMS (Formula: C₇₇₇H₁₂₇₃N₁₉₁O₂₄₀S₂, found: 17194,3931 Da, theoretical: 17194.2999 Da).

Folded protein: The linear protein was rearranged, folded and purified following protocol 19. The folded protein was analyzed using HPLC method 6 (tR=13.308 min) and by HRMS (Formula: C₇₇₇H₁₂₇₁N₁₉₁O₂₄₀S₂, found: 17192.2843 Da, theoretical: 17193.3661 Da).

### Example 39: Synthesis of CMP-157

Segment 1: Loading of the first KAHA monomer 7 was performed following protocol 1. Elongation of the peptide chain was performed following protocol 3 until position PEG4 from the linker part. Side chain All deprotection of Glu (position 19) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.725 min) and by HRMS (Formula: C₂₃₈H₄₀₁N₆₃O₇₃, found: 5313.0138 Da, theoretical: 5312.9688 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.708 min) and by HRMS (Formula: C₄₅₆H₇₆₄N₁₀₈O₁₄₂S, found: 10063.6125 Da, theoretical: 10062.5745 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.225 min) and by HRMS (Formula: C₇₈₁H₁₂₈₀N₁₉₂O₂₄₁S₂, found: 17279.3246 Da, theoretical: 17279.3527 Da).

Linear protein: Acm protection on cysteine residues were removed using protocol 18. The ligation sample was purified by preparative HPLC (protocol 20). The purified linear protein was analyzed using HPLC method 2 (tR=13.292 min) and by HRMS (Formula: C₇₇₅H₁₂₇₀N₁₉₀O₂₃₉S₂, found: 17137.2785 Da, theoretical: 17137.3669 Da).

Folded protein: The linear protein was rearranged, folded and purified following protocol 19. The folded protein was analyzed using HPLC method 6 (tR=13.342 min) and by HRMS (Formula: C₇₇₅H₁₂₆₈N₁₉₀O₂₃₉S₂, found: 17135.3369 Da, theoretical: 17135.2628 Da).

### Example 40: Synthesis of CMP-158

Segment 1: Loading of the first KAHA monomer 7 was performed following protocol 1. Elongation of the peptide chain was performed following protocol 3 until position Gly from the linker part. Side chain All deprotection of Glu (position 22) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 5 (tR=4.725 min) and by HRMS (Formula: C₂₄₁H₄₀₇N₆₃O₇₃, found: 5355,0386 Da, theoretical: 5355.0158 Da).

Segments 2, 3, 4 and ligated segment 34 were synthesized as described in Synthesis of—CMP-118.

For segment 12: Ligation of segments 1 and 2 and photodeprotection were performed as described in protocol 15. The ligation/deprotection sample was purified by preparative HPLC (protocol 20). The purified segment 12 was analyzed using HPLC method 5 (tR=4.529 min) and by HRMS (Formula: C₄₇₀H₇₈₃N₁₀₉O₁₄₅S, found: 10104,6301 Da, theoretical: 10105,6241 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 5 (tR=5.105 min) and by HRMS (Formula: C₇₈₄H₁₂₈₆N₁₉₂O₂₄₁S₂, found: 17321,3931 Da, theoretical: 17321,3997 Da).

Linear protein: Acm protection on cysteine residues were removed using protocol 18. The ligation sample was purified by preparative HPLC (protocol 20). The purified linear protein was analyzed using HPLC method 2 (tR=12.792 min) and by HRMS (Formula: C₇₇₈H₁₂₇₆N₁₉₀O₂₃₉S₂, found: 17179,3934 Da, theoretical: 17179,3254 Da).

Folded protein: The linear protein was rearranged, folded and purified following protocol 19. The folded protein was analyzed using HPLC method 6 (tR=13.408 min) and by HRMS (Formula: C₇₇₈H₁₂₇₄N₁₉₀O₂₃₉S₂, found: 17178.3777 Da, theoretical: 17177.3098 Da).

### Example 41: Synthesis of CMP-159

Segment 1: Loading of the first KAHA monomer 7 was performed following protocol 1. Elongation of the peptide chain was performed following protocol 3 until position PEG4 from the linker part. Side chain All deprotection of Glu (position 22) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.858 min) and by HRMS (Formula: C₂₃₉H₄₀₄N₆₂O₇₂, found: 5297.0400 Da, theoretical: 5297.9943 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.825 min) and by HRMS (Formula: C₄₅₇H₇₆₇N₁₀₇O₁₄₁S, found: 10048.6371 Da, theoretical: 10048.6027 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.225 min) and by HRMS (Formula: C₇₈₂H₁₂₈₃N₁₉₁O₂₄₀S₂, found: 17264.3550 Da, theoretical: 17264.3782 Da).

Linear protein: Acm protection on cysteine residues were removed using protocol 18. The ligation sample was purified by preparative HPLC (protocol 20). The purified linear protein was analyzed using HPLC method 2 (tR=13.325 min) and by HRMS (Formula: C₇₇₆H₁₂₇₃N₁₈₉O₂₃₈S₂, found: 17122.3802 Da, theoretical: 17122.3040 Da).

Folded protein: The linear protein was rearranged, folded and purified following protocol 19. The folded protein was analyzed using HPLC method 6 (tR=13.342 min) and by HRMS (Formula: C₇₇₆H₁₂₇₁N₁₈₉O₂₃₈S₂, found: 17120.3456 Da, theoretical: 17120.2883 Da).

### Example 42: Synthesis of CMP-160

Segment 1: Loading of the first KAHA monomer 7 was performed following protocol 1. Elongation of the peptide chain was performed following protocol 3 until position Gly from the linker part. Side chain All deprotection of Glu (position 26) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 5 (tR=4.491 min) and by HRMS (Formula: C₂₅₂H₄₂₉N₆₃O₇₈, found: 5589.2053 Da, theoretical: 5589.1626 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 5 (tR=4.301 min) and by HRMS (Formula: C₄₇₀H₇₉₂N₁₀₈O₁₄₇S, found: 10339.8027 Da, theoretical: 10339.7709 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=12.642 min) and by HRMS (Formula: C₇₉₅H₁₃₀₈N₁₉₂O₂₄₆S₂, found: 17555.5332 Da, theoretical: 17555.5465 Da).

Linear protein: Acm protection on cysteine residues were removed using protocol 18. The ligation sample was purified by preparative HPLC (protocol 20). The purified linear protein was analyzed using HPLC method 2 (tR=12.692 min) and by HRMS (Formula: C₇₈₉H₁₂₉₈N₁₉₀O₂₄₄S₂, found: 17413.5417 Da, theoretical: 17413.4722 Da).

Folded protein: The linear protein was rearranged, folded and purified following protocol 19. The folded protein was analyzed using HPLC method 6 (tR=13.325 min) and by HRMS (Formula: C₇₈₉H₁₂₉₆N₁₉₀O₂₄₄S₂, found: 17412.5439 Da, theoretical: 17411.4566 Da).

### Example 43: Synthesis of CMP-161

Segment 1: Loading of the first KAHA monomer 7 was performed following protocol 1. Elongation of the peptide chain was performed following protocol 3 until position PEG9 from the linker part. Side chain All deprotection of Glu (position 26) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.392 min) and by HRMS (Formula: C₂₅₀H₄₂₆N₆₂O₇₇, found: 5532.1709 Da, theoretical: 5532.1411 Da).

Segments 2, 3, 4 and ligated segment 34 were synthesized as described in Synthesis of—CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.875 min) and by HRMS (Formula: C₄₆₈H₇₈₉N₁₀₇O₁₄₆S, found: 10282.7801 Da, theoretical: 10282.7495 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.258 min) and by HRMS (Formula: C₇₉₃H₁₃₀₅N₁₉₁O₂₄₅S₂, found: 17498.5078 Da, theoretical: 17498.5250 Da).

Linear protein: Acm protection on cysteine residues were removed using protocol 18. The ligation sample was purified by preparative HPLC (protocol 20). The purified linear protein was analyzed using HPLC method 2 (tR=13.342 min) and by HRMS (Formula: C₇₈₇H₁₂₉₅N₁₈₉O₂₄₃S₂, found: 17356.5255 Da, theoretical: 17356.4508 Da).

Folded protein: The linear protein was rearranged, folded and purified following protocol 19. The folded protein was analyzed using HPLC method 6 (R=13.342 min) and by HRMS (Formula: C₇₈₇H₁₂₉₃N₁₈₉O₂₄₃S₂, found: 17355.5218 Da, theoretical: 17354.4351 Da).

### Example 44: Synthesis of CMP-151

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until PEG4 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.708 min) and by HRMS (Formula: C₂₅₄H₄₃₂N₆₄O₇₉, found: 5646.2349 Da, theoretical: 5646.184 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.158 min) and by HRMS (Formula: C₄₇₂H₇₉₅N₁₀₉O₁₄₈S, found: 10396.8346 Da, theoretical: 10396.7924 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.392 min) and by HRMS (Formula: C₇₉₇H₁₃₁₁N₁₉₃O₂₄₇S₂, found: 17612.5541 Da, theoretical: 17612.5679 Da).

Linear protein: Acm protection on cysteine residues were removed using protocol 18. The ligation sample was purified by preparative HPLC (protocol 20). The purified linear protein was analyzed using HPLC method 2 (tR=13.475 min) and by HRMS (Formula: C₇₉₁H₁₃₀₁N₁₉₁O₂₄₅S₂, found: 17470.4669 Da, theoretical: 17470.4937 Da).

Folded protein: The linear protein was rearranged, folded and purified following protocol 19. The folded protein was analyzed using HPLC method 6 (tR=13.642 min) and by HRMS (Formula: C₇₉₁H₁₂₉₉N₁₉₁O₂₄₅S₂, found: 17468.4077 Da, theoretical: 17468.478 Da).

### Example 45: Synthesis of CMP-163

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until PEG4 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using method 2 (tR=11.408 min) and by HRMS (Formula: C₂₄₄H₄₁₂N₆₄O₇₄, found: 5426.0601 Da, theoretical: 5426.0529 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using method 2 (tR=10.792 min) and by HRMS (Formula: C₄₆₂H₇₇₅N₁₀₉O₁₄₃S, found: 10175.6731 Da, theoretical: 10176.6613 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using method 2 (tR=13.492 min) and by HRMS (Formula: C₇₈₇H₁₂₉₁N₁₉₃O₂₄₂S₂, found: 17392.4924 Da, theoretical: 17392.4368 Da).

Linear protein: Acm protection on cysteine residues were removed using protocol 18. The ligation sample was purified by preparative HPLC (protocol 20). The purified linear protein was analyzed using method 2 (tR=13.425 min) and by HRMS (Formula: C₇₈₁H₁₂₈₁N₁₉₁O₂₄₀S₂, found: 17250.4162 Da, theoretical: 17250.3625 Da).

Folded protein: The linear protein was rearranged, folded and purified following protocol 19. The folded protein was analyzed using HPLC method 6 (tR=13.675 min) and by HRMS (Formula: C₇₈₁H₁₂₇₉N₁₉₁O₂₄₀S₂, found: 17249.3681 Da, theoretical: 17249.3469 Da).

### Example 46: Synthesis of CMP-164

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until Gly from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using method 2 (tR=11.392 min) and by HRMS (Formula: C₂₃₅H₃₉₄N₆₄O₇₀, found: 5234.9369 Da, theoretical: 5235.9323 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using method 2 (tR=10.942 min) and by HRMS (Formula: C₄₅₃H₇₅₇N₁₀₉O₁₃₉S, found: 9986.5585 Da, theoretical: 9985.538 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using method 2 (tR=13.358 min) and by HRMS (Formula: C₇₇₈H₁₂₇₃N₁₉₃O₂₃₈S₂, found: 17202.3594 Da, theoretical: 17202.3162 Da).

Linear protein: Acm protection on cysteine residues were removed using protocol 18. The ligation sample was purified by preparative HPLC (protocol 20). The purified linear protein was analyzed using method 2 (tR=13.425 min) and by HRMS (Formula: C₇₇₂H₁₂₆₃N₁₉₁O₂₃₆S₂, found: 17060.2794 Da, theoretical: 17060.242 Da).

The linear protein was rearranged, folded and purified following protocol 19. The folded protein was analyzed using HPLC method 6 (tR=13.675 min) and by HRMS (Formula: C₇₇₂H₁₂₆₁N₁₉₁O₂₃₆S₂, found: 17058.2263 Da, theoretical: 17058.2263 Da).

### Example 47: Synthesis of CMP-165

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until Gly from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using method 2 (tR=10.525 min) and by HRMS (Formula: C₂₇₂H₄₅₁N₇₉O₈₅, found: 6187.3571 Da, theoretical: 6187.3481 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using method 2 (tR=10.492 min) and by HRMS (Formula: C₄₉₀H₈₁₄N₁₂₄O₁₅₄S, found: 10937.9718 Da, theoretical: 10937.9565 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using method 2 (tR=13.075 min) and by HRMS (Formula: C₈₁₅H₁₃₃₀N₂₀₈O₂₅₃S₂, found: 18153.7813 Da, theoretical: 18153.7320 Da).

Linear protein: Acm protection on cysteine residues were removed using protocol 18. The ligation sample was purified by preparative HPLC (protocol 20). The purified linear protein was analyzed using method 2 (tR=13.142 min) and by HRMS (Formula: C₈₀₉H₁₃₂₀N₂₀₆O₂₅₁S₂, found: 18011.6885 Da, theoretical: 18011.6578 Da).

The linear protein was rearranged, folded and purified following protocol 19. The folded protein was analyzed using HPLC method 6 (tR=13.508 min) and by HRMS (C₈₀₉H₁₃₁₈N₂₀₆O₂₅₁S₂, found: 18010.6228 Da, theoretical: 18009.6421 Da)

### Example 48: Synthesis of CMP-166

Segment 1: Loading of the first KAHA monomer 7 was performed following protocol 1. Elongation of the peptide chain was performed following protocol 3 until position 1 (Ala). The free N-terminus Ala1 was Alloc protected using protocol 5 Side chain ivDde protection of Lys (position 23) was removed following protocol 8 and elongation was pursued on the free amine side chain following protocol 3 until PEG4 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using analytical method 2 (RT=10.642 min) and by HRMS (Formula: C₂₈₃H₄₇₂N₈₀O₉₀, found: 6434.5081 Da, theoretical: 6434.4901 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.525 min) and by HRMS (Formula: C₅₀₁H₈₃₅N₁₂₅O₁₅₉S, found: 11185.1118 Da, theoretical: 11185.0985 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 11 (tR=16.542 min) and by HRMS (Formula: C₈₂₆H₁₃₅₁N₂₀₉O₂₅₈S₂, found: 18401.8681 Da, theoretical: 18401.8762 (M+1) Da).

Linear protein: Acm protection on cysteine residues were removed using protocol 18. The ligation sample was purified by preparative HPLC (protocol 20). The purified linear protein was analyzed using HPLC method 11 (tR=16.492 min) and by HRMS (Formula: C₈₂₀H₁₃₄₁N₂₀₇O₂₅₆S₂, found: 18258.7752 Da, theoretical: 18258.7998 Da).

Folded protein: The linear protein was rearranged, folded and purified following protocol 19. The folded protein was analyzed using HPLC method 6 (tR=13.442 min) and by HRMS (Formula: C₈₂₀H₁₃₃₉N₂₀₇O₂₅₆S₂, found: 18257.7739 Da, theoretical: 18256.7842 Da).

### Example 49: Synthesis of CMP-167

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until PEG9 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using analytical method 2 (tR=10.642 min) and by HRMS (Formula: C₂₉₃H₄₉₂N₈₀O₉₅, found: 6654.6213 Da, theoretical: 6654.6361 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.525 min) and by HRMS (Formula: C₅₁₁H₈₅₅N₁₂₅O₁₆₄S, found: 11406.2329 Da, theoretical: 11405.2297).

Segment 1234 (Acm protected cysteines): Ligation of segments 12 and 34 was performed as described in protocol 17. The purified segment 1234 (Acm protected cysteines) was analyzed using HPLC method 11 (tR=16.525 min) and by HRMS (Formula: C₈₃₆H₁₃₇₁N₂₀₉O₂₆₃S₂, found: 18621.9821 Da, theoretical: 18622.0073 Da).

Linear protein: Acm protection on cysteine residues were removed using protocol 18. The ligation sample was purified by preparative HPLC (protocol 20). The purified linear protein was analyzed using HPLC method 11 (RT=16.492 min) and by HRMS (Formula: C₈₃₀H₁₃₆₁N₂₀₇O₂₆₁S₂, found: 18478.9172 Da, theoretical: 18479.9336 Da).

Folded protein: The linear protein was rearranged, folded and purified following protocol 19. The folded protein was analyzed using HPLC method 6 (tR=13.442 min) and by HRMS (Formula: C₈₃₀H₁₃₅₉N₂₀₇O₂₆₁S₂, found: 18476.9162 Da, theoretical: 18477.918 Da).

### Example 50: Synthesis of CMP-168

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side chain Alloc deprotection of Lys (position 9) was carried out using protocol 7. The linker was elongated on the Lys side chain until residue PEG4 kept Fmoc protected. 2-Cl-Trt deprotection on Glu (position 23) was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.558 min) and by HRMS (Formula: C₂₃₈H₄₀₁N₆₃O₇₃, found: 5312.9824 Da, theoretical: 5312.9688 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.008 min) and by HRMS (Formula: C₄₅₆H₇₆₄N₁₀₈O₁₄₂S, found: 10062.5919 Da, theoretical: 10062.5745 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.358 min) and by HRMS (Formula: C₇₈₁H₁₂₈₀N₁₉₂O₂₄₁S₂, found: 17280.3857 Da, theoretical: 17279.3527 Da).

Linear protein: Acm protection on cysteine residues were removed using protocol 18. The ligation sample was purified by preparative HPLC (protocol 20). The purified linear protein was analyzed using HPLC method 2 (tR=13.408 min) and by HRMS (Formula: C₇₇₅H₁₂₇₀N₁₉₀O₂₃₉S₂, found: 17137.3029 Da, theoretical: 17137.2785 Da).

Folded protein: The linear protein was rearranged, folded and purified following protocol 19. The folded protein was analyzed using HPLC method 6 (tR=13.658 min) and by HRMS (Formula: C₇₇₅H₁₂₆₈N₁₉₀O₂₃₉S₂, found: 17136.2903 Da, theoretical: 17135.2628 Da).

### Example 51: Synthesis of CMP-169

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side-chain Alloc deprotection of Lys (position 9) was carried out using protocol 7. The linker was elongated on the Lys side chain until residue Gly kept Fmoc protected. 2-Cl-Trt deprotection on Glu23 was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.408 min) and by HRMS (Formula: C₂₄₀H₄₀₄N₆₄O₇₄, found: 5368.9927 Da, theoretical: 5369.9903 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=11.042 min) and by HRMS (Formula: C₄₅₈H₇₆₇N₁₀₉O₁₄₃S, found: 10120.6058 Da, theoretical: 10120.5986 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 2 (tR=13.292 min) and by HRMS (Formula: C₇₈₃H₁₂₈₃N₁₉₃O₂₄₂S₂, found: 17336.3845 Da, theoretical: 17336.3741 Da).

Linear protein: Acm protection on cysteine residues were removed using protocol 18. The ligation sample was purified by preparative HPLC (protocol 20). The purified linear protein was analyzed using HPLC method 2 (tR=13.375 min) and by HRMS (Formula: C₇₇₇H₁₂₇₃N₁₉₁O₂₄₀S₂, found: 17194.2984 Da, theoretical: 17194.2999 Da).

Folded protein: The linear protein was rearranged, folded and purified following protocol 19. The folded protein was analyzed using HPLC method 6 (tR=13.625 min) and by HRMS (Formula: C₇₇₇H₁₂₇₁N₁₉₁O₂₄₀S₂, found: 17193.302 Da, theoretical: 17192.2843 Da).

### Example 52: Synthesis of CMP-130

Segment 1: Loading of the first KAHA monomer 5 was performed following protocol 1. Elongation of the peptide chain was performed following protocol 3 until position 1 (Ala1). Segment 1 was then released from the resin and the side chain deprotected following protocol 14. The crude peptide was purified by preparative HPLC (protocol 20). The purified segment 1 was analyzed using HPLC method 2 (tR=11.008 min) and by HRMS (Formula: C₂₀₂H₃₄₇N₅₅O₆₁, found: 4521.6100 Da, theoretical: 4521.5799 Da).

Segments 2, 3, 4 and ligated segment 34 were synthesized as described in Synthesis of—CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 8 (tR=14.792 min) and by HRMS (Formula: C₄₂₀H₇₁₀N₁₀₀O₁₃₀S, found: 9273.1906 Da, theoretical: 9272.1883 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using 13.408 (tR=13.408 min) and by HRMS (Formula: C₇₄₅H₁₂₂₆N₁₈₄O₂₂₉S₂, found: 16488.931 Da, theoretical: 16487.9638 Da).

Linear protein: Acm protection on cysteine residues were removed using protocol 18. The ligation sample was purified by preparative HPLC (protocol 20). The purified linear protein was analyzed using HPLC method 2 (tR=13.425 min) and by HRMS (Formula: C₇₃₉H₁₂₁₆N₁₈₂O₂₂₇S₂, found: 16345.8812 Da, theoretical: 16345.8896 Da).

Folded protein: The linear protein was rearranged, folded and purified following protocol 19. The folded protein was analyzed using HPLC method 6 (tR=13.675 min) and by HRMS (Formula: C₇₃₉H₁₂₁₄N₁₈₂O₂₂₇S₂, found: 16344.8579 Da, theoretical: 16343.8739 Da)

### Example 53: Synthesis of CMP-131

Segment 1: Loading of the first KAHA monomer 5 was performed following protocol 1. Elongation of the peptide chain was performed following protocol 3 until position 1 (Boc-Ala). Side chain Alloc deprotection of Dab (position 16) was carried out following protocol 7. Building block 8 (2 equiv.) was then added to the resin in presence of DIPEA (4 equiv.) in DMF (25 mL/mmol resin substitution) for 18 h. The resin was then washed with DMF. After Fmoc deprotection, Fmoc-Glu (OtBu)-OH was coupled using protocol 3. After Fmoc deprotection, the free amine was acetylated (see procedure in protocol 3). Segment 1 was then released from the resin and the side chain deprotected following protocol 14 for only 30 min instead 2h30. The crude peptide was purified by preparative HPLC (protocol 20). The purified segment 1 was analyzed using HPLC method 2 (tR=11.392 min) and by HRMS (Formula: C₂₂₈H₃₈₃N₆₁O₇₀, found: 5097.8669 Da, theoretical: 5097.8343 Da).

Segments 2, 3, 4 and ligated segment 34 were synthesized as described in Synthesis of—CMP-118. For segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.975 min) and by HRMS (Formula: C₄₄₆H₇₄₆N₁₀₆O₁₃₉S, found: 9848.4435 Da, theoretical: 9848.4427 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using 13.375 (tR=13.375 min) and by HRMS (Formula: C₇₇₁H₁₂₆₂N₁₉₀O₂₃₈S₂, found: 17065.1877 Da, theoretical: 17065.2209 Da).

Linear protein: Acm protection on cysteine residues were removed using protocol 18. The ligation sample was purified by preparative HPLC (protocol 20). The purified linear protein was analyzed using HPLC method 2 (tR=13.725 min) and by HRMS (Formula: C₇₆₅H₁₂₅₂N₁₈₈O₂₃₆S₂, found: 16923.1382 Da, theoretical: 16923.1467 Da).

Folded protein: The linear protein was rearranged, folded and purified following protocol 19. The folded protein was analyzed using HPLC method 6 (tR=13.708 min) and by HRMS (Formula: C₇₆₅H₁₂₅₀N₁₈₈O₂₃₆S₂, found: 16921.0886 Da, theoretical: 16921.131 Da).

### Example 54A: Synthesis of CMP-132

Segment 1: Loading of the first KAHA monomer 5 was performed following protocol 1. Elongation of the peptide chain was performed following protocol 3 until position 1 using building block 9 in position 6. Segment 1 was then released from the resin and the side chain deprotected following protocol 14 with only 30 min instead 2h30. The crude peptide was purified by preparative HPLC (protocol 20). The purified segment 1 was analyzed using HPLC method 2 (tR=11.008 min) and by HRMS (Formula: C₂₂₉H₃₈₂N₆₂O₆₈, found: 5090.8791 Da, theoretical: 5090.8397 Da).

Segments 2, 3, 4 and ligated segment 34 were synthesized as described in Synthesis of—CMP-118.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 2 (tR=10.708 min) and by HRMS (Formula: C₄₄₇H₇₄₅N₁₀₇O₁₃₇S, found: 9841.4448 Da, theoretical: 9841.4481 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using 13.158 (tR=13.158 min) and by HRMS (Formula: C₇₇₂H₁₂₆₁N₁₉₁O₂₃₆S₂, found: 17058.2861 Da, theoretical: 17058.2263 Da).

Linear protein: Acm protection on cysteine residues were removed using protocol 18. The ligation sample was purified by preparative HPLC (protocol 20). The purified linear protein was analyzed using HPLC method 2 (tR=13.342 min) and by HRMS (Formula: C₇₆₆H₁₂₅₁N₁₈₉O₂₃₄S₂, found: 16916.2089 Da, theoretical: 16916.1521 Da).

Folded protein: The linear protein was rearranged, folded and purified following protocol 19. The folded protein was analyzed using HPLC method 6 (tR=13.442 min) and by HRMS (Formula: C₇₆₆H₁₂₄₉N₁₈₉O₂₃₄S₂, found: 16914.1606 Da, theoretical: 16914.1364 Da).

### Example 54B: Synthesis of CMP-310

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=10.225 min) and by HRMS (Formula: C₄₇₃H₈₇₅N₇₃O₁₈₉, found: 10609.1397 Da, theoretical: 10609.1282 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (tR=11.725 min) and by HRMS (Formula: C₆₉₁H₁₂₃₈N₁₁₈O₂₅₈S, found: 15358.7502 Da, theoretical: 15359.7366 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (tR=15.342 min) and by HRMS (Formula: C₁₀₁₇H₁₇₅₆N₂₀₂O₃₅₇S₂, found: 22575.306 Da, theoretical: 22575.5121 Da).

Linear protein: Acm protection on cysteine residues were removed using protocol 18. The ligation sample was purified by preparative HPLC (protocol 20). The purified linear protein was analyzed using HPLC method 12 (tR=15.675 min) and by HRMS (Formula: C₁₀₁₀H₁₇₄₄N₂₀₀O₃₅₅S₂, found: 22433.4378 Da, theoretical: 22433.3063 Da).

Folded protein: The linear protein was rearranged, folded and purified following protocol 19. The folded protein was analyzed using HPLC method 6 (tR=13.408 min) and by HRMS (Formula: C₁₀₁₀H₁₇₄₂N₂₀₀O₃₅₅S₂, found: 22431.3198 Da, theoretical: 22431.4222 Da).

### Example 54C: Synthesis of CMP-311

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 2 (tR=9.958 min) and by HRMS (Formula: C₄₉₄H₉₁₄N₈₀O₁₉₁, found: 11030.4674 Da, theoretical: 11030.4447 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (tR=11.792 min) and by HRMS (Formula: C₇₁₂H₁₂₇₇N₁₂₅O₂₆₀S, found: 15781.0666 Da, theoretical: 15.781.0531 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (tR=15.492 min) and by HRMS (Formula: C₁₀₃₈H₁₇₉₄N₂₀₈O₃₅₉S₂, found: 17394.3791 Da, theoretical: 17393.3579 Da).

Linear protein: Acm protection on cysteine residues were removed using protocol 18. The ligation sample was purified by preparative HPLC (protocol 20). The purified linear protein was analyzed using HPLC method 12 (tR=15.658 min) and by HRMS (Formula: C₁₀₃₂H₁₇₈₄N₂₀₆O₃₅₇S₂, found: 22854.6262 Da, theoretical: 22853.7591 Da).

Folded protein: The linear protein was rearranged, folded and purified following protocol 19. The folded protein was analyzed using HPLC method 6 (tR=13.375 min) and by HRMS (Formula: C₁₀₃₂H₁₇₈₂N₂₀₆O₃₅₇S₂, found: 22852.5944 Da, theoretical: 22851.7435 Da).

### Example 54D: Synthesis of CMP-312

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 14 (t_{R}=13.375 min) and by MS (ESI) (Formula: C₃₅₀H₆₂₆N₈₀O₁₁₉, found: 1098.82 Da, theoretical [M+7H]^{7+/}7:1098.58 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (t_{R}=11.625 min) and by MS (ESI). Formula: C₅₆₀H₉₇₃N₁₂₅O₁₈₄S, found: 1555.72 Da, theoretical [M+8H]^{8+/}8:1555.21 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.492 min) and by HRMS (Formula: C₈₈₅H₁₄₈₉N₂₀₉O₂₈₃S₂, found: 19648.7444 Da, theoretical: 19648.8299 Da).

Linear protein: Acm protection on cysteine residues were removed using protocol 18. The ligation sample was purified by preparative HPLC (protocol 20). The purified linear protein was analyzed using HPLC method 12 (t_{R}=14.958 min) and by HRMS (Formula: C₈₇₉H₁₄₇₉N₂₀₇O₂₈₁S₂, found: 19506.726 Da, theoretical: 19506.7260 Da).

Folded protein: The linear protein was rearranged, folded and purified following protocol 19. The folded protein was analyzed using HPLC method 6 (t_{R}=13.208 min) and by HRMS (Formula: C₈₇₉H₁₄₇₇N₂₀₇O₂₈₁S₂, found: 19504.7154 Da, theoretical: 19504.7400 Da).

### Example 54E: Synthesis CMP-313

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 13 (t_{R}=14.108 min) and by MS (ESI) (Formula: C₅₀₃H₉₃₃N₈₇O₁₉₁, found: 1408.21 Da, theoretical [M+8H]^{8+/}8:1408.05 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 13 (t_{R}=14.008 min) and by MS (ESI) (Formula: C₇₂₁H₁₂₉₆N₁₃₂O₂₆₀S, found: 1601.89 Da, theoretical [M+10H]^{10+/}10:1601.70 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.525 min) and by HRMS (Formula: C₁₀₄₆H₁₈₁₂N₂₁₆O₃₅₉S₂, found: 23222.7736 Da, theoretical: 23221.9987 Da).

Linear protein: Acm protection on cysteine residues were removed using protocol 18. The ligation sample was purified by preparative HPLC (protocol 20). The purified linear protein was analyzed using HPLC method 12 (t_{R}=15.342 min) and by HRMS (Formula: C₁₀₄₀H₁₈₀₂N₂₁₄O₃₅₇S₂, found: 23079.7430 Da, theoretical: 23079.9245 Da).

Folded protein: The linear protein was rearranged, folded and purified following protocol 19. The folded protein was analyzed using HPLC method 6 (t_{R}=13.142 min) and by HRMS (Formula: C₁₀₄₀H₁₈₀₀N₂₁₄O₃₅₇S₂, found: 23078.6502 Da, theoretical: 23077.901 Da).

### Example 54F: Synthesis of CMP-314

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 13 (t_{R}=13.125 min) and by MS (ESI) (Formula: C₃₅₁H₆₂₉N₈₇O₁₁₅, found: 1131.28 Da, theoretical [M+7H]^{7+/}7:1130.77 Da).

Segment 2, 3, 4 and ligated segment 34 were synthesized as described in Example 3.

Segment 12: Ligation of segment 1 and 2 and photodeprotection were performed as described in protocol 15. The ligation/deprotection sample was purified by preparative HPLC (protocol 20). The purified segment 12 was analyzed using HPLC method 13 (t_{R}=13.708 min) and by MS (ESI) (Formula: C₅₆₉H₉₉₂N₁₃₂O₁₈₄S, found: 1407.88 Da, theoretical [M+9H]⁹⁺/9:1407.55 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.542 min) and by HRMS (Formula: C₈₉₄H₁₅₀₈N₂₁₆O₂₈₃S₂, found: 19873.9230 Da, theoretical: 19874.0000 Da).

Linear protein: Acm protection on cysteine residues were removed using protocol 18. The ligation sample was purified by preparative HPLC (protocol 20). The purified linear protein was analyzed using HPLC method 12 (t_{R}=15.658 min) and by HRMS (Formula: C₈₈₈H₁₄₉₈N₂₁₄O₂₈₁S₂, found: 19731.8554 Da, theoretical: 19731.9258 Da).

Folded protein: The linear protein was rearranged, folded and purified following protocol 19. The folded protein was analyzed using HPLC method 6 (t_{R}=13.192 min) and by HRMS (Formula: C₈₈₈H₁₄₉₆N₂₁₄O₂₈₁S₂, found: 19730.7738 Da, theoretical: 19729.9101 Da).

### Example 54G: Synthesis of CMP-315

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 14 (t_{R}=13.192 min) and by MS (ESI) (Formula: C₃₄₇H₆₁₇N₈₁O₁₁₆, found: 1297.68 Da, theoretical [M+6H]⁶⁺/6:1297.70 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 13 (t_{R}=13.908 min) and by MS (ESI) (Formula: C₅₆₅H₉₇₉N₁₂₅O₁₈₆S, found: 1393.09, Da, theoretical [M+9H]⁹⁺/9:1393.42 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.325 min) and by HRMS (Formula: C₈₉₀H₁₄₉₅N₂₀₉O₂₈₅S₂, found: 19746.7758 Da, theoretical: 19745.8826 Da).

Linear protein: Acm protection on cysteine residues were removed using protocol 18. The ligation sample was purified by preparative HPLC (protocol 20). The purified linear protein was analyzed using HPLC method 12 (t_{R}=14.942 min) and by HRMS (Formula: C₈₈₄H₁₄₈₅N₂₀₇O₂₈₃S₂, found: 19604.7570 Da, theoretical: 19604.7924 Da).

Folded protein: The linear protein was rearranged, folded and purified following protocol 19. The folded protein was analyzed using HPLC method 6 (t_{R}=13.208 min) and by HRMS (Formula: C₈₈₄H₁₄₈₃N₂₀₇O₂₈₃S₂, found: 19602.6939 Da, theoretical: 19602.7768 Da).

### Example 54H: Synthesis of CMP-316

Segment 1: Loading of the first KAHA monomer 7 was performed following protocol 1. Elongation of the peptide chain was performed following protocol 3 until position 1 (Boc-Ala). Side chain Alloc deprotection of Lys (position 23) was performed following protocol 7 and elongation was pursued on the free amine side chain following protocol 3. Segment 1 was then released from the resin and the side chain deprotected following protocol 14. The crude peptide was purified by preparative HPLC (protocol 20). The purified segment 1 was analyzed using HPLC method 14 (t_{R}=13.142 min) and by MS (ESI) (Formula: C₃₂₆H₅₇₉N₇₅O₁₁₆, found: 1235.15 Da, theoretical [M+6H]⁶⁺/6:1235.27 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 13 (t_{R}=13.825 min) and by MS (ESI) (Formula: C₅₄₄H₉₄₂N₁₂₀O₁₈₅S, found: 1351.30 Da, theoretical [M+9H]⁹⁺/9:1351.69 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.375 min) and by HRMS (Formula: C₈₆₉H₁₄₅₈N₂₀₄O₂₈₄S₂, found: 19371.5298 Da, theoretical: 19371.5668 Da).

Linear protein: Acm protection on cysteine residues were removed using protocol 18. The ligation sample was purified by preparative HPLC (protocol 20). The purified linear protein was analyzed using HPLC method 12 (t_{R}=15.142 min) and by HRMS (Formula: C₈₆₃H₁₄₄₈N₂₀₂O₂₈₂S₂, found: 19229.4146 Da, theoretical: 19229.4926 Da).

Folded protein: The linear protein was rearranged, folded and purified following protocol 19. The folded protein was analyzed using HPLC method 6 (t_{R}=13.225 min) and by HRMS (Formula: C₈₆₃H₁₄₄₆N₂₀₂O₂₈₂S₂, found: 19227.4310 Da, theoretical: 19227.4769 Da).

### Example 541: Synthesis of CMP-317

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 13 (t_{R}=15.058 min) and by MS (ESI) (Formula: C₂₃₁H₃₈₈N₆₄O₇₇, found: 1324.10 Da, theoretical [M+4H]⁴⁺/4:1324.50 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=14.475 min) and by MS (ESI) (Formula: C₄₄₉H₇₅₁N₁₀₉O₁₄₆S, found: 1675.61 Da, theoretical [M+6H]⁶⁺/6:1675.10 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.675 min) and by HRMS (Formula: C₇₇₄H₁₂₆₇N₁₉₃O₂₄₅S₂, found: 17261.2197 Da, theoretical: 17260.2336 Da).

Linear protein: Acm protection on cysteine residues were removed using protocol 18. The ligation sample was purified by preparative HPLC (protocol 20). The purified linear protein was analyzed using HPLC method 12 (t_{R}=15.658 min) and by HRMS (Formula: C₇₆₈H₁₂₅₇N₁₉₁O₂₄₃S₂, found: 17119.1326 Da, theoretical: 17118.1594 Da).

Folded protein: The linear protein was rearranged, folded and purified following protocol 19. The folded protein was analyzed using HPLC method 6 (t_{R}=13.558 min) and by HRMS (Formula: C₇₆₈H₁₂₅₅N₁₉₁O₂₄₃S₂, found: 17116.1994 Da, theoretical: 17116.7020 Da).

### Example 54J: Synthesis of CMP-318

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 11 (t_{R}=12.672 min) and by MS (ESI) (Formula: C₂₃₉H₄₀₃N₆₉O₇₃, found: 1353.81 [M+4H]⁴⁺/4 Da, theoretical: 1353.81 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=14.542 min) and by MS (ESI) (Formula: C₄₅₇H₇₆₆N₁₁₄O₁₄₂S, found: 1695.63 [M+6H]⁶⁺/6, Da, theoretical: 1694.64 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.608 min) and by HRMS (Formula: C₇₈₂H₁₂₈₂N₁₉₈O₂₄₁S₂, found: 17377.3389 Da, theoretical: 17377.3867 Da).

Linear protein: Acm protection on cysteine residues were removed using protocol 18. The ligation sample was purified by preparative HPLC (protocol 20). The purified linear protein was analyzed using HPLC method 12 (t_{R}=15.875 min) and by HRMS (Formula: C₇₇₆H₁₂₇₂N₁₉₆O₂₃₉S₂, found: 17235.2297 Da, theoretical: 17235.3124 Da).

Folded protein: The linear protein was rearranged, folded and purified following protocol 19. The folded protein was analyzed using HPLC method 6 (t_{R}=13.525 min) and by HRMS (Formula: C₇₇₆H₁₂₇₀N₁₉₆O₂₃₉S₂, found: 17233.3606 Da, theoretical: 17233.9490 Da).

### Example 54K: Synthesis of CMP-319

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 13 (t_{R}=13.775 min) and by MS (ESI) (Formula: C₂₄₈H₄₂₂N₇₆O₇₃, found: 1410.36, Da, theoretical [M+4H]⁴⁺/4:1410.14 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=13.925 min) and by MS (ESI) (Formula: C₄₆₆H₇₈₅N₁₂₁O₁₄₂S, found: 1484.88, Da, theoretical [M+7]⁷⁺/7:1484.88 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.292 min) and by HRMS (Formula: C₇₉₁H₁₃₀₁N₂₀₅O₂₄₁S₂, found: 17602.5510 Da, theoretical: 17602.5568 Da).

Linear protein: Acm protection on cysteine residues were removed using protocol 18. The ligation sample was purified by preparative HPLC (protocol 20). The purified linear protein was analyzed using HPLC method 12 (t_{R}=15.408 min) and by HRMS (Formula: C₇₈₅H₁₂₉₁N₂₀₃O₂₃₉S₂, found: 17460.4515 Da, theoretical: 17460.4826 Da).

Folded protein: The linear protein was rearranged, folded and purified following protocol 19. The folded protein was analyzed using HPLC method 6 (t_{R}=13.425 min) and by HRMS (Formula: C₇₈₅H₁₂₈₉N₂₀₃O₂₃₉S₂, found: 17460.4743 Da, theoretical: 17459.2490 Da).

### Example 54L: Synthesis of CMP-320

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 13 (t_{R}=14.725 min) and by MS (ESI) (Formula: C₂₅₂H₄₂₁N₇₁O₇₇, found: 1420.76, Da, theoretical [M+4H]⁴⁺/4:1420.39 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=14.392 min) and by MS (ESI) (Formula: C₄₇₀H₇₈₄N₁₁₆O₁₄₆S, found: 1490.53 Da, theoretical [M+7H]⁷⁺/7:1490.74 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.592 min) and by HRMS (Formula: C₇₉₅H₁₃₀₀N₂₀₀O₂₄₅S₂, found: 17643.5075 Da, theoretical: 17643.5134 Da).

Linear protein: Acm protection on cysteine residues were removed using protocol 18. The ligation sample was purified by preparative HPLC (protocol 20). The purified linear protein was analyzed using HPLC method 12 (t_{R}=15.825 min) and by HRMS (Formula: C₇₈₉H₁₂₉₀N₁₉₈O₂₄₃S₂, found: 17501.4212 Da, theoretical: 17501.4391 Da).

Folded protein: The linear protein was rearranged, folded and purified following protocol 19. The folded protein was analyzed using HPLC method 6 (t_{R}=13.525 min) and by HRMS (Formula: C₇₈₉H₁₂₈₈N₁₉₈O₂₄₃S₂, found: 17500.4351 Da, theoretical: 17500.2460 Da).

### Example 54M: Synthesis of CMP-321

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 11 (t_{R}=12.425 min) and by MS (ESI) (Formula: C₂₄₄H₄₀₉N₆₉O₇₅, found: 1378.45 Da, theoretical [M+4H]⁴⁺/4:1378.34 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=14.525 min) and by MS (ESI) (Formula: C₄₆₂H₇₇₂N₁₁₄O₁₄₄S, found: 1467.25 Da, theoretical [M+7H]⁷⁺/7:1466.71 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.725 min) and by HRMS (Formula: C₇₈₇H₁₂₈₈N₁₉₈O₂₄₃S₂, found: 17475.3816 Da, theoretical: 17475.4235 Da).

Linear protein: Acm protection on cysteine residues were removed using protocol 18. The ligation sample was purified by preparative HPLC (protocol 20). The purified linear protein was analyzed using HPLC method 12 (t_{R}=15.592 min) and by HRMS (Formula: C₇₈₁H₁₂₇₈N₁₉₆O₂₄₁S₂, found: 17333.3239 Da, theoretical: 17333.3493 Da).

Folded protein: The linear protein was rearranged, folded and purified following protocol 19. The folded protein was analyzed using HPLC method 6 (t_{R}=13.525 min) and by HRMS (Formula: C₇₈₁H₁₂₇₆N₁₉₆O₂₄₁S₂, found: 17331.3375 Da, theoretical: 17332.0500 Da).

### Example 54N: Synthesis of CMP-322

Example 54N contains a modification on the side chain of the Glu23 that required a prior synthesis of a short, protected peptide. The protected peptide with structure 10 was prepared as follows:

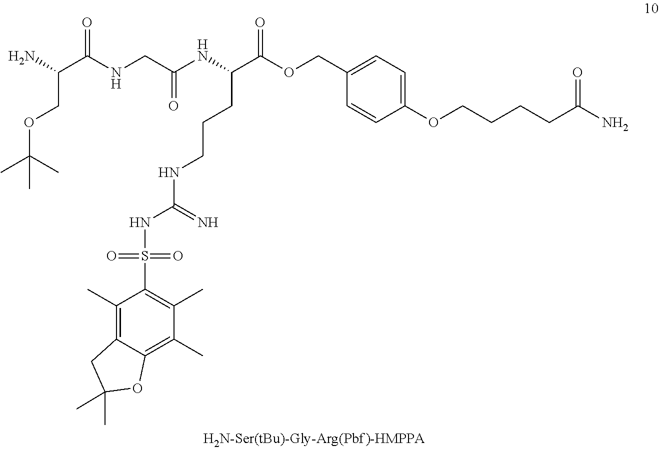 [SMILES: Cc1c(C)c(S(=O)(=O)NC(=N)NCCC[C@H](NC(=O)CNC(=O)[C@@H](N)COC(C)(C)C)C(=O)OCc2ccc(OCCCCC(N)=O)cc2)c(C)c2c1OC(C)(C)C2]

Loading of 5-(4-(hydroxymethyl) phenoxy) pentanoic acid (HMPPA) on Sieber resin: 8.2 g of Fmoc-protected Sieber resin (5 mmol scale) was swollen in DMF for 30 min. Fmoc-deprotection was performed by treating the resin twice with 20% piperidine in DMF (v/v) at room temperature for 10 min followed by several washes with DMF and a final wash with DCM. 5-(4-(hydroxymethyl) phenoxy) pentanoic acid (HMPPA) (4 g, 25 mmol, 5 equiv.) was dissolved in a mixture of DCM/DMF (8:2), and DIC (7.83 mL, 50 mmol, 10 equiv.) was added to the solution. The mixture was preactivated for 45 min at room temperature before addition to the resin and it was let to react for 16 h at room temperature under gentle agitation. The resin was rinsed thoroughly with DMF and DCM. Then ester formation with Fmoc-Arg (Pbf)-OH was done by firstly preactivating the amino acid (12 mmol, 8 equiv.) dissolved in 15 mL DMF and 35 mL DCM, addition of DIC (3.73 mL, 24 mmol, 16 equiv.) and stirring the solution for 30 min at room temperature before transferring it to the resin. A solution of DMAP (36.7 mg, 0.3 mmol, 0.2 equiv.) in DMF was then added to the resin and the reaction was gently shaken for 16 h. The resin was rinsed thoroughly with DMF and DCM. After ester formation, elongation of the sequence was done according to the conditions described in protocol 3. Resin cleavage of protected peptide was carried out according to protocol 12. The crude peptide was purified by preparative HPLC (protocol 20).

Segment 1: Loading of the first KAHA monomer 5 was performed following protocol 1. Elongation of the peptide chain was performed following protocol 3 until position Leu from the linker part. Side chain Allyl deprotection of Glu (Glu23) was performed following protocol 7. Protected tripeptide 10 was coupled to side chain of Glu23 by adding a solution of the protected peptide 10 (1.3 equiv.), HATU (1 equiv.) and DIPEA (4 equiv.) in DMF to the resin that was previously swollen in DMF. After gently shaking overnight at room temperature, the resin was rinsed with IPA and DCM.

Protected segment 1 was then released from the resin and side chains deprotected following protocol 14. The crude peptide was purified by preparative HPLC (protocol 20). The purified segment 1 was analyzed using HPLC method 13 (t_{R}=14.358 min) and by MS (ESI): (Formula: C₂₂₇H₃₈₅N₆₅O₇₀, found: 1030.16 Da, theoretical [M+5H]⁵⁺/5:1030.0 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=14.625 min) and by MS (ESI) (Formula: C₄₄₅H₇₄₈N₁₁₀O₁₃₉S, found: 1650.15 Da, theoretical [M+6H]⁶⁺/6:1649.7 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.542 min) and by MS (ESI) (Formula: C₇₇₀H₁₂₆₄N₁₉₄O₂₃₈S₂, found: Da, theoretical 1070.27 [M+16H]¹⁶⁺/16:1070.5 Da).

Linear protein: Acm protection on cysteine residues were removed using protocol 18. The ligation sample was purified by preparative HPLC (protocol 20). The purified linear protein was analyzed using HPLC method 12 (t_{R}=13.558 min) and by HRMS (Formula: C₇₆₄H₁₂₅₄N₁₉₂O₂₃₆S₂, found: 16969.0801 Da, theoretical: 16969.1745 Da).

Folded protein: The linear protein was rearranged, folded and purified following protocol 19. The folded protein was analyzed using HPLC method 6 (t_{R}=13.558 min) and by HRMS (Formula: C₇₆₄H₁₂₅₂N₁₉₂O₂₃₆S₂, found: 16967.0578 Da, theoretical: 16967.1589 Da).

### Example 540: Synthesis of CMP-323

Example 540 contains a modification on the side chain of the Glu23 that required a prior synthesis of a short, protected peptide. The protected peptide with structure 11 was prepared following the protocol described in example 54N.

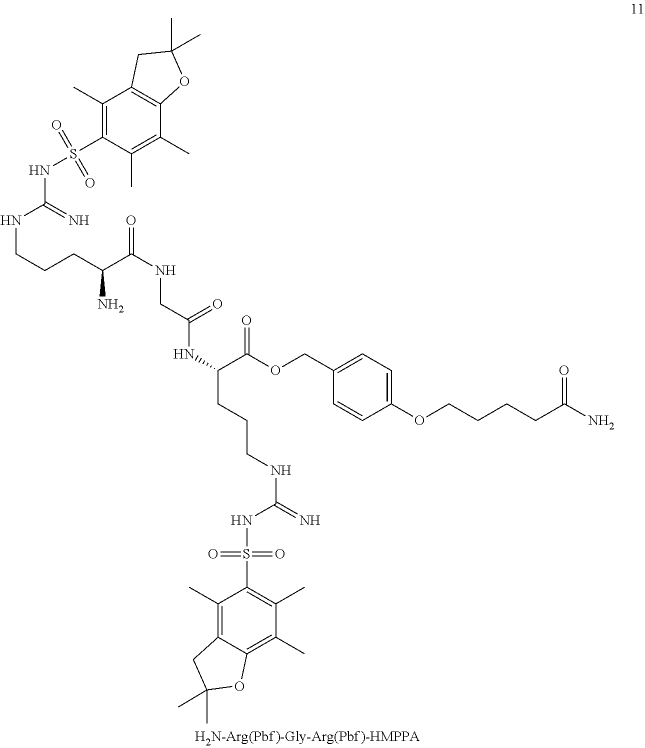 [SMILES: Cc1c(C)c(S(=O)(=O)NC(=N)NCCC[C@H](NC(=O)CNC(=O)[C@@H](N)CCCNC(=N)NS(=O)(=O)c2c(C)c(C)c3c(c2C)CC(C)(C)O3)C(=O)OCc2ccc(OCCCCC(N)=O)cc2)c(C)c2c1OC(C)(C)C2]

Segment 1: Loading of the first KAHA monomer 5 was performed following protocol 1. Elongation of the peptide chain was performed following protocol 3 until position Leu from the linker part. Side chain Allyl deprotection of Glu (Glu23) was performed following protocol 7. Protected tripeptide 11 was coupled to side chain of Glu23 by adding a solution of the protected peptide 11 (1.3 equiv.), HATU (1 equiv.) and DIPEA (4 equiv.) in DMF to the resin that was previously swollen in DMF. After gently shaking overnight at room temperature, the resin was rinsed with IPA and DCM. Protected segment 1 was then released from the resin and side chains deprotected following protocol 14. The crude peptide was purified by preparative HPLC (protocol 20). The purified segment 1 was analyzed using HPLC method 13 (t_{R}=13.442 min) and by MS (ESI) (Formula: C₂₃₀H₃₉₂N₆₈O₆₉, found: 1304.29 Da, theoretical [M+4H]⁴⁺/4:1304.5 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=13.592 min) and by MS (ESI) (Formula: C₄₄₈H₇₅₅N₁₁₃O₁₃₈, found: 1424.91 Da, theoretical [M+7H]⁷⁺/7:1424.53 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=15.342 min) and by HRMS (Formula: C₇₇₃H₁₂₇₁N₁₉₇O₂₃₇S₂, found: 17180.2327 Da, theoretical: 17180.3178 Da).

Linear protein: Acm protection on cysteine residues were removed using protocol 18. The ligation sample was purified by preparative HPLC (protocol 20). The purified linear protein was analyzed using HPLC method 12 (t_{R}=15.583 min) and by HRMS (Formula: C₇₆₇H₁₂₆₁N₁₉₅O₂₃₅S₂, found: 17038.1721 Da, theoretical: 17038.2436 Da).

Folded protein: The linear protein was rearranged, folded and purified following protocol 19. The folded protein was analyzed using HPLC method 6 (t_{R}=13.508 min) and by HRMS (Formula: C₇₆₇H₁₂₅₉N₁₉₅O₂₃₅S₂, found: 17036.0969 Da, theoretical: 17036.2279 Da).

### Example 54P: Synthesis of CMP-324

Example 54P contains a modification on the side chain of the Glu23 that required a prior synthesis of a short, protected peptide. The protected peptide with structure 12 (SEQ ID NO: 382) was prepared following the protocol described in example 54N.

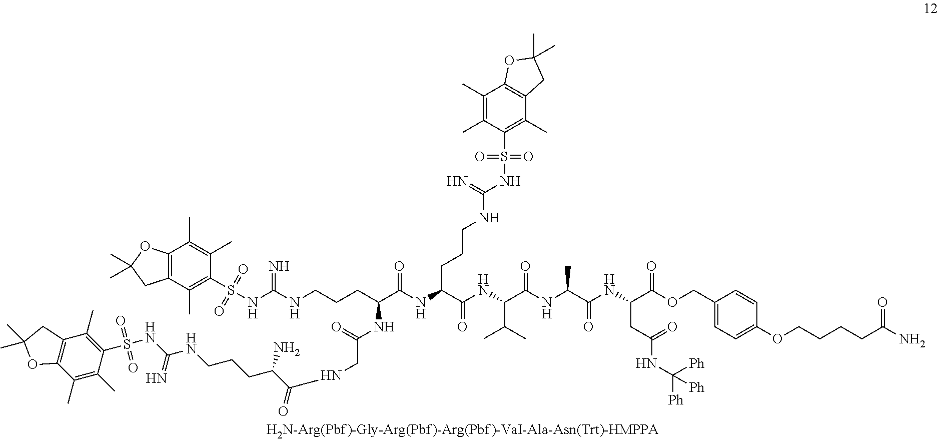 [SMILES: Cc1c(C)c(S(=O)(=O)NC(=N)NCCC[C@H](NC(=O)CNC(=O)[C@@H](N)CCCNC(=N)NS(=O)(=O)c2c(C)c(C)c3c(c2C)CC(C)(C)O3)C(=O)N[C@@H](CCCNC(=N)NS(=O)(=O)c2c(C)c(C)c3c(c2C)CC(C)(C)O3)C(=O)N[C@H](C(=O)N[C@@H](C)C(=O)N[C@@H](CC(=O)NC(c2ccccc2)(c2ccccc2)c2ccccc2)C(=O)OCc2ccc(OCCCCC(N)=O)cc2)C(C)C)c(C)c2c1OC(C)(C)C2]

Segment 1: Loading of the first KAHA monomer 5 was performed following protocol 1. Elongation of the peptide chain was performed following protocol 3 until position Leu from the linker part. Side chain Allyl deprotection of Glu (Glu23) was performed following protocol 7. Protected segment 1 was released from the Sieber resin following protocol 12 to keep the side chains protected. The coupling of the protected peptide 12 on the Seg1 was performed in solution. To do so, the cleaved Seg1 (1 equiv.) and Oxyma (4 equiv.) were dissolved in DMF. DIC (2.7 equiv.) was added to the resulting solution and the protected peptide 12 dissolved in DMF was immediately incorporated into the mixture. The pH of the reaction was adjusted to pH 8 by DIPEA addition. After 1 h at room temperature, a second portion of DIC (1.3 equiv.) was added to the reaction and the mixture was gently stirred at 40° C. for 17 h. The solution was then precipitated with H₂O, filtered off and washed twice with H₂O. The side chains on the resulting precipitate peptide were deprotected following protocol 14. The crude peptide was purified by preparative HPLC (protocol 20). The purified segment 1 was analyzed using HPLC method 13 (t_{R}=12.742 min) and by MS (ESI) (Formula: C₂₄₈H₄₂₄N₇₆O₇₄, found: 1414.80 Da, theoretical [M+4H]⁴⁺/4:1414.65 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=13.292 min) and by MS (ESI) (Formula: C₄₆₆H₇₈₇N₁₂₁O₁₄₃S, found: 1041.80 Da, theoretical [M+10H]¹⁰⁺/10:1041.52 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 14 (t_{R}=17.692 min) and by MS (ESI) (Formula: C₇₉₁H₁₃₀₃N₂₀₅O₂₄₂S₂, found: 1469.53 Da, theoretical [M+12H]¹²⁺/12:1469.45 Da).

Linear protein: Acm protection on cysteine residues were removed using protocol 18. The ligation sample was purified by preparative HPLC (protocol 20). The purified linear protein was analyzed using HPLC method 14 (t_{R}=17.775 min) and by HRMS (Formula: C₇₈₅H₁₂₉₃N₂₀₃O₂₄₀S₂, found: 17478.4426 Da, theoretical [M+1H]⁺: 17478.4926 Da).

Folded protein: The linear protein was rearranged, folded and purified following protocol 19. The folded protein was analyzed using HPLC method 6 (t_{R}=13.358 min) and by HRMS (Formula: C₇₈₅H₁₂₉₁N₂₀₃O₂₄₀S₂, found: 17476.4433 Da, theoretical: 17476.4775 Da).

### Example 54Q: Synthesis of CMP-325

Example 54Q contains a modification on the side chain of the Glu23 that required a prior synthesis of a short, protected peptide. The protected peptide with structure 11 was prepared following the protocol described in example 54N.

Segment 1: Loading of the first KAHA monomer 5 was performed following protocol 1. Elongation of the peptide chain was performed following protocol 3 until position Arg from the linker part. Side chain Allyl deprotection of Glu (Glu23) was performed following protocol 7. Protected tripeptide 11 was coupled to side chain of Glu23 by adding a solution of the protected peptide 11 (1.3 equiv.), HATU (1 equiv.) and DIPEA (4 equiv.) in DMF to the resin that was previously swollen in DMF. After gently shaking overnight at room temperature, the resin was rinsed with IPA and DCM. Protected segment 1 was then released from the resin and side chains deprotected following protocol 14. The crude peptide was purified by preparative HPLC (protocol 20). The purified segment 1 was analyzed using HPLC method 13 (t_{R}=13.075 min) and by MS (ESI) (Formula: C₂₄₈H₄₂₄N₇₆O₇₄, found: 1414.74 Da, theoretical [M+4H]⁴⁺/4:1414.64 Da).

Segment 2, 3, 4 and ligated segment 34 were synthesized as described in Example 3. Segment 12: Ligation of segments 1 and 2 and photodeprotection were performed as described in protocol 15. The ligation/deprotection sample was purified by preparative HPLC (protocol 20). The purified segment 12 was analyzed using HPLC method 13 (t_{R}=13.292 min) and by MS (ESI) (Formula: C₄₆₆H₇₈₇N₁₂₁O₁₄₃S, found: 1041.70 Da, theoretical [M+10H]¹⁰⁺/10:1041.52 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (t_{R}=14.842 min) and by MS (ESI) (Formula: C₇₉₁H₁₃₀₃N₂₀₅O₂₄₂S₂, found: 1602.99 Da, theoretical [M+11H]¹¹⁺/11:1602.95 Da).

Linear protein: Acm protection on cysteine residues were removed using protocol 18. The ligation sample was purified by preparative HPLC (protocol 20). The purified linear protein was analyzed using HPLC method 12 (t_{R}=15.175 min) and by HRMS (Formula: C₇₈₅H₁₂₉₃N₂₀₃O₂₄₀S₂, found: 17478.4390 Da, theoretical [M+1H]: 17478.4926 Da).

Folded protein: The linear protein was rearranged, folded and purified following protocol 19. The folded protein was analyzed using HPLC method 6 (t_{R}=13.358 min) and by HRMS (Formula: C₇₈₅H₁₂₉₁N₂₀₃O₂₄₀S₂, found: 17476.4409 Da, theoretical: 17476.4775 Da).

### Example 54R: Synthesis of CMP-326

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side-chain Alloc deprotection of Lys (position 9) was carried out using protocol 7. The linker was elongated on the Lys side chain until residue Gly kept Fmoc protected. 2-Cl-Trt deprotection on Glu23 was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (t_{R}=11.042 min) and by HRMS (Formula: C₂₂₉H₃₈₅N₆₃O₇₆, found: 5235.8248 Da, theoretical: 5235.8255 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (t_{R}=12.442 min) and by HRMS (Formula: C₄₄₇H₇₄₈N₁₀₈O₁₄₅S, found: 9986.4494 Da, theoretical: 9986.4339 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.725 min) and by HRMS (Formula: C₇₇₂H₁₂₆₄N₁₉₂O₂₄₄S₂, found: 17203.2217 Da, theoretical: 17203.2122 Da).

Linear protein: Acm protection on cysteine residues were removed using protocol 18. The ligation sample was purified by preparative HPLC (protocol 20). The purified linear protein was analyzed using HPLC method 12 (t_{R}=16.058 min) and by HRMS (Formula: C₇₆₆H₁₂₅₄N₁₉₀O₂₄₂S₂, found: 17061.1535 Da, theoretical: 17061.1379 Da).

Folded protein: The linear protein was rearranged, folded and purified following protocol 19. The folded protein was analyzed using HPLC method 6 (t_{R}=13.742 min) and by HRMS (Formula: C₇₆₆H₁₂₅₂N₁₉₀O₂₄₂S₂, found: 17059.1514 Da, theoretical: 17059.1223 Da).

### Example 54S-Synthesis of CMP-327

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side-chain Alloc deprotection of Lys (position 9) was carried out using protocol 7. The linker was elongated on the Lys side chain until residue Gly kept Fmoc protected. 2-Cl-Trt deprotection on Glu23 was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (t_{R}=10.842 min) and by HRMS (Formula: C₂₃₉H₄₀₃N₆₉O₇₃, found: 5410.9949 Da, theoretical: 5411.0392 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (t_{R}=12.525 min) and by HRMS (Formula: C₄₅₇H₇₆₆N₁₁₄O₁₄₂S, found: 10161.6196 Da, theoretical: 10161.6111 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.708 min) and by HRMS (Formula: C₇₈₂H₁₂₈₂N₁₉₈O₂₄₁S₂, found: 17377.3695 Da, theoretical: 17377.3867 Da).

Linear protein: Acm protection on cysteine residues were removed using protocol 18. The ligation sample was purified by preparative HPLC (protocol 20). The purified linear protein was analyzed using HPLC method 12 (t_{R}=16.058 min) and by HRMS (Formula: C₇₇₆H₁₂₇₂N₁₉₆O₂₃₉S₂, found: 17235.2999 Da, theoretical: 17235.3124 Da).

Folded protein: The linear protein was rearranged, folded and purified following protocol 19. The folded protein was analyzed using HPLC method 6 (t_{R}=13.742 min) and by HRMS (Formula: C₇₇₆H₁₂₇₀N₁₉₆O₂₃₉S₂, found: 17233.3213 Da, theoretical: 17233.2968 Da).

### Example 54T-Synthesis of CMP-328

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side-chain Alloc deprotection of Lys (position 9) was carried out using protocol 7. The linker was elongated on the Lys side chain until residue Gly kept Fmoc protected. 2-Cl-Trt deprotection on Glu23 was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 13 (t_{R}=14.575 min) and by MS (ESI) (Formula: C₂₄₈H₄₂₂N₇₆O₇₃, found: 1410.26 Da, theoretical [M+4H]⁴⁺/4:1410.14 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 14 (t_{R}=14.025 min) and by MS (ESI) (Formula: C₄₆₆H₇₈₅N₁₂₁O₁₄₂S, found: 1484.77 Da, theoretical [M+7H]⁷⁺/7:1484.88 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.325 min) and by HRMS (Formula: C₇₉₁H₁₃₀₁N₂₀₅O₂₄₁S₂, found: 17602.5214 Da, theoretical: 17602.5568 Da).

Linear protein: Acm protection on cysteine residues were removed using protocol 18. The ligation sample was purified by preparative HPLC (protocol 20). The purified linear protein was analyzed using HPLC method 12 (t_{R}=15.225 min) and by HRMS (Formula: C₇₈₅H₁₂₉₁N₂₀₃O₂₃₉S₂, found: 17460.4614 Da, theoretical: 17460.4826 Da).

Folded protein: The linear protein was rearranged, folded and purified following protocol 19. The folded protein was analyzed using HPLC method 6 (t_{R}=13.425 min) and by HRMS (Formula: C₇₈₅H₁₂₈₉N₂₀₃O₂₃₉S₂, found: 17458.4672 Da, theoretical: 17458.4669 Da).

### Example 54U-Synthesis of CMP-329

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side-chain Alloc deprotection of Lys (position 9) was carried out using protocol 7. The linker was elongated on the Lys side chain until residue Gly kept Fmoc protected. 2-Cl-Trt deprotection on Glu23 was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 13 (t_{R}=15.492 min) and by MS (ESI) (Formula: C₂₅₂H₄₂₁N₇₁O₇₇, found: 1420.12 Da, theoretical [M+4H]⁴⁺/4:1420.39 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 14 (t_{R}=14.608 min) and by MS (ESI) (Formula: C₄₇₀H₇₈₄N₁₁₆O₁₄₆S, found: 1490.66 Da, theoretical [M+7H]⁷⁺/7:1490.74 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 14 (t_{R}=18.208 min) and by HRMS (Formula: C₇₉₅H₁₃₀₀N₂₀₀O₂₄₅S₂, found: 17643.4525 Da, theoretical: 17643.5134 Da).

Linear protein: Acm protection on cysteine residues were removed using protocol 18. The ligation sample was purified by preparative HPLC (protocol 20). The purified linear protein was analyzed using HPLC method 12 (t_{R}=15.225 min) and by HRMS (Formula: C₇₈₉H₁₂₉₀N₁₉₈O₂₄₃S₂, found: 17501.4154 Da, theoretical: 17501.4391 Da).

Folded protein: The linear protein was rearranged, folded and purified following protocol 19. The folded protein was analyzed using HPLC method 6 (t_{R}=13.525 min) and by HRMS (Formula: C₇₈₉H₁₂₈₈N₁₉₈O₂₄₃S₂, found: 17499.4076 Da, theoretical: 17499.4235 Da).

### Example 54V-Synthesis of CMP-330

Segment 1: Loading of the first KAHA monomer 7 was performed following protocol 1. Elongation of the peptide chain was performed following protocol 3 until position 23 keeping the Fmoc protecting group. Side chain All deprotection of Glu (position 23) was carried out following protocol 7 and on-resin protection of the acid moiety with 2-Cl-Trt was performed using protocol 10. After Fmoc deprotection, the elongation of the segment was performed until position 1 (Boc-Ala). Side-chain Alloc deprotection of Lys (position 9) was carried out using protocol 7. The linker was elongated on the Lys side chain until residue Gly kept Fmoc protected. 2-Cl-Trt deprotection on Glu23 was realized according to protocol 11 followed by Fmoc deprotection. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 13 (t_{R}=15.692 min) and by MS (ESI) (Formula: C₂₄₄H₄₀₉N₆₉O₇₅, found: 1378.02 Da, theoretical [M+4H]⁴⁺/4:5509.36 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 14 ((R=14.725 min) and by MS (ESI) (Formula: C₄₆₃H₇₇₄N₁₁₄O₁₄₄S, found: 1467.29 [M+7H]⁷⁺/7 Da, theoretical: 1468.71 Da).

Segment 1234 (Acm protected cysteines): Ligation of segment 12 and 34 was performed as described in protocol 17. The ligation sample was purified by preparative HPLC (protocol 20). The purified segment 1234 (Acm protected cysteines) was analyzed using HPLC method 12 (t_{R}=15.958 min) and by HRMS (Formula: C₇₈₇H₁₂₈₈N₁₉₈O₂₄₃S₂, found: 17475.3523 Da, theoretical: 17475.4235 Da).

Linear protein: Acm protection on cysteine residues were removed using protocol 18. The ligation sample was purified by preparative HPLC (protocol 20). The purified linear protein was analyzed using HPLC method 12 (t_{R}=15.825 min) and by HRMS (Formula: C₇₈₁H₁₂₇₈N₁₉₆O₂₄₁S₂, found: 17333.3211 Da, theoretical: 17333.3493 Da).

Folded protein: The linear protein was rearranged, folded and purified following protocol 19. The folded protein was analyzed using HPLC method 6 (t_{R}=13.525 min) and by HRMS (Formula: C₇₈₁H₁₂₇₆N₁₉₆O₂₄₁S₂, found: 17331.3336 Da, theoretical: 17331.3336 Da).

### Example 54W-Synthesis of CMP-331

Segment 1: Loading of the first KAHA monomer 7 was performed following protocol 1. Elongation of the peptide chain was performed following protocol 3 until position 1 (Ala). The free N-terminus Ala1 was Alloc protected using protocol 5. Side chain ivDde protection of Lys (position 23) was removed following protocol 8 and elongation was pursued on the free amine side chain following protocol 3 until PEG9 from the linker. Glutaric acid was then incorporated using protocol 9. N-terminus Alloc deprotection of Ala1 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using analytical method 2 (t_{R}=10.925 min) and by HRMS (Formula: C₂₄₅H₄₁₆N₆₄O₈₂, found: 5570.0370 Da, theoretical: 5570.0435 Da).

Segment 2, 3, 4 and ligated segment 34 were synthesized as described in Example 3. Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (Rt=12.575 min) and by HRMS (Formula: C₄₆₃H₇₇₉N₁₀₉O₁₅₁S, found: 10320.6540 Da, theoretical: 10320.6519 Da). Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 12 (RT=15.875 min) and by HRMS (Formula: C₇₈₉H₁₂₉₆N₁₉₂O₂₅₀S₂, found: 17536.4276 Da, theoretical: 15535.4322 Da).

Linear protein: Acm protection on cysteine residues were removed using protocol 18. The ligation sample was purified by preparative HPLC (protocol 20). The purified linear protein was analyzed using HPLC method 12 (RT=16.092 min) and by HRMS (Formula: C₇₈₃H₁₂₈₆N₁₉₀O₂₄₈S₂, found: 17394.3791 Da, theoretical: 17393.3579 Da).

Folded protein: The linear protein was rearranged, folded and purified following protocol 19. The folded protein was analyzed using HPLC method 6 (t_{R}=13.792 min) and by HRMS (Formula: C₇₈₃H₁₂₈₄N₁₉₀O₂₄₈S₂, found: 17392.3675 Da, theoretical: 17391.3423 Da).

### Example 55: IL-2 Receptor By Reporter Assay in HEK-Blue™ Cells

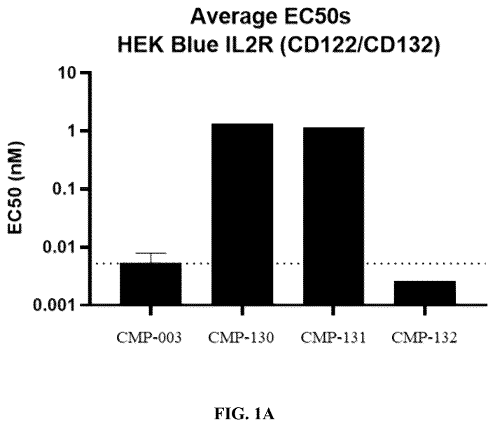

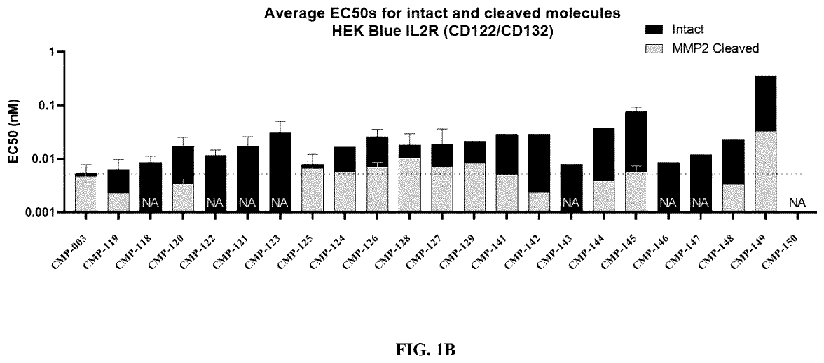

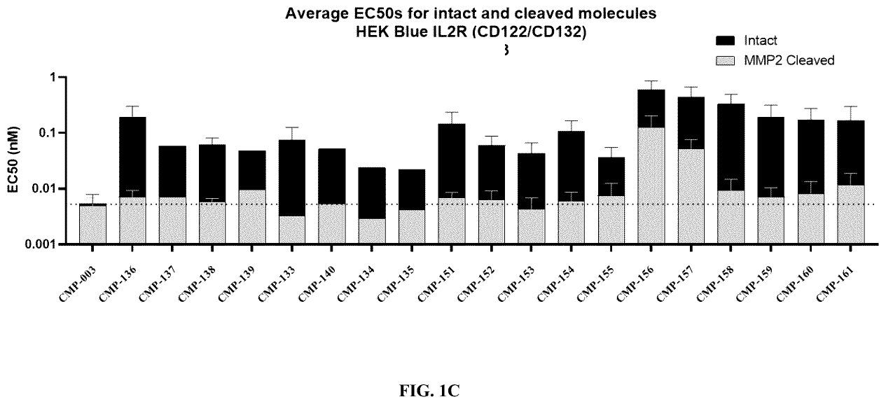

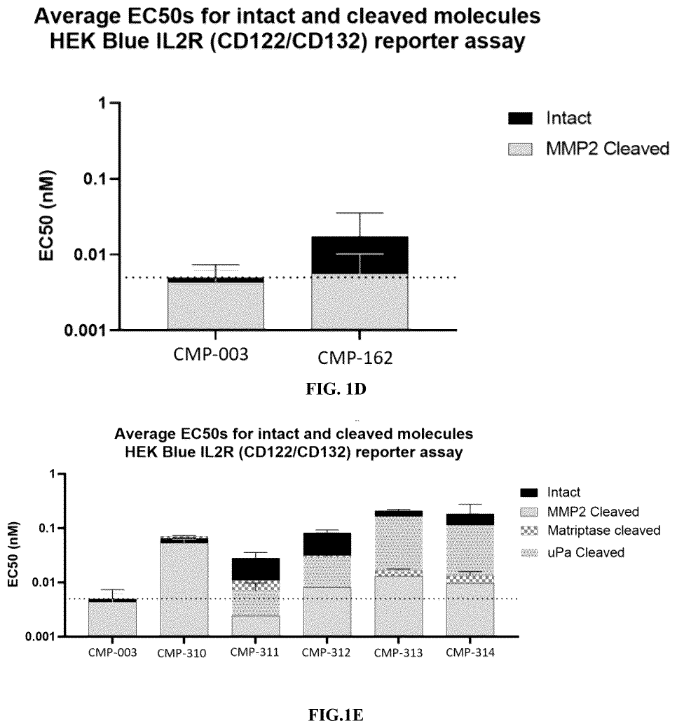

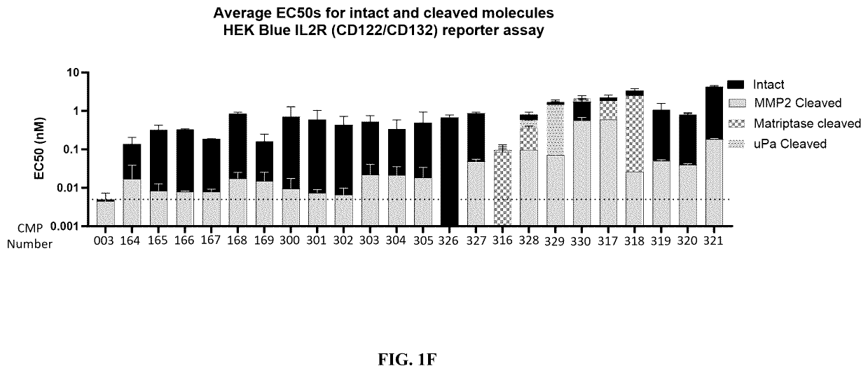

An IL-2Rβγ positive HEK-Blue reporter cell line was used to determine binding of activatable IL-2 polypeptides (and activated versions thereof) to IL-2Rβγ and subsequent downstream signaling that resulted in activation of a STAT5 transcriptional reporter. Activatable IL-2 polypeptides were either intact or MMP2 treated for activation. The general protocol was as follows: 5×10⁴ cells HEK-Blue™ IL-2Rβγ reporter cells (InvivoGen, ##hkb-il2bg) were seeded into each well of a 96 well plate and stimulated with 0-100 nM of IL-2 polypeptide variants at 37° C. and 5% CO2. After a 20h incubation, 20 μL of cell culture supernatant was then taken from each well and mixed with 180 μL QUANTI-Blue™ media in a 96 well plate, then incubated for 1 hour at 37° C. and 5% CO2. The absorbance signal at 620 nm was then measured on an Enspire® plate reader with 680 and 615 nm as excitation and emission wavelengths, respectively. Half Maximal Effective dose (EC50) was calculated based on a variable slope, four parameter analysis using GraphPad PRISM® software (FIGS. 1A, 1B, 1C). Results of additional tested molecules can be found in FIG. 1D, FIG. 1E, and FIG. 1F.

TABLE 17
EC₅₀ in HEK-Blue IL-2R (CD122⁺,
CD132⁺) Reporter Assay for selected molecules

| | Intact molecule | | |
|---|---|---|---|
| | Average EC50 | | |
| Molecule # | (nM) | Std Deviation | n= |
| CMP-003 | 0.0054 | 0.0025 | 17 |
| CMP-130 | 1.3420 | NA | 1 |
| CMP-131 | 1.1470 | NA | 1 |
| CMP-132 | 0.0026 | NA | 1 |

TABLE 18
EC50 in HEK-Blue IL-2R (CD122+, CD132+) Reporter
Assay for selected molecules

| | Intact molecule | | | MMP2 cleaved molecule | | |
|---|---|---|---|---|---|---|
| | Average | Std | | Average | Std | |
| Molecule # | EC50 (nM) | Deviation | n = | EC50 (nM) | Deviation | n = |
| CMP-003 | 0.0054 | 0.0025 | 17 | 0.0049 | 0.0020 | 10 |
| CMP-119 | 0.0063 | 0.0035 | 4 | 0.0023 | NA | 1 |
| CMP-118 | 0.0086 | 0.0027 | 3 | NA | NA | 0 |
| CMP-120 | 0.0172 | 0.0083 | 7 | 0.0034 | 0.0008 | 3 |
| CMP-122 | 0.0117 | 0.0030 | 3 | NA | NA | 0 |
| CMP-121 | 0.0174 | 0.0089 | 3 | NA | NA | 0 |
| CMP-123 | 0.0313 | 0.0196 | 3 | NA | NA | 0 |
| CMP-125 | 0.0079 | 0.0043 | 2 | 0.0067 | 0.0023 | 2 |
| CMP-124 | 0.0166 | NA | 1 | 0.0057 | NA | 1 |
| CMP-126 | 0.0261 | 0.0098 | 2 | 0.0070 | 0.0016 | 2 |
| CMP-128 | 0.0182 | 0.0114 | 4 | 0.0105 | NA | 1 |
| CMP-127 | 0.0189 | 0.0176 | 4 | 0.0073 | NA | 1 |
| CMP-129 | 0.0217 | NA | 1 | 0.0084 | NA | 1 |
| CMP-141 | 0.0288 | NA | 1 | 0.0051 | NA | 1 |
| CMP-142 | 0.0287 | NA | 1 | 0.0024 | NA | 1 |
| CMP-143 | 0.0079 | NA | 1 | NA | NA | 0 |
| CMP-144 | 0.0375 | NA | 1 | 0.0040 | NA | 1 |
| CMP-145 | 0.0759 | 0.0172 | 2 | 0.0060 | 0.0015 | 2 |
| CMP-146 | 0.0086 | NA | 1 | NA | NA | 0 |
| CMP-147 | 0.0121 | NA | 1 | NA | NA | 0 |
| CMP-148 | 0.0230 | NA | 1 | 0.0034 | NA | 1 |
| CMP-149 | 0.3603 | NA | 1 | 0.0333 | NA | 1 |
| CMP-150 | NA | NA | 0 | NA | NA | 0 |

TABLE 19A
EC50 in HEK-Blue IL-2R (CD122+, CD132+) Reporter
Assay for selected molecules

| | Intact molecule | | | MMP2 cleaved molecule | | |
|---|---|---|---|---|---|---|
| | Average | Std | | Average | Std | |
| Molecule # | EC50 (nM) | Deviation | n = | EC50 (nM) | Deviation | n = |
| CMP-003 | 0.0054 | 0.0025 | 17 | 0.0049 | 0.0020 | 10 |
| CMP-136 | 0.1925 | 0.1089 | 3 | 0.0071 | 0.0022 | 3 |
| CMP-137 | 0.0580 | NA | 1 | 0.0071 | NA | 1 |
| CMP-138 | 0.0614 | 0.0199 | 3 | 0.0058 | 0.0008 | 3 |
| CMP-139 | 0.0483 | NA | 1 | 0.0096 | NA | 1 |
| CMP-133 | 0.0745 | 0.0508 | 2 | 0.0032 | NA | 1 |
| CMP-140 | 0.0515 | NA | 1 | 0.0054 | NA | 1 |
| CMP-134 | 0.0237 | NA | 1 | 0.0029 | NA | 1 |
| CMP-135 | 0.0219 | NA | 1 | 0.0042 | NA | 1 |
| CMP-151 | 0.1446 | 0.0902 | 6 | 0.0068 | 0.0017 | 4 |
| CMP-152 | 0.0597 | 0.0285 | 4 | 0.0064 | 0.0027 | 4 |
| CMP-153 | 0.0432 | 0.0225 | 4 | 0.0043 | 0.0025 | 4 |
| CMP-154 | 0.1060 | 0.0588 | 4 | 0.0059 | 0.0027 | 4 |
| CMP-155 | 0.0359 | 0.0190 | 4 | 0.0075 | 0.0050 | 4 |
| CMP-156 | 0.5960 | 0.2609 | 2 | 0.1276 | 0.0755 | 2 |
| CMP-167 | 0.4441 | 0.2210 | 2 | 0.0521 | 0.0240 | 2 |
| CMP-158 | 0.3294 | 0.1610 | 2 | 0.0093 | 0.0054 | 2 |
| CMP-159 | 0.1921 | 0.1238 | 2 | 0.0071 | 0.0033 | 2 |
| CMP-160 | 0.1723 | 0.1025 | 2 | 0.0082 | 0.0053 | 2 |
| CMP-161 | 0.1683 | 0.1309 | 2 | 0.0118 | 0.0071 | 2 |

TABLE 19B
EC50 in HEK-Blue IL-2R (CD122+, CD132+) Reporter
Assay for additional molecules

| | Intact molecule | | | MMP2 cleaved molecule | | |
|---|---|---|---|---|---|---|
| | Average | | | Average | | |
| CMP | EC50 | Std | | EC50 | Std | |
| # | (nM) | Deviation | n = | (nM) | Deviation | n = |
| 3 | 0.0049 | 0.0022 | 31 | 0.0042 | 0.0019 | 23 |
| 163 | 0.1185 | 0.09178 | 2 | 0.0054 | 0.0045 | 2 |
| 164 | 0.1388 | 0.0664 | 2 | 0.0168 | 0.0220 | 2 |
| 165 | 0.3163 | 0.1071 | 7 | 0.0082 | 0.0045 | 6 |
| 166 | 0.3295 | 0.01301 | 2 | 0.0079 | 0.0004 | 2 |
| 167 | 0.1889 | 0.001626 | 2 | 0.0078 | 0.0014 | 2 |
| 168 | 0.8489 | 0.07022 | 2 | 0.0175 | 0.0078 | 2 |
| 169 | 0.1610 | 0.08704 | 2 | 0.0147 | 0.0108 | 2 |
| 300 | 0.711 | 0.5757 | 2 | 0.00940 | 0.008018 | 2 |
| 301 | 0.5906 | 0.4445 | 2 | 0.00726 | 0.001738 | 2 |
| 302 | 0.4361 | 0.2855 | 2 | 0.00646 | 0.003352 | 2 |
| 303 | 0.521 | 0.2366 | 2 | 0.02179 | 0.01902 | 2 |
| 304 | 0.34 | 0.2409 | 2 | 0.02098 | 0.01433 | 2 |
| 305 | 0.4974 | 0.439 | 2 | 0.01845 | 0.01564 | 2 |
| 326 | 0.6724 | 0.1154 | 2 | NA | NA | |
| 327 | 0.8687 | 0.0613 | 2 | 0.0481 | 0.0070 | 2 |
| 315 | 0.0554 | 0.0136 | 2 | 0.0418 | 0.0073 | 2 |
| 316 | 0.0968 | 0.0343 | 2 | 0.0963 | 0.0307 | 2 |
| 328 | 0.8089 | 0.1105 | 2 | 0.0964 | 0.0019 | 2 |
| 329 | 1.7040 | 0.2348 | 2 | 0.0702 | 0.0026 | 2 |
| 330 | 1.7250 | 0.3847 | 2 | 0.5580 | 0.1174 | 2 |
| 317 | 2.2355 | 0.338704148 | 2 | 0.58785 | 0.05522504 | 2 |
| 318 | 3.3735 | 0.400929545 | 2 | 0.02589 | 0.00304763 | 2 |
| 319 | 1.05445 | 0.514137341 | 2 | 0.04902 | 0.004737615 | 2 |
| 320 | 0.7746 | 0.124450793 | 2 | 0.03921 | 0.002983991 | 2 |
| 321 | 4.196 | 0.032526912 | 2 | 0.1814 | 0.011737973 | 2 |

| | Matriptase cleaved molecule | | | uPa cleaved molecule | | |
|---|---|---|---|---|---|---|
| | Average | | | Average | | |
| CMP | EC50 | Std | | EC50 | Std | |
| # | (nM) | Deviation | n = | (nM) | Deviation | n = |
| 3 | | | | | | |
| 163 | | | | | | |
| 164 | | | | | | |
| 165 | | | | | | |
| 166 | | | | | | |
| 167 | | | | | | |
| 168 | | | | | | |
| 169 | | | | | | |
| 300 | | | | | | |
| 301 | | | | | | |
| 302 | | | | | | |
| 303 | | | | | | |
| 304 | | | | | | |
| 305 | | | | | | |
| 326 | | | | | | |
| 327 | | | | | | |
| 315 | 0.03494 | 0.00291328 | 2 | 0.05811 | 0.0043982 | 2 |
| 316 | 0.08276 | 0.02013840 | 2 | 0.088435 | 0.0276690 | 2 |
| 328 | 0.3521 | 0.049356053 | 2 | 0.57125 | 0.0102530 | 2 |
| 329 | 1.4675 | 0.275064538 | 2 | 1.4055 | 0.0035355 | 2 |
| 330 | 2.15 | 0.33799704 | 2 | 1.7325 | 0.224152 | 2 |
| 317 | 1.844 | 0 | 2 | | | |
| 318 | 2.466 | 0.49356053 | 2 | | | |
| 319 | 0.050665 | 7.07107E−06 | 2 | | | |
| 320 | 0.8007 | 0.044547727 | 2 | | | |
| 321 | 4.217 | 0.326683333 | 2 | | | |

TABLE 19C
EC50 in HEK-Blue IL-2R (CD122+, CD132+) Reporter Assay for activatable IL-2 polypeptides

| | | | | MMP2 cleaved | | | Matriptase cleaved | | | uPa cleaved | | |
|---|---|---|---|---|---|---|---|---|---|---|---|---|
| | Intact molecule | | | molecule | | | molecule | | | molecule | | |
| | Average | | | Average | | | Average | | | Average | | |
| CMP | EC50 | Std | | EC50 | Std | | EC50 | Std | | EC50 | Std | |
| # | (nM) | Deviation | n = | (nM) | Deviation | n = | (nM) | Deviation | n = | (nM) | Deviation | n = |
| 3 | 0.0049 | 0.0022 | 31 | 0.0042 | 0.0019 | 23 | | | | | | |
| 310 | 0.0648 | 0.0016 | 2 | 0.0532 | 0.0091 | 2 | 0.071385 | 0.003259762 | 2 | 0.07229 | 0.000141421 | 2 |
| 311 | 0.0285 | 0.0071 | 2 | 0.0024 | 0.0008 | 2 | 0.01088 | 0.005473006 | 2 | 0.006924 | 0.002771859 | 2 |
| 312 | 0.0831 | 0.0098 | 2 | 0.0081 | 0.0013 | 2 | 0.031155 | 0.005876057 | 2 | 0.029385 | 0.004787113 | 2 |
| 313 | 0.2087 | 0.0146 | 2 | 0.0130 | 0.0028 | 2 | 0.01669 | 0.000975807 | 2 | 0.16505 | 0.008414571 | 2 |
| 314 | 0.1842 | 0.0916 | 2 | 0.0097 | 0.0004 | 2 | 0.013205 | 0.002538513 | 2 | 0.11385 | 0.000212132 | 2 |
| 162 | 0.0174 | 0.0179 | 2 | 0.0056 | 0.0046 | 2 | | | | | | |

### Example 56: Biolayer Interferometry Measurement of Protein-Protein Interactions

Bilayer interferometry (BLI) assays were used to measure the binding constants between activatable IL-2 polypeptides (and activated versions thereof) to IL-2 receptor subunits or receptor complexes. BLI assays were conducted on an Octet® R8 instrument. Assay plates were 96 well, polypropylene, F-bottom (chimney well), black microplates (Greiner Bio-one, Cat. No: 655209). The biosensors were Octet® SAX2 (Sartorius, Cat. No: 18-5136). The assay buffer was 1X Kinetic Buffer (Sartorius, Cat. no: 18-5136) or Phosphate Buffered Saline (PBS) that contained 0.02% (v: v) Tween 20 (Polysorbate 20) and 0.1% (w/v) bovine serum albumin (BSA). The sensor was regenerated between measurements in 10 mM glycine pH 2.7 buffer.

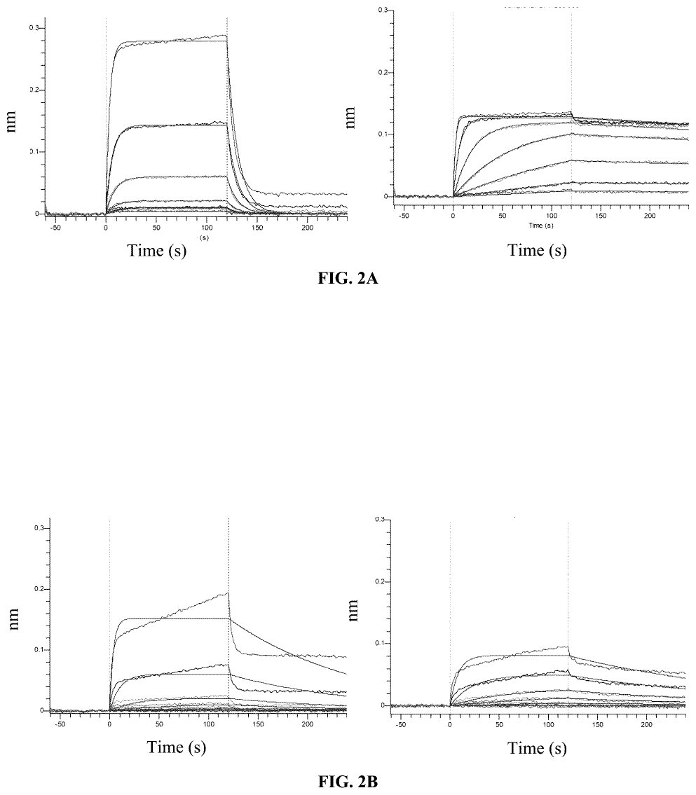

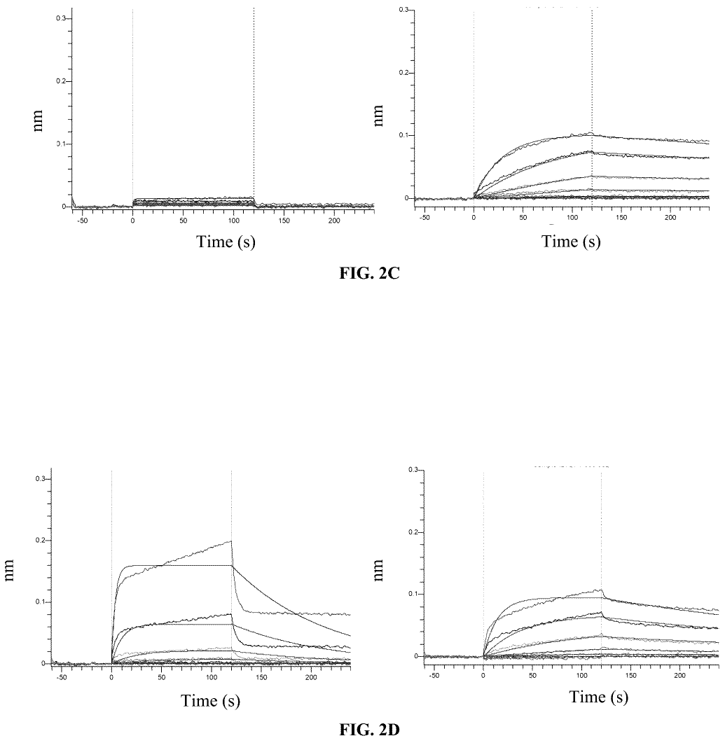

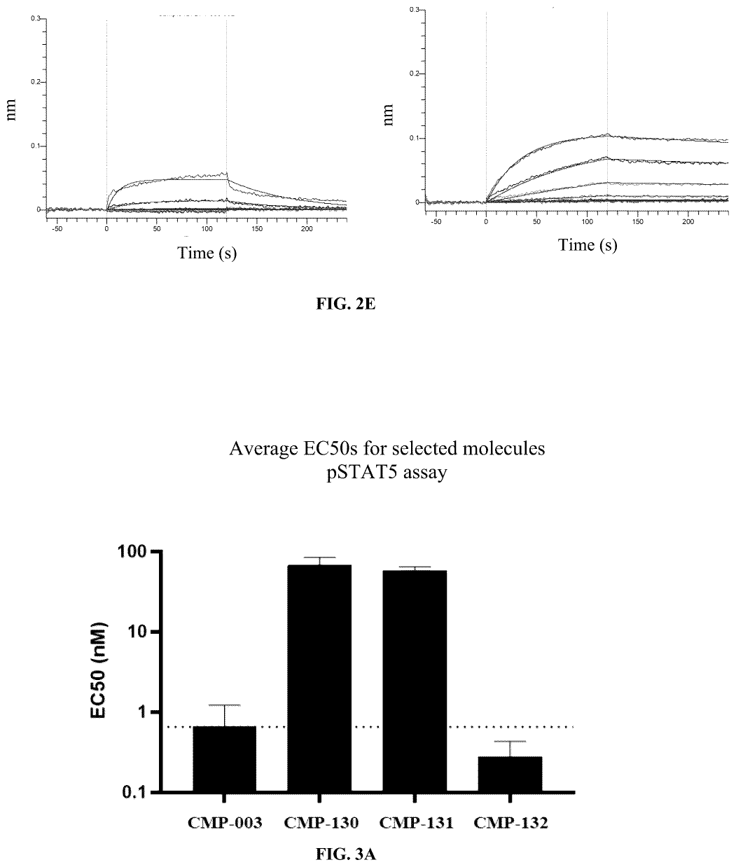

Sensor attached receptors were either recombinant human IL-2 receptor beta containing a His-tag and Avi-tag™ (R&D Systems™) or an Avi-tag™ Fc with human IL-2R beta and IL-2R gamma attached (Acrobiosystems Cat. no: ACRB-ILG-H₈₂F3-25UG). Receptors were reconstituted from lyophilized powder to 500 μg/ml in PBS. Analytes were diluted in Kinetic buffer to 300 nM, then serially diluted to lower concentrations. IL-2 compositions were tested for binding to CD122 or the CD122/CD132 complex: IL-2 polypeptide CMP-003 (FIG. 2A); CMP-145 (FIG. 2B); and CMP-136 (FIG. 2C), CMP-138 (FIG. 2D), and CMP-151 (FIG. 2D).

TABLE 20
Binding Constants for IL-2 Compositions and IL-2 Receptor Subunit/Complex

| | CD122 Binding (IL-2R β) | | | CD122 CD132 Binding (IL-2R βγ) | | |
|---|---|---|---|---|---|---|
| IL-2 Construct | K_{D} (M) | K_{ON}(M⁻¹S⁻¹) | K_{OFF} (S⁻¹) | K_{D} (M) | K_{ON}(M⁻¹S⁻¹) | K_{OFF} (S⁻¹) |
| CMP-003 | 1.51E−07 | 6.24E+05 | 9.41E−02 | 5.39E−10 | 1.53E+06 | 8.25E−04 |
| CMP-136 | 8.44E−09 | 9.09E+05 | 7.67E−03 | 1.26E−08 | 3.96E+05 | 4.98E−03 |
| CMP-145 | 1.83E−06 | 6.26E+05 | 1.15E+00 | 1.02E−08 | 1.15E+05 | 1.17E−03 |
| CMP-138 | 1.17E−08 | 8.98E−05 | 1.05E−02 | 1.23E−08 | 2.32E+05 | 2.84E−03 |
| CMP-151 | 6.54E−08 | 2.36E+05 | 1.54E−02 | 1.25E−08 | 8.62E+04 | 1.08E−03 |
| CMP-168 | 2.507E−05 | 2.876E+03 | 7.209E−02 | 1.364E−07 | 1.617E+04 | 2.205E−03 |
| CMP-168 MMP2 | 1.368E−03 | 7.888E+02 | 1.079E00 | 2.2721E−09 | 6.019E+05 | 1.638E−03 |
| CMP-165 | 4.42E−05 | 1.43E+04 | 6.33E−01 | 2.615E−11 | 1.202E+04 | 3.142E−07 |
| CMP-165 MMP2 | 1.11E−06 | 2.13E+05 | 2.37E−01 | 1.875E−09 | 6.084E+05 | 1.141E−03 |
| CMP-166 | 6.14E−07 | 9.98E+05 | 6.13E−01 | 1.61E−08 | 2.56E+04 | 4.12E−04 |
| CMP-166 MMP2 | 7.94E−07 | 2.83E+05 | 2.25E−01 | 1.21E−09 | 5.67E+05 | 6.84E−04 |
| CMP-167 | 2.15E−07 | 4.16E+06 | 9.93E−01 | 2.38E−08 | 3.64E+04 | 8.67E−04 |
| CMP-167 MMP2 | 6.58E−07 | 3.45E+05 | 2.27E−01 | 1.55E−09 | 5.94E+05 | 9.23E−04 |
| CMP-313 | 1.632E−07 | 8.139E04 | 1.329E−02 | 3.398E−09 | 4.360E05 | 1.481E−03 |
| CMP-328 | 3.401E−11 | 4.524E07 | 1.539E−03 | 2.487E−08 | 7.783E04 | 1.936E−03 |
| CMP- 319 | 5.947E−08 | 2.055E05 | 1.222E−02 | 9.193E−09 | 8.552E04 | 7.861E−04 |

### Example 57A: Assay for STAT5 Phosphorylation (pSTAT5)

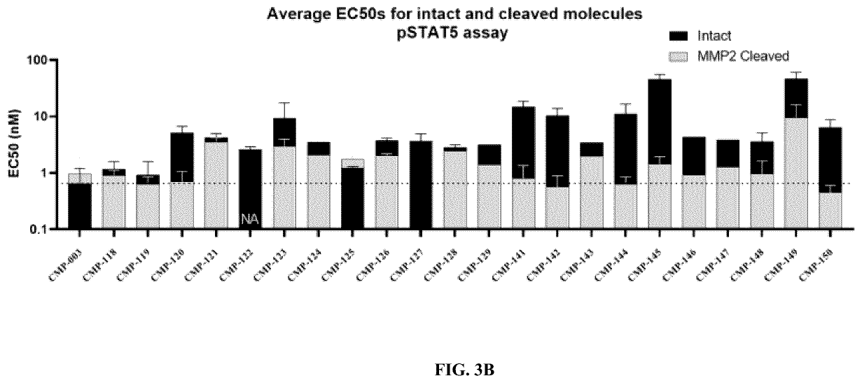

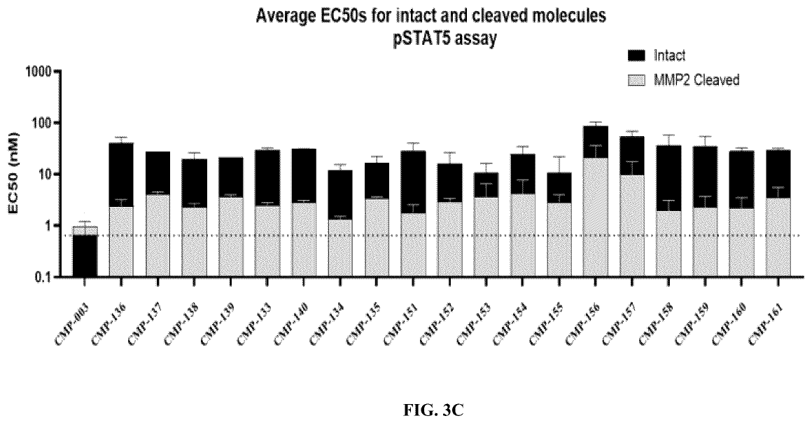

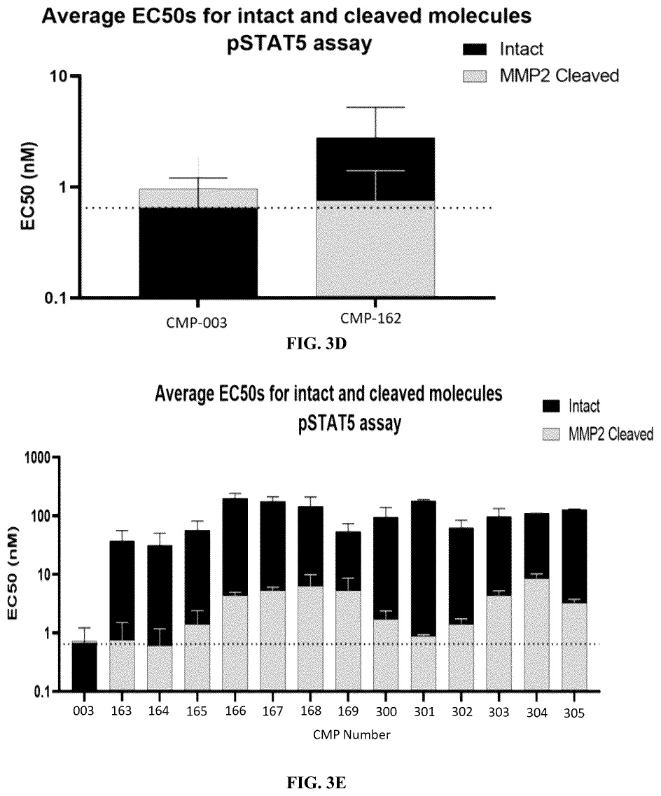

Assays were conducted to determine the effect of IL-2 compositions on STAT5 phosphorylation (pSTAT5) in T cells. Compositions of IL-2 were either intact or treated with MMP2 for activation. For pSTAT5 assays, frozen pan T cells were thawed and cultured overnight in RPMI 10% FCS, 1% Glutamine, 1% NEAA, 25 μM 2-mercaptoethanol (bMeoH), 1% NaPyruvate at 37° C., 5% CO2, 95% humidity. The next day cells were washed and resuspended in PBS. Cells were stimulated with serial dilutions of activatable IL-2 (intact) or protease cleaved IL-2 compositions for 40 minutes at 37° C., 5% CO2, 95% humidity followed by cell fixation and permeabilization with Transcription Factor Phospho Buffer Set (BD Biosciences). Then, cells were stained for 1 hour on ice with antibodies detecting human Phospho-Stat5-PE (Tyr694; 1:50, clone 47/Stat5pY694), CD25-BV421 (1:100, clone M-A251), CD45RA-BV711 (1:100, clone HI100), CD4-FITC (1:400, clone RPA-T4), CD8-APC/Cy7 (1:100, clone SK1), FOXP3-AF647 (1:50, clone 259D). Then cells were washed twice with ice cold FACS buffer. Data were acquired on a multi-color flow cytometer and data were analyzed with FlowJo software. EC50s were determined with GraphPadPrism. Plotted results of the pSTAT5 assay results for selected molecules were shown in FIG. 3A, FIG. 3B, and FIG. 3C. pSTAT5 assay results for additional molecules are shown in FIG. 3D and FIG. 3 E.

TABLE 21
Average EC50 For selected Molecules in the pSTAT5 Assay

| | | Intact Molecule | | |
|---|---|---|---|---|
| | | Average EC50 | | |
| Strategy | Molecule # | (nM) | Std Deviation | n= |
| Ref | CMP-003 | 0.65 | 0.56 | 28 |
| 1 | CMP-130 | 66.845 | 17.685 | 2 |
| 1 | CMP-131 | 56.92 | 7.28 | 2 |
| 1 | CMP-132 | 0.28 | 0.15 | 2 |

TABLE 22A
Average EC50 for selectedMolecules in the pSTAT5 Assay

| | Intact Molecule | | | MMP2 Cleaved Molecule | | |
|---|---|---|---|---|---|---|
| | Average | Std | | Average | Std | |
| Molecule # | EC50 (nM) | Deviation | n = | EC50 (nM) | Deviation | n = |
| CMP-003 | 0.65 | 0.56 | 28 | 0.96 | 0.90 | 19 |
| CMP-118 | 1.18 | 0.40 | 6 | 0.90 | 0.22 | 2 |
| CMP-119 | 0.91 | 0.67 | 8 | 0.63 | 0.22 | 4 |
| CMP-120 | 5.11 | 1.57 | 13 | 0.70 | 0.36 | 9 |
| CMP-122 | 4.22 | 0.75 | 6 | 3.55 | 0.68 | 2 |
| CMP-121 | 2.60 | 0.31 | 4 | NA | NA | 0 |
| CMP-123 | 9.26 | 8.26 | 6 | 2.93 | 1.05 | 2 |
| CMP-124 | 3.52 | NA | 1 | 2.07 | 0.00 | 1 |
| CMP-125 | 1.23 | 0.07 | 3 | 1.73 | 0.28 | 3 |
| CMP-126 | 3.74 | 0.37 | 3 | 2.03 | 0.17 | 3 |
| CMP-128 | 3.62 | 1.28 | 5 | 3.68 | NA | 1 |
| CMP-127 | 2.81 | 0.37 | 5 | 2.43 | NA | 1 |
| CMP-129 | 3.15 | NA | 1 | 1.40 | NA | 1 |
| CMP-141 | 14.72 | 3.95 | 2 | 0.80 | 0.55 | 2 |
| CMP-142 | 10.30 | 3.53 | 2 | 0.56 | 0.33 | 2 |
| CMP-143 | 3.41 | NA | 1 | 1.99 | NA | 1 |
| CMP-144 | 11.22 | 5.41 | 2 | 0.63 | 0.22 | 2 |
| CMP-145 | 45.34 | 10.31 | 5 | 1.431 | 0.5248 | 5 |
| CMP-146 | 4.29 | NA | 1 | 0.92 | NA | 1 |
| CMP-147 | 3.86 | NA | 1 | 1.29 | NA | 1 |
| CMP-148 | 3.57 | 1.54 | 2 | 0.95 | 0.68 | 2 |
| CMP-149 | 46.69 | 13.51 | 2 | 9.56 | 6.73 | 2 |
| CMP-150 | 6.38 | 2.39 | 2 | 0.45 | 0.15 | 2 |

TABLE 22B
Average EC50 for selected Molecules in the pSTAT5 Assay

| | Intact molecule | | | MMP2 cleaved molecule | | |
|---|---|---|---|---|---|---|
| | Average | Std | | Average | Std | |
| Molecule # | EC50 (nM) | Deviation | n = | EC50 (nM) | Deviation | n = |
| CMP-003 | 0.6697 | 0.5436 | 34 | 0.96 | 0.9027 | 30 |
| CMP-163 | 37.06 | 18.88 | 4 | 0.76 | 0.76 | 4 |
| CMP-164 | 31.35 | 19.3 | 4 | 0.61 | 0.57 | 4 |
| CMP-165 | 57.41 | 24.29 | 4 | 1.42 | 1.00 | 4 |
| CMP-166 | 198.4 | 44.41 | 2 | 4.36 | 0.58 | 2 |
| CMP-167 | 176.5 | 34.44 | 2 | 5.31 | 0.74 | 2 |
| CMP-168 | 143.9 | 65.84 | 4 | 6.30 | 3.59 | 4 |
| CMP-169 | 53.4 | 20.4 | 4 | 5.39 | 3.30 | 4 |
| CMP-300 | 94.68 | 44.48 | 4 | 1.732 | 0.6666 | 4 |
| CMP-301 | 179.9 | 9.475 | 2 | 0.8826 | 0.05317 | 2 |
| CMP-302 | 63.41 | 20.67 | 4 | 1.427 | 0.3146 | 4 |
| CMP-303 | 97.93 | 35.85 | 4 | 4.404 | 0.8286 | 4 |
| CMP-304 | 109.5 | 0.4243 | 2 | 8.606 | 1.556 | 2 |
| CMP-305 | 128.1 | 1.838 | 2 | 3.263 | 0.4957 | 2 |

TABLE 23
Average EC50 for selectedMolecules in the pSTAT5 Assay

| | Intact molecule | | | MMP2 cleaved molecule | | |
|---|---|---|---|---|---|---|
| | Average | Std | | Average | Std | |
| Molecule # | EC50 (nM) | Deviation | n = | EC50 (nM) | Deviation | n = |
| CMP-003 | 0.65 | 0.5576 | 28 | 0.96 | 0.9027 | 19 |
| CMP-136 | 40.52 | 12.09 | 5 | 2.40 | 0.84 | 5 |
| CMP-137 | 27.08 | 0.09 | 2 | 4.09 | 0.46 | 2 |
| CMP-138 | 20.06 | 6.05 | 5 | 2.32 | 0.40 | 5 |
| CMP-139 | 21.29 | NA | 1 | 3.61 | 0.43 | 2 |
| CMP-133 | 29.86 | 3.07 | 2 | 2.51 | 0.34 | 2 |
| CMP-140 | 31.56 | 0.16 | 2 | 2.86 | 0.29 | 2 |
| CMP-134 | 12.11 | 3.42 | 2 | 1.32 | 0.22 | 2 |
| CMP-135 | 16.23 | 6.10 | 2 | 3.46 | 0.18 | 2 |
| CMP-151 | 28.16 | 12.75 | 10 | 1.81 | 0.78 | 8 |
| CMP-152 | 16.07 | 10.58 | 4 | 2.95 | 0.48 | 4 |
| CMP-153 | 10.83 | 5.55 | 4 | 3.64 | 2.94 | 4 |
| CMP-154 | 24.69 | 10.27 | 4 | 4.20 | 3.52 | 4 |
| CMP-155 | 10.68 | 11.51 | 4 | 2.90 | 1.14 | 4 |
| CMP-156 | 85.97 | 18.50 | 4 | 21.29 | 15.34 | 4 |
| CMP-157 | 54.55 | 14.76 | 4 | 10.02 | 7.66 | 4 |
| CMP-158 | 36.59 | 21.92 | 4 | 1.95 | 1.21 | 4 |
| CMP-159 | 35.50 | 19.34 | 4 | 2.27 | 1.47 | 4 |
| CMP-160 | 27.76 | 5.21 | 4 | 2.21 | 1.35 | 4 |
| CMP-161 | 29.43 | 2.81 | 4 | 3.59 | 2.02 | 4 |
| CMP-162 | 2.794 | 2.435 | 2 | 0.7578 | 0.6489 | 2 |

### Example 57B: Assay for STAT5 Phosphorylation (pSTAT5) in PBMCs

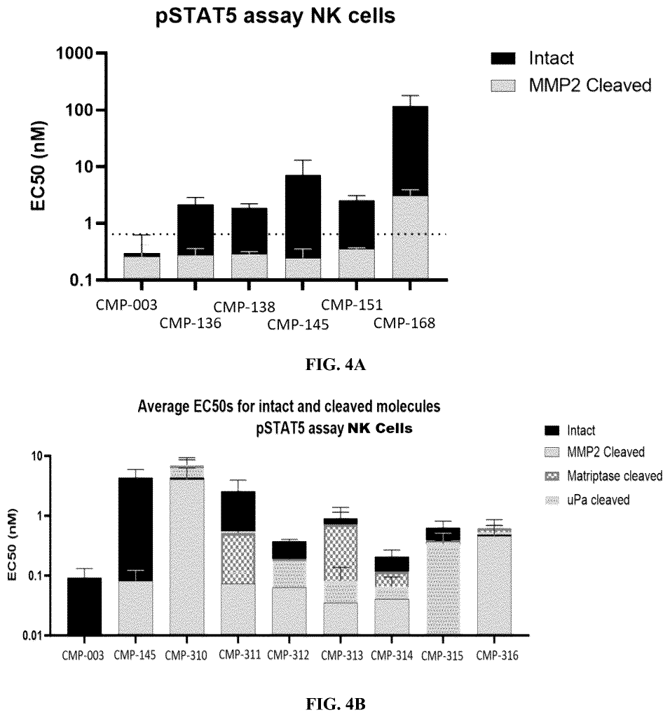

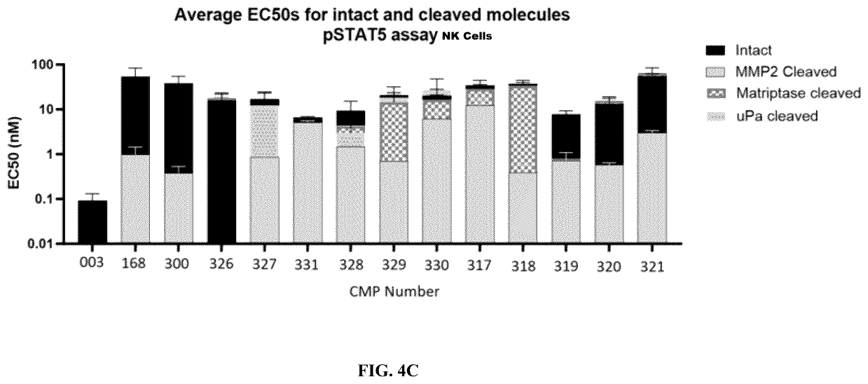

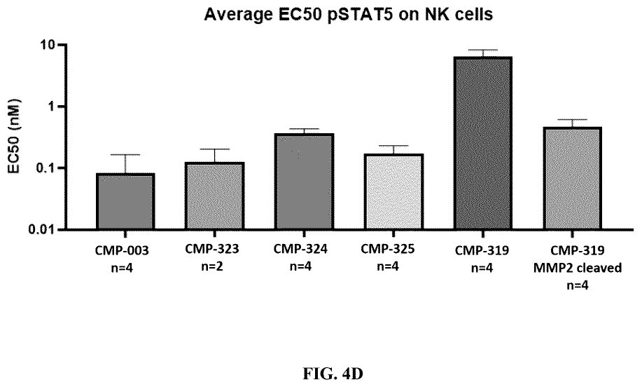

PBMCs were thawed and cultured overnight in RPMI 10% FCS, 1% Glutamine, 1% NEAA, 25UM 2BME, 1% NaPyrovate at 37° C., 5% CO2, 95% humidity. The next day cells were washed and re-suspended in PBS. Cells were stimulated with serial dilutions of activatable IL-2 for 40 minutes at 37° C., 5% CO2, 95% humidity followed by cell fixation and permeabilization with Transcription Factor Phospho Buffer Set (BD Biosciences). Then, cells were stained for 1 hour on ice with antibodies detecting human Phospho-Stat5-PE (Tyr694; 1:50, clone 47/Stat5pY694), CD25-BV421 (1:100, clone M-A251), CD45RA-BV711 (1:100, clone HI100), CD4-PE texas red (1:800, clone RPA-T4), FOXP3-AF647 (1:50, clone 259D), CD3-PE/Cy7 (1:20, cloneOkt3), CD56-AF488 (1:400, clone 5.1H11). Then cells were washed twice with ice cold FACS buffer. Data were acquired on a multi-color flow cytometer and data were analyzed with FlowJo software. EC₅₀s were determined with GraphPadPrism. Results are shown in the table below (NK cell activation) and in FIG. 4A, FIG. 4B, FIG. 4C and FIG. 4D.

TABLE 24
Average EC50 of Selected molecules in the pSTAT5 Assay in NK cells

| | | | | MMP2 cleaved | | | Matriptase cleaved | | | uPa cleaved | | |
|---|---|---|---|---|---|---|---|---|---|---|---|---|
| | Intact molecule | | | molecule | | | molecule | | | molecule | | |
| | Average | | | Average | | | Average | | | Average | | |
| Molecule | EC50 | Std | | EC50 | Std | | EC50 | Std | | EC50 | Std | |
| (CMP) # | (nM) | Deviation | n = | (nM) | Deviation | n = | (nM) | Deviation | n = | (nM) | Deviation | n = |
| 003 | 0.092 | 0.040 | 9 | | | | | | | | | |
| 145 | 4.329 | 1.596 | 4 | 0.082 | 0.040 | 4 | | | | | | |
| 310 | 4.297 | 1.992 | 4 | 4.066 | 2.880 | 2 | 6.898 | 1.702 | 2 | 6.787 | 2.563 | 2 |
| 311 | 2.563 | 1.379 | 4 | 0.073 | 0.039 | 4 | 0.512 | 0.231 | 4 | 0.548 | 0.230 | 4 |
| 313 | 0.898 | 0.488 | 2 | 0.035 | 0.007 | 2 | 0.714 | 0.428 | 2 | 0.081 | 0.056 | 2 |
| 314 | 0.206 | 0.062 | 2 | 0.040 | 0.005 | 2 | 0.117 | 0.006 | 2 | 0.065 | 0.029 | 2 |
| 315 | 0.638 | 0.179 | 2 | 0.369 | 0.142 | 2 | 0.387 | 0.060 | 2 | 0.335 | 0.052 | 2 |
| 316 | 0.484 | 0.204 | 2 | 0.460 | 0.214 | 2 | 0.617 | 0.245 | 2 | 0.533 | 0.155 | 2 |
| 168 | 53.854 | 29.338 | 10 | 0.981 | 0.454 | 10 | | | | | | |
| 300 | 38.103 | 16.549 | 4 | 0.379 | 0.156 | 4 | | | | | | |
| 326 | 15.515 | 6.430 | 8 | 15.995 | 2.652 | 2 | 17.605 | 5.706 | 2 | 18.020 | 0.467 | 2 |
| 327 | 16.596 | 6.635 | 8 | 0.850 | 0.386 | 8 | 17.265 | 7.290 | 2 | 12.075 | 2.171 | 2 |
| 331 | 6.594 | 0.289 | 2 | 5.094 | 0.484 | 2 | | | | | | |
| 328 | 9.235 | 5.902 | 6 | 1.478 | 0.936 | 6 | 4.281 | 2.455 | 2 | 2.965 | 1.744 | 2 |
| 329 | 20.550 | 11.130 | 2 | 0.687 | 0.335 | 2 | 14.387 | 6.312 | 2 | 18.105 | 5.452 | 2 |
| 330 | 20.335 | 7.686 | 2 | 6.118 | 2.640 | 2 | 16.410 | 4.483 | 2 | 26.995 | 20.980 | 2 |
| 317 | 34.078 | 10.412 | 4 | 12.156 | 4.682 | 4 | 28.485 | 7.357 | 4 | | | |
| 318 | 37.370 | 7.284 | 4 | 0.388 | 0.193 | 4 | 33.680 | 6.177 | 4 | | | |
| 319 | 7.725 | 1.562 | 4 | 0.713 | 0.377 | 4 | 0.804 | 0.452 | 4 | | | |
| 320 | 13.247 | 4.377 | 4 | 0.571 | 0.071 | 4 | 15.668 | 3.447 | 4 | | | |
| 321 | 55.093 | 7.348 | 4 | 2.938 | 0.427 | 4 | 65.225 | 19.411 | 4 | | | |
| 136 | 2.174 | 0.693 | 2 | 0.2769 | 0.0853 | 2 | | | | | | |
| 138 | 1.8825 | 0.342 | 2 | 0.2877 | 0.0316 | 2 | | | | | | |
| 145 | 7.087 | 6.029 | 2 | 0.2459 | 0.1107 | 2 | | | | | | |
| 151 | 2.509 | 0.563 | 2 | 0.3524 | 0.0201 | 2 | | | | | | |
| 323* | 0.13 | 0.076 | | | | | | | | | | |
| 324* | 0.38 | 0.061 | | | | | | | | | | |
| 325* | 0.18 | 0.057 | | | | | | | | | | |

*CMP-323, CMP-324, and CMP-325 were tested with no protease treatment, but these represent the cleavage products of CMP-319

### Example 57D: Assay for STAT5 Phosphorylation (pSTAT5) in mouse splenocyte

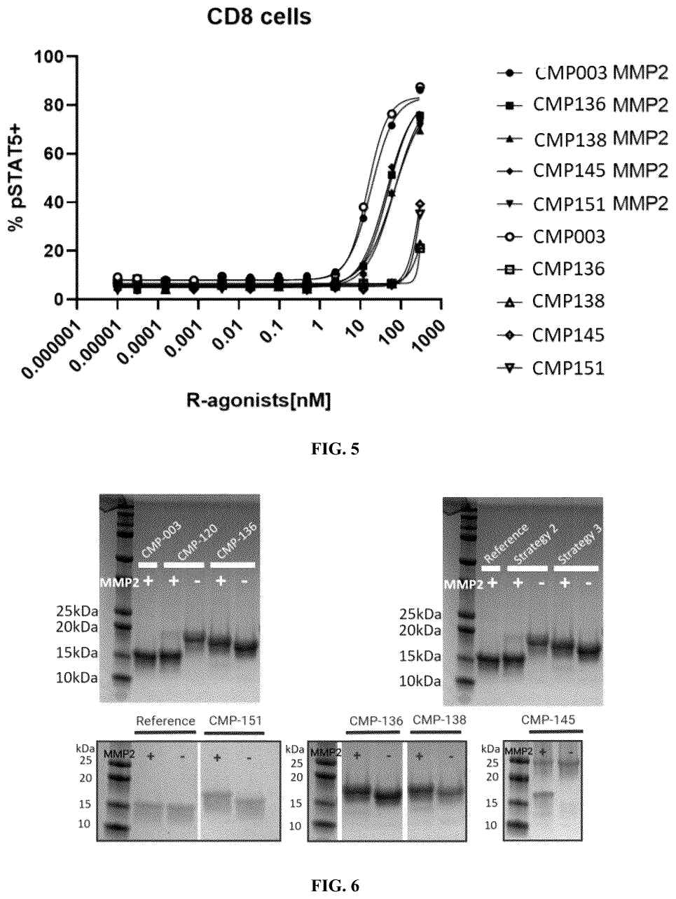

Frozen C57BL/6 mouse splenocytes were thawed and left to recover for 2h in RPMI 10% FCS, 1% Glutamine, 1% NEAA, 25 μM 2BME, 1% NaPyrovate at 37° C., 5% CO2, 95% humidity. Afterwards, cells were washed and re-suspended in PBS. Cells were stimulated with serial dilutions of activatable IL2 for 40 minutes at 37° C., 5% CO2, 95% humidity followed by cell fixation and permeabilization with Transcription Factor Phospho Buffer Set (BD Biosciences). Then, cells were stained for 1 hour on ice with antibodies detecting mouse Phospho-Stat5-AF488 (Tyr694; 1:200, clone 47/Stat5pY694), CD25-BV421 (1:100, clone PC61), CD45R/B220-BV711 (1:100, clone 53-6.7), CD3-BV650 (1:100, clone 17A2), FOXP3-PE (1:200, clone FJK-16s), NK1.1-AF700 (1:200, clone PK136). Data were acquired on a multi-color flow cytometer and data were analyzed with FlowJo software. EC₅₀s were determined with GraphPadPrism. Results are shown in the table below and in FIG. 5. Efficacy plateau could not be reached for all intact and cleaved molecules.

TABLE 25
Average EC50 of selected molecules in
the pSTAT5 Assay in mouse splenocytes

| | Intact molecule | | | MMP2 cleaved molecule | | |
|---|---|---|---|---|---|---|
| | Average | Std | | Average | Std | |
| CMP # | EC50 (nM) | Deviation | n = | EC50 (nM) | Deviation | n = |
| 003 | 24.035 | 8.725 | 2 | 45.725 | 26.995 | 2 |
| 136 | ND | ND | | 84.685 | 35.415 | 2 |
| 138 | ND | ND | | 75.125 | 7.185 | 2 |
| 145 | ND | ND | | 55.205 | 11.105 | 2 |
| 151 | ND | ND | | 89.22 | 21.38 | 2 |

### Example 58: Protease Cleavage and Activation Methods

In experiments where activatable IL-2 polypeptides were first subjected to treatment with MMP protease, the following protocol or one substantially analogous was used. Compositions of IL-2 were diluted to a final concentration of 30 μM in TRIS Buffer (50 mM TRIS, 10 mM CaCl2, 150 mM NaCl, 0.05% Brij35 pH 7.5). Compositions of IL-2 were further diluted to a final concentration of 15 UM with either TRIS buffer or TRIS Buffer containing pre-activated MMP at a concentration of 2 g/ml. The final reaction volume of 40 μl was transferred in sealed tubes and incubated at 37° C. under shaking conditions for 16 hours. Cleaved/activated compositions of IL-2 and respective intact controls were used in assays or stored frozen at −80° C.

### Example 59: Preparation of Additional Molecules

### Example 59A-CMP-300

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.392 min) and by HRMS (Formula: C₂₄₀H₄₀₄N₆₄O₇₄, found: 5370.0037 Da, theoretical: 5369.9903 Da).

Segment 2, 3, 4 and ligated segment 34 were synthesized as described elsewhere herein.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 11 (t_{R}=12.908 min) and by HRMS (Formula: C₄₅₈H₇₆₇N₁₀₉O₁₄₃S, found: 10121.6191 Da, theoretical: 10120.5986 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 11 (t_{R}=16.525 min) and by HRMS (Formula: C₇₈₃H₁₂₈₃N₁₉₃O₂₄₂S₂, found: 17337.3684 Da, theoretical: 17336.3741 Da).

Linear protein: Acm protection on cysteine residues were removed using protocol 18. The ligation sample was purified by preparative HPLC (protocol 20). The purified linear protein was analyzed using HPLC method 11 (t_{R}=16.592 min) and by HRMS (Formula: C₇₇₇H₁₂₇₃N₁₉₁O₂₄₀S₂, found: 17195.3045 Da, theoretical: 17194.2999 Da).

Folded protein: The linear protein was rearranged, folded and purified following protocol 19. The folded protein was analyzed using HPLC method 6 (t_{R}=13.675 min) and by HRMS (Formula: C₇₇₇H₁₂₇₁N₁₉₁O₂₄₀S₂, found: 17192.311 Da, theoretical: 17192.2843 Da).

### Example 59B-CMP-301

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.508 min) and by ESI-MS (Formula: C₂₅₀H₄₂₄N₆₄O₇₉, found: 1119.0328 Da (M+5H)⁺/5, theoretical: 1119.316 Da (M+5H)⁺/5).

Segment 2, 3, 4 and ligated segment 34 were synthesized as described elsewhere herein.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 11 (t_{R}=13.092 min) and by HRMS (Formula: C₄₆₈H₇₈₇N₁₀₉O₁₄₈S, found: 10339.7452 Da, theoretical: 10340.7298 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 11 (t_{R}=16.542 min) and by HRMS (Formula: C₇₉₃H₁₃₀₃N₁₉₃O₂₄₇S₂, found: 17556.4722 Da, theoretical: 17556.5053 Da).

Linear protein: Acm protection on cysteine residues were removed using protocol 18. The ligation sample was purified by preparative HPLC (protocol 20). The purified linear protein was analyzed using HPLC method 11 (R=16.525 min) and by HRMS (Formula: C₇₈₇H₁₂₉₃N₁₉₁O₂₄₅S₂, found: 17415.4268 Da, theoretical: 17414.4311 Da).

Folded protein: The linear protein was rearranged, folded and purified following protocol 19. The folded protein was analyzed using HPLC method 6 (t_{R}=13.658 min) and by HRMS (Formula: C₇₈₇H₁₂₉₁N₁₉₁O₂₄₅S₂, found: 17413.4191 Da, theoretical: 17412.4154 Da).

### Example 59C-CMP-302

Segment 1: Loading of the first KAHA monomer 5 was performed following protocol 1. Elongation of the peptide chain was performed following protocol 3 until position Gly from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (t_{R}=11.442 min) and by HRMS (Formula: C₂₂₉H₃₈₃N₆₃O₆₉, found: 5121.8711 Da, theoretical: 5121.8455 Da).

Segment 2, 3, 4 and ligated segment 34 were synthesized as described in Example 1.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 11 (t_{R}=13.008 min) and by HRMS (Formula: C₄₄₇H₇₄₆N₁₀₈O₁₃₈S, found: 9874.5004 Da, theoretical: 9872.4539 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 11 (t_{R}=16.558 min) and by HRMS (Formula: C₇₇₂H₁₂₆₂N₁₉₂O₂₃₇S₂, found: 17089.2238 Da, theoretical: 17089.2321 Da).

Linear protein: Acm protection on cysteine residues were removed using protocol 18. The ligation sample was purified by preparative HPLC (protocol 20). The purified linear protein was analyzed using HPLC method 11 (t_{R}=16.475 min) and by HRMS (Formula: C₇₆₆H₁₂₅₂N₁₉₀₀₂₃₅S₂, found: 16947.1635 Da, theoretical: 16947.1579 Da).

Folded protein: The linear protein was rearranged, folded and purified following protocol 19. The folded protein was analyzed using HPLC method 6 (t_{R}=13.675 min) and by HRMS (Formula: C₇₆₆H₁₂₅₀N₁₉₀O₂₃₅S₂, found: 16947.2155 Da, theoretical: 16945.1422 Da).

### Example 59D-CMP-303

Segment 1: Loading of the first KAHA monomer 5 was performed following protocol 1. Elongation of the peptide chain was performed following protocol 3 until position PEG4 from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (t_{R}=11.475 min) and by HRMS (Formula: C₂₃₈H₄₀₁N₆₃O₇₃, found: 5312.9771 Da, theoretical: 5312.9688 Da).

Segment 2, 3, 4 and ligated segment 34 were synthesized as described elsewhere herein.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 11 (t_{R}=12.342 min) and by HRMS (Formula: C₄₁₈H₇₅₁N₁₀₉O₁₃₆S, found: 9871.527 Da, theoretical: 9866.4921 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 11 (t_{R}=16.175 min) and by HRMS (Formula: C₇₇₃H₁₂₆₇N₁₉₃O₂₃₅S₂, found: 17088.2399 Da, theoretical: 17088.2845 Da).

Linear protein: Acm protection on cysteine residues were removed using protocol 18. The ligation sample was purified by preparative HPLC (protocol 20). The purified linear protein was analyzed using HPLC method 11 (t_{R}=16.275 min) and by HRMS (Formula: C₇₆₇H₁₂₅₇N₁₉₁O₂₃₃S₂, found: 16946.1504 Da, theoretical: 16946.2102 Da).

Folded protein: The linear protein was rearranged, folded and purified following protocol 19. The folded protein was analyzed using HPLC method 6 (t_{R}=13.675 min) and by HRMS (Formula: C₇₇₅H₁₂₆₈N₁₉₀O₂₃₉S₂, found: 17138.3536 Da, theoretical: 17135.2628 Da).

### Example 59E-CMP-304

Segment 1: Loading of the first KAHA monomer 5 was performed following protocol 1. Elongation of the peptide chain was performed following protocol 3 until position PEG9 from the linker part. Side chain All deprotection of Glu (Glu23) was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=11.608 min) and by HRMS (Formula: C₂₄₈H₄₂₁N₆₃O₇₈, found: 5533.1221 Da, theoretical: 5533.1000 Da).

Segment 2, 3, 4 and ligated segment 34 were synthesized as described elsewhere herein.

Segment 12: Ligation of segments 1 and 2 and photodeprotection were performed as described in protocol 15. The ligation/deprotection sample was purified by preparative HPLC (protocol 20). The purified segment 12 was analyzed using HPLC method 11 (t_{R}=13.008 min) and by HRMS (Formula: C₄₅₆H₇₆₄N₁₀₈O₁₄₂S, found: 10063.6034 Da, theoretical: 10062.5745 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using HPLC method 11 (t_{R}=16.508 min) and by HRMS (Formula: C₇₈₁H₁₂₈₀N₁₉₂O₂₄₁S₂, found: 17279.3412 Da, theoretical: 17279.3527 Da).

Linear protein: Acm protection on cysteine residues were removed using protocol 18. The ligation sample was purified by preparative HPLC (protocol 20). The purified linear protein was analyzed using HPLC method 11 (t_{R}=16.658 min) and by HRMS (Formula: C₇₇₅H₁₂₇₀N₁₉₀O₂₃₉S₂, found: 17138.2836 Da, theoretical: 17137.2785 Da).

Folded protein: The linear protein was rearranged, folded and purified following protocol 19. The folded protein was analyzed using HPLC method 6 6 (t_{R}=13.675 min) and by HRMS (Formula: C₇₈₅H₁₂₈₈N₁₉₀O₂₄₄S₂, found: 17356.4252 Da, theoretical: 17355.3939 Da).

### Example 59F-CMP-305

Segment 1: Loading of the first KAHA monomer 7 was performed following protocol 1. Elongation of the peptide chain was performed following protocol 3 until position 1 (Ala) with Glu (OAll) in position 15 and Lys (ivDde) in position 23. Side chain ivDde protection of Lys (position 23) was removed following protocol 8 and elongation was pursued on the free amine side chain following protocol 3 until Pro from the linker. All deprotection of Glu (OAll) in position 15 was performed following protocol 7. Protected segment 1 was then released from the resin using protocol 12 and linear protected segment 1 was cyclized following protocol 13. The side chains were deprotected following protocol 14. The crude peptide was purified by preparative HPLC (protocol 20). The purified segment 1 was analyzed using HPLC method 2 (tR=10.742 min) and by HRMS (Formula: C₂₃₀H₃₈₈N₆₄O₆₇, found: 5121.9245 Da, theoretical: 5120.8978 Da).

Segment 2, 3, 4 and ligated segment 34 were synthesized as described elsewhere herein.

Segment 12: Ligation of segment 1 and 2 and photodeprotection were performed as described in protocol 15. The ligation/deprotection sample was purified by preparative HPLC (protocol 20). The purified segment 12 was analyzed using HPLC method 11 (t_{R}=13.175 min) and by HRMS (Formula: C₄₆₆H₇₈₄N₁₀₈O₁₄₇S, found: 10283.7253 Da, theoretical: 10283.7083 Da).

Segment 1234 (Acm protected cysteines): Ligation of segments 12 and 34 was performed as described in protocol 17. The ligation sample was purified by preparative HPLC (protocol 20). The purified segment 1234 (Acm protected cysteines) was analyzed using 16.592 (t_{R}=16.592 min) and by HRMS (Formula: C₇₉₁H₁₃₀₀N₁₉₂O₂₄₆S₂, found: 17499.407 Da, theoretical: 17499.4838 Da).

1-24. (canceled)

25. An activatable interleukin-2 (IL-2) polypeptide, comprising:
an IL-2 polypeptide comprising protease cleavable peptide attached to a residue in the region of residues 1-35 of the IL-2 polypeptide, wherein residue position numbering is based on SEQ ID NO: 1 as a reference sequence; and
wherein the IL-2 polypeptide exhibits a greater affinity for the IL-2 receptor beta subunit after cleavage of the cleavable moiety compared to activatable IL-2 polypeptide before cleavage of the cleavable moiety.

26. (canceled)

27. (canceled)

28. The activatable IL-2 polypeptide of claim 25, wherein the protease cleavable peptide is cleavable by a protease selected from a kallikrein, thrombin, chymase, carboxypeptidase A, an elastase, proteinase 3 (PR-3), granzyme M, a calpain, a matrix metalloproteinase (MMP), a disintegrin and metalloproteinase (ADAM), a fibroblast activation protein alpha (FAP), a plasminogen activator, a cathepsin, a caspase, a tryptase, a matriptase, and a tumor cell surface protease, or any combination thereof, or any combination thereof.

29. The activatable IL-2 polypeptide of claim 25, wherein the cleavable peptide is cleavable by multiple proteases.

30. The activatable IL-2 polypeptide of claim 25, wherein cleavage of the cleavable peptide leaves 1, 2, 3, 4, 5, 6, 7, 8, 9, or 10 amino acids attached to the side chain of the amino acid residue to which the cleavable peptide is attached.

31. (canceled)

32. The activatable IL-2 polypeptide of claim 25, wherein the C-terminus of the cleavable peptide is attached to the side chain of the amino acid residue of the IL-2 polypeptide, optionally through a linking group.

33. (canceled)

34. The activatable IL-2 polypeptide of claim 25, wherein the amino acid residue to which the cleavable moiety is attached is a lysine or glutamate.

35. The activatable IL-2 polypeptide of claim 25, wherein the amino acid residue to which the cleavable peptide is attached is substituted relative to the corresponding residue in SEQ ID NO: 1.

36. (canceled)

37. (canceled)

38. The activatable IL-2 polypeptide of claim 25, wherein the cleavable peptide is attached to residue 9, 11, 13, 15, 16, 19, 22, 23, 29, or 32 of the IL-2 polypeptide, wherein residue position numbering is based on SEQ ID NO: 1 as a reference sequence.

39. The activatable IL-2 polypeptide of claim 25, wherein the cleavable peptide is attached to the IL-2 polypeptide at an additional point of attachment.

40. The activatable IL-2 polypeptide of claim 39, wherein the additional point of attachment is to the N-terminus of the IL-2 polypeptide.

41. The activatable IL-2 polypeptide of claim 39, wherein the additional point of attachment is to a side chain of another amino acid residue of the IL-2 polypeptide

42. The activatable IL-2 polypeptide of claim 39, wherein the additional point of attachment is to residue 9 of the IL-2 polypeptide, wherein residue position numbering is based on SEQ ID NO: 1 as a reference sequence.

43. The activatable IL-2 polypeptide of claim 39, wherein the cleavable moiety is attached to residue 23 of the IL-2 polypeptide and the additional point of attachment is to the N-terminus of the IL-2 polypeptide.

44. The activatable IL-2 polypeptide of claim 43, wherein the N-terminus of the IL-2 polypeptide is the amino acid residue at a position corresponding to the first residue of SEQ ID NO: 1.

45. (canceled)

46. The activatable IL-2 polypeptide of claim 25, wherein the C-terminus of the cleavable peptide is attached to the N-terminus of the IL-2 polypeptide and the N-terminus of the cleavable peptide is attached to residue 23 of the IL-2 polypeptide.

47. The activatable IL-2 polypeptide of claim 25, wherein the cleavable peptide comprises the sequence set forth in SEQ ID NO: 317 or 333.

48. (canceled)

49. (canceled)

50. (canceled)

51. (canceled)

52. The activatable IL-2 polypeptide of claim 25, wherein the IL-2 polypeptide exhibits reduced binding to the IL-2 receptor alpha subunit compared to wild type IL-2.

53. (canceled)

54. (canceled)

55. The activatable IL-2 polypeptide of claim 25, wherein the IL-2 polypeptide comprises at least one polymer covalently attached to a residue selected from residue 42 and 45, wherein residue position numbering is based on SEQ ID NO: 1 as a reference sequence.

56. The activatable IL-2 polypeptide of claim 25, wherein the IL-2 polypeptide comprises polymers covalently attached at residues 42 and 45, wherein residue position numbering is based on SEQ ID NO: 1 as a reference sequence.

57. (canceled)

58. The activatable IL-2 polypeptide of claim 25, wherein the IL-2 polypeptide comprises an amino acid sequence having at least about 80%, at least about 85%, at least about 90%, or at least about 95% sequence identity SEQ ID NO: 2.

59-74. (canceled)


SEQUENCE LISTING (442 sequences)

SEQ ID NO: 1 - protein, 133 aa, unidentified [sequence omitted]

SEQ ID NO: 2 - protein, 133 aa, synthetic construct
  SITE 23: note = Norleucine
  SITE 39: note = Norleucine
  SITE 41: note = Homoserine
  SITE 46: note = Norleucine
  SITE 71: note = Homoserine
  SITE 104: note = Homoserine
APTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 3 - protein, 133 aa, synthetic construct; identical to SEQ ID NO: 2
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 4 - protein, 133 aa, synthetic construct
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQKILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 5 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 6 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 7 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 8 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 9 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 10 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 11 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 12 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 13 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 14 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 15 - protein, 133 aa, synthetic construct; identical to SEQ ID NO: 4
  SITE 23: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 16 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 17 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 18 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 19 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 20 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 21 - protein, 133 aa, synthetic construct
  SITE 11: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTKLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 22 - protein, 133 aa, synthetic construct
  SITE 13: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLKLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 23 - protein, 133 aa, synthetic construct
  SITE 15: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLKHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 24 - protein, 133 aa, synthetic construct
  SITE 19: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLKDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 25 - protein, 133 aa, synthetic construct
  SITE 22: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLKXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 26 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 27 - protein, 133 aa, synthetic construct; identical to SEQ ID NO: 2
  SITE 1: note = Attached to another sequence via 9x polyethylene glycol
  SITE 23: note = Norleucine
  SITE 32: note = Side chain attached to another sequence via a 1,5-pentanedioic acid-9x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 28 - protein, 133 aa, synthetic construct; identical to SEQ ID NO: 2
  SITE 1: note = Attached to another sequence via 4x polyethylene glycol
  SITE 23: note = Norleucine
  SITE 32: note = Side chain attached to another sequence via a 1,5-pentanedioic acid-4x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 29 - protein, 133 aa, synthetic construct; identical to SEQ ID NO: 2
  SITE 1: note = Attached to another sequence via 16x polyethylene glycol
  SITE 23: note = Norleucine
  SITE 32: note = Side chain attached to another sequence via a 1,5-pentanedioic acid-16x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 30 - protein, 133 aa, synthetic construct; identical to SEQ ID NO: 2
  SITE 1: note = Attached to another sequence via 24x polyethylene glycol
  SITE 23: note = Norleucine
  SITE 32: note = Side chain attached to another sequence via a 1,5-pentanedioic acid-9x polyethylene glycol linker
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 31 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..38: note = Residues linked via a 1,5-pentanedioic acid-9x polyethylene glycol linker
PLGLAGAPTSSSTKKTQLQLEHLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 32 - protein, 133 aa, synthetic construct; identical to SEQ ID NO: 2
  SITE 1: note = Attached to another sequence via 9x polyethylene glycol
  SITE 23: note = Norleucine
  SITE 32: note = Side chain attached to another sequence via 1,5-pentanedioic acid
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 33 - protein, 133 aa, synthetic construct
  SITE 1: note = Attached to another sequence via 9x polyethylene glycol
  SITE 23: note = Norleucine
  SITE 29: note = Side chain attached to another sequence via 9x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQXILNGIENYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 34 - protein, 133 aa, synthetic construct; identical to SEQ ID NO: 33
  SITE 1: note = Attached to another sequence via 24x polyethylene glycol
  SITE 23: note = Norleucine
  SITE 29: note = Side chain attached to another sequence via 24x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 35 - protein, 133 aa, synthetic construct; identical to SEQ ID NO: 33
  SITE 1: note = Attached to another sequence via 4x polyethylene glycol
  SITE 23: note = Norleucine
  SITE 29: note = Side chain attached to another sequence via 4x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 36 - protein, 133 aa, synthetic construct; identical to SEQ ID NO: 33
  SITE 1: note = Attached to another sequence via 16x polyethylene glycol
  SITE 23: note = Norleucine
  SITE 29: note = Side chain attached to another sequence via 16x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 37 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..35: note = Residues linked via 9x polyethylene glycol
PLGLAGAPTSSSTKKTQLQLEHLLLDLQXILNGIENYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 38 - protein, 133 aa, synthetic construct; identical to SEQ ID NO: 33
  SITE 1: note = Attached to another sequence via 9x polyethylene glycol
  SITE 23: note = Norleucine
  SITE 29: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 39 - protein, 133 aa, synthetic construct
  SITE 1: note = Attached to another sequence via 4x polyethylene glycol
  SITE 19: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLEDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 40 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..25: note = Residues linked via 4x polyethylene glycol
PLGLAGAPTSSSTKKTQLQLEHLLEDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 41 - protein, 133 aa, synthetic construct
  SITE 1: note = N-terminus attached to another sequence via 4x polyethylene glycol
  SITE 22: note = Side chain attached to another sequence
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLEXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 42 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..28: note = Residues linked via 4x polyethylene glycol
PLGLAGAPTSSSTKKTQLQLEHLLLDLEXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 43 - protein, 133 aa, synthetic construct
  SITE 1: note = Attached to another sequence via 9x polyethylene glycol
  SITE 23: note = Norleucine
  SITE 26: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQXILEGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 44 - protein, 139 aa, synthetic construct
  SITE 29: note = Norleucine
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..32: note = Residues linked via 9x polyethylene glycol
PLGLAGAPTSSSTKKTQLQLEHLLLDLQXILEGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 45 - protein, 133 aa, synthetic construct; identical to SEQ ID NO: 4
  SITE 1: note = Attached to another sequence via a 1,5-pentanedioic acid-9x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 46 - protein, 133 aa, synthetic construct; identical to SEQ ID NO: 4
  SITE 1: note = Attached to another sequence via a 1,5-pentanedioic acid-4x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 47 - protein, 133 aa, synthetic construct; identical to SEQ ID NO: 4
  SITE 1: note = Attached to another sequence via 1,5-pentanedioic acid
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 48 - protein, 133 aa, synthetic construct; identical to SEQ ID NO: 4
  SITE 1: note = Attached to another sequence via 1,5-pentanedioic acid
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 49 - protein, 133 aa, synthetic construct; identical to SEQ ID NO: 4
  SITE 1: note = Attached to another sequence via a 1,5-pentanedioic acid-4x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 50 - protein, 133 aa, synthetic construct; identical to SEQ ID NO: 4
  SITE 1: note = Attached to another sequence via a 1,5-pentanedioic acid-9x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 51 - protein, 133 aa, synthetic construct
  SITE 9: note = Side chain attached to another sequence
  SITE 23: note = Side chain attached to another sequence via 4x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 52 - protein, 133 aa, synthetic construct; identical to SEQ ID NO: 51
  SITE 9: note = Side chain attached to another sequence via 4x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 53 - protein, 133 aa, synthetic construct
  SITE 16: note = 2,4-diamino butyric acid
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine
APTSSSTKKTQLQLEXLLLDLQXILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 54 - protein, 133 aa, synthetic construct; identical to SEQ ID NO: 53
  SITE 16: note = N-(acetyl-glutyl-valinyl-citrullyl-(N-(4-aminobenzy l alcohol))-formyl)-L-lysine
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 55 - protein, 133 aa, synthetic construct; identical to SEQ ID NO: 2
  SITE 15: note = N-(acetyl-glutyl-valinyl-citrullyl-(N-(4-aminobenzy l alcohol)))ester-L-glutamate
  SITE 23: note = Norleucine
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 56 - protein, 133 aa, synthetic construct; identical to SEQ ID NO: 51
  SITE 1: note = Attached to another sequence via 4x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 57 - protein, 133 aa, synthetic construct; identical to SEQ ID NO: 51
  SITE 1: note = Attached to another sequence via 9x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 58 - protein, 140 aa, synthetic construct
  SITE 46: note = Norleucine
  SITE 48: note = Homoserine
  SITE 49: note = O-(9x polyethylene glycol-azido)-tyrosine
  SITE 52: note = O-(8x polyethylene glycol-amino)-tyrosine
  SITE 53: note = Norleucine
  SITE 78: note = Homoserine
  SITE 111: note = Homoserine
  SITE 1..30: note = Residues linked via the side chain
GPLGLAGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 59 - protein, 139 aa, synthetic construct
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..29: note = Residues linked via 4x polyethylene glycol
PLGLAGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 60 - protein, 139 aa, synthetic construct; identical to SEQ ID NO: 59
  SITE 45: note = Norleucine
  SITE 47: note = Homoserine
  SITE 48: note = O-(9x polyethylene glycol-azido)-tyrosine
  SITE 51: note = O-(8x polyethylene glycol-amino)-tyrosine
  SITE 52: note = Norleucine
  SITE 77: note = Homoserine
  SITE 110: note = Homoserine
  SITE 1..29: note = Residues linked via 9x polyethylene glycol

SEQ ID NO: 61 - protein, 133 aa, synthetic construct; identical to SEQ ID NO: 4
  SITE 15: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 62 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 63 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 64 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 65 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 66 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 67 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 68 - protein, 133 aa, synthetic construct; identical to SEQ ID NO: 4
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 69 - protein, 133 aa, synthetic construct; identical to SEQ ID NO: 51
  SITE 1: note = Attached to another sequence via 4x polyethylene glycol
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 70 - protein, 142 aa, synthetic construct
  SITE 48: note = Norleucine
  SITE 50: note = Homoserine
  SITE 51: note = O-(9x polyethylene glycol-azido)-tyrosine
  SITE 54: note = O-(8x polyethylene glycol-amino)-tyrosine
  SITE 55: note = Norleucine
  SITE 80: note = Homoserine
  SITE 113: note = Homoserine
  SITE 1..32: note = Residues linked via the side chain
SGRVANLVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 71 - protein, 143 aa, synthetic construct
  SITE 49: note = Norleucine
  SITE 51: note = Homoserine
  SITE 52: note = O-(9x polyethylene glycol-azido)-tyrosine
  SITE 55: note = O-(8x polyethylene glycol-amino)-tyrosine
  SITE 56: note = Norleucine
  SITE 81: note = Homoserine
  SITE 114: note = Homoserine
  SITE 1..33: note = Residues linked via the side chain
RGRRVANLVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 72 - protein, 144 aa, synthetic construct
  SITE 50: note = Norleucine
  SITE 52: note = Homoserine
  SITE 53: note = O-(9x polyethylene glycol-azido)-tyrosine
  SITE 56: note = O-(8x polyethylene glycol-amino)-tyrosine
  SITE 57: note = Norleucine
  SITE 82: note = Homoserine
  SITE 115: note = Homoserine
  SITE 1..34: note = Residues linked via the side chain
SGRVAPQPLVDAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 73 - protein, 142 aa, synthetic construct
  SITE 48: note = Norleucine
  SITE 50: note = Homoserine
  SITE 51: note = O-(9x polyethylene glycol-azido)-tyrosine
  SITE 54: note = O-(8x polyethylene glycol-amino)-tyrosine
  SITE 55: note = Norleucine
  SITE 80: note = Homoserine
  SITE 113: note = Homoserine
  SITE 1..32: note = Residues linked via the side chain
SGRVQPLVDAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 74 - protein, 136 aa, synthetic construct
  SITE 26: note = (alpha-N-seryl-glycyl-arginyl)-amido-L-glutamate
  SITE 42: note = Norleucine
  SITE 44: note = Homoserine
  SITE 45: note = O-(9x polyethylene glycol-azido)-tyrosine
  SITE 48: note = O-(8x polyethylene glycol-amino)-tyrosine
  SITE 49: note = Norleucine
  SITE 74: note = Homoserine
  SITE 107: note = Homoserine
LVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 75 - protein, 136 aa, synthetic construct; identical to SEQ ID NO: 74
  SITE 26: note = (alpha-N-arginyl-glycyl-arginyl)-amido-L-glutamate
  SITE 42: note = Norleucine
  SITE 44: note = Homoserine
  SITE 45: note = O-(9x polyethylene glycol-azido)-tyrosine
  SITE 48: note = O-(8x polyethylene glycol-amino)-tyrosine
  SITE 49: note = Norleucine
  SITE 74: note = Homoserine
  SITE 107: note = Homoserine

SEQ ID NO: 76 - protein, 136 aa, synthetic construct; identical to SEQ ID NO: 74
  SITE 26: note = Side chain attached to another sequence
  SITE 42: note = Norleucine
  SITE 44: note = Homoserine
  SITE 45: note = O-(9x polyethylene glycol-azido)-tyrosine
  SITE 48: note = O-(8x polyethylene glycol-amino)-tyrosine
  SITE 49: note = Norleucine
  SITE 74: note = Homoserine
  SITE 107: note = Homoserine

SEQ ID NO: 77 - protein, 140 aa, synthetic construct
  SITE 30: note = (alpha-N-arginyl-glycyl-arginyl)-amido-L-glutamate
  SITE 46: note = Norleucine
  SITE 48: note = Homoserine
  SITE 49: note = O-(9x polyethylene glycol-azido)-tyrosine
  SITE 52: note = O-(8x polyethylene glycol-amino)-tyrosine
  SITE 53: note = Norleucine
  SITE 78: note = Homoserine
  SITE 111: note = Homoserine
RVANLVGAPTSSSTKKTQLQLEHLLLDLQEILNGINNYKNPKLTRXLXYKFYXPKKATELKHLQCLEEELKPLEEVLXLAQSKNFHLRPRDLISNINVIVLELKGSETTFXCEYADETATIVEFLNRWITFSQSIISTLT

SEQ ID NO: 78 - protein, 133 aa, synthetic construct; identical to SEQ ID NO: 51
  SITE 9: note = Side chain attached to another sequence
  SITE 23: note = Side chain attached to another sequence via 4x polyethylene glycol
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 79 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 80 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 81 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 82 - protein, 133 aa, synthetic construct; identical to SEQ ID NO: 51
  SITE 9: note = Side chain attached to another sequence
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 83 - protein, 133 aa, synthetic construct; identical to SEQ ID NO: 4
  SITE 1: note = N-terminus attached to another sequence via a 1,5-pentanedioic acid-9x polyethylene glycol linker
  SITE 23: note = Side chain attached to another sequence
  SITE 39: note = Norleucine
  SITE 41: note = Homoserine
  SITE 42: note = O-(9x polyethylene glycol-azido)-tyrosine
  SITE 45: note = O-(8x polyethylene glycol-amino)-tyrosine
  SITE 46: note = Norleucine
  SITE 71: note = Homoserine
  SITE 104: note = Homoserine

SEQ ID NO: 84 - protein, 20 aa, synthetic construct
  VARIANT 1..20: note = This sequence may encompass 1-10 GS repeating units
GSGSGSGSGSGSGSGSGSGS

SEQ ID NO: 85 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 86 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 87 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 88 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 89 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 90 - protein, 15 aa, synthetic construct
GGGGSGGGGSGGGGS

SEQ ID NO: 91 - protein, 20 aa, synthetic construct
GGGGSGGGGSGGGGSGGGGS

SEQ ID NO: 92-100 - skipped (9 entries)

SEQ ID NO: 101 - protein, 15 aa, synthetic construct
SGGPGPAGMKGLPGS

SEQ ID NO: 102 - protein, 16 aa, synthetic construct
EAGRSANHEPLGLVAT

SEQ ID NO: 103 - protein, 10 aa, synthetic construct
PQASTGRSGG

SEQ ID NO: 104 - protein, 10 aa, synthetic construct
PQGSTGRAAG

SEQ ID NO: 105 - protein, 10 aa, synthetic construct
PPASSGRAGG

SEQ ID NO: 106 - protein, 16 aa, synthetic construct
RSGVPLSLYSGSGGGK

SEQ ID NO: 107 - protein, 14 aa, synthetic construct
RSGMPYDLYHPSGK

SEQ ID NO: 108 - protein, 14 aa, synthetic construct
RGPDSGGFMLTSGK

SEQ ID NO: 109 - protein, 15 aa, synthetic construct
RGSGHEQLTVSGGSK

SEQ ID NO: 110 - protein, 14 aa, synthetic construct
RSGRAAAVKSPSGK

SEQ ID NO: 111 - protein, 20 aa, synthetic construct
RGSGISSGLLSGRSDNHSGK

SEQ ID NO: 112 - protein, 14 aa, synthetic construct
RGDLLAVVAASGGK

SEQ ID NO: 113 - protein, 15 aa, synthetic construct
RGGISSGLLSGRSGK

SEQ ID NO: 114 - protein, 15 aa, synthetic construct
SGGGKKLADEPEGGS

SEQ ID NO: 115 - protein, 13 aa, synthetic construct
GGGKFLADEPEGG

SEQ ID NO: 116 - protein, 8 aa, synthetic construct
ARLQSAAP

SEQ ID NO: 117 - protein, 14 aa, synthetic construct
ARLQSAAPAGLKGA

SEQ ID NO: 118 - protein, 17 aa, synthetic construct
GSGGPGPAGMHGLPGGS

SEQ ID NO: 119 - protein, 16 aa, synthetic construct
GGGSHTGRSAYFGGGS

SEQ ID NO: 120 - protein, 15 aa, synthetic construct
SGGPGPAGLKGAPGS

SEQ ID NO: 121 - protein, 8 aa, synthetic construct
VPLSLYSG

SEQ ID NO: 122 - protein, 11 aa, synthetic construct
SGLLSGRSDNH

SEQ ID NO: 123 - protein, 11 aa, synthetic construct
SGRSDNIGGGS

SEQ ID NO: 124 - protein, 15 aa, synthetic construct
LQESLRSKESGRSDI

SEQ ID NO: 125 - protein, 13 aa, synthetic construct
ISSGLLSGRSDNH

SEQ ID NO: 126 - protein, 8 aa, synthetic construct
LSGRSDDH

SEQ ID NO: 127 - protein, 13 aa, synthetic construct
ISSGLLSGRSDQH

SEQ ID NO: 128 - protein, 13 aa, synthetic construct
ISSGLLSGRSDNI

SEQ ID NO: 129 - protein, 6 aa, synthetic construct
PLGLAG

SEQ ID NO: 130 - protein, 8 aa, synthetic construct
GPAGMKGL

SEQ ID NO: 131 - protein, 8 aa, synthetic construct
LSGRSDQH

SEQ ID NO: 132 - protein, 8 aa, synthetic construct
LSGRSDNI

SEQ ID NO: 133 - protein, 13 aa, synthetic construct
ISSGLLSGRSDNH

SEQ ID NO: 134 - protein, 7 aa, synthetic construct
GPLGVRG

SEQ ID NO: 135 - protein, 7 aa, synthetic construct
GPLGLAR

SEQ ID NO: 136 - protein, 8 aa, synthetic construct
GPAALVGA

SEQ ID NO: 137 - protein, 8 aa, synthetic construct
GPAALIGG

SEQ ID NO: 138 - protein, 8 aa, synthetic construct
GPLNLVGR

SEQ ID NO: 139 - protein, 8 aa, synthetic construct
GPAGLVAD

SEQ ID NO: 140 - protein, 8 aa, synthetic construct
GPANLVAP

SEQ ID NO: 141 - protein, 8 aa, synthetic construct
VPLSLYSG

SEQ ID NO: 142 - protein, 10 aa, synthetic construct
SGESPAYYTA

SEQ ID NO: 143 - protein, 7 aa, synthetic construct
GGPRGLG

SEQ ID NO: 144 - protein, 6 aa, synthetic construct
HSSKLQ

SEQ ID NO: 145 - protein, 7 aa, synthetic construct
HSSKLQL

SEQ ID NO: 146 - protein, 8 aa, synthetic construct
KRALGLPG

SEQ ID NO: 147 - protein, 5 aa, synthetic construct
LEATA

SEQ ID NO: 148 - protein, 10 aa, synthetic construct
GGAANLVRGG

SEQ ID NO: 149 - protein, 10 aa, synthetic construct
SGRIGFLRTA

SEQ ID NO: 150 - protein, 5 aa, synthetic construct
PLGLA

SEQ ID NO: 151 - protein, 8 aa, synthetic construct
ESPAYYTA

SEQ ID NO: 152 - protein, 6 aa, synthetic construct
RLQLKL

SEQ ID NO: 153 - protein, 7 aa, synthetic construct
RLQLKAC

SEQ ID NO: 154 - protein, 5 aa, synthetic construct
SGRSA

SEQ ID NO: 155 - skipped

SEQ ID NO: 156 - protein, 5 aa, synthetic construct
GGGRR

SEQ ID NO: 157 - protein, 4 aa, synthetic construct
GFLG

SEQ ID NO: 158 - protein, 9 aa, synthetic construct
HSSKLQEDA

SEQ ID NO: 159 - protein, 10 aa, synthetic construct
LVLASSSFGY

SEQ ID NO: 160 - protein, 10 aa, synthetic construct
GVSQNYPIVG

SEQ ID NO: 161 - protein, 10 aa, synthetic construct
GVVQASCRLA

SEQ ID NO: 162 - protein, 6 aa, synthetic construct
DPRSFL

SEQ ID NO: 163 - protein, 6 aa, synthetic construct
PPRSFL

SEQ ID NO: 164 - protein, 4 aa, synthetic construct
DEVD

SEQ ID NO: 165 - protein, 5 aa, synthetic construct
DEVDP

SEQ ID NO: 166 - protein, 8 aa, synthetic construct
KGSGDVEG

SEQ ID NO: 167 - protein, 6 aa, synthetic construct
GWEHDG

SEQ ID NO: 168 - protein, 7 aa, synthetic construct
EDDDDKA

SEQ ID NO: 169 - protein, 9 aa, synthetic construct
KQEQNPGST

SEQ ID NO: 170 - protein, 6 aa, synthetic construct
GKAFRR

SEQ ID NO: 171 - protein, 4 aa, synthetic construct
DAFK

SEQ ID NO: 172 - protein, 4 aa, synthetic construct
DVLK

SEQ ID NO: 173 - protein, 4 aa, synthetic construct
DAFK

SEQ ID NO: 174 - protein, 7 aa, synthetic construct
ALLLALL

SEQ ID NO: 175 - protein, 10 aa, synthetic construct
SGAKPRALTA

SEQ ID NO: 176 - protein, 10 aa, synthetic construct
SGLRLAAITA

SEQ ID NO: 177 - protein, 10 aa, synthetic construct
SGESLAYYTA

SEQ ID NO: 178 - protein, 10 aa, synthetic construct
SGESPAYYTA

SEQ ID NO: 179 - protein, 10 aa, synthetic construct
SGESLRYYTA

SEQ ID NO: 180 - protein, 10 aa, synthetic construct
SGRSLSRLTA

SEQ ID NO: 181 - protein, 10 aa, synthetic construct
SGRSLRRLTA

SEQ ID NO: 182 - protein, 10 aa, synthetic construct
SGAVSWLLTA

SEQ ID NO: 183 - protein, 10 aa, synthetic construct
SGAPSWLLTA

SEQ ID NO: 184 - protein, 10 aa, synthetic construct
SGAVRWLLTA

SEQ ID NO: 185 - protein, 10 aa, synthetic construct
SGANISDLTA

SEQ ID NO: 186 - protein, 10 aa, synthetic construct
SGNRYSSLTA

SEQ ID NO: 187 - protein, 10 aa, synthetic construct
SGHMHKALTA

SEQ ID NO: 188 - protein, 10 aa, synthetic construct
SGHMHKALTA

SEQ ID NO: 189 - skipped

SEQ ID NO: 190 - protein, 12 aa, synthetic construct
GEEGEEPLGLAG

SEQ ID NO: 191 - protein, 7 aa, synthetic construct
GPLGLAG

SEQ ID NO: 192 - protein, 16 aa, synthetic construct
EAGRSANHTPAGLTGP

SEQ ID NO: 193 - protein, 17 aa, synthetic construct
GEAGRSANHTPAGLTGP

SEQ ID NO: 194-200 - skipped (7 entries)

SEQ ID NO: 201 - protein, 11 aa, synthetic construct
AGDKSPLGLAG

SEQ ID NO: 202 - protein, 11 aa, synthetic construct
AGDRSPLGLAG

SEQ ID NO: 203 - protein, 12 aa, synthetic construct
AGDRSAPLGLAG

SEQ ID NO: 204 - protein, 11 aa, synthetic construct
AWGRSPLGLAG

SEQ ID NO: 205 - protein, 12 aa, synthetic construct
AWGRSAPLGLAG

SEQ ID NO: 206 - protein, 11 aa, synthetic construct
AWGKSPLGLAG

SEQ ID NO: 207 - protein, 11 aa, synthetic construct
GAFKSPLGLAG

SEQ ID NO: 208 - protein, 11 aa, synthetic construct
SGRSAPLALAG

SEQ ID NO: 209 - protein, 10 aa, synthetic construct
SGRSPLGLAG

SEQ ID NO: 210-213 - skipped (4 entries)

SEQ ID NO: 214 - protein, 12 aa, synthetic construct
RSGRSAPLGLAG

SEQ ID NO: 215 - protein, 12 aa, synthetic construct
RSGKSAPLGLAG

SEQ ID NO: 216 - protein, 12 aa, synthetic construct
FTARSAPLGLAG

SEQ ID NO: 217 - skipped

SEQ ID NO: 218 - protein, 11 aa, synthetic construct
FTAKSPLGLAG

SEQ ID NO: 219 - protein, 12 aa, synthetic construct
RYGRSAPLGLAG

SEQ ID NO: 220 - protein, 11 aa, synthetic construct
RYGRSPLGLAG

SEQ ID NO: 221 - skipped

SEQ ID NO: 222 - protein, 12 aa, synthetic construct
KYGRSAPLGLAG

SEQ ID NO: 223 - protein, 12 aa, synthetic construct
KWGRSAPLGLAG

SEQ ID NO: 224 - skipped

SEQ ID NO: 225 - protein, 11 aa, synthetic construct
KWGRSPLGLAG

SEQ ID NO: 226-235 - skipped (10 entries)

SEQ ID NO: 236 - protein, 13 aa, synthetic construct
SSRGRRGPLGLAG

SEQ ID NO: 237 - protein, 10 aa, synthetic construct
SSRGPLGLAG

SEQ ID NO: 238 - protein, 10 aa, synthetic construct
SSRGPRGLAG

SEQ ID NO: 239-245 - skipped (7 entries)

SEQ ID NO: 246 - protein, 11 aa, synthetic construct
SGRVLPLNLSG

SEQ ID NO: 247-249 - skipped (3 entries)

SEQ ID NO: 250 - protein, 11 aa, synthetic construct
SGRVLPLGLAG

SEQ ID NO: 251 - protein, 11 aa, synthetic construct
SGRVLPMSLRM

SEQ ID NO: 252 - skipped

SEQ ID NO: 253 - protein, 11 aa, synthetic construct
SGRVLPLGLRA

SEQ ID NO: 254 - skipped

SEQ ID NO: 255 - protein, 11 aa, synthetic construct
SGRVLPLGFMG

SEQ ID NO: 256-300 - skipped (45 entries)

SEQ ID NO: 301 - protein, 17 aa, synthetic construct
SSRGPASNRRLPLGLAG

SEQ ID NO: 302 - protein, 17 aa, synthetic construct
SSRAVFRKNLGPLGLAG

SEQ ID NO: 303 - protein, 15 aa, synthetic construct
SGRVLRKAGPQPLVD

SEQ ID NO: 304 - protein, 16 aa, synthetic construct
SGRVLTLRKAPWGLLE

SEQ ID NO: 305 - protein, 12 aa, synthetic construct
SGRVLGPAGLVG

SEQ ID NO: 306 - protein, 12 aa, synthetic construct
SGRVLGPANLVG

SEQ ID NO: 307 - protein, 11 aa, synthetic construct
SGRVLPAGLVG

SEQ ID NO: 308 - protein, 12 aa, synthetic construct
SGRVLGPLNLSG

SEQ ID NO: 309 - protein, 12 aa, synthetic construct
RQRRSAPLGLAG

SEQ ID NO: 310 - protein, 11 aa, synthetic construct
RQRRSPLGLAG

SEQ ID NO: 311 - protein, 17 aa, synthetic construct
SGRVLTLRKAGPAGLVG

SEQ ID NO: 312 - protein, 17 aa, synthetic construct
SGRVLTLRKAGPANLVG

SEQ ID NO: 313 - protein, 15 aa, synthetic construct
SGRVLRKAGPAGLVG

SEQ ID NO: 314 - protein, 15 aa, synthetic construct
SGRVLRKAGPANLVG

SEQ ID NO: 315 - protein, 11 aa, synthetic construct
SGRVLPANLVG

SEQ ID NO: 316 - protein, 9 aa, synthetic construct
SGRVAGLVG

SEQ ID NO: 317 - protein, 9 aa, synthetic construct
SGRVANLVG

SEQ ID NO: 318 - protein, 13 aa, synthetic construct
PASNRRLPLGLAG

SEQ ID NO: 319 - protein, 13 aa, synthetic construct
SSRVFRKPANLAG

SEQ ID NO: 320 - protein, 16 aa, synthetic construct
SGRVLTLRKAALPLAM

SEQ ID NO: 321 - protein, 13 aa, synthetic construct
SSRGRRGPYMLQG

SEQ ID NO: 322 - protein, 10 aa, synthetic construct
SSRGPYMLQG

SEQ ID NO: 323 - protein, 11 aa, synthetic construct
SGRVLPLGMRA

SEQ ID NO: 324 - protein, 11 aa, synthetic construct
SGRVLPYAMTA

SEQ ID NO: 325 - protein, 12 aa, synthetic construct
  SITE 6: note = Norleucine
RQRRSXPLGLAG

SEQ ID NO: 326 - protein, 12 aa, synthetic construct
KWGKSAPLGLAG

SEQ ID NO: 327 - protein, 12 aa, synthetic construct
RYGKSAPLGLAG

SEQ ID NO: 328 - protein, 13 aa, synthetic construct
RQRRSAAPLGLAG

SEQ ID NO: 329 - protein, 9 aa, synthetic construct
RQRRSVVGG

SEQ ID NO: 330 - protein, 8 aa, synthetic construct
SPLGLAGS

SEQ ID NO: 331 - protein, 10 aa, synthetic construct
RGRKVANLVG

SEQ ID NO: 332 - protein, 10 aa, synthetic construct
RQRKVANLVG

SEQ ID NO: 333 - protein, 10 aa, synthetic construct
RGRRVANLVG

SEQ ID NO: 334 - protein, 11 aa, synthetic construct
RGRKSPANLVG

SEQ ID NO: 335 - protein, 10 aa, synthetic construct
RGRKPYMLQG

SEQ ID NO: 336 - protein, 10 aa, synthetic construct
  SITE 7: note = Norleucine
RGRKPYXLQG

SEQ ID NO: 337 - protein, 11 aa, synthetic construct
RGRKSPYMLQG

SEQ ID NO: 338 - protein, 11 aa, synthetic construct
  SITE 8: note = Norleucine
RGRKSPYXLQG

SEQ ID NO: 339 - protein, 10 aa, synthetic construct
RGRKPQPLVD

SEQ ID NO: 340 - protein, 11 aa, synthetic construct
RGRKSPQPLVD

SEQ ID NO: 341 - protein, 10 aa, synthetic construct
RGRKSQPLVD

SEQ ID NO: 342 - protein, 11 aa, synthetic construct
SGRVAPYMLQG

SEQ ID NO: 343 - protein, 11 aa, synthetic construct
  SITE 8: note = Norleucine
SGRVAPYXLQG

SEQ ID NO: 344 - protein, 9 aa, synthetic construct
SGRVYMLQG

SEQ ID NO: 345 - protein, 9 aa, synthetic construct
  SITE 6: note = Norleucine
SGRVYXLQG

SEQ ID NO: 346 - protein, 11 aa, synthetic construct
SGRVAPQPLVD

SEQ ID NO: 347 - protein, 9 aa, synthetic construct
SGRVQPLVD

SEQ ID NO: 348 - protein, 6 aa, synthetic construct
RGRRGP

SEQ ID NO: 349 - protein, 10 aa, synthetic construct
RGRRPLGLAG

SEQ ID NO: 350 - protein, 15 aa, synthetic construct
RGRRVANPLGLAGSG

SEQ ID NO: 351 - protein, 13 aa, synthetic construct
RGRRPLGLAGGSG

SEQ ID NO: 352 - protein, 10 aa, synthetic construct
RGRRHSSKLQ

SEQ ID NO: 353 - protein, 12 aa, synthetic construct
SGRVANPLGGSG

SEQ ID NO: 354 - protein, 11 aa, synthetic construct
SGRVANYFGKL

SEQ ID NO: 355 - protein, 12 aa, synthetic construct
RGRRVANYFGKL

SEQ ID NO: 356 - protein, 11 aa, synthetic construct
SGRPLGYFGKL

SEQ ID NO: 357 - protein, 12 aa, synthetic construct
RGRRPLGYFGKL

SEQ ID NO: 358 - protein, 15 aa, synthetic construct
RGRRVANPLGYFGKL

SEQ ID NO: 359 - protein, 21 aa, synthetic construct
RGRRSGRAANLVRPLGYFGKL

SEQ ID NO: 360 - protein, 18 aa, synthetic construct
RGRRAANLVRPLGYFGKL

SEQ ID NO: 361 - protein, 11 aa, synthetic construct
HSSKLQYFGKL

SEQ ID NO: 362 - protein, 18 aa, synthetic construct
RGRRHSSKLQPLGYFGKL

SEQ ID NO: 363 - protein, 17 aa, synthetic construct
SGRHSSKLQPLGYFGKL

SEQ ID NO: 364 - protein, 8 aa, synthetic construct
GSGSGSGS

SEQ ID NO: 365 - protein, 8 aa, synthetic construct
SSLYSSPG

SEQ ID NO: 366 - protein, 8 aa, synthetic construct
SSLQSSPG

SEQ ID NO: 367 - protein, 8 aa, synthetic construct
SQYQSSPG

SEQ ID NO: 368 - protein, 8 aa, synthetic construct
SQLYSSPG

SEQ ID NO: 369 - protein, 8 aa, synthetic construct
SSQYSSPG

SEQ ID NO: 370 - protein, 8 aa, synthetic construct
ISQYSSAT

SEQ ID NO: 371 - protein, 7 aa, synthetic construct
KLYSSKQ

SEQ ID NO: 372 - protein, 7 aa, synthetic construct
KLFSSKQ

SEQ ID NO: 373 - protein, 7 aa, synthetic construct
RRLHYSL

SEQ ID NO: 374 - protein, 7 aa, synthetic construct
RRLNYSL

SEQ ID NO: 375 - protein, 7 aa, synthetic construct
RSSYRSL

SEQ ID NO: 376 - protein, 7 aa, synthetic construct
RSSYYSL

SEQ ID NO: 377 - protein, 7 aa, synthetic construct
KSKQHSL

SEQ ID NO: 378 - protein, 7 aa, synthetic construct
HSSKLQL

SEQ ID NO: 379 - protein, 8 aa, synthetic construct
GSSYYSGA

SEQ ID NO: 380 - protein, 8 aa, synthetic construct
GSSVYSGR

SEQ ID NO: 381 - protein, 8 aa, synthetic construct
  SITE 3: note = Norleucine
SSXYSSAG

SEQ ID NO: 382 - protein, 7 aa, synthetic construct
  SITE 1: note = (2,2,4,6,7-pentamethyldihydrobenzofuran-5-sulfonyl) -L-arginine
  SITE 3: note = (2,2,4,6,7-pentamethyldihydrobenzofuran-5-sulfonyl) -L-arginine
  SITE 4: note = (2,2,4,6,7-pentamethyldihydrobenzofuran-5-sulfonyl) -L-arginine
  SITE 7: note = 5-(4-(hydroxymethyl)phenoxy)pentanoic acid modified (2S)-2-(9H-fluoren-9-ylmethoxycarbonylamino)-4-oxo-4-(trit ylamino)butanoate
RGRRVAX

SEQ ID NO: 383 - protein, 6 aa, synthetic construct
  SITE 1: note = Acetylated residue
  SITE 6: note = C-terminus attached to another sequence
PLGLAG

SEQ ID NO: 384 - protein, 6 aa, synthetic construct
  SITE 6: note = C-terminus attached to another sequence
PLGLAG

SEQ ID NO: 385 - protein, 6 aa, synthetic construct
  SITE 1: note = Acetylated residue
  SITE 6: note = C-terminus attached to another sequence via 9x polyethylene glycol
PLGLAG

SEQ ID NO: 386 - protein, 6 aa, synthetic construct
  SITE 6: note = C-terminus attached to another sequence via 9x polyethylene glycol
PLGLAG

SEQ ID NO: 387 - protein, 15 aa, synthetic construct
  SITE 1: note = Acetylated residue
  SITE 15: note = C-terminus attached to another sequence
SGGPGPAGMKGLPGS

SEQ ID NO: 388 - protein, 7 aa, synthetic construct
  SITE 7: note = C-terminus attached to another sequence
MKGLPGS

SEQ ID NO: 389 - protein, 15 aa, synthetic construct
  SITE 15: note = C-terminus attached to another sequence
SGGPGPAGMKGLPGS

SEQ ID NO: 390 - protein, 15 aa, synthetic construct
  SITE 1: note = Acetylated residue
  SITE 15: note = C-terminus attached to another sequence via 9x polyethylene glycol
SGGPGPAGMKGLPGS

SEQ ID NO: 391 - protein, 7 aa, synthetic construct
  SITE 7: note = C-terminus attached to another sequence via 9x polyethylene glycol
MKGLPGS

SEQ ID NO: 392 - protein, 15 aa, synthetic construct
  SITE 15: note = C-terminus attached to another sequence via 9x polyethylene glycol
SGGPGPAGMKGLPGS

SEQ ID NO: 393 - protein, 6 aa, synthetic construct
  SITE 6: note = C-terminus attached to another sequence
PLGLAG

SEQ ID NO: 394 - protein, 6 aa, synthetic construct
  SITE 6: note = C-terminus attached to another sequence
PLGLAG

SEQ ID NO: 395 - protein, 12 aa, synthetic construct
  SITE 12: note = C-terminus attached to another sequence
GEEGEEPLGLAG

SEQ ID NO: 396 - protein, 7 aa, synthetic construct
  SITE 1: note = Lysine attached to 27x polyethylene glycol
  SITE 7: note = C-terminus attached to another sequence
KPLGLAG

SEQ ID NO: 397 - protein, 8 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 8: note = C-terminus attached to another sequence
KKPLGLAG

SEQ ID NO: 398 - protein, 6 aa, synthetic construct
  SITE 6: note = C-terminus attached to another sequence
PLGLAG

SEQ ID NO: 399 - protein, 6 aa, synthetic construct
  SITE 6: note = C-terminus attached to another sequence
PLGLAG

SEQ ID NO: 400 - protein, 6 aa, synthetic construct
  SITE 6: note = C-terminus attached to another sequence
PLGLAG

SEQ ID NO: 401 - protein, 6 aa, synthetic construct
  SITE 6: note = C-terminus attached to another sequence
PLGLAG

SEQ ID NO: 402 - protein, 6 aa, synthetic construct
  SITE 6: note = C-terminus attached to another sequence
PLGLAG

SEQ ID NO: 403 - protein, 16 aa, synthetic construct
  SITE 16: note = C-terminus attached to another sequence
EAGRSANHTPAGLTGP

SEQ ID NO: 404 - protein, 6 aa, synthetic construct
  SITE 1: note = Attached to another sequence via a 1,5-pentanedioic acid-9x polyethylene glycol linker
  SITE 6: note = Attached to another sequence via 9x polyethylene glycol
PLGLAG

SEQ ID NO: 405 - protein, 15 aa, synthetic construct
  SITE 1: note = Attached to another sequence via a 1,5-pentanedioic acid-4x polyethylene glycol linker
  SITE 15: note = Attached to another sequence via 4x polyethylene glycol
SGGPGPAGMKGLPGS

SEQ ID NO: 406 - protein, 15 aa, synthetic construct
  SITE 1: note = Attached to another sequence via a 1,5-pentanedioic acid-16x polyethylene glycol linker
  SITE 15: note = Attached to another sequence via 16x polyethylene glycol
SGGPGPAGMKGLPGS

SEQ ID NO: 407 - protein, 6 aa, synthetic construct
  SITE 1: note = Attached to another sequence via a 1,5-pentanedioic acid-9x polyethylene glycol linker
  SITE 6: note = Attached to another sequence via 24x polyethylene glycol
PLGLAG

SEQ ID NO: 408 - protein, 7 aa, synthetic construct
  SITE 1: note = Attached to another sequence via 1,5-pentanedioic acid
  SITE 7: note = Attached to another sequence via 9x polyethylene glycol
GPLGLAG

SEQ ID NO: 409 - protein, 6 aa, synthetic construct
  SITE 1: note = Attached to another sequence via 9x polyethylene glycol
  SITE 6: note = Attached to another sequence via 9x polyethylene glycol
PLGLAG

SEQ ID NO: 410 - protein, 15 aa, synthetic construct
  SITE 1: note = Attached to another sequence via 24x polyethylene glycol
  SITE 15: note = Attached to another sequence via 24x polyethylene glycol
SGGPGPAGMKGLPGS

SEQ ID NO: 411 - protein, 15 aa, synthetic construct
  SITE 1: note = Attached to another sequence via 4x polyethylene glycol
  SITE 15: note = Attached to another sequence via 4x polyethylene glycol
SGGPGPAGMKGLPGS

SEQ ID NO: 412 - protein, 15 aa, synthetic construct
  SITE 1: note = Attached to another sequence via 16x polyethylene glycol
  SITE 15: note = Attached to another sequence via 16x polyethylene glycol
SGGPGPAGMKGLPGS

SEQ ID NO: 413 - protein, 7 aa, synthetic construct
  SITE 1: note = N-terminus attached to another sequence
  SITE 7: note = Attached to another sequence via 9x polyethylene glycol
GPLGLAG

SEQ ID NO: 414 - protein, 7 aa, synthetic construct
  SITE 1: note = N-terminus attached to another sequence
  SITE 7: note = Attached to another sequence via 4x polyethylene glycol
GPLGLAG

SEQ ID NO: 415 - protein, 7 aa, synthetic construct
  SITE 1: note = N-terminus attached to another sequence
  SITE 7: note = Attached to another sequence via 4x polyethylene glycol
GPLGLAG

SEQ ID NO: 416 - protein, 7 aa, synthetic construct
  SITE 1: note = N-terminus attached to another sequence
  SITE 7: note = Attached to another sequence via 9x polyethylene glycol
GPLGLAG

SEQ ID NO: 417 - protein, 6 aa, synthetic construct
  SITE 1: note = Attached to another sequence via a 1,5-pentanedioic acid-9x polyethylene glycol linker
  SITE 6: note = C-terminus attached to another sequence
PLGLAG

SEQ ID NO: 418 - protein, 6 aa, synthetic construct
  SITE 1: note = Attached to another sequence via a 1,5-pentanedioic acid-4x polyethylene glycol linker
  SITE 6: note = C-terminus attached to another sequence
PLGLAG

SEQ ID NO: 419 - protein, 7 aa, synthetic construct
  SITE 1: note = Attached to another sequence via 1,5-pentanedioic acid
  SITE 7: note = C-terminus attached to another sequence
GPLGLAG

SEQ ID NO: 420 - protein, 17 aa, synthetic construct
  SITE 1: note = Attached to another sequence via 1,5-pentanedioic acid
  SITE 17: note = C-terminus attached to another sequence
GEAGRSANHTPAGLTGP

SEQ ID NO: 421 - protein, 16 aa, synthetic construct
  SITE 1: note = Attached to another sequence via a 1,5-pentanedioic acid-4x polyethylene glycol linker
  SITE 16: note = C-terminus attached to another sequence
EAGRSANHTPAGLTGP

SEQ ID NO: 422 - protein, 16 aa, synthetic construct
  SITE 1: note = Attached to another sequence via a 1,5-pentanedioic acid-9x polyethylene glycol linker
  SITE 16: note = C-terminus attached to another sequence
EAGRSANHTPAGLTGP

SEQ ID NO: 423 - protein, 6 aa, synthetic construct
  SITE 1: note = N-terminus attached to another sequence
  SITE 6: note = Attached to another sequence via 4x polyethylene glycol
PLGLAG

SEQ ID NO: 424 - protein, 6 aa, synthetic construct
  SITE 1: note = Attached to another sequence via 4x polyethylene glycol
  SITE 6: note = C-terminus attached to another sequence
PLGLAG

SEQ ID NO: 425 - protein, 7 aa, synthetic construct
  SITE 1: note = N-terminus attached to another sequence
  SITE 7: note = Attached to another sequence via 4x polyethylene glycol
GPLGLAG

SEQ ID NO: 426 - protein, 7 aa, synthetic construct
  SITE 1: note = N-terminus attached to another sequence
  SITE 7: note = Attached to another sequence via 9x polyethylene glycol
GPLGLAG

SEQ ID NO: 427 - protein, 7 aa, synthetic construct
  SITE 1: note = N-terminus attached to another sequence
  SITE 7: note = C-terminus attached to another sequence
GPLGLAG

SEQ ID NO: 428 - protein, 8 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 8: note = C-terminus attached to another sequence
KKGSGSGS

SEQ ID NO: 429 - protein, 11 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 11: note = C-terminus attached to another sequence
KKSGRVANLVG

SEQ ID NO: 430 - protein, 11 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 11: note = C-terminus attached to another sequence
KKSGRVANLVG

SEQ ID NO: 431 - protein, 12 aa, synthetic construct
  SITE 1: note = Lysine attached to 27x polyethylene glycol
  SITE 2: note = Lysin[epsilon-N-[lysin(alpha-N-27x polyethylene glycol)(epsilon-N-27x polyethylene glycol)yl]]yl modified residue
  SITE 12: note = C-terminus attached to another sequence
KKRGRRVANLVG

SEQ ID NO: 432 - protein, 12 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 12: note = C-terminus attached to another sequence
KKRGRRVANLVG

SEQ ID NO: 433 - protein, 11 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 11: note = C-terminus attached to another sequence
KKSGRVQPLVD

SEQ ID NO: 434 - protein, 10 aa, synthetic construct
  SITE 1: note = Lysine attached to 9x polyethylene glycol
  SITE 2: note = Lysin[epsilon-N-[lysin(alpha-N-9x polyethylene glycol)(epsilon-N-9x polyethylene glycol)yl]]yl modified residue
  SITE 10: note = C-terminus attached to another sequence
KKGSGSGSGS

SEQ ID NO: 435 - protein, 7 aa, synthetic construct
  SITE 1: note = N-terminus attached to another sequence
  SITE 7: note = Attached to another sequence via 4x polyethylene glycol
GSGSGSG

SEQ ID NO: 436 - protein, 7 aa, synthetic construct
  SITE 1: note = N-terminus attached to another sequence
RGRRVAN

SEQ ID NO: 437 - protein, 6 aa, synthetic construct
  SITE 1: note = N-terminus attached to another sequence via 4x polyethylene glycol
  SITE 6: note = C-terminus attached to another sequence
GSGSGS

SEQ ID NO: 438 - protein, 9 aa, synthetic construct
  SITE 1: note = N-terminus attached to another sequence
  SITE 9: note = C-terminus attached to another sequence
SGRVANLVG

SEQ ID NO: 439 - protein, 10 aa, synthetic construct
  SITE 1: note = N-terminus attached to another sequence
  SITE 10: note = C-terminus attached to another sequence
RGRRVANLVG

SEQ ID NO: 440 - protein, 11 aa, synthetic construct
  SITE 1: note = N-terminus attached to another sequence
  SITE 11: note = C-terminus attached to another sequence
SGRVAPQPLVD

SEQ ID NO: 441 - protein, 9 aa, synthetic construct
  SITE 1: note = N-terminus attached to another sequence
  SITE 9: note = C-terminus attached to another sequence
SGRVQPLVD

SEQ ID NO: 442 - protein, 6 aa, synthetic construct
  SITE 1: note = Attached to another sequence via a 1,5-pentanedioic acid-9x polyethylene glycol linker
  SITE 6: note = C-terminus attached to another sequence
GSGSGS